SEQUENCE LISTING (8 sequences)

SEQ ID NO: 1 - DNA, 39 bp, Artificial Sequence
  ESR Y537S Mutation Forward Primer
aagaacgtggtgcccctctctgacctgctgctggagatg

SEQ ID NO: 2 - DNA, 39 bp, Artificial Sequence
  ESR Y537S Mutation Reverse Primer
catctccagcagcaggtcagagaggggcaccacgttctt

SEQ ID NO: 3 - DNA, 36 bp, Artificial Sequence
  ESR Y537N Mutation Forward Primer
aagaacgtggtgcccctcaatgacctgctgctggag

SEQ ID NO: 4 - DNA, 36 bp, Artificial Sequence
  ESR Y537N Mutant Reverse Primer
catctccagcagcaggtcattgaggggcaccacgtt

SEQ ID NO: 5 - DNA, 39 bp, Artificial Sequence
  ESR Y537C mutant forward primer
aagaacgtggtgcccctctgtgacctgctgctggagatg

SEQ ID NO: 6 - DNA, 39 bp, Artificial Sequence
  ESR Y537C mutant reverse primer
catctccagcagcaggtcacagaggggcaccacgttctt

SEQ ID NO: 7 - DNA, 39 bp, Artificial Sequence
  ESR D538G mutant forward primer
aacgtggtgcccctctatggcctgctgctggagatgctg

SEQ ID NO: 8 - DNA, 39 bp, Artificial Sequence
  ESR D538G Mutant Reverse primer
cagcatctccagcagcaggccatagaggggcaccacgtt

(19) United States
(12) Patent Application Publication
(10) Pub. No.: US 2020/0255415 A1
(43) Pub. Date: Aug. 13, 2020

(54) TETRASUBSTITUTED ALKENE COMPOUNDS AND THEIR USE FOR THE TREATMENT OF BREAST CANCER

(71) Applicant: Eisai R&D Management Co., Ltd., Tokyo (JP)

(72) Inventors: Mark Bock, Boston, MA (US); Ming-Hong Hao, Quincy, MA (US); Manav Korpal, Winchester, MA (US); Vijay Kumar Nyavanandi, Andhra Pradesh (IN); Xiaoling Puyang, Cambridge, MA (US); Susanta Samajdar, Karnataka (IN); Peter Gerard Smith, Arlington, MA (US); John Wang, Andover, MA (US); Guo Zhu Zheng, Lexington, MA (US); Ping Zhu, Acton, MA (US); Lorna Helen Mitchell, Cambridge, MA (US); Nicholas Larsen, Needham, MA (US); Nathalie Rioux, Woburn, MA (US); Sudeep Prajapati, Somerville, MA (US); Dominic Reynolds, Stoneham, MA (US); Morgan O'Shea, Waltham, MA (US); Thiwanka Samarakoon, Westwood, MA (US)

(73) Assignee: Eisai R&D Management Co., Ltd., Tokyo (JP)

(21) Appl. No.: 16/349,479

(22) PCT Filed: Nov. 22, 2017
(86) PCT No.: PCT/US2017/063047

§ 371 Date: May 13, 2019

(30) Foreign Application Priority Data
Nov. 24, 2016 (IN) 201641040196
May 26, 2017 (IN) 201741018583

(51) Int. Cl. C07D 413/14; A61P 35/00; C07D 231/56; C07D 401/10; C07D 401/14; C07D 401/12; C07D 413/12; C07D 403/10

(52) U.S. Cl. CPC C07D 413/14; A61P 35/00; C07D 231/56; C07D 403/10; C07D 401/14; C07D 401/12; C07D 413/12; C07D 401/10

(57) ABSTRACT

Disclosed herein are compounds, or pharmaceutically acceptable salts thereof, and methods of using the compounds for treating breast cancer by administration to a subject in need thereof a therapeutically effective amount of the compounds or pharmaceutically acceptable salts thereof. The breast cancer may be an ER-positive breast cancer and/or the subject in need of treatment may express a mutant ER-α protein.

### CROSS-REFERENCE TO RELATED APPLICATIONS

This application claims the benefit of priority of Indian Patent Application No. 201741018583, filed on May 26, 2017, and Indian Patent Application No. 201641040196, filed on Nov. 24, 2016. Both of those applications are incorporated by reference as if fully rewritten herein.

### BACKGROUND

Breast cancer is the most commonly diagnosed malignancy among women today with nearly 200,000/1.7 million new cases diagnosed in the US/worldwide each year respectively. Since about 70% of breast tumors are positive for the estrogen receptor alpha (ERα)—a key oncogenic driver in this subset of tumors—several classes of therapies have been developed to antagonize ERα function, including 1) selective estrogen receptor downregulators (SERDs) of which fulvestrant is an example, 2) selective estrogen receptor modulators (SERMs) of which tamoxifen is an example and 3) aromatase inhibitors that reduce systemic levels of estrogen. These therapies have been largely effective in the clinic reducing occurrence and progression of ERα+ breast tumors. However there are on-target liabilities associated with these different classes of compounds. For example, tamoxifen has been shown to activate signaling activity in the endometrium leading to an increase in risk of endometrial cancers in the clinic (Fisher et al., (1994) J Natl Cancer Inst. April 6; 86(7):527-37; van Leeuwen et al., (1994) Lancet February 19:343(8895):448-52). In contrast, since fulvestrant is a pure antagonist, it can lead to loss of bone density in post-menopausal women as ERα activity is critical for bone building. In addition to on-target side effects, clinical resistance is also beginning to emerge to these classes of ERα antagonists highlighting the need to develop next-generation compounds.

Several mechanisms of resistance have been identified using in vitro and in vivo models of resistance to various endocrine therapies. These include increased ERα/HER2 “crosstalk” (Shou et al., (2004)J Natl Cancer Inst. June 16; 96(12):926-35), aberrant expression of ERα coactivators/corepressors (Osbome et al., (2003) J Natl Cancer Inst. March 5; 95(5):353-61) or loss of ERα altogether to allow ER-independent growth (Osbome C K, Schiff R (2011) Annu Rev Med 62: 233-47).

In the hopes of identifying clinically relevant mechanisms of resistance, great effort has also recently gone into deeply characterizing the genetics of endocrine-therapy resistant metastases isolated from patients. Several independent labs have recently published the multitude of genetic lesions observed in the resistant vs the primary tumors (Li et al., (2013) Cell Rep. September 26; 4(6): 1116-30; Robinson et al., (2013) Nat Genet. December; 45(12): 1446-51; Toy et al., (2013) Nat Genet. 2013 December; 45(12): 1439-45). Among these are the highly recurrent mutations in the ligand-binding domain of ESR1 (gene which encodes ERα protein) found to be significantly enriched in about 20% of resistant tumors relative to endocrine therapy naïve tumors (Jeselsohn et al., (2014) Clin Cancer Res. April 1; 20(7): 1757-67; Toy et al., (2013) Nat Genet. 2013 December; 45(12): 1439-45; Robinson et al., (2013) Nat Genet. December; 45(12): 1446-51; Merenbakh-Lamin et al., (2013) Cancer Res. December 1; 73(23):6856-64; Yu et al., (2014) Science July 11; 345(6193):216-20; Segal and Dowsett (2014). Clin Cancer Res April 1:20(7):1724-6), suggesting the potential for these mutations to functionally drive clinical resistance. In contrast to the enrichment in ESR1 mutations observed in therapy-resistant tumors, mutations in other cancer-related genes failed to show such a robust enrichment strongly implying the importance of ERα mutations in promoting resistance (Jeselsohn et al., (2014) Clin Cancer Res. April 1:20(7):1757-67).

ER+ breast cancer patients on average are treated with seven independent therapies including chemotherapies and various anti-estrogen therapies such as tamoxifen, fulvestrant and aromatase inhibitors. Recent genomic profiling has revealed that the ERα pathway remains a critical driver of tumor growth in the resistant setting as activating mutations in ERα have emerged. Thus, it is critical that more potent ER-directed therapies be developed that can overcome resistance in the clinical setting. Hence, there is a need for novel compounds that can potently suppress the growth of both wild-type (WT) and ER α-mutant positive tumors.

Most inhibitory drug interactions with cytochrome (CYP) P450 enzymes are reversible, but in some cases the inhibitory effect increases over time and is not promptly reversible. This effect is due to irreversible covalent binding or quasi-irreversible noncovalent tight binding of a chemically reactive intermediate to the enzyme that catalyzes its formation. This class of inhibitory drug interactions is called Time-Dependent Inhibition (“TDI”). When TDI is the mode of inhibition, the inhibitory interaction will generally be greater over time following multiple dosing and be longer lasting after discontinuation of the inhibitor than in a situation when the inhibitory interaction is reversible. Therefore, TDI should be studied in standard in vitro screening protocols by pre-incubating the drug (a potential inhibitor) before the addition of a substrate (Food and Drug Administration (FDA) guidance: Cf. fda.gov/downloads/drugs/guidances/ucm292362.pdf (FDA guidance, In Vitro Metabolism-and Transporter-Mediated Drug-Drug Interaction Studies. Draft Guidance, Oct. 24, 2017)). Whether an investigational drug inhibits CYP enzymes is usually investigated in vitro using human liver tissues such as human liver microsomes to determine the inhibition mechanisms (e.g., reversible or TDI) and inhibition potency. Id.

Citing to Grimm et al., (“The conduct of in vitro studies to address time-dependent inhibition of drug-metabolizing enzymes: a perspective of the Pharmaceutical Research and Manufacturers of America,” Drug Metab Dispos. 37:1355-1370, 2009), the FDA recently described how pharmaceutical companies should evaluate investigational drugs for TDI potential. In particular, the FDA indicated that pharmaceutical companies “should routinely study TDI in standard in vitro screening protocols by pre-incubating the investigational drug (e.g., for at least 30 min) before adding any substrate. Any significant time-dependent and co-factor-dependent (e.g., NADPH for CYPs) loss of initial product formation may indicate TDI. In these circumstances, the sponsor should conduct definitive in vitro studies to obtain TDI parameters (i.e., k_{inact} and K_{I}).” See FDA guidance, In Vitro Metabolism-and Transporter-Mediated Drug-Drug Interaction Studies Guidance for Industry, Draft Guidance. Oct. 24, 2017, pg. 24, lines 854-858.

Patients frequently use more than one medication at a time. Unanticipated, unrecognized, or mismanaged drug-drug interactions (DDIs) are an important cause of morbidity and mortality associated with prescription drug use and have occasionally caused the withdrawal of approved drugs from the market. Determination of an investigational drug's potential to inhibit CYPs in both a reversible manner (i.e., reversible inhibition) and time-dependent manner (i.e., TDI) will allow for better characterization of potentially clinically relevant DDI. Hence, there is a need to identify and develop investigational drugs that further mitigate or remove the TDI potential.

### SUMMARY

Described herein are novel compounds useful for treating cancer. Embodiments may provide a compound given by Formula I:

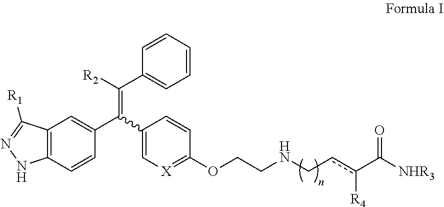

wherein:
R₁ is —H or —F;
R₂ is —CH₂CH₃, —CH₂CF₃, or cyclobutyl;
R₃ is
   i) selected from —H, —CH₃, and —CH₂CH₂OH, or
   ii) forms a 5-7 membered heterocycloalkyl ring with R; and the N to which R₃ is attached;
wherein R₄ is —H when it does not form said 5-7-membered heterocycloalkyl ring with R₃;
X is N or C;
n is 1-2; and
[symbol] represents a singe bond or a double bond;
or a pharmaceutically acceptable salt thereof.

In some embodiment. R₁ is —H, —CH₃, or —F.

Embodiments of Formula I may have the following stereochemistry:

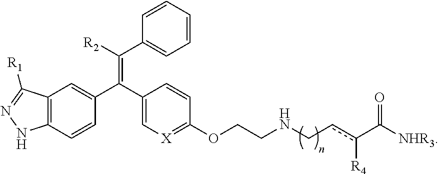

Further embodiments may provide a compound given by Formula II:

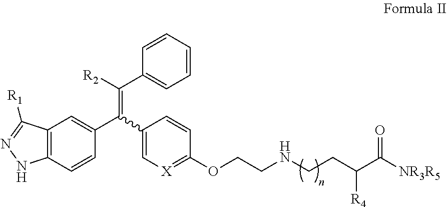

wherein:
R₁ is —H or —F;
R₂ is —CH₂CH₃, —CH₂CF₃, or cyclobutyl;
R₃
   i) is selected from —H, —CH₃, and —CH₂CH₂OH, or
   ii) forms a 4-6 membered heterocycloalkyl ring with R₅ and the N to
which R₃ and R₅ are attached, optionally with an additional heteroatom in the 4-6 membered ring;
   iii) forms a 5-7 membered heterocycloalkyl ring with R₄ and the N to which R₃ is attached;
wherein R₄ is —H when it does not form said 5-7-membered heterocycloalkyl ring with R₃;
wherein R₅ is —H, —CH₃, and —CH₂CH₂OH when it does not form said 4-6 membered heterocycloalkyl ring with R₃;
X is N or C; and
n is 1-2; or a pharmaceutically acceptable salt thereof.

Embodiments of Formula II may have the following stereochemistry:

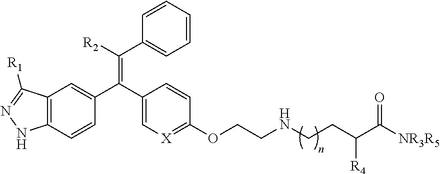

In further embodiments of Formula I or Formula II, R₁ is —F. In further embodiments, R₁ is —H.

In still further embodiments, R₂ is —CH₂—CF₃. In yet still further embodiments. R₂ is —CH₂CH₃. In further embodiments of Formula I, [symbol] represents a single bond. In further embodiments of Formula I or Formula II, n is 1. In still further embodiments, R₃ is —CH₃.

Other embodiments may provide one of the following compounds: N,N-dimethyl-4-[(2-[4-[(1E)-4,4,4-trifluoro-1-(3-fluoro-1H-indazol-5-yl)-1-phenylbut-1-en-2-yl]phenoxy]ethyl)amino]butanamide; (Z)—N,N-dimethyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide; (E)-N-methyl-4-(2-(5-((Z)-4,4,4-trifluoro-1-(3-fluoro-1H-indazol-5-yl)-2-phenylbut-1-enyl)pyridin-2-yloxy)ethylamino)but-2-enamide; (E)-4-((2-(4-(1-(1H-indazol-5-yl)-2-phenylbut-1-en-1-yl)phenoxy)ethyl)amino)-N,N-dimethylbutanamide; (E)-N-methyl-4-((2-((5-((E)-4,4,4-trifluoro-1-(3-fluoro-1H-indazol-5-yl)-1-phenylbut-1-en-2-yl)pyridin-2-yl)oxy)ethyl)amino)but-2-enamide; (E)-N-methyl-5-((2-((5-((Z)-4,4,4-trifluoro-1-(3-fluoro-1H-indazol-5-yl)-2-phenylbut-1-en-1-yl)pyridin-2-yl)oxy)ethyl)amino)pent-2-enamide; (E)-N-(2-hydroxyethyl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (Z)—N-methyl-5-((2-((5-(4,4,4-trifluoro-1-(3-fluoro-1H-indazol-5-yl)-2-phenylbut-1-en-1-yl)pyridin-2-yl)oxy)ethyl)amino)pentanamide; (E)-N-methyl-4-((2-((5-((Z)-4,4,4-trifluoro-1-(1H-indazol-5-yl)-2-phenylbut-1-en-1-yl)pyridin-2-yl)oxy)ethyl)amino)but-2-enamide; (E)-N-methyl-4-((2-(4-((E)-4,4,4-trifluoro-1-(1H-indazol-5-yl)-2-phenylbut-1-en-1-yl)phenoxy)ethyl)amino)but-2-enamide; (E)-4-((2-(4-((E)-2-cyclobutyl-1-(1H-indazol-5-yl)-2-phenylvinyl)phenoxy)ethyl)amino)-N-methylbut-2-enamide; (2)-1-(2-((5-(4,4,4-trifluoro-1-(3-fluoro-1H-indazol-5-yl)-2-phenylbut-1-en-1-yl)pyridin-2-yl)oxy)ethyl)pyrrolidin-2-one; (E)-1-(pyrrolidin-1-yl)-4-((2-(4-((E)-4,4,4-trifluoro-1-(3-fluoro-1H-indazol-5-yl)-2-phenylbut-1-en-1-yl)phenoxy)ethyl)amino)but-2-en-1-one; (E)-1-(pyrrolidin-1-yl)-4-((2-(4-((E)-4,4,4-trifluoro-1-(1H-indazol-5-yl)-2-phenylbut-1-en-1-yl)phenoxy)ethyl)amino)but-2-en-1-one; (E)-1-(pyrrolidin-1-yl)-4-((2-((5-((Z)-4,4,4-trifluoro-1-(3-fluoro-1H-indazol-5-yl)-2-phenylbut-1-en-1-yl)pyrindin-2-yl)oxy)ethyl)amino)but-2-en-1-one; (E)-1-(pyrrolidin-1-yl)-4-((2-((5-((Z)-4,4,4-trifluoro-1-(1H-indazol-5-yl)-2-phenylbut-1-en-1-yl)pyridin-2-yl)oxy)ethyl)amino)but-2-en-1-one; (E)-1-morpholino-4-((2-(4-((E)-4,4,4-trifluoro-1-(3-fluoro-1H-indazol-5-yl)-2-phenylbut-1-en-1-yl)phenoxy)ethyl)amino)but-2-en-1-one; (E)-1-morpholino-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-en-1-one; (E)-1-morpholino-4-((2-((5-((Z)-4,4,4-trifluoro-1-(11H-indazol-5-yl)-2-phenylbut-1-en-1-yl)pyridin-2-yl)oxy)ethyl)amino)but-2-en-1-one; (E)-N-(2-methoxyethyl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-N-methyl-4-((2-(4-((E)-4,4,4-trifluoro-1-(3-fluoro-1H-indazol-5-yl)-2-phenylbut-1-en-1-yl)phenoxy-ethyl)amino)but-2-enamide; (E)-N,N-di(²H₃)methyl-4-((2-(4-((E)-4,4,4-trifluoro-1-(3-fluoro-1H-indazol-5-yl)-2-phenylbut-1-en-1-yl)phenoxy)ethyl)amino)but-2-enamide; (E)-N,N-di(²H₃)methyl-4-((2-(4-((E)-4,4,4-trifluoro-1-(1H-indazol-5-yl)-2-phenylbut-1-en-1-yl)phenoxy)ethyl)amino)but-2-enamide; (E)-N,N-di(²H₃)methyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-N,N-di(²H₃)methyl-4-((2-((5-((Z)-4,4,4-trifluoro-1-(1H-indazol-5-yl)-2-phenylbut-1-en-1-yl)pyridin-2-yl)oxy)ethyl)amino)but-2-enamide; (E)-4-((2-(4-((E)-1-(3-fluoro-1H-indazol-5-yl)-2-phenylbut-1-en-1-yl)phenoxy)ethyl)amino)-N-methylbut-2-enamide; (E)-4-((2-((5-((Z)-1-(3-fluoro-1H-indazol-5-yl)-2-phenylbut-1-en-1-yl)pyridin-2-yl)oxy)ethyl)amino)-N-methylbut-2-enamide; (E)-4-((2-((5-((Z)-1-(1H-indazol-5-yl)-2-phenylbut-1-en-1-yl)pyridin-2-yl)oxy)ethyl)amino)-N-methylbut-2-enamide; (E)-4-((2-(4-((E)-1-(3-fluoro-1H-indazol-5-yl)-2-phenylbut-1-en-1-yl)phenoxy)ethyl)amino)-1-(pyrrolidin-1-yl)but-2-en-1-one; (E)-4-((2-((5-((Z)-1-(3-fluoro-1H-indazol-5-yl)-2-phenylbut-1-en-1-yl)pyridin-2-yl)oxy)ethyl)amino)-1-(pyrrolidin-1-yl)but-2-en-1-one; (E)-4-((2-(4-((E)-2-cyclobutyl-1-(3-fluoro-1H-indazol-5-yl)-2-phenylvinyl)phenoxy)ethyl)amino)-N-methylbut-2-enamide; (E)-4-((2-((5-((Z)-2-cyclobutyl-1-(3-fluoro-1H-indazol-5-yl)-2-phenylvinyl)pyridin-2-yl)oxy)ethyl)amino)-N-methylbut-2-enamide; (E)-4-((2-((5-((Z)-2-cyclobutyl-1-(1H-indazol-5-yl)-2-phenylvinyl)pyridin-2-yl)oxy)ethyl)amino)-N-methylbut-2-enamide; (E)-4-((2-(4-((E)-2-cyclobutyl-1-(3-fluoro-1H-indazol-5-yl)-2-phenylvinyl)phenoxy)ethyl)amino)-1-(pyrrolidin-1-yl)but-2-en-1-one; (E)-4-((2-(4-((E)-2-cyclobutyl-1-(1H-indazol-5-yl)-2-phenylvinyl)phenoxy)ethyl)amino)-1-(pyrrolidin-1-yl)but-2-en-1-one; (E)-4-((2-((5-((Z)-2-cyclobut-1-(3-fluoro-1H-indazol-5-yl)-2-phenylvinyl)pyridin-2-yl)oxy)ethyl)amino)-1-(pyrrolidin-1-yl)but-2-en-1-one; (E)-4-((2-((5-((Z)-2-cyclobutyl-1-(1H-indazol-5-yl)-2-phenylvinyl)pyridin-2-yl)oxy)ethyl)amino)-1-(pyrrolidin-1-yl)but-2-en-1-one; (E)-N-methyl-4-((2-(4-(4,4,4-trifluoro-1-(1H-indazol-5-yl)-2-phenylbut-1-en-1-vi)phenoxy)ethyl)amino)butanamide; (E)-N-methyl-4-((2-(4-(4,4,4-trifluoro-1-(3-fluoro-1H-indazol-5-yl)-2-phen)but-1-en-1-yl)phenoxy)ethyl)amino)butanamide; (Z)—N-methyl-4-((2-((5-(4,4,4-trifluoro-1-(1H-indazol-5-yl)-2-phenylbut-1-en-1-yl)pyridin-2-yl)oxy)ethyl)amino)butanamide; (E)-1-(pyrrolidin-1-yl)-4-((2-(4-(4,4,4-trifluoro-1-(3-fluoro-1H-indazol-5-yl)-2-phenylbut-1-en-1-yl)phenoxy)ethyl)amino)butan-1-one; (E)-1-(pyrrolidin-1-vi)-4-((2-(4-(4,4,4-trifluoro-14 1H-indazol-5-yl)-2-phenylbut-1-en-1-yl)phenoxy)ethyl)amino)butan-1-one; (Z)-1-(pyrrolidin-1-yl)-4-((2-((5-(4,4,4-trifluoro-1-(3-fluoro-1H-indazol-5-yl)-2-phenylbut-1-en-1-yl)pyridin-2-yl)oxy)ethyl)amino)butan-1-one; (E)-N-methyl-4-((2-((6-methyl-5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-N-methyl-4-((2-((5-((Z)-4,4,4-trifluoro-1-(3-fluoro-1H-indazol-5-yl)-2-phenylbut-1-en-1-yl)pyrimidin-2-yl)oxy)ethyl)amino)but-2-enamide; (E)-4-((2-(4-((E)-2-(2-chloro-4-fluorophenyl)-4,4,4-trifluoro-1-(1H-indazol-5-yl)but-1-en-1-yl)phenoxy)ethyl)amino)-N-methylbut-2-enamide; (E)-4-((2-(4-((E)-2-(2-chloro-4-fluorophenyl)-4,4,4-trifluoro-1-(3-fluoro-1H-indazol-5-yl)but-1-en-1-yl)phenoxy)ethyl)amino)-N-methylbut-2-enamide; (E)-4-((2-(4-((E)-2-(2-chloro-4-fluorophenyl)-1-(3-fluoro-1H-indazol-5-yl)but-1-en-1-yl)phenoxy)ethyl)amino)-N-methylbut-2-enamide; (E)-N-methyl-4-((2-((5-((Z)-1-(3-methyl-1H-indazol-5-yl)-2-phenylbut-1-en-1-yl)pyridin-2-yl)oxy)ethyl)amino)but-2-enamide; (E)-4-((2-(4-((E)-1-(1H-indazol-5-yl)-2-phenylbut-1-en-1-yl)phenoxy)ethyl)amino)-N-methylbut-2-enamide; (E)-4-((2-(4-(1-(1H-indazol-5-yl)-2-phenylbut-1-en-1-yl)phenoxy)ethyl)amino)-N-methylbutanamide; (E)-1-(piperidin-1-yl)-4-((2-(4-((E)-4,4,4-trifluoro-1-(3-fluoro-1H-indazol-5-yl)-2-phenylbut-1-en-1-yl)phenoxy)ethyl)amino)but-2-en-1-one; (Z)-3-(2-((2-((5-(4,4,4-trifluoro-1-(3-fluoro-1H-indazol-5-yl)-2-phenylbut-1-en-1-yl)pyridin-2-yl)oxy)ethyl)amino)ethyl)pyrrolidin-2-one; (E)-N-methyl-4-((2-((6-((Z)-4,4,4-trifluoro-1-(3-fluoro-1H-indazol-5-yl)-2-phenylbut-1-en-1-yl)pyridazin-3-yl)oxy)ethyl)amino)but-2-enamide; (E)-1-(piperidin-1-yl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-en-1-one; (E)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-4-((2-(4-((E)-4,4,4-trifluoro-1-(3-fluoro-1H-indazol-5-yl)-2-phenylbut-1-en-1-yl)phenoxy)ethyl)amino)but-2-enamide; (E)-4-((2-((5-((Z)-2-(2-chloro-4-fluorophenyl)-4,4,4-trifluoro-1-(1H-indazol-5-yl)but-1-en-1-yl)pyridin-2-yl)oxy)ethyl)amino)-N-methylbut-2-enamide; (E)-4-((2-((5-((Z)-2-(2-chloro-4-fluorophenyl)-1-(1H-indazol-5-yl)but-1-en-1-yl)pyridin-2-yl)oxy)ethyl)amino)-N-methylbut-2-enamide; (E)-1-(azetidin-1-yl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-en-1-one; (E)-N-methyl-4-((3-((5-((Z)-4,4,4-trifluoro-1-(3-fluoro-1H-indazol-5-yl)-2-phenylbut-1-en-1-yl)pyridin-2-yl)oxy)propyl)amino)but-2-enamide; (Z)-4-((2-((5-(1-(1H-indazol-5-yl)-2-phenylbut-1-en-1-yl)pyridin-2-yl)oxy)ethyl)amino)-N-methylbutanamide; (E)-4-((2-(4-((E)-2-cyclopropyl-1-(3-fluoro-1H-indazol-5-yl)-2-phenyl)phenoxy)ethyl)amino)-N-methylbut-2-enamide; (E)-4-((2-(4-((E)-1-(3-fluoro-1H-indazol-5-yl)-4-hydroxy-2-phenylbut-1-en-1-yl)phenoxy)ethyl)amino)-N-methylbut-2-enamide; (E)-4-((2-(4-((E)-1-(3-fluoro-1H-indazol-5-yl)-4-methoxy-2-phenylbut-1-en-1-yl)phenoxy)ethyl)amino)-N-methylbut-2-enamide; (E)-4-((2-(4-((E)-4-chloro-1-(3-fluoro-1H-indazol-5-yl)-2-phenylbut-1-en-1-yl)phenoxy)ethyl)amino)-N-methylbut-2-enamide; (E)-4-((2-(4-((E)-1-(3-fluoro-1H-indazol-5-yl)-2-phenylpent-1-en-1-yl)phenoxy)ethyl)amino)-N-methylbut-2-enamide; (E)-4-((2-(4-((E)-1-(3-fluoro-1H-indazol-5-yl)-3-methyl-2-phenylbut-1-en-1-yl)phenoxy)ethyl)amino)-N-methylbut-2-enamide; (E)-N-methyl-4-((2-((6-((E)-4,4,4-trifluoro-1-(3-fluoro-1H-indazol-5-yl)but-1-en-1-yl)pyridazin-3-yl)oxy)ethyl)amino)but-2-enamide; (E)-1-(2-(4-(4,4,4-trifluoro-1-(3-fluoro-1H-indazol-5-yl)-2-phenylbut-1-en-1-yl)phenoxy)ethyl)pyrrolidin-2-one; (Z)—N-methyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide; (E)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enoic acid; (E)-4-((2-(4-((E)-1-(1H-indazol-5-yl)-2-phenylbut-1-en-1-yl)phenoxy)ethyl)amino)but-2-enoic acid; (E)-N-methyl-4-((2-((5-((Z)-4,4,4-trifluoro-1-(3-fluoro-1H-indazol-5-yl)-2-phenylbut-1-en-1-yl)pyrazin-2-yl)oxy)ethyl)amino)but-2-enamide; (E)-N-methyl-4-((2-((6-((Z)-4,4,4-trifluoro-1-(3-fluoro-1H-indazol-5-yl)-2-phenylbut-1-en-1-yl)pyridin-3-yl)oxy)ethyl)amino)but-2-enamide; (Z)—N,N-dimethyl-4-((2-(4-(4,4,4-trifluoro-1-(3-fluoro-1H-indazol-5-yl)-2-phenylbut-1-en-1-yl)phenoxy)ethyl)amino)butanamide; (Z)—N-(2-hydroxyethyl)-N-methyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide; (E)-N-(2-hydroxyethyl)-5-((2-((5-((Z)-4,4,4-trifluoro-1-(3-fluoro-1H-indazol-5-yl)-2-phenylbut-1-en-1-yl)pyridin-2-yl)oxy)ethyl)amino)pent-2-enamide; (E)-N-methyl-4-((2-((5-((E)-4,4,4-trifluoro-1-(3-fluoro-1H-indazol-5-yl)-2-phenylbut-1-en-1-yl)pyridin-2-yl)oxy)ethyl)amino)but-2-enamide; (E)-N-(2-hydroxyethyl)-N-methyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-N-(2-hydroxyethyl)-N-methyl-5-((2-((5-((Z)-4,4,4-trifluoro-1-(3-fluoro-11H-indazol-5-yl)-2-phenylbut-1-en-1-yl)pyridin-2-yl)oxy)ethyl)amino)pent-2-enamide; (E)-1-morpholino-4-((2-(4-((E)-4,4,4-trifluoro-1-(1H-indazol-5-yl)-2-phenylbut-1-en-1-yl)phenoxy)ethyl)amino)but-2-en-1-one; (E)-N,N-dimethyl-4-((2-(4-(4,4,4-trifluoro-1-(1H-indazol-5-yl)-2-phenylbut-1-en-1-yl)phenoxy)ethyl)amino)butanamide; (E)-N-(2-hydroxyethyl)-N-methyl-4-((2-(4-(4,4,4-trifluoro-1-(3-fluoro-1H-indazol-5-yl)-2-phenylbut-1-en-1-yl)phenoxy)ethyl)amino)butanamide; (E)-1-morpholino-4-((2-(4-(4,4,4-trifluoro-1-(3-fluoro-1H-indazol-5-yl)-2-phenylbut-1-en-1-yl)phenoxy)ethyl)amino)butan-1-one; (Z)-1-morpholino-4-((2-((5-(4,4,4-trifluoro-1-(3-fluoro-1H-indazol-5-yl)-2-phenylbut-1-en-1-yl)pyridin-2-yl)oxy)ethyl)amino)butan-1-one; (E)-3-(2-((2-((5-((Z)-4,4,4-trifluoro-1-(3-fluoro-1H-indazol-5-yl)-2-phenylbut-1-en-1-yl)pyridin-2-yl)oxy)ethyl)amino)ethylidene)pyrrolidin-2-one; (E)-N-methyl-4-((3-((5-((Z)-4,4,4-trifluoro-1-(3-fluoro-1H-indazol-5-yl)-2-phenylbut-1-en-1-yl)pyridin-2-yl)oxy)propyl)amino)but-2-enamide; and (E)-N-(2-hydroxyethyl)-5-((2-(4-((E)-4,4,4-trifluoro-1-(3-fluoro-1H-indazol-5-yl)-2-phenylbut-1-en-1-yl)phenoxy)ethyl)amino)pent-2-enamide: or a pharmaceutically acceptable salt thereof.

A further embodiment provides a compound having the following formula:

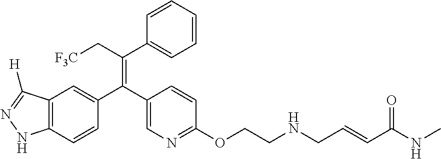 [SMILES: CNC(=O)/C=C/CNCCOc1ccc(/C(=C(/CC(F)(F)F)c2ccccc2)c2ccc3[nH]ncc3c2)cn1]

or a pharmaceutically acceptable salt thereof.

A further embodiment provides a compound having the following formula:

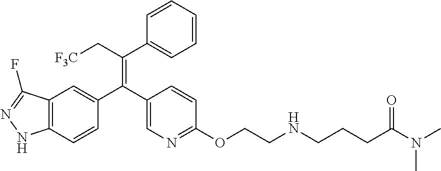 [SMILES: CN(C)C(=O)CCCNCCOc1ccc(/C(=C(/CC(F)(F)F)c2ccccc2)c2ccc3[nH]nc(F)c3c2)cn1]

or a pharmaceutically acceptable salt thereof.

A further embodiment provides a compound having the following formula:

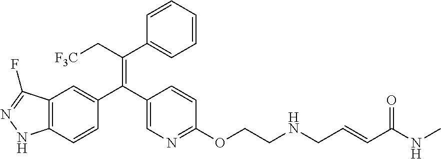 [SMILES: CNC(=O)/C=C/CNCCOc1ccc(/C(=C(/CC(F)(F)F)c2ccccc2)c2ccc3[nH]nc(F)c3c2)cn1]

or a pharmaceutically acceptable salt thereof.

A further embodiment provides a compound of the formula:

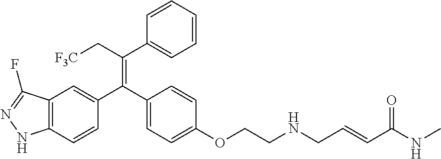 [SMILES: CNC(=O)/C=C/CNCCOc1ccc(/C(=C(/CC(F)(F)F)c2ccccc2)c2ccc3[nH]nc(F)c3c2)cc1]

or a pharmaceutically acceptable salt thereof.

A further embodiment provides a compound of Formula III:

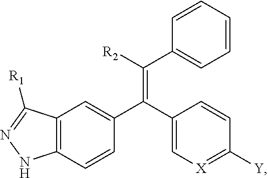

wherein R₁ is H or F:

R₂ is —CH₂CH₃, —CH₂CF₃, or cyclobutyl;

X is C or N;

and Y is one of the following:

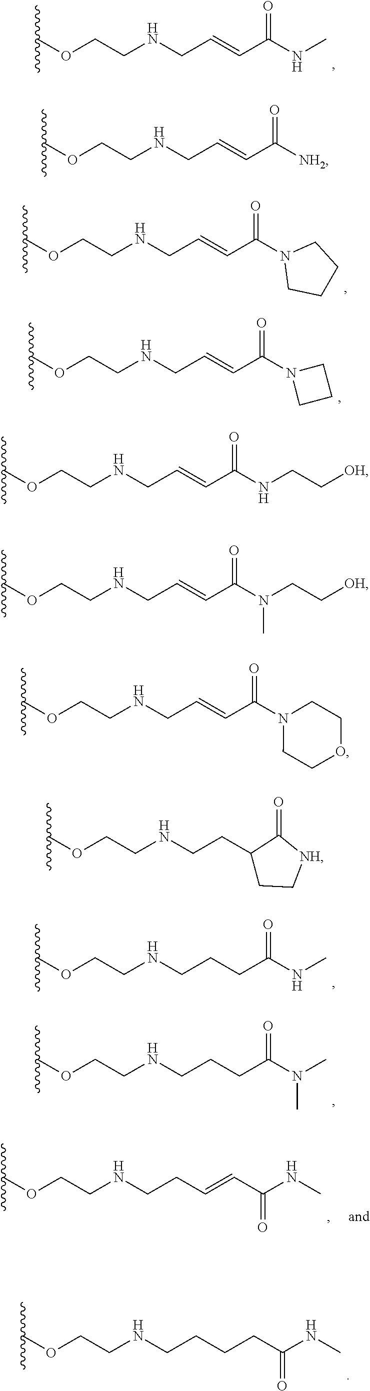

In a further embodiment Y in Formula III may be one of the options for Y in the preceding paragraph and additionally any of the following:

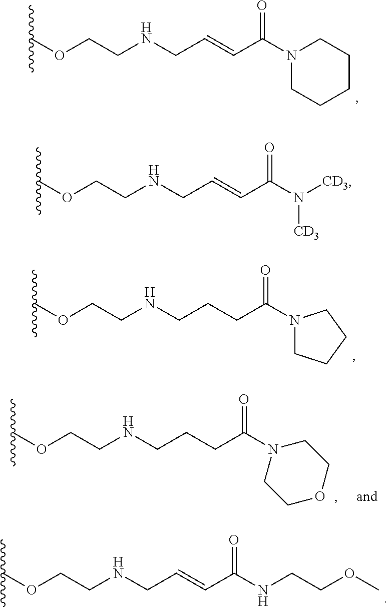 [SMILES: *OCCNC/C=C/C(=O)N1CCCCC1 ; *OCCNC/C=C/C(=O)N(C([2H])([2H])[2H])C([2H])([2H])[2H] ; *OCCNCCCC(=O)N1CCCC1 ; *OCCNCCCC(=O)N1CCOCC1 ; *OCCNC/C=C/C(=O)NCCOC]

A further embodiment may provide a method of treating breast cancer comprising administering to a subject a compound according to any one of the preceding paragraphs.

The breast cancer may be an ER-positive breast cancer. The subject may express a mutant ER-α protein. An embodiment may provide use of a compound as in the paragraphs above for treating breast cancer. In some embodiments the breast cancer is an ER-positive breast cancer. In some embodiments said subject expresses a mutant ER-α protein. In some embodiments a compound or pharmaceutically acceptable salt as presented above is used in the preparation of a medicament for treatment of breast cancer.

In embodiments, the compounds disclosed herein are useful for inhibiting the cell culture growth of MCF7 ER-alpha (wildtype) and MCF7 ER-alpha (Y537S mutant) cells. Other compounds (e.g., tamoxifen, raloxifene and fulvestrant) known to inhibit the cell culture growth of MCF7 ER-alpha (wildtype) cells are currently used to treat breast cancer in human patients. Hence, the compounds disclosed herein are useful for treating ER-alpha expressing breast cancer in human patients, and are useful for treating Y537S mutant ER-alpha expressing breast cancer in human patients.

In embodiments, the compounds disclosed herein are useful for treating breast cancer. In embodiments, the breast cancer is ER-α+. In embodiments, the breast cancer expresses an ER-α mutation, which is L536Q (Robinson et al. Nat Genet. 2013 December; 45(12)), L536R (Toy et al. Nat Genet. 2013 December; 45(12): 1439-45), Y537S (Toy et al. Nat Genet. 2013 December; 45(12): 1439-45; Robinson et al. Nat Genet. 2013 December; 45(12); Jeselsohn et al. Clin Cancer Res. 2014 Apr. 1; 20(7):1757-67), Y537N (Toy et al. Nat Genet. 2013 December; 45(12): 1439-45; Jeselsohn et al. Clin Cancer Res. 2014 Apr. 1; 20(7):1757-67), Y537C (Toy et al. Nat Genet. 2013 December; 45(12): 1439-45: Jeselsohn et al. Clin Cancer Res. 2014 Apr. 1; 20(7):1757-67) and D538G (Toy et al. Nat Genet. 2013 December; 45(12):1439-45: Robinson et al. Nat Genet. 2013 December; 45(12); Jeselsohn et al. Clin Cancer Res. 2014 Apr. 1; 20(7):1757-67; Merenbakh-Lamin et al. Cancer Res. 2013 Dec. 1; 73(23):6856-64); and Yu et al., (2014) Science July 11; 345(6193):216-20, all of which are incorporated by reference in their entireties for their teachings of ER-α mutations.

### BRIEF DESCRIPTION OF THE DRAWINGS

FIG. 1 shows in vitro proliferation effects of wild-type and mutant ER-bearing MCF7 lines to clinical therapies 4-hydroxytamoxifen (4-OHT), raloxifene and fulvestrant, where phenotypic resistance observed in mutant-bearing lines relative to control lines to existing clinical compounds, whereby MCF7 cells engineered to overexpress various ERα^{MUT} showed partial resistance to various endocrine therapies.

FIG. 2 shows antitumor and body weight effects of oral Compound 3 as a hydrochloride salt in ST941 PDX-Y537S xenograft bearing female Balb/c nude mice.

FIG. 3 shows antitumor and body weight effects of oral Compound 21 as a hydrochloride salt in ST941 PDX-Y537S xenograft bearing athymic nude female mice.

FIG. 4 shows the anti-tumor and body weight effects of Compound 21, prepared as an HCl salt, in the MCF7 tumor model bearing ERα^{WT/WT} xenograft.

FIG. 5 shows the anti-tumor and body weight effects of Compound 21, prepared as an HCl salt, in a ST1799 PDX model bearing ERα^{WT/WT} xenograft.

### DETAILED DESCRIPTION

Described herein are novel compounds useful for treating cancer. Embodiments may provide a compound given by Formula I:

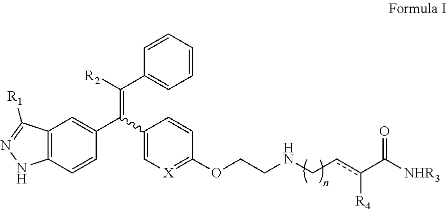

wherein:
R₁ is —H or —F;
R₂ is —CH₂CH₃, —CH₂CF₃, or cyclobutyl;
R₃ is
   i) selected from —H, —CH₃, and —CH₂CH₂OH, or
   ii) forms a 4-7 membered ring with R₄ and the N to which R₃ is attached:
wherein R₄ is —H when it does not form said 5-7-membered ring with R₃
X is N or C;
n is 1-2; and
[symbol] represents a single bond or a double bond;
or a pharmaceutically acceptable salt thereof.

Embodiments of Formula I may have the following stereochemistry:

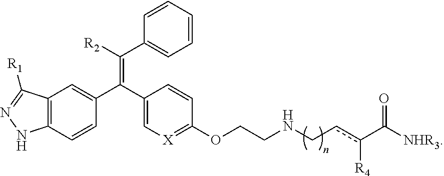

Further embodiments may provide a compound given by Formula II:

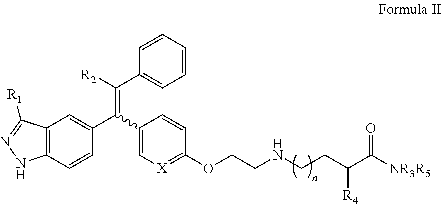

wherein:
R₁ is —H or —F;
R₂ is —CH₂CH₃, —CH₂CF₃, or cyclobutyl;
R₃
   i) is selected from —H, —CH₃, and —CH₂CH₂OH, or
   ii) forms a 4-6 membered ring with R₅ and the N to which R₃ and R₅ are attached, optionally with an additional heteroatom in the 4-6 membered ring;
   iii) forms a 5-7 membered ring with R₄ and the N to which R₃ is attached;
wherein R₄ is —H when it does not form said 5-7-membered ring with R₃;
wherein R₅ is —H, —CH₃, and —CH₂CH₂OH when it does not form said 4-6 membered ring with R₃;
X is N or C; and
n is 1-2; or a pharmaceutically acceptable salt thereof.

Embodiments of Formula II may have the following stereochemistry:

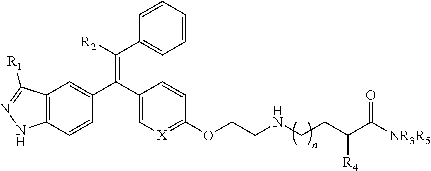

In further embodiments of Formula I or Formula II, R₁ is —F. In further embodiments, R₁ is —H.

In still further embodiments, R₂ is —CH₂—CF₃. In yet still further embodiments, R₂ is —CH₂CH₃. In further embodiments of Formula I, [symbol] represents a single bond. In further embodiments of Formula I or Formula II, n is 1. In still further embodiments, R₃ is —CH₃.

Other embodiments may provide one of the following compounds: N,N-dimethyl-4-[(2-[4-[(1E)-4,4,4-trifluoro-1-(3-fluoro-1H-indazol-5-yl)-1-phenylbut-1-en-2-yl]phenoxy]ethyl)amino]butanamide; (Z)—N,N-dimethyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide; (E)-N-methyl-4-(2-(5-((Z)-4,4,4-trifluoro-1-(3-fluoro-1H-indazol-5-yl)-2-phenylbut-1-enyl)pyridin-2-yloxy)ethylamino)but-2-enamide; (E)-4-((2-(4-(1-(1H-indazol-5-yl)-2-phenylbut-1-en-1-yl)phenoxy)ethyl)amino)-N,N-dimethylbutanamide; (E)-N-methyl-4-((2-((5-((E)-4,4,4-trifluoro-1-(3-fluoro-1H-indazol-5-yl)-1-phenylbut-1-en-2-yl)pyridin-2-yl)oxy)ethyl)amino)but-2-enamide; (E)-N-methyl-5-((2-((5-((Z)-4,4,4-trifluoro-1-(3-fluoro-1H-indazol-5-yl)-2-phenylbut-1-en-1-yl)pyridin-2-yl)oxy)ethyl)amino)pent-2-enamide; (E)-N-(2-hydroxyethyl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (Z)—N-methyl-5-((2-((5-(4,4,4-trifluoro-1-(3-fluoro-1H-indazol-5-yl)-2-phenylbut-1-en-1-yl)pyridin-2-yl)oxy)ethyl)amino)pentanamide; (E)-N-methyl-4-((2-((5-((Z)-4,4,4-trifluoro-1-(1H-indazol-5-yl)-2-phenylbut-1-en-1-yl)pyridin-2-yl)oxy)ethyl)amino)but-2-enamide; (E)-N-methyl-4-((2-(4-((E)-4,4,4-trifluoro-1-(1H-indazol-5-yl)-2-phenylbut-1-en-1-yl)phenoxy)ethyl)amino)but-2-enamide; (E)-4-((2-(4-((E)-2-cyclobutyl-1-(1H-indazol-5-yl)-2-phenylvinyl)phenoxy)ethyl)amino)-N-methylbut-2-enamide; (Z)-1-(2-((5-(4,4,4-trifluoro-1-(3-fluoro-1H-indazol-5-yl)-2-phenylbut-1-en-1-yl)pyridin-2-yl)oxy)ethyl)pyrrolidin-2-one; (E)-1-(pyrrolidin-1-yl)-4-((2-(4-((E)-4,4,4-trifluoro-1-(3-fluoro-1H-indazol-5-yl)-2-phenylbut-1-en-1-yl)phenoxy)ethyl)amino)but-2-en-1-one; (E)-1-(pyrrolidin-1-yl)-4-((2-(4-((E)-4,4,4-trifluoro-1-(1H-indazol-5-yl)-2-phenylbut-1-en-1-yl)phenoxy)ethyl)amino)but-2-en-1-one; (E)-1-(pyrrolidin-1-yl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-en-1-one; (E)-1-(pyrrolidin-1-yl)-4-((2-((5-((Z)-4,4,4-trifluoro-1-(1H-indazol-5-yl)-2-phenylbut-1-en-1-yl)pyridin-2-yl)oxy)ethyl)amino)but-2-en-1-one; (E)-1-morpholino-4-((2-(4-((E)-4,4,4-trifluoro-1-(3-fluoro-1H-indazol-5-yl)-2-phenylbut-1-en-1-yl)phenoxy)ethyl)amino)but-2-en-1-one; (E)-1-morpholino-4-((2-((5-((Z)-4,4,4-trifluoro-1-(3-fluoro-1H-indazol-5-yl)-2-phenylbut-1-en-1-yl)pyridin-2-yl)ox)ethyl)amino)but-2-en-1-one; (E)-1-morpholino-4-((2-((5-((Z)-4,4,4-trifluoro-1-(1H-indazol-5-yl)-2-phenylbut-1-en-1-yl)pyridin-2-yloxy)ethyl)amino)but-2-en-1-one; (E)-N-(2-methoxyethyl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-N-methyl-4-((2-(4-((E)-4,4,4-trifluoro-1-(3-fluoro-1H-indazol-5-yl)-2-phenylbut-1-en-1-yl)phenoxy)ethyl)amino)but-2-enamide; (E)-N,N-di(²H₃)methyl-4-((2-(4-((E)-4,4,4-trifluoro-1-(3-fluoro-1H-indazol-5-yl)-2-phenylbut-1-en-1-yl)phenoxy)ethyl)amino)but-2-enamide; (E)-N,N-di(²H₃)methyl-4-((2-(4-((E)-4,4,4-trifluoro-1-(1H-indazol-5-yl)-2-phenylbut-1-en-1-trifluoro-1-(3-fluoro-1H-indazol-5-yl)-2-phenylbut-1-en-1-yl)pyridin-2-yl)oxy)ethyl)amino)but-2-enamide; (E)-N,N-di(²H₃)methyl-4-((2-((5-((Z)-4,4,4-trifluoro-1-(1H-indazol-5-yl)-2-phenylbut-1-en-1-yl)pyridin-2-yl)oxy)ethyl)amino)but-2-enamide; (E)-4-((2-(4-((E)-1-(3-fluoro-1H-indazol-5-yl)-2-phenylbut-1-en-1-yl)phenoxy)ethyl)amino)-N-methylbut-2-enamide; (E)-4-((2-((5-((Z)-1-(3-fluoro-1H-indazol-5-yl)-2-phenylbut-1-en-1-yl)pyridin-2-yl)oxy)ethyl)amino)-N-methylbut-2-enamide; (E)-4-((2-((5-((Z)-1-(1H-indazol-5-yl)-2-phenylbut-1-en-1-yl)pyridin-2-yl)oxy)ethyl)amino)-N-methylbut-2-enamide; (E)-4-((2-(4-((E)-1-(3-fluoro-1H-indazol-5-yl)-2-phenylbut-1-en-1-yl)phenoxy)ethyl)amino)-1-(pyrrolidin-1-yl)but-2-en-1-one; (E)-4-((2-((5-((Z)-1-(3-fluoro-1H-indazol-5-yl)-2-phenylbut-1-en-1-yl)pyridin-2-yl)oxy)ethyl)amino)-1-(pyrrolidin-1-yl)but-2-en-1-one; (E)-4-((2-(4-((E)-2-cyclobutyl-1-(3-fluoro-1H-indazol-5-yl)-2-phenylvinyl)phenoxy)ethyl)amino)-N-methylbut-2-enamide; (E)-4-((2-((5-((Z)-2-cyclobutyl-1-(3-fluoro-1H-indazol-5-yl)-2-phenylvinyl)pyridin-2-yl)oxy)ethyl)amino)-N-methylbut-2-enamide; (E)-4-((2-((5-((Z)-2-cyclobutyl-1-(1H-indazol-5-yl)-2-phenylvinyl)pyridin-2-yl)oxy)ethyl)amino)-N-methylbut-2-enamide; (E)-4-((2-(4-((E)-2-cyclobutyl-1-(3-fluoro-1H-indazol-5-yl)-2-phenylvinyl)phenoxy)ethyl)amino)-1-(pyrrolidin-1-yl)but-2-en-1-one; (E)-4-((2-(4-((E)-2-cyclobutyl-1-(1H-indazol-5-yl)-2-phenylvinyl)phenoxy)ethyl)amino)-1-(pyrrolidin-1-yl)but-2-en-1-one; (E)-4-((2-((5-((Z)-2-cyclobutyl-1-(3-fluoro-1H-indazol-5-yl)-2-phenylvinyl)pyridin-2-yl)oxy)ethyl)amino)-1-(pyrrolidin-1-yl)but-2-en-1-one; (E)-4-((2-((5-((Z)-2-cyclobutyl-1-(1H-indazol-5-yl)-2-phenylvinyl)pyridin-2-yl)oxy)ethyl)amino)-1-(pyrrolidin-1-yl)but-2-en-1-one; (E)-N-methyl-4-((2-(4-(4,4,4-trifluoro-1-(1H-indazol-5-yl)-2-phenylbut-1-en-1-yl)phenoxy)ethyl)amino)butanamide; (E)-N-methyl-4-((2-(4-(4,4,4-trifluoro-1-(3-fluoro-1H-indazol-5-yl)-2-phenylbut-1-en-1-yl)phenoxy)ethyl)amino)butanamide; (Z)—N-methyl-4-((2-((5-(4,4,4-trifluoro-1-(1H-indazol-5-yl)-2-phenylbut-1-en-1-yl)pyridin-2-yl)oxy)ethyl)amino)butanamide; (E)-1-(pyrrolidin-1-yl)-4-((2-(4-(4,4,4-trifluoro-1-(3-fluoro-1H-indazol-5-yl)-2-phenylbut-1-en-1-yl)phenoxy)ethyl)amino)butan-1-one; (E)-1-(pyrrolidin-1-yl)-4-((2-(4-(4,4,4-trifluoro-1 (1H-indazol-5-yl)-2-phenylbut-1-en-1-yl)phenoxy)ethyl)amino)butan-1-one; (Z)-1-(pyrrolidin-1-yl)-4-((2-((5-(4,4,4-trifluoro-1-(3-fluoro-1H-indazol-5-yl)-2-phenylbut-1-en-1-yl)pyridin-2-yl)oxy)ethyl)amino)butan-1-one; (E)-N-methyl-4-((2-((6-methyl-5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-N-methyl-4-((2-((5-((Z)-4,4,4-trifluoro-1-(3-fluoro-1H-indazol-5-yl)-2-phenylbut-1-en-1-yl)pyrimidin-2-yl)oxy)ethyl)amino)but-2-enamide; (E)-4-((2-(4-((E)-2-(2-chloro-4-fluorophenyl)-4,4,4-trifluoro-1-(1H-indazol-5-yl)but-1-en-1-yl)phenoxy)ethyl)amino)-N-methylbut-2-enamide; (E)-4-((2-(4-((E)-2-(2-chloro-4-fluorophenyl)-4,4,4-trifluoro-1-(3-fluoro-1H-indazol-5-yl)but-1-en-1-yl)phenoxy)ethyl)amino)-N-methylbut-2-enamide; (E)-4-((2-(4-((E)-2-(2-chloro-4-fluorophenyl)-1-(3-fluoro-1H-indazol-5-yl)but-1-en-1-yl)phenoxy)ethyl)amino)-N-methylbut-2-enamide; (E)-N-methyl-4-((2-((5-((Z)-1-(3-methyl-1H-indazol-5-yl)-2-phenylbut-1-en-1-yl)pyridin-2-yl)oxy)ethyl)amino)but-2-enamide; (E)-4-((2-(4-((E)-1-(1H-indazol-5-yl)-2-phenylbut-1-en-1-yl)phenoxy)ethyl)amino)-N-methylbut-2-enamide; (E)-4-((2-(4-(1-(1H-indazol-5-yl)-2-phenylbut-1-en-1-yl)phenoxy)ethyl)amino)-N-methylbutanamide; (E)-1-(piperidin-1-yl)-4-((2-(4-((E)-4,4,4-trifluoro-1-(3-fluoro-1H-indazol-5-yl)-2-phenylbut-1-en-1-yl)phenoxy)ethyl)amino)but-2-en-1-one; (Z)-3-(2-((2-((5-(4,4,4-trifluoro-1-(3-fluoro-1H-indazol-5-yl)-2-phenylbut-1-en-1-yl)pyridin-2-yl)oxy)ethyl)amino)ethyl)pyrrolidin-2-one; (E)-N-methyl-4-((2-((6-((Z)-4,4,4-trifluoro-1-(3-fluoro-1H-indazol-5-yl)-2-phenylbut-1-en-1-yl)pyridazin-3-yl)oxy)ethyl)amino)but-2-enamide; (E)-1-(piperidin-1-yl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-en-1-one; (E)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-4-((2-(4-((E)-4,4,4-trifluoro-1-(3-fluoro-1H-indazol-5-yl)-2-phenylbut-1-en-1-yl)phenoxy)ethyl)amino)but-2-enamide; (E)-4-((2-((5-((Z)-2-(2-chloro-4-fluorophenyl)-4,4,4-trifluoro-1-(1H-indazol-5-yl)but-1-en-1-yl)pyridin-2-yl)oxy)ethyl)amino)-N-methylbut-2-enamide; (E)-4-((2-((5-((Z)-2-(2-chloro-4-fluorophenyl)-1-(1H-indazol-5-yl)but-1-en-1-yl)pyridin-2-yl)oxy)ethyl)amino)-N-methylbut-2-enamide; (E)-1-(azetidin-1-yl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-en-1-one; (E)-N-methyl-4-((3-((5-((Z)-4,4,4-trifluoro-1-(3-fluoro-1H-indazol-5-yl)-2-phenylbut-1-en-1-yl)pyridin-2-yl)oxy)propyl)amino)but-2-enamide; (Z)-4-((2-((5-(1-(1H-indazol-5-yl)-2-phenylbut-1-en-1-yl)pyridin-2-yl)oxy)ethyl)amino)-N-methylbutanamide; (E)-4-((2-(4-((E)-2-cyclopropyl-1-(3-fluoro-1H-indazol-5-yl)-2-phenylvinyl)phenoxy)ethyl)amino)-N-methylbut-2-enamide; (E)-4-((2-(4-((E)-1-(3-fluoro-1H-indazol-5-yl)-4-hydroxy-2-phenylbut-1-en-1-yl)phenoxy)ethyl)amino)-N-methylbut-2-enamide; (E)-4-((2-(4-((E)-1-(3-fluoro-1H-indazol-5-yl)-4-methoxy-2-phenylbut-1-en-1-yl)phenoxy)ethyl)amino)-N-methylbut-2-enamide; (E)-4-((2-(4-((E)-4-chloro-1-(3-fluoro-1H-indazol-5-yl)-2-phenylbut-1-en-1-yl)phenoxy)ethyl)amino)-N-methylbut-2-enamide; (E)-4-((2-(4-((E)-1-(3-fluoro-1H-indazol-5-yl)-2-phenylpent-1-en-1-yl)phenoxy)ethyl)amino)-N-methylbut-2-enamide; (E)-4-((2-(4-((E)-1-(3-fluoro-1H-indazol-5-yl)-3-methyl-2-phenylbut-1-en-1-yl)phenoxy)ethyl)amino)-N-methylbut-2-enamide; (E)-N-methyl-4-((2-((6-((E)-4,4,4-trifluoro-1-(3-fluoro-1H-indazol-5-yl)but-1-en-1-yl)pyridazin-3-yl)oxy)ethyl)amino)but-2-enamide; (E)-1-(2-(4-(4,4,4-trifluoro-1-(3-fluoro-1H-indazol-5-yl)-2-phenylbut-1-en-1-yl)phenoxy)ethyl)pyrrolidin-2-one; (Z)—N-methyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide; (E)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enoic acid; (E)-4-((2-(4-((E)-1-(1H-indazol-5-yl)-2-phenylbut-1-en-1-yl)phenoxy)ethyl)amino)but-2-enoic acid; (E)-N-methyl-4-((2-((5-((Z)-4,4,4-trifluoro-1-(3-fluoro-1H-indazol-5-yl)-2-phenylbut-1-en-1-yl)pyrazin-2-yl)oxy)ethyl)amino)but-2-enamide; (E)-N-methyl-4-((2-((6-((Z)-4,4,4-trifluoro-1-(3-fluoro-1H-indazol-5-yl)-2-phenylbut-1-en-1-yl)pyridin-3-yl)oxy)ethyl)amino)but-2-enamide; (Z)—N,N-dimethyl-4-((2-(4-(4,4,4-trifluoro-1-(3-fluoro-1H-indazol-5-yl)-2-phenylbut-1-en-1-yl)phenoxy)ethyl)amino)butanamide; (Z)—N-(2-hydroxyethyl)-N-methyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide; (E)-N-(2-hydroxyethyl)-5-((2-((5-((Z)-4,4,4-trifluoro-1-(3-fluoro-1H-indazol-5-yl)-2-phenylbut-1-en-1-yl)pyridin-2-yl)oxy)ethyl)amino)pent-2-enamide; (E)-N-methyl-4-((2-((5-((E)-4,4,4-trifluoro-1-(3-fluoro-1H-indazol-5-yl)-2-phenylbut-1-en-1-yl)pyridin-2-yl)oxy)ethyl)amino)but-2-enamide; (E)-N-(2-hydroxyethyl)-N-methyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide; (E)-N-(2-hydroxyethyl)-N-methyl-5-((2-((5-((Z)-4,4,4-trifluoro-1-(3-fluoro-1H-indazol-5-yl)-2-phenylbut-1-en-1-yl)pyridin-2-yl)oxy)ethyl)amino)pent-2-enamide; (E)-1-morpholino-4-((2-(4-((E)-4,4,4-trifluoro-1-(1H-indazol-5-yl)-2-phenylbut-1-en-1-yl)phenoxy)ethyl)amino)but-2-en-1-one; (E)-N,N-dimethyl-4-((2-(4-(4,4,4-trifluoro-1-(1H-indazol-5-yl)-2-phenylbut-1-en-1-yl)phenoxy)ethyl)amino)butanamide; (E)-N-(2-hydroxyethyl)-N-methyl-4-((2-(4-(4,4,4-trifluoro-1-(3-fluoro-1H-indazol-5-yl)-2-phenylbut-1-en-1-yl)phenoxy)ethyl)amino)butanamide; (E)-1-morpholino-4-((2-(4-(4,4,4-trifluoro-1-(3-fluoro-1H-indazol-5-yl)-2-phenylbut-1-en-1-yl)phenoxy)ethyl)amino)butan-1-one; (Z)-1-morpholino-4-((2-((5-(4,4,4-trifluoro-1-(3-fluoro-1H-indazol-5-yl)-2-phenylbut-1-en-1-yl)pyridin-2-yl)oxy)ethyl)amino)butan-1-one; (E)-3-(2-((2-((5-((Z)-4,4,4-trifluoro-1-(3-fluoro-1H-indazol-5-yl)-2-phenylbut-1-en-1-yl)pyridin-2-yl)oxy)ethyl)amino)ethylidene)pyrrolidin-2-one; (E)-N-methyl-4-((3-((5-((Z)-4,4,4-trifluoro-1-(3-fluoro-1H-indazol-5-yl)-2-phenylbut-1-en-1-yl)pyridin-2-yl)oxy)propyl)amino)but-2-enamide; and (E)-N-(2-hydroxyethyl)-5-((2-(4-((E)-4,4,4-trifluoro-1-(3-fluoro-1H-indazol-5-yl)-2-phenylbut-1-en-1-yl)phenoxy)ethyl)amino)pent-2-enamide; or a pharmaceutically acceptable salt thereof.

A further embodiment provides a compound having the following formula:

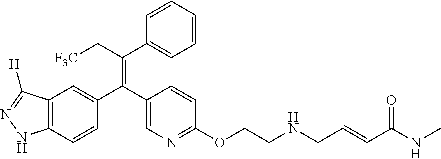 [SMILES: CNC(=O)/C=C/CNCCOc1ccc(/C(=C(/CC(F)(F)F)c2ccccc2)c2ccc3[nH]ncc3c2)cn1]

or a pharmaceutically acceptable salt thereof.

A further embodiment provides a compound having the following formula:

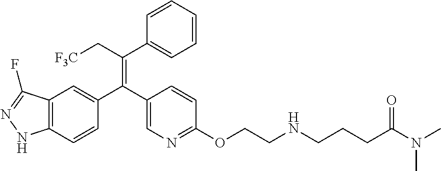 [SMILES: CN(C)C(=O)CCCNCCOc1ccc(/C(=C(/CC(F)(F)F)c2ccccc2)c2ccc3[nH]nc(F)c3c2)cn1]

or a pharmaceutically acceptable salt thereof.

A further embodiment provides a compound having the following formula:

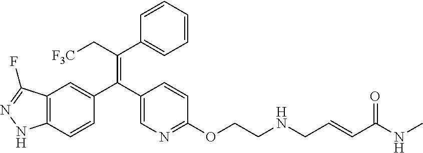 [SMILES: CNC(=O)/C=C/CNCCOc1ccc(/C(=C(/CC(F)(F)F)c2ccccc2)c2ccc3[nH]nc(F)c3c2)cn1]

or a pharmaceutically acceptable salt thereof.

A further embodiment provides a compound of the formula:

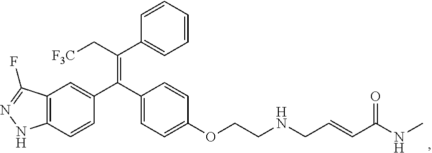 [SMILES: CNC(=O)/C=C/CNCCOc1ccc(/C(=C(/CC(F)(F)F)c2ccccc2)c2ccc3[nH]nc(F)c3c2)cc1]

or a pharmaceutically acceptable salt thereof.

A further embodiment provides a compound of Formula III:

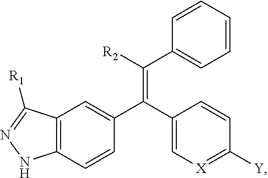

wherein R₁ is —H or —F:

R₂ is —CH₂CH₃, —CH₂CF₃, or cyclobutyl;

X is C or N;

and Y is one of the following:

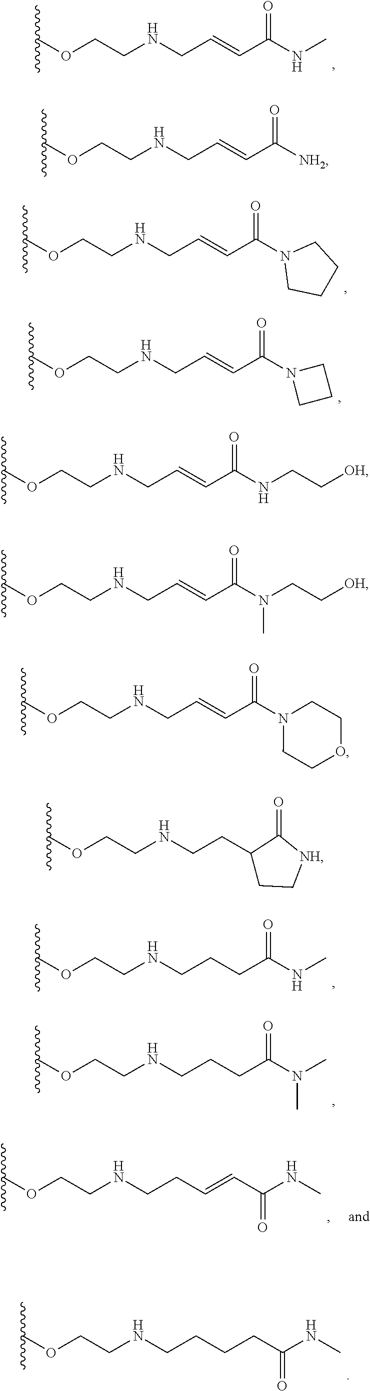

In a further embodiment Y in Formula III may be one of the options for Y in the preceding paragraph and additionally any of the following:

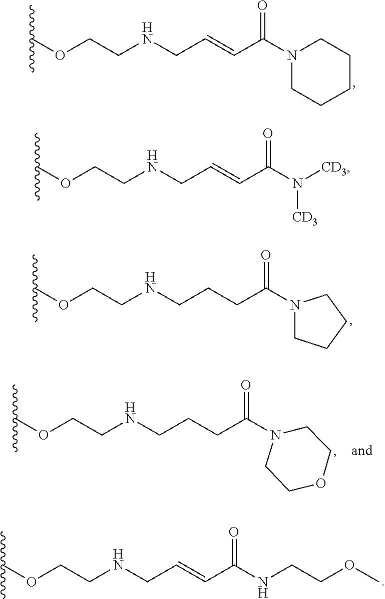

A further embodiment may provide a method of treating breast cancer comprising administering to a subject a compound or pharmaceutically acceptable salt according to any one of the preceding paragraphs. The breast cancer may be an ER-positive breast cancer. The subject may express a mutant ER-α protein. An embodiment may provide use of a compound as in the paragraphs above for treating breast cancer. In some embodiments the breast cancer is an ER-positive breast cancer. In some embodiments said subject expresses a mutant ER-α protein. In some embodiments a compound or pharmaceutically acceptable salt as presented above is used in the preparation of a medicament for treatment of breast cancer.

All publications and patent documents cited herein are incorporated herein by reference as if each such publication or document was specifically and individually indicated to be incorporated herein by reference. Where the text of this disclosure and the text of one or more documents incorporated by reference conflicts, this disclosure controls. Citation of publications and patent documents is not intended as an admission that any is pertinent prior art, nor does it constitute any admission as to the contents or date of the same. The embodiments described herein having now been described by way of written description, those of skill in the art will recognize that the embodiments described herein may be practiced in a variety of embodiments and that the description and examples provided herein are for purposes of illustration and not limitation of the claims.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon ring having 3 to 7 carbon atoms (e.g., C₃-C₇). Examples of cycloalkyl include, but are not limited to, cyclopropyl, cyclobutyl, cyclopentyl, cyclohexyl, cycloheptyl, cyclopentenyl, cyclohexenyl, and cycloheptenyl.

The term “heterocycloalkyl” refers to saturated or unsaturated nonaromatic 3-8 membered monocyclic groups or 7-10 membered fused bicyclic groups (or, where indicated, groups having other specified numbers of members) having one or more heteroatoms (such as O, N, or S), unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, thiazolidinyl, oxiranyl, azetidinyl, oxetanyl, thietanyl, 1,2,3,6-tetrahydropyridinyl, tetrahydropyranyl, tetrahydrothiophene, dihydropyranyl, pyranyl, morpholinyl, 1,4-diazepanyl, 1,4-oxazepanyl, and the like.

Additional examples of heterocycloalkyl groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl. 1,3,4-thiadiazolyl, thianthrenyl, thiazolyl, thienyl, thienothiazolyl, thienooxazolyl, thienoimidazolyl, thiophenyl, triazinyl, 1,2,3-triazolyl. 1,2,4-triazolyl, 1,2,5-triazolyl, 1,3,4-triazolyl and xanthenyl.

The term “optionally substituted alkyl” refers to unsubstituted alkyl or alkyl having designated substituents replacing one or more hydrogen atoms on one or more carbons of the hydrocarbon backbone. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl(benzyl)). An “alkylaryl” moiety is an aryl substituted with an alkyl (e.g., methylphenyl).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety. “Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR′ wherein R′ is H or other substituents, as defined).

The nitrogen and sulfur heteroatoms may optionally be oxidized (i.e., N—O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., bicyclic. Non-limiting example of such aryl groups include, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline).

The cycloalkyl, heterocycloalkyl, aryl, or heteroaryl ring may be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups may also be fused with alicyclic or heterocyclic rings, which are not aromatic so as to form a multicyclic system (e.g., tetralin, methylenedioxyphenyl).

When a bond to a substituent is shown to cross a bond connecting two atoms in a ring (as shown by the examples below with substituent R), then such substituent may be bonded to any atom in the ring.

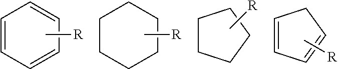

When any variable (e.g., R1) occurs more than one time in any constituent or formula for a compound, its definition at each occurrence is independent of its definition at every other occurrence. Thus, for example, if a group is shown to be substituted with 0-2 R₁ moieties, then the group may optionally be substituted with up to two R₁ moieties and R₁ at each occurrence is selected independently from the definition of R₁.

The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups may be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.” A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Calm et al., Angew. Chem. Inter. Edit. 1966, 5, 385: errata 511; Cahn et al., Angew. Chem. 1966, 78, 413; Cahn and Ingold, J. Chem. Soc. 1951 (London), 612; Calm et al., Experientia 1956, 12, 81; Cahn, J. Chem. Educ. 1964, 41, 116).

In the present specification, each incidence of a chiral center within a structural formula, such as the non-limiting example shown here:

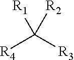

is meant to depict all possible stereoisomers. In contrast, a chiral center drawn with hatches and wedges, such as the non-limiting example shown here:

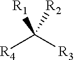

is meant to depict the stereoisomer as indicated (here in this sp³ hybridized carbon chiral center, R₃ and R₄ are in the plane of the paper, R₁ is above the plane of paper, and R₂ is behind the plane of paper).

“Geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

In the present specification, each incidence within a structural formula including a wavy line adjacent to a double bond as shown:

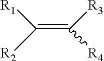

is meant to depict both geometric isomers. In contrast, such structures drawn without a wavy line is meant to depict a compound having the geometric configuration as drawn.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Where the present specification depicts a compound prone to tautomerization, but only depicts one of the tautomers, it is understood that all tautomers are included as part of the meaning of the chemical depicted. It is to be understood that the compounds disclosed herein may be depicted as different tautomers. It should also be understood that when compounds have tautomeric forms, all tautomeric forms are intended to be included, and the naming of the compounds does not exclude any tautomer form.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

Furthermore, the structures and other compounds disclosed herein include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The term “crystal polymorphs”, “polymorphs” or “crystal forms” means crystal structures in which a compound (or a salt or solvate thereof) may crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds may be prepared by crystallization under different conditions. It is understood that the compounds disclosed herein may exist in crystalline form, crystal form mixture, or anhydride or hydrate thereof.

The compounds disclosed herein include the compounds themselves, as well as their salts and solvates, if applicable. A salt, for example, may be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt may also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms.

Additionally, the compounds disclosed herein, for example, the salts of the compounds, may exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, “pharmaceutically acceptable salts” refer to derivatives of the compounds disclosed herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt may be 1:1, or any ratio other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

Chemicals as named or depicted are intended to include all naturally occurring isotopes of atoms occurring in the present compounds. Isotopes include those atoms having the same atomic number but different mass numbers. By way of general example and without limitation, isotopes of ¹H hydrogen include tritium and deuterium, and isotopes of ¹²C carbon include ¹³C and ¹⁴C.

It will be understood that some compounds, and isomers, salts, esters and solvates thereof, of the compounds disclosed herein may exhibit greater in vivo or in vitro activity than others. It will also be appreciated that some cancers may be treated more effectively than others, and may be treated more effectively in certain species of subjects that others, using the compounds, and isomers, salts, esters and solvates thereof, of the compounds disclosed herein.

As used herein, “treating” means administering to a subject a pharmaceutical composition to ameliorate, reduce or lessen the symptoms of a disease. As used herein, “treating” or “treat” describes the management and care of a subject for the purpose of combating a disease, condition, or disorder and includes the administration of a compound disclosed herein, or a pharmaceutically acceptable salt, polymorph or solvate thereof, to alleviate the symptoms or complications of a disease, condition or disorder, or to eliminate the disease, condition or disorder. The term “treat” may also include treatment of a cell in vitro or an animal model.

Treating cancer may result in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×. 4×, 5×, 10×, or 50×.

Treating cancer may result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

As used herein, “subject” or “subjects” refers to any animal, such as mammals including rodents (e.g., mice or rats), dogs, primates, lemurs or humans.

Treating cancer may result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in increase in average survival time of a population of treated subjects in comparison to a population receiving monotherapy with a drug that is not a compound disclosed herein, or a pharmaceutically acceptable salt thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in a decrease in the mortality rate of a population of treated subjects in comparison to a population receiving carrier alone. Treating cancer may result in a decrease in the mortality rate of a population of treated subjects in comparison to an untreated population. Treating cancer may result in a decrease in the mortality rate of a population of treated subjects in comparison to a population receiving monotherapy with a drug that is not a compound disclosed herein, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer may result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate may be measured according to a change in tumor diameter per unit time.

Treating cancer may result in a decrease in tumor regrowth, for example, following attempts to remove it surgically. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder may result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder may result in a reduction in the proportion of proliferating cells. Preferably, after treatment, the proportion of proliferating cells is reduced by at least 5%; more preferably, by at least 10%; more preferably, by at least 20%; more preferably, by at least 30% more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells may be equivalent to the mitotic index.

Treating or preventing a cell proliferative disorder may result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder may result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology may be measured by microscopy, e.g., using an inverted tissue culture microscope. An abnormal cellular morphology may take the form of nuclear pleiomorphism.

As used herein, the term “alleviate” is meant to describe a process by which the severity of a sign or symptom of a disorder is decreased. Importantly, a sign or symptom may be alleviated without being eliminated. In a preferred embodiment, the administration of pharmaceutical compositions disclosed herein leads to the elimination of a sign or symptom, however, elimination is not required. Effective dosages are expected to decrease the severity of a sign or symptom. For instance, a sign or symptom of a disorder such as cancer, which may occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the Amerimay Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute. www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of embodiments described herein, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by non-health-care professionals.

A “pharmaceutical composition” is a formulation containing a compound disclosed herein in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound disclosed herein include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit-risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The present disclosure also provides pharmaceutical compositions comprising any compound disclosed herein in combination with at least one pharmaceutically acceptable excipient or carrier.

A pharmaceutical composition disclosed herein is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

A compound or pharmaceutical composition disclosed herein may be administered to a subject in many of the well-known methods currently used for chemotherapeutic treatment. For example, for treatment of cancers, a compound disclosed herein may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect may be detected by any assay method known in the art. The precise effective amount for a subject will depend upon the subject's body weight, size, and health: the nature and extent of the condition; and the therapeutic or combination of therapeutics selected for administration. Therapeutically effective amounts for a given situation may be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount may be estimated initially either in cell culture assays, e.g., of neoplastic cells, or in animal models, usually rats, mice, rabbits, dogs, or pigs. The animal model may also be used to determine the appropriate concentration range and route of administration. Such information may then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days. Every week, or once every two weeks depending on half-life and clearance rate of the particular formulation.

The pharmaceutical compositions containing active compounds disclosed herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that may be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water. Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like may contain any of the following ingredients, or compounds of a similar nature: a binder such as microcrystalline cellulose, gum tragacanth or gelatin: an excipient such as starch or lactose, a disintegrating agent such as alginic acid, Primogel, or corn starch: a lubricant such as magnesium stearate or Sterotes; a glidant such as colloidal silicon dioxide: a sweetening agent such as sucrose or saccharin; or a flavoring agent such as peppermint, methyl salicylate, or orange flavoring.

The active compounds may be prepared with pharmaceutically acceptable carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the compounds disclosed herein are dictated by and directly dependent on the unique characteristics of the active compound and the particular therapeutic effect to be achieved.

In therapeutic applications, the dosages of the pharmaceutical compositions used in accordance with embodiments described herein vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages may range from about 0.01 mg/kg per day to about 5000 mg/kg per day. In preferred aspects, dosages may range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions may be included in a container, pack, or dispenser together with instructions for administration.

Techniques for formulation and administration of the compounds disclosed herein may be found in Remington: the Science and Practice of Pharmacy, 19^{th} edition, Mack Publishing Co., Easton, Pa. (1995). In an embodiment, the compounds described herein, and the pharmaceutically acceptable salts thereof, may be used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Exemplary cancers that may be treated using one or more compounds disclosed herein include, but are not limited to. Breast cancer, uterine endometrial, ovarian carcinoma, sarcoma, thyroid carcinoma, prostate, lung adenocarcinoma, and hepatocellular carcinoma. In embodiments, the compounds disclosed herein may be useful for treating breast cancer. In embodiments, the breast cancer is ER-α+.

Thus, the compounds disclosed herein may be also useful for additional indications and genotypes. ESR1 mutations (Y537C/N) were recently discovered in 4 of 373 cases of endometrial cancers (Kandoth et al. Nature 2013 May 2; 497(7447):67-73; Robinson et al. Nat Genet. 2013 December; 45(12)). Since it has been shown that ESR1 mutations Y537C/N significantly drive resistance to currently marketed SOC therapies, the compounds disclosed herein may be useful for treating ERα™ endometrial cancers.

Exemplary cell proliferative disorders that may be treated using one or more compounds disclosed herein include, but are not limited to breast cancer, a precancer or precancerous condition of the breast, benign growths or lesions of the breast, and malignant growths or lesions of the breast, and metastatic lesions in tissue and organs in the body other than the breast. Cell proliferative disorders of the breast may include hyperplasia, metaplasia, and dysplasia of the breast.

A breast cancer that is to be treated may arise in a male or female subject. A breast cancer that is to be treated may arise in a premenopausal female subject or a postmenopausal female subject. A breast cancer that is to be treated may arise in a subject 30 years old or older, or a subject younger than 30 years old. A breast cancer that is to be treated has arisen in a subject 50 years old or older, or a subject younger than 50 years old. A breast cancer that is to be treated may arise in a subject 70 years old or older, or a subject younger than 70 years old.

A compound disclosed herein, or a pharmaceutically acceptable salt thereof, may be used to treat or prevent a cell proliferative disorder of the breast, or to treat or prevent breast cancer, in a subject having an increased risk of developing breast cancer relative to the population at large, or used to identify suitable candidates for such purposes. A subject with an increased risk of developing breast cancer relative to the population at large is a female subject with a family history or personal history of breast cancer. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old.

A cancer that is to be treated may include a tumor that has been determined to be less than or equal to about 2 centimeters in diameter. A cancer that is to be treated may include a tumor that has been determined to be from about 2 to about 5 centimeters in diameter. A cancer that is to be treated may include a tumor that has been determined to be greater than or equal to about 3 centimeters in diameter. A cancer that is to be treated may include a tumor that has been determined to be greater than 5 centimeters in diameter. A cancer that is to be treated may be classified by microscopic appearance as well differentiated, moderately differentiated, poorly differentiated, or undifferentiated. A cancer that is to be treated may be classified by microscopic appearance with respect to mitosis count (e.g., amount of cell division) or nuclear pleiomorphism (e.g., change in cells). A cancer that is to be treated may be classified by microscopic appearance as being associated with areas of necrosis (e.g., areas of dying or degenerating cells). A cancer that is to be treated may be classified as having an abnormal karyotype, having an abnormal number of chromosomes, or having one or more chromosomes that are abnormal in appearance. A cancer that is to be treated may be classified as being aneuploid, triploid, tetraploid, or as having an altered ploidy. A cancer that is to be treated may be classified as having a chromosomal translocation, or a deletion or duplication of an entire chromosome, or a region of deletion, duplication or amplification of a portion of a chromosome.

The compounds, or pharmaceutically acceptable salts thereof may be administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds may be selected in accordance with a variety of factors including type, species, age, weight, sex and medical condition of the patient: the severity of the condition to be treated; the route of administration; the renal and hepatic function of the patient; and the particular compound or salt thereof employed. An ordinarily skilled physician or veterinarian may readily determine and prescribe the effective amount of the drug required to prevent, counter, or arrest the progress of the condition.

### EXAMPLES

Hereby are provided non-limiting examples of embodiments of compounds disclosed herein. If there is any discrepancy between a compound's depicted chemical structure and its chemical name, the depicted chemical structure will control.

TABLE 1
Examples

| Com- | | |
|---|---|---|
| pound | | |
| # | Structure | Name |
| 1 | 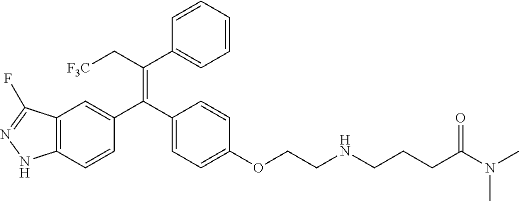 [SMILES: CN(C)C(=O)CCCNCCOc1ccc(/C(=C(/CC(F)(F)F)c2ccccc2)c2ccc3[nH]nc(F)c3c2)cc1] | N,N-dimethyl-4-[(2-[4- [(1E)-4,4,4-trifluoro-1- (3-fluoro-1H-indazol-5- yl)-1-phenylbut-1-en-2- yl]phenoxy]ethyl)amino] butanamide |
| 2 | 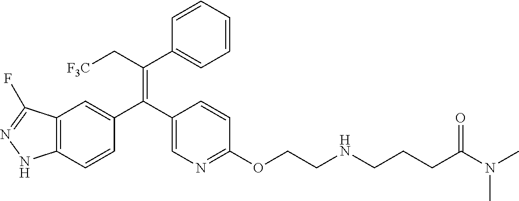 [SMILES: CN(C)C(=O)CCCNCCOc1ccc(/C(=C(/CC(F)(F)F)c2ccccc2)c2ccc3[nH]nc(F)c3c2)cn1] | (Z)-N,N-dimethyl-4-((2- ((5-(4,4,4-trifluoro-1-(3- fluoro-1H-indazol-5- yl)-2-phenylbut-1-en-1- yl)pyridin-2- yl)oxy)ethyl)amino) butanamide |
| 3 | 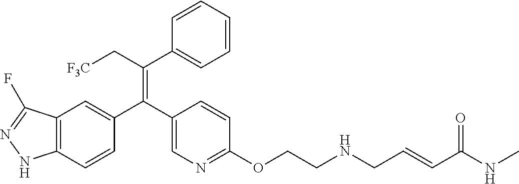 [SMILES: CNC(=O)/C=C/CNCCOc1ccc(/C(=C(/CC(F)(F)F)c2ccccc2)c2ccc3[nH]nc(F)c3c2)cn1] | (N,N-methyl-4-(2-(5- ((Z)-4,4,4-trifluoro-1- (3-fluoro-1H-indazol-5- yl)-2-phenylbut-1- enyl)pyridin-2- yloxy)ethylamino)but- 2-enamide |
| 4 | 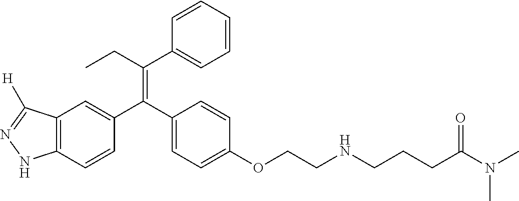 [SMILES: CC/C(=C(/c1ccc(OCCNCCCC(=O)N(C)C)cc1)c1ccc2[nH]ncc2c1)c1ccccc1] | (E)-4-((2-(4-(1H- indazol-5-yl)-2- phenylbut-1-en-1- yl)phenoxy)ethyl)amino)- N,N- dimethylbutanamide |
| 5 | 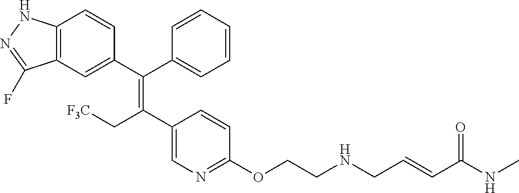 [SMILES: CNC(=O)/C=C/CNCCOc1ccc(/C(CC(F)(F)F)=C(\c2ccccc2)c2ccc3[nH]nc(F)c3c2)cn1] | (E)-N-methyl-4-((2-((5- ((E)-4,4,4-trifluoro-1- (3-fluoro-1H-indazol-5- yl)-1-phenylbut-1-en-2- yl)pyridin-2- yl)oxy)ethyl)amino)but- 2-enamide |
| 6 | 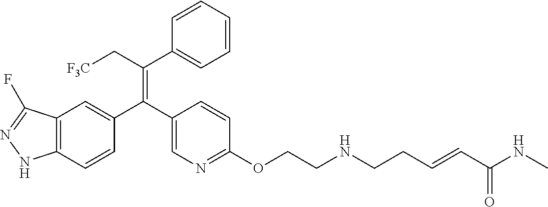 [SMILES: CNC(=O)/C=C/CCNCCOc1ccc(/C(=C(/CC(F)(F)F)c2ccccc2)c2ccc3[nH]nc(F)c3c2)cn1] | (E)-N-methyl-5-((2-((5- ((Z)-4,4,4-trifluoro-1- (3-fluoro-1H-indazol-5- yl)-2-phenylbut-1-en-1- yl)pyridin-2- yl)oxy)ethyl)amino) pent-2-enamide |
| 7 | 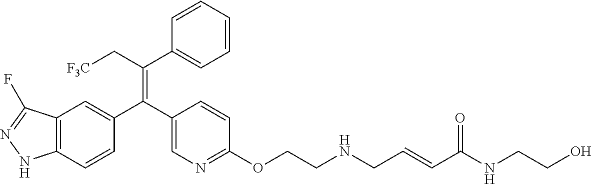 [SMILES: O=C(/C=C/CNCCOc1ccc(/C(=C(/CC(F)(F)F)c2ccccc2)c2ccc3[nH]nc(F)c3c2)cn1)NCCO] | (E)-N-(2-hydroxyethyl)- 4-((2-((5-((Z)-4,4,4- trifluoro-1-(3-fluoro- 1H-indazol-5-yl)-2- phenylbut-1-en-1- yl)pyridin-2- yl)oxy)ethyl)amino) but-2-enamide |
| 8 | 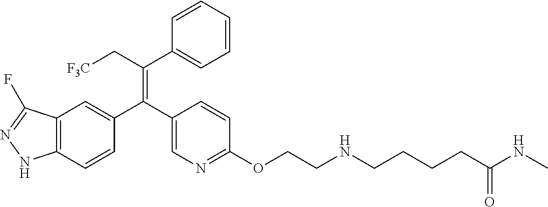 [SMILES: CNC(=O)CCCCNCCOc1ccc(/C(=C(/CC(F)(F)F)c2ccccc2)c2ccc3[nH]nc(F)c3c2)cn1] | (Z)-N-methyl-5-((2-((5- (4,4,4-trifluoro-1-(3- fluoro-1H-indazol-5- yl)-2-phenylbut-1-en-1- yl)pyridin-2- yl)oxy)ethyl)amino) pentanamide |
| 9 | 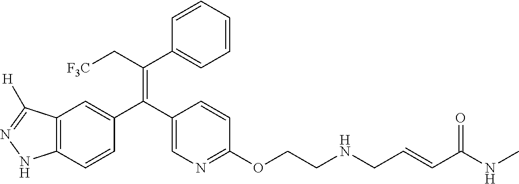 [SMILES: CNC(=O)/C=C/CNCCOc1ccc(/C(=C(/CC(F)(F)F)c2ccccc2)c2ccc3[nH]ncc3c2)cn1] | (E)-N-methyl-4-((2-((5- ((Z)-4,4,4-trifluoro-1- (1H-indazol-5-yl)-2- phenylbut-1-en-1- yl)pyridin-2- yl)oxy)ethyl)amino)but- 2-enamide |
| 10 | 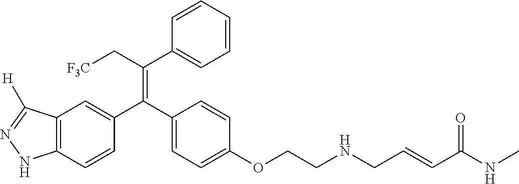 [SMILES: CNC(=O)/C=C/CNCCOc1ccc(/C(=C(/CC(F)(F)F)c2ccccc2)c2ccc3[nH]ncc3c2)cc1] | (E)-N-methyl-4-((2-(4- ((E)-4,4,4-trifluoro-1- (1H-indazol-5-yl)-2- phenylbut-1-en-1- yl)phenoxy)ethyl)amino) but-2-enamide |
| 11 | 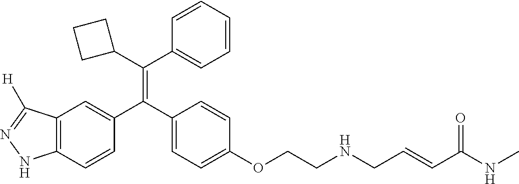 [SMILES: CNC(=O)/C=C/CNCCOc1ccc(/C(=C(\c2ccccc2)C2CCC2)c2ccc3[nH]ncc3c2)cc1] | (E)-4-((2-(4-((E)-2- cyclobutyl-1-(1H- indazol-5-yl)-2- phenylvinyl)phenoxy) ethyl)amino)-N- methylbut-2-enamide |
| 12 | 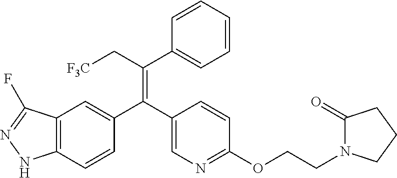 [SMILES: O=C1CCCN1CCOc1ccc(/C(=C(/CC(F)(F)F)c2ccccc2)c2ccc3[nH]nc(F)c3c2)cn1] | (Z)-1-(2-((5-(4,4,4- trifluoro-1-(3-fluoro- 1H-indazol-5-yl)-2- phenylbut-1-en-1- yl)pyridin-2- yl)oxy)ethyl)pyrrolidin- 2-one |
| 13 | 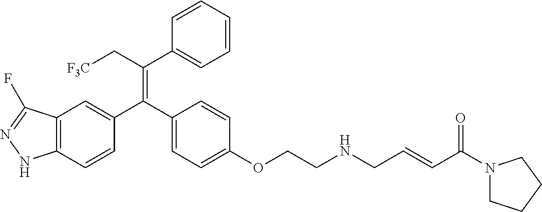 [SMILES: O=C(/C=C/CNCCOc1ccc(/C(=C(/CC(F)(F)F)c2ccccc2)c2ccc3[nH]nc(F)c3c2)cc1)N1CCCC1] | (E)-1-(pyrrolidin-1-yl)- 4-((2-(4-((E)-4,4,4- trifluoro-1-(3-fluoro- 1H-indazol-5-yl)-2- phenylbut-1-en-1- yl)phenoxy)ethyl)amino) but-2-en-1-one |
| 14 | 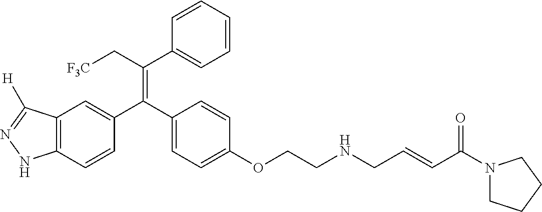 [SMILES: O=C(/C=C/CNCCOc1ccc(/C(=C(/CC(F)(F)F)c2ccccc2)c2ccc3[nH]ncc3c2)cc1)N1CCCC1] | (E)-1-(pyrrolidin-1-yl)- 4-((2-(4-((E)-4,4,4- trifluoro-1-(1H-indazol- 5-yl)-2-phenylbut-1-en- 1- yl)phenoxy)ethyl)amino) but-2-en-1-one |
| 15 | 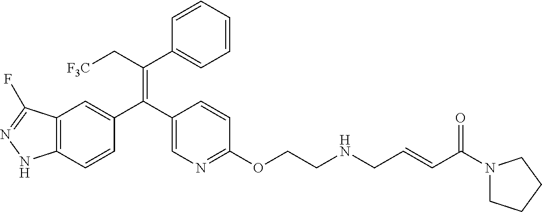 [SMILES: O=C(/C=C/CNCCOc1ccc(/C(=C(/CC(F)(F)F)c2ccccc2)c2ccc3[nH]nc(F)c3c2)cn1)N1CCCC1] | (E)-1-(pyrrolidin-1-yl)- 4-((2-((5-((Z)-4,4,4- trifluoro-1-(3-fluoro- 1H-indazol-5-yl)-2- phenylbut-1-en-1- yl)pyridin-2- yl)oxy)ethyl)amino)but- 2-en-1-one |
| 16 | 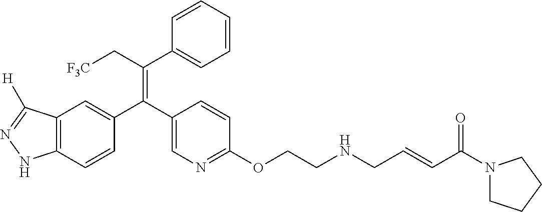 [SMILES: O=C(/C=C/CNCCOc1ccc(/C(=C(/CC(F)(F)F)c2ccccc2)c2ccc3[nH]ncc3c2)cn1)N1CCCC1] | (E)-1-(pyrrolidin-1-yl)- 4-((2-((5-((Z)-4,4,4- trifluoro-1-(1H-indazol- 5-yl)-2-phenylbut-1-en- 1-yl)pyridin-2- yl)oxy)ethyl)amino)but- 2-en-1-one |
| 17 | 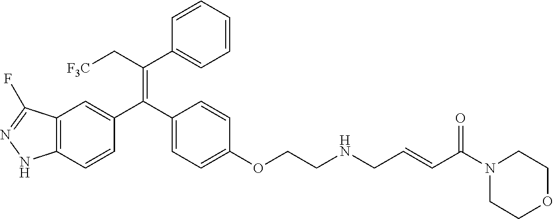 [SMILES: O=C(/C=C/CNCCOc1ccc(/C(=C(/CC(F)(F)F)c2ccccc2)c2ccc3[nH]nc(F)c3c2)cc1)N1CCOCC1] | (E)-1-morpholino-4-((2- (4-((E)-4,4,4-trifluoro- 1-(3-fluoro-1H-indazol- 5-yl)-2-phenylbut-1-en- 1- yl)phenoxy)ethyl) amino)but-2-en-1-one |
| 18 | 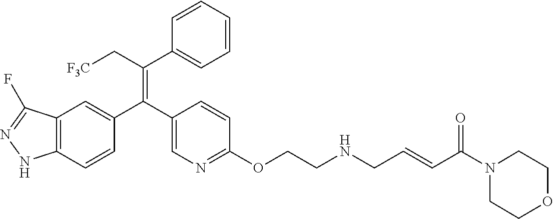 [SMILES: O=C(/C=C/CNCCOc1ccc(/C(=C(/CC(F)(F)F)c2ccccc2)c2ccc3[nH]nc(F)c3c2)cn1)N1CCOCC1] | (E)-1-morpholino-4-((2- ((5-((Z)-4,4,4-trifluoro- 1-(3-fluoro-1H-indazol- 5-yl)-2-phenylbut-1-en- 1-yl)pyridin-2- yl)oxy)ethyl)amino)but- 2-en-1-one |
| 19 | 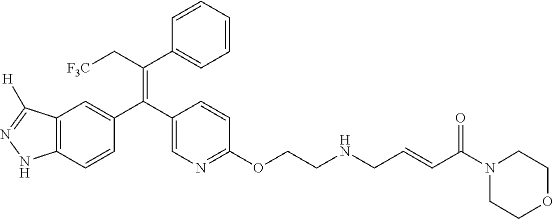 [SMILES: O=C(/C=C/CNCCOc1ccc(/C(=C(/CC(F)(F)F)c2ccccc2)c2ccc3[nH]ncc3c2)cn1)N1CCOCC1] | (E)-1-morpholino-4-((2- ((5-((Z)-4,4,4-trifluoro- 1-(1H-indazol-5-yl)-2- phenylbut-1-en-1- yl)pyridin-2- yl)oxy)ethyl)amino)but- 2-en-1-one |
| 20 | 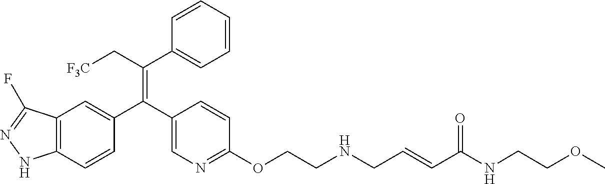 [SMILES: COCCNC(=O)/C=C/CNCCOc1ccc(/C(=C(/CC(F)(F)F)c2ccccc2)c2ccc3[nH]nc(F)c3c2)cn1] | (E)-N-(2- methoxyethyl)-4-((2- ((5-((Z)-4,4,4-trifluoro- 1-(3-fluoro-1H-indazol- 5-yl)-2-phenylbut-1-en- 1-yl)pyridin-2- yl)oxy)ethyl)amino)but- 2-enamide |
| 21 | 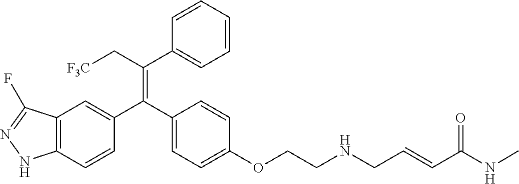 [SMILES: CNC(=O)/C=C/CNCCOc1ccc(/C(=C(/CC(F)(F)F)c2ccccc2)c2ccc3[nH]nc(F)c3c2)cc1] | (E)-N-methyl-4-((2-(4- ((E)-4,4,4-trifluoro-1- (3-fluoro-1H-indazol-5- yl)-2-phenylbut-1-en-1- yl)phenoxy)ethyl)amino) but-2-enamide |
| 22 | 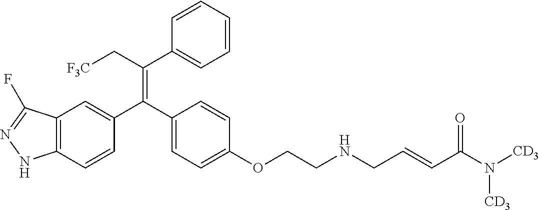 [SMILES: [2H]C([2H])([2H])N(C(=O)/C=C/CNCCOc1ccc(/C(=C(/CC(F)(F)F)c2ccccc2)c2ccc3[nH]nc(F)c3c2)cc1)C([2H])([2H])[2H]] | (E)-N,N-di(²H₃)methyl- 4-((2-(4-((E)-4,4,4- trifluoro-1-(3-fluoro- 1H-indazol-5-yl)-2- phenylbut-1-en-1- yl)phenoxy)ethyl)amino) but-2-enamide |
| 23 | 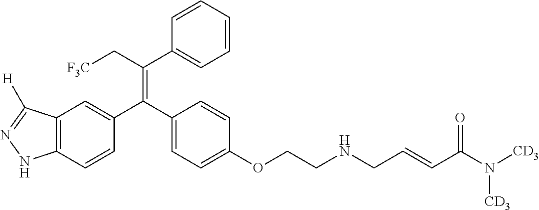 [SMILES: [2H]C([2H])([2H])N(C(=O)/C=C/CNCCOc1ccc(/C(=C(/CC(F)(F)F)c2ccccc2)c2ccc3[nH]ncc3c2)cc1)C([2H])([2H])[2H]] | (E)-N,N-di(²H₃)methyl- 4-((2-(4-((E)-4,4,4- trifluoro-1-(1H-indazol- 5-yl)-2-phenylbut-1-en- 1- yl)phenoxy)ethyl)amino) but-2-enamide |
| 24 | 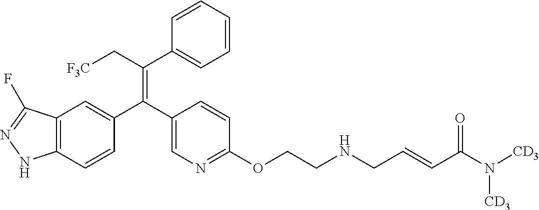 [SMILES: [2H]C([2H])([2H])N(C(=O)/C=C/CNCCOc1ccc(/C(=C(/CC(F)(F)F)c2ccccc2)c2ccc3[nH]nc(F)c3c2)cn1)C([2H])([2H])[2H]] | (E)-N,N-di(²H₃)methyl- 4-((2-((5-((Z)-4,4,4- trifluoro-1-(3-fluoro- 1H-indazol-5-yl)-2- phenylbut-1-en-1- yl)pyridin-2- yl)oxy)ethyl)amino)but- 2-enamide |
| 25 | 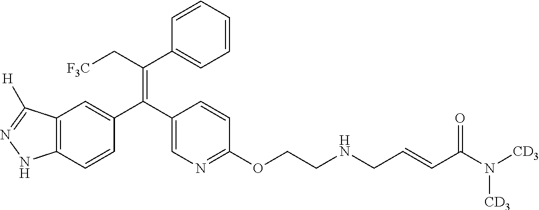 [SMILES: [2H]C([2H])([2H])N(C(=O)/C=C/CNCCOc1ccc(/C(=C(/CC(F)(F)F)c2ccccc2)c2ccc3[nH]ncc3c2)cn1)C([2H])([2H])[2H]] | (E)-N,N-di(²H₃)methyl- 4-((2-((5-((Z)-4,4,4- trifluoro-1-(1H-indazol- 5-yl)-2-phenylbut-1-en- 1-yl)pyridin-2- yl)oxy)ethyl)amino)but- 2-enamide |
| 26 | 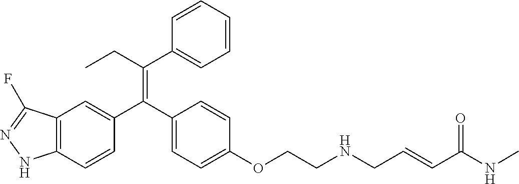 [SMILES: CC/C(=C(/c1ccc(OCCNC/C=C/C(=O)NC)cc1)c1ccc2[nH]nc(F)c2c1)c1ccccc1] | (E)-4-((2-(4-((E)-1-(3- fluoro-1H-indazol-5- yl)-2-phenylbut-1-en-1- yl)phenoxy)ethyl)amino)- N-methylbut-2- enamide |
| 27 | 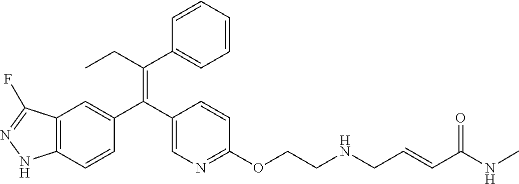 [SMILES: CC/C(=C(/c1ccc(OCCNC/C=C/C(=O)NC)nc1)c1ccc2[nH]nc(F)c2c1)c1ccccc1] | (E)-4-((2-((5-((Z)-1-(3- fluoro-1H-indazol-5- yl)-2-phenylbut-1-en-1- yl)pyridin-2- yl)oxy)ethyl)amino)-N- methylbut-2-enamide |
| 28 | 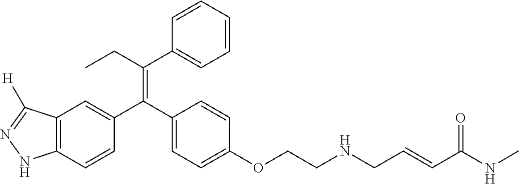 [SMILES: CC/C(=C(/c1ccc(OCCNC/C=C/C(=O)NC)cc1)c1ccc2[nH]ncc2c1)c1ccccc1] | (E)-4-((2-((5-((Z)-1- (1H-indazol-5-yl)-2- phenylbut-1-en-1- yl)pyridin-2- yl)oxy)ethyl)amino)-N- methylbut-2-enamide |
| 29 | 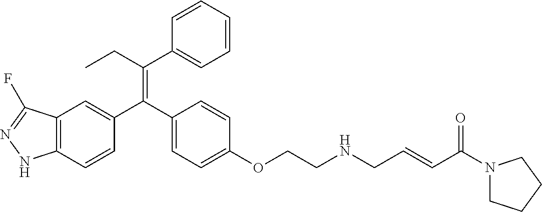 [SMILES: CC/C(=C(/c1ccc(OCCNC/C=C/C(=O)N2CCCC2)cc1)c1ccc2[nH]nc(F)c2c1)c1ccccc1] | (E)-4-((2-(4-((E)-1-(3- fluoro-1H-indazol-5- yl)-2-phenylbut-1-en-1- yl)phenoxy)ethyl)amino)- 1-(pyrrolidin-1-yl)but- 2-en-1-one |
| 30 | 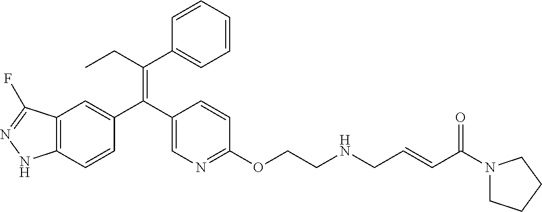 [SMILES: CC/C(=C(/c1ccc(OCCNC/C=C/C(=O)N2CCCC2)nc1)c1ccc2[nH]nc(F)c2c1)c1ccccc1] | (E)-4-((2-((5-((Z)-1-(3- fluoro-1H-indazol-5- yl)-2-phenylbut-1-en-1- yl)pyridin-2- yl)oxy)ethyl)amino)-1- (pyrrolidin-1-yl)but-2- en-1-one |
| 31 | 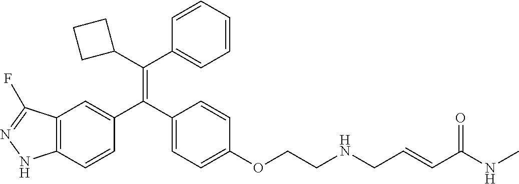 [SMILES: CNC(=O)/C=C/CNCCOc1ccc(/C(=C(\c2ccccc2)C2CCC2)c2ccc3[nH]nc(F)c3c2)cc1] | (E)-4-((2-(4-((E)-2- cyclobutyl-1-(3-fluoro- 1H-indazol-5-yl)-2- phenylvinyl)phenoxy) ethyl)amino)-N- methylbut-2-enamide |
| 32 | 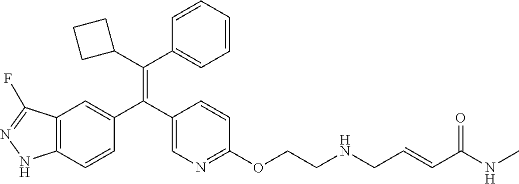 [SMILES: CNC(=O)/C=C/CNCCOc1ccc(/C(=C(\c2ccccc2)C2CCC2)c2ccc3[nH]nc(F)c3c2)cn1] | (E)-4-((2-((5-((Z)-2- cyclobutyl-1-(3-fluoro- 1H-indazol-5-yl)-2- phenylvinyl)pyridin-2- yl)oxy)ethyl)amino)-N- methylbut-2-enamide |
| 33 | 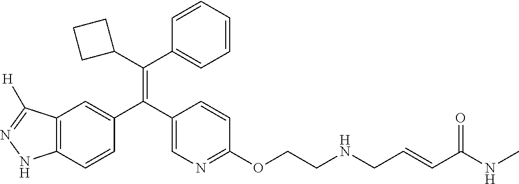 [SMILES: CNC(=O)/C=C/CNCCOc1ccc(/C(=C(\c2ccccc2)C2CCC2)c2ccc3[nH]ncc3c2)cn1] | (E)-4-((2-((5-((Z)-2- cyclobutyl-1-(1H- indazol-5-yl)-2- phenylvinyl)pyridin-2- yl)oxy)ethyl)amino)-N- methylbut-2-enamide |
| 34 | 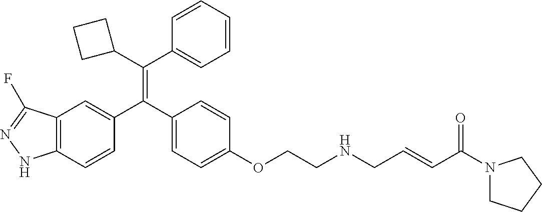 [SMILES: O=C(/C=C/CNCCOc1ccc(/C(=C(\c2ccccc2)C2CCC2)c2ccc3[nH]nc(F)c3c2)cc1)N1CCCC1] | (E)-4-((2-(4-((E)-2- cyclobutyl-1-(3-fluoro- 1H-indazol-5-yl)-2- phenylvinyl)phenoxy) ethyl)amino)-1- (pyrrolidin-1-yl)but-2- en-1-one |
| 35 | 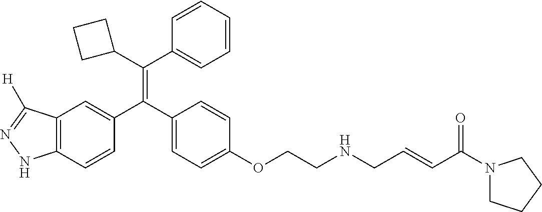 [SMILES: O=C(/C=C/CNCCOc1ccc(/C(=C(\c2ccccc2)C2CCC2)c2ccc3[nH]ncc3c2)cc1)N1CCCC1] | (E)-4-((2-(4-((E)-2- cyclobutyl-1-(1H- indazol-5-yl)-2- phenylvinyl)phenoxy) ethyl)amino)-1- (pyrrolidin-1-yl)but-2- en-1-one |
| 36 | 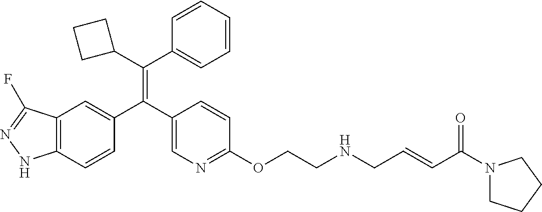 [SMILES: O=C(/C=C/CNCCOc1ccc(/C(=C(\c2ccccc2)C2CCC2)c2ccc3[nH]nc(F)c3c2)cn1)N1CCCC1] | (E)-4-((2-((5-((Z)-2- cyclobutyl-1-(3-fluoro- 1H-indazol-5-yl)-2- phenylvinyl)pyridin-2- yi)oxy )ethy3)amino)-1- (pyrrolidin-1-yl)but-2- en-1-one |
| 37 | 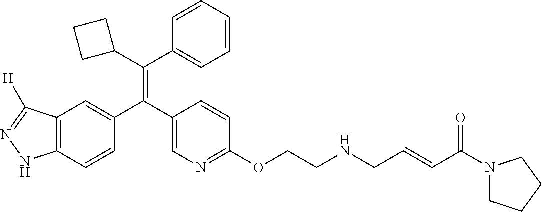 [SMILES: O=C(/C=C/CNCCOc1ccc(/C(=C(\c2ccccc2)C2CCC2)c2ccc3[nH]ncc3c2)cn1)N1CCCC1] | (E)-4-((2-((5-((2)-2- cyclobutyl-1-(1H- indazol-5-yl)-2- phenylvinyl)pyridin-2- yl)oxy)ethyl)amino)-1- (pyrrolidin-1-yl)but-2- en-1-one |
| 38 | 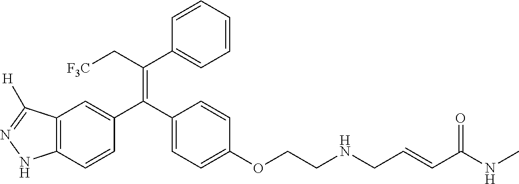 [SMILES: CNC(=O)/C=C/CNCCOc1ccc(/C(=C(/CC(F)(F)F)c2ccccc2)c2ccc3[nH]ncc3c2)cc1] | (E)-N-methyl-4-((2-(4- (4,4,4-trifluoro-1-(1H- indazol-5-yl)-2- phenylbut-1-en-1- yl)phenoxy)ethyl) amino)butanamide |
| 39 | 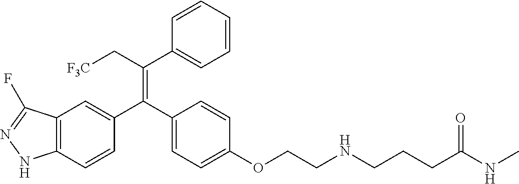 [SMILES: CNC(=O)CCCNCCOc1ccc(/C(=C(/CC(F)(F)F)c2ccccc2)c2ccc3[nH]nc(F)c3c2)cc1] | (E)-N-methyl-4-((2-(4- (4,4,4-trifluoro-1-(3- fluoro-1H-indazol-5- yl)-2-phenylbut-1-en-1- yl)phenoxy)ethyl) amino)butanamide |
| 40 | 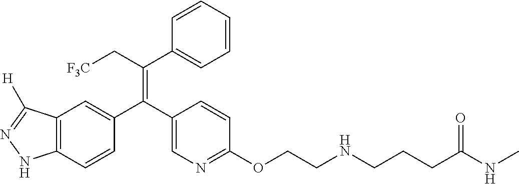 [SMILES: CNC(=O)CCCNCCOc1ccc(/C(=C(/CC(F)(F)F)c2ccccc2)c2ccc3[nH]ncc3c2)cn1] | (Z)-N-methyl-4-((2-((5- (4,4,4-trifluoro-1-(1H- indazol-5-yl)-2- phenylbut-1-en-1- yl)pyridin-2- yl)oxy)ethyl)amino) butanamide |
| 41 | 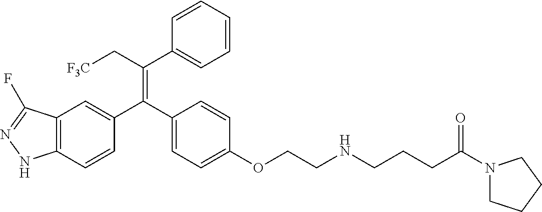 [SMILES: O=C(CCCNCCOc1ccc(/C(=C(/CC(F)(F)F)c2ccccc2)c2ccc3[nH]nc(F)c3c2)cc1)N1CCCC1] | (E)-1-(pyrrolidin-1-yl)- 4-((2-(4-(4,4,4-trifluoro- 1-(3-fluoro-1H-indazol- 5-yl)-2-phenylbut-1-en- 1- yl)phenoxy)ethyl) amino)butan-1-one |
| 42 | 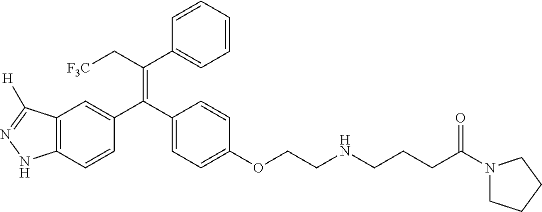 [SMILES: O=C(CCCNCCOc1ccc(/C(=C(/CC(F)(F)F)c2ccccc2)c2ccc3[nH]ncc3c2)cc1)N1CCCC1] | (E)-1-(pyrrolidin-1-yl)- 4-((2-(4-(4,4,4-trifluoro- 1-(1H-indazol-5-yl)-2- phenylbut-1-en-1- yl)phenoxy)ethyl) amino)butan-1-one |
| 43 | 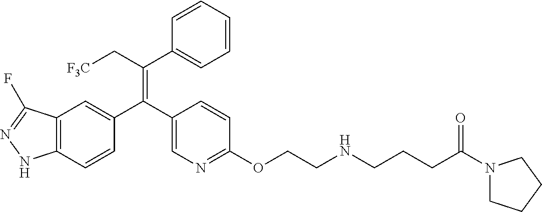 [SMILES: O=C(CCCNCCOc1ccc(/C(=C(/CC(F)(F)F)c2ccccc2)c2ccc3[nH]nc(F)c3c2)cn1)N1CCCC1] | (Z)-1-(pyrrolidin-1-yl)- 4-((2-((5-(4,4,4- trifluoro-1-(3-fluoro- 1H-indazol-5-yl)-2- phenylbut-1-en-1- yl)pyridin-2- yl)oxy)ethyl)amino) butan-1-one |
| 44 | 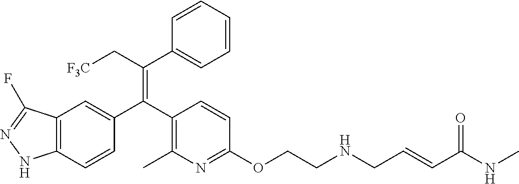 [SMILES: CNC(=O)/C=C/CNCCOc1ccc(/C(=C(/CC(F)(F)F)c2ccccc2)c2ccc3[nH]nc(F)c3c2)c(C)n1] | (E)-N-methyl-4-((2-((6- methyl-5-((Z)-4,4,4- trifluoro-1-(3-fluoro- 1H-indazol-5-yl)-2- phenylbut-1-en-1- yl)pyridin-2- yl)oxy)ethyl)amino)but- 2-enamide |
| 45 | 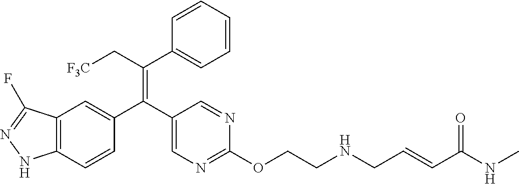 [SMILES: CNC(=O)/C=C/CNCCOc1ncc(/C(=C(/CC(F)(F)F)c2ccccc2)c2ccc3[nH]nc(F)c3c2)cn1] | (E)-N-methyl-4-((2-((5- ((Z)-4,4,4-trifluoro-1- (3-fluoro-1H-indazol-5- yl)-2-phenylbut-1-en-1- yl)pyrimidin-2- yl)oxy)ethyl)amino)but- 2-enamide |
| 46 | 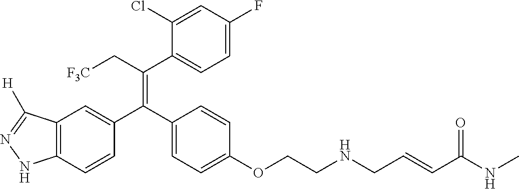 [SMILES: CNC(=O)/C=C/CNCCOc1ccc(/C(=C(/CC(F)(F)F)c2ccc(F)cc2Cl)c2ccc3[nH]ncc3c2)cc1] | (E)-4-((2-(4-((E)-2-(2- chloro-4-fluorophenyl)- 4,4,4-trifluoro-1-(1H- indazol-5-yl)but-1-en-1- yl)phenoxy)ethyl) amino)-N-methylbut-2- enamide |
| 47 | 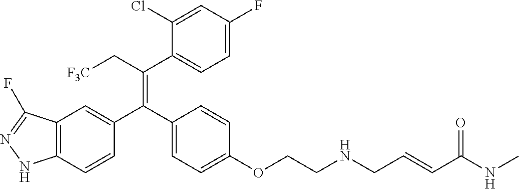 [SMILES: CNC(=O)/C=C/CNCCOc1ccc(/C(=C(/CC(F)(F)F)c2ccc(F)cc2Cl)c2ccc3[nH]nc(F)c3c2)cc1] | (E)-4-((2-(4-((E)-2-(2- chloro-4-fluorophenyl)- 4,4,4-trifluoro-1-(3- fluoro-1H-indazol-5- yl)but-1-en-1- yl)phenoxy)ethyl) amino)-N-methylbut-2- enamide |
| 48 | 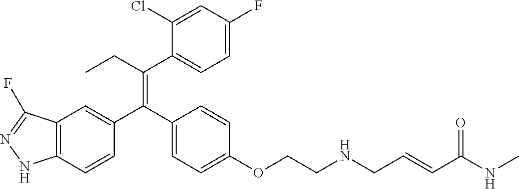 [SMILES: CC/C(=C(/c1ccc(OCCNC/C=C/C(=O)NC)cc1)c1ccc2[nH]nc(F)c2c1)c1ccc(F)cc1Cl] | (E)-4-((2-(4-((E)-2-(2- chloro-4-fluorophenyl)- 1-(3-fluoro-1H-indazol- 5-yl)but-1-en-1- yl)phenoxy)ethyl) amino)-N-methylbut-2- enamide |
| 49 | 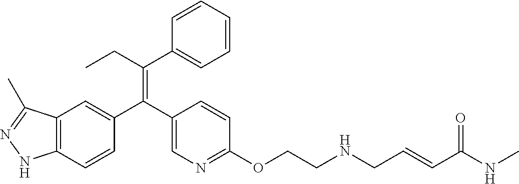 [SMILES: CC/C(=C(/c1ccc(OCCNC/C=C/C(=O)NC)nc1)c1ccc2[nH]nc(C)c2c1)c1ccccc1] | (E)-N-methyl-4-((2-((5- ((Z)-1-(3-methyl-1H- indazol-5-yl)-2- phenylbut-1-en-1- yl)pyridin-2- yl)oxy)ethyl)amino)but- 2-enamide |
| 50 | 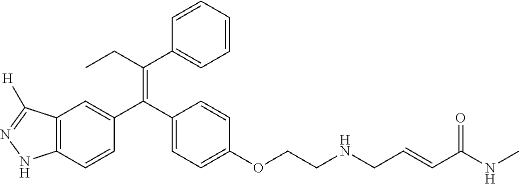 [SMILES: CC/C(=C(/c1ccc(OCCNC/C=C/C(=O)NC)cc1)c1ccc2[nH]ncc2c1)c1ccccc1] | (E)-4-((2-(4-((E)-1-(1H- indazol-5-yl)-2- phenylbut-1-en-1- yl)phenoxy)ethyl) amino)-N-methylbut-2- enannde |
| 51 | 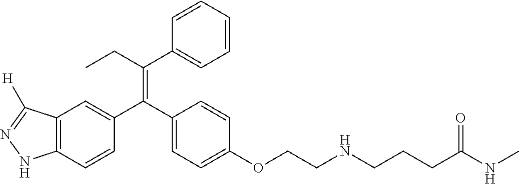 [SMILES: CC/C(=C(/c1ccc(OCCNCCCC(=O)NC)cc1)c1ccc2[nH]ncc2c1)c1ccccc1] | (E)-4-((2-(4-(1-(1H- indazol-5-yl)-2- phenylbut-1-en-1- yl)phenoxy)ethyl)amino)- N-methylbutanamide |
| 52 | 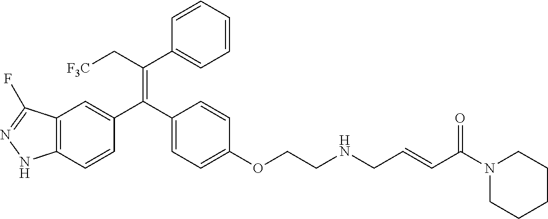 [SMILES: O=C(/C=C/CNCCOc1ccc(/C(=C(/CC(F)(F)F)c2ccccc2)c2ccc3[nH]nc(F)c3c2)cc1)N1CCCCC1] | (E)-1-(piperidin-1-yl)-4- ((2-(4-((E)-4,4,4- trifluoro-1-(3-fluoro- 1H-indazol-5-yl)-2- phenylbut-1-en-1- yl)phenoxy)ethyl) amino)but-2-en-1-one |
| 53 | 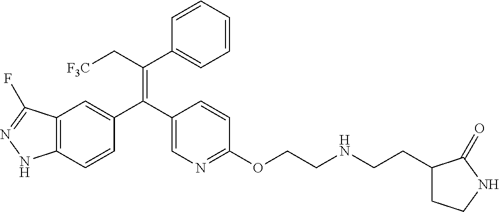 [SMILES: O=C1NCCC1CCNCCOc1ccc(/C(=C(/CC(F)(F)F)c2ccccc2)c2ccc3[nH]nc(F)c3c2)cn1] | (Z)-3-(2-((2-((5-(4,4,4- trifluoro-1-(3-fluoro- 1H-indazol-5-yl)-2- phenylbut-1-en-1- yl)pyridin-2- yl)oxy)ethyl)amino) ethyl)pyrrolidin-2-one |
| 54 | 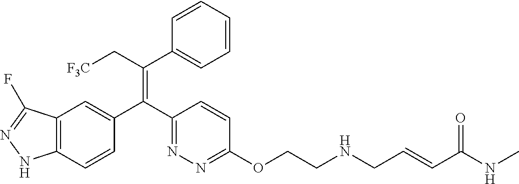 [SMILES: CNC(=O)/C=C/CNCCOc1ccc(/C(=C(/CC(F)(F)F)c2ccccc2)c2ccc3[nH]nc(F)c3c2)nn1] | (E)-N-methyl-4-((2-((6- ((Z)-4,4,4-trifluoro-1- (3-fluoro-1H-indazol-5- yl)-2-phenylbut-1-en-1- yl)pyridazin-3- yl)oxy)ethyl)amino)but- 2-enamide |
| 55 | 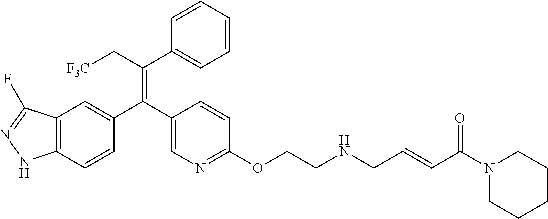 [SMILES: O=C(/C=C/CNCCOc1ccc(/C(=C(/CC(F)(F)F)c2ccccc2)c2ccc3[nH]nc(F)c3c2)cn1)N1CCCCC1] | (E)-1-(piperidin-1-yl)-4- ((2-((5-((Z)-4,4,4- trifluoro-1-(3-fluoro- 1H-indazol-5-yl)-2- phenylbut-1-en-1- yl)pyridin-2- yl)oxy)ethyl)amino)but- 2-en-1-one |
| 56 | 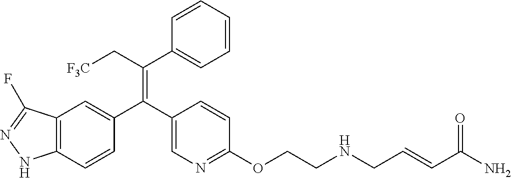 [SMILES: NC(=O)/C=C/CNCCOc1ccc(/C(=C(/CC(F)(F)F)c2ccccc2)c2ccc3[nH]nc(F)c3c2)cn1] | (E)-4-((2-((5-((Z)-4,4,4- trifluoro-1-(3-fluoro- 1H-indazol-5-yl)-2- phenylbut-1-en-1- yl)pyridin-2- yl)oxy)ethyl)amino)but- 2-enamide |
| 57 | 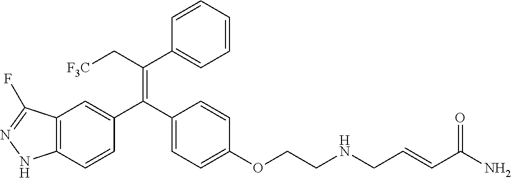 [SMILES: NC(=O)/C=C/CNCCOc1ccc(/C(=C(/CC(F)(F)F)c2ccccc2)c2ccc3[nH]nc(F)c3c2)cc1] | (E)-4-((2-(4-((E)-4,4,4- trifluoro-1-(3-fluoro- 1H-indazol-5-yl)-2- phenylbut-1-en-1- yl)phenoxy)ethyl) amino)but-2-enamide |
| 58 | 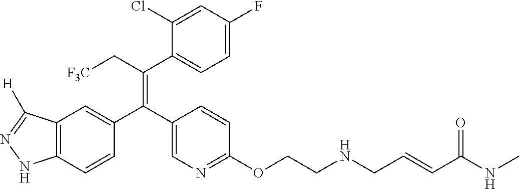 [SMILES: CNC(=O)/C=C/CNCCOc1ccc(/C(=C(/CC(F)(F)F)c2ccc(F)cc2Cl)c2ccc3[nH]ncc3c2)cn1] | (E)-4-((2-((5-((Z)-2-(2- chloro-4-fluorophenyl)- 4,4,4-trifluoro-1-(1H- indazol-5-yl)but-1-en-1- yl)pyridin-2- yl)oxy)ethyl)amino)-N- methylbut-2-enamide |
| 59 | 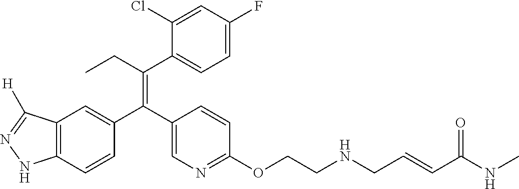 [SMILES: CC/C(=C(/c1ccc(OCCNC/C=C/C(=O)NC)nc1)c1ccc2[nH]ncc2c1)c1ccc(F)cc1Cl] | (E)-4-((2-((5-((Z)-2-(2- chloro-4-fluorophenyl)- 1-(1H-indazol-5-yl)but- 1-en-1-yl)pyridin-2- yl)oxy)ethyl)amino)-N- methylbut-2-enamide |
| 60 | 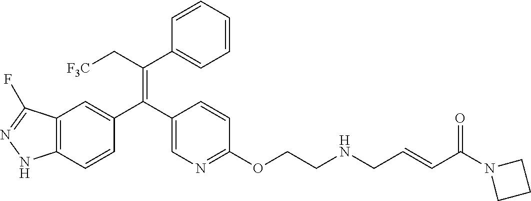 [SMILES: O=C(/C=C/CNCCOc1ccc(/C(=C(/CC(F)(F)F)c2ccccc2)c2ccc3[nH]nc(F)c3c2)cn1)N1CCC1] | (E)-1-(azetidin-1-yl)-4- ((2-((5-((Z)-4.4,4- trifluoro-1-(3-fluoro- 1H-indazol-5-yl)-2- phenylbut-1-en-1- yl)pyridin-2- yl)oxy)ethyl)amino)but- 2-en-1-one |
| 61 | 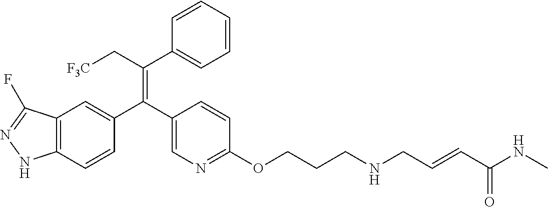 [SMILES: CNC(=O)/C=C/CNCCCOc1ccc(/C(=C(/CC(F)(F)F)c2ccccc2)c2ccc3[nH]nc(F)c3c2)cn1] | (E)-N-methyl-4-((3-((5- ((Z)-4,4,4-trifluoro-1- (3-fluoro-1H-indazol-5- yl)-2-phenylbut-1-en-1- yl)pyridin-2- yl)oxy)propyl)amino) but-2-enamide |
| 62 | 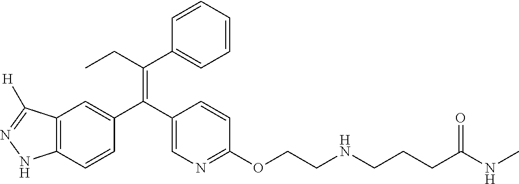 [SMILES: CC/C(=C(/c1ccc(OCCNCCCC(=O)NC)nc1)c1ccc2[nH]ncc2c1)c1ccccc1] | (Z)-4-((2-((5-(1-(1H- indazol-5-yl)-2- phenylbut-1-en-1- yl)pyridin-2- yl)oxy)ethyl)amino)-N- methylbutanamide |
| 63 | 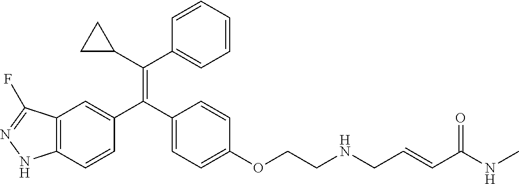 [SMILES: CNC(=O)/C=C/CNCCOc1ccc(/C(=C(\c2ccccc2)C2CC2)c2ccc3[nH]nc(F)c3c2)cc1] | (E)-4-((2-(4-((E)-2- cyclopropyl-1-(3- fluoro-1H-indazol-5- yl)-2- phenylvinyl)phenoxy) ethyl)amino)-N- methylbut-2-enamide |
| 64 | 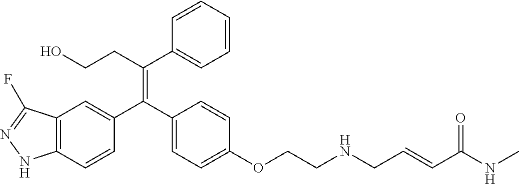 [SMILES: CNC(=O)/C=C/CNCCOc1ccc(/C(=C(/CCO)c2ccccc2)c2ccc3[nH]nc(F)c3c2)cc1] | (E)-4-((2-(4-((E)-1-(3- fluoro-1H-indazol-5- yl)-4-hydroxy-2- phenylbut-1-en-1- yl)phenoxy)ethyl) amino)-N-methylbut-2- enamide |
| 65 | 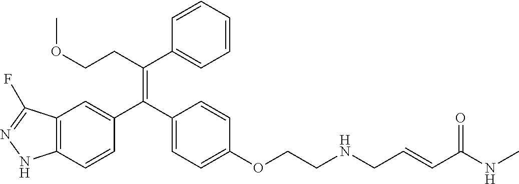 [SMILES: CNC(=O)/C=C/CNCCOc1ccc(/C(=C(/CCOC)c2ccccc2)c2ccc3[nH]nc(F)c3c2)cc1] | (E)-4-((2-(4-((E)-1-(3- fluoro-1H-indazol-5- yl)-4-methoxy-2- phenylbut-1-en-1- yl)phenoxy)ethyl) amino)-N-methylbut-2- enamide |
| 66 | 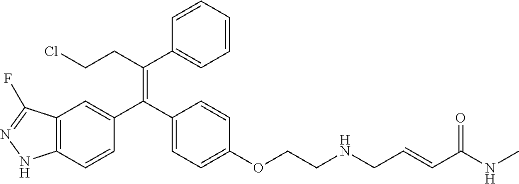 [SMILES: CNC(=O)/C=C/CNCCOc1ccc(/C(=C(/CCCl)c2ccccc2)c2ccc3[nH]nc(F)c3c2)cc1] | (E)-4-((2-(4-((E)-4- chloro-1-(3-fluoro-1H- indazol-5-yl)-2- phenylbut-1-en-1- yl)phenoxy)ethyl) amino)-N-methylbut-2- enamide |
| 67 | 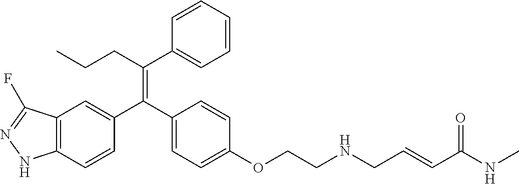 [SMILES: CCC/C(=C(/c1ccc(OCCNC/C=C/C(=O)NC)cc1)c1ccc2[nH]nc(F)c2c1)c1ccccc1] | (E)-4-((2-(4-((E)-1-(3- fluoro-1H-indazol-5- yl)-2-phenylpent-1-en- 1- yl)phenoxy)ethyl) amino)-N-methylbut-2- enamide |
| 68 | 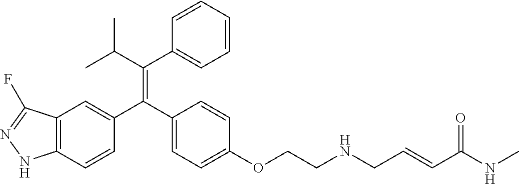 [SMILES: CNC(=O)/C=C/CNCCOc1ccc(/C(=C(\c2ccccc2)C(C)C)c2ccc3[nH]nc(F)c3c2)cc1] | (E)-4-((2-(4-((E)-1-(3- fluoro-1H-indazol-5- yl)-3-methyl-2- phenylbut-1-en-1- yl)phenoxy)ethyl) amino)-N-methylbut-2- enamide |
| 69 | 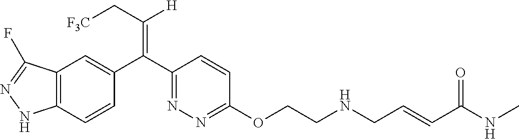 [SMILES: CNC(=O)/C=C/CNCCOc1ccc(/C(=C/CC(F)(F)F)c2ccc3[nH]nc(F)c3c2)nn1] | (E)-N-methyl-4-((2-((6- ((E)-4,4,4-trifluoro-1- (3-fluoro-1H-indazol-5- yl)but-1-en-1- yl)pyridazin-3- yl)oxy)ethyl)amino)but- 2-enamide |
| 70 | 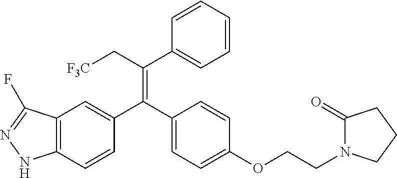 [SMILES: O=C1CCCN1CCOc1ccc(/C(=C(/CC(F)(F)F)c2ccccc2)c2ccc3[nH]nc(F)c3c2)cc1] | (E)-1-(2-(4-(4,4,4- trifluoro-1-(3-fluoro- 1H-indazol-5-yl)-2- phenylbut-1-en-1- yl)phenoxy)ethyl) pyrrolidin-2-one |
| 71 | 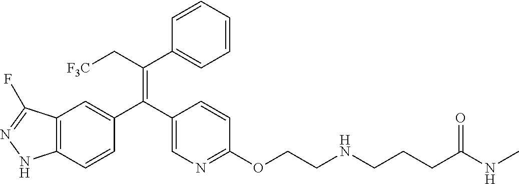 [SMILES: CNC(=O)CCCNCCOc1ccc(/C(=C(/CC(F)(F)F)c2ccccc2)c2ccc3[nH]nc(F)c3c2)cn1] | (Z)-N-methyl-4-((2-((5- (4,4,4-trifluoro-1-(3- fluoro-1H-indazol-5- yl)-2-phenylbut-1-en-1- yl)pyridin-2- yl)oxy)ethyl)amino) butanamide |
| 72 | 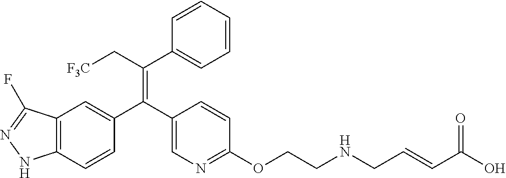 [SMILES: O=C(O)/C=C/CNCCOc1ccc(/C(=C(/CC(F)(F)F)c2ccccc2)c2ccc3[nH]nc(F)c3c2)cn1] | (E)-4-((2-((5-((Z)-4,4,4- trifluoro-1-(3-fluoro- 1H-indazol-5-yl)-2- phenylbut-1-en-1- yl)pyridin-2- yl)oxy)ethyl)amino)but- 2-enoic acid |
| 73 | 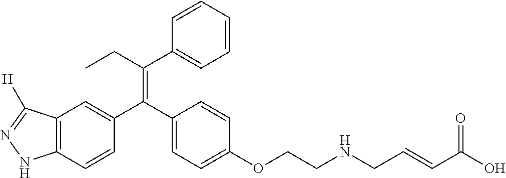 [SMILES: CC/C(=C(/c1ccc(OCCNC/C=C/C(=O)O)cc1)c1ccc2[nH]ncc2c1)c1ccccc1] | (E)-4-((2-(4-((E)-1-(1H- indazol-5-yl)-2- phenylbut-1-en-1- yl)phenoxy)ethyl) amino)but-2-enoic acid |
| 74 | 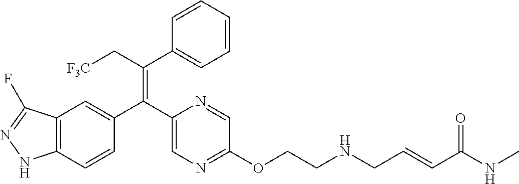 [SMILES: CNC(=O)/C=C/CNCCOc1cnc(/C(=C(/CC(F)(F)F)c2ccccc2)c2ccc3[nH]nc(F)c3c2)cn1] | (E)-N-methyl-4-((2-((5- ((Z)-4,4,4-trifluoro-1- (3-fluoro-1H-indazol-5- yl)-2-phenylbut-1-en-1- yl)pyrazin-2- yl)oxy)ethyl)amino)but- 2-enamide |
| 75 | 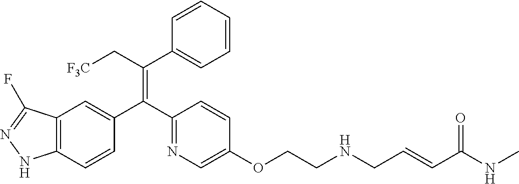 [SMILES: CNC(=O)/C=C/CNCCOc1ccc(/C(=C(/CC(F)(F)F)c2ccccc2)c2ccc3[nH]nc(F)c3c2)nc1] | (E)-N-methyl-4-((2-((6- ((Z)-4,4,4-trifluoro-1- (3-fluoro-1H-indazol-5- yl)-2-phenylbut-1-en-1- yl)pyridin-3- yl)oxy)ethyl)amino)but- 2-enamide |
| 76 | 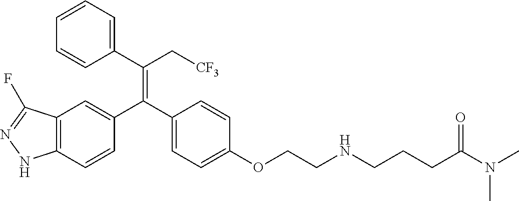 [SMILES: CN(C)C(=O)CCCNCCOc1ccc(/C(=C(\CC(F)(F)F)c2ccccc2)c2ccc3[nH]nc(F)c3c2)cc1] | (Z)-N,N-dimethyl-4-((2- (4-(4,4,4-trifluoro-1-(3- fluoro-1H-indazol-5- yl)-2-phenylbut-1-en-1- yl)phenoxy)ethyl) amino)butanamide |
| 77 | 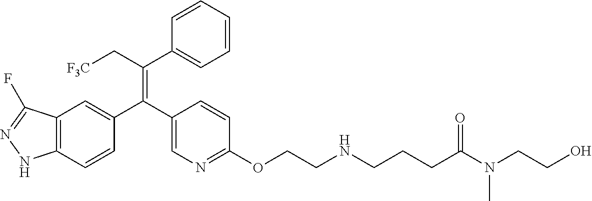 [SMILES: CN(CCO)C(=O)CCCNCCOc1ccc(/C(=C(/CC(F)(F)F)c2ccccc2)c2ccc3[nH]nc(F)c3c2)cn1] | (Z)-N-(2-hydroxyethyl)- N-methyl-4-((2-((5- (4,4,4-trifluoro-1-(3- fluoro-1H-indazol-5- yl)-2-phenylbut-1-en-1- yl)pyridin-2- yl)oxy)ethyl)amino) butanamide |
| 78 | 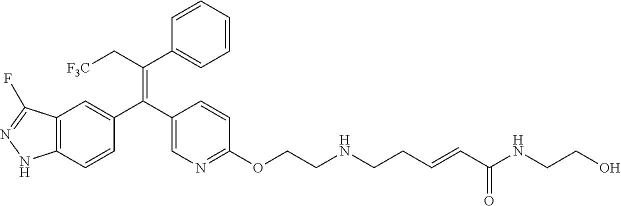 [SMILES: O=C(/C=C/CCNCCOc1ccc(/C(=C(/CC(F)(F)F)c2ccccc2)c2ccc3[nH]nc(F)c3c2)cn1)NCCO] | (E)-N-(2-hydroxuethyl)- 5-((2-((5-((Z)-4,4,4- trifluoro-1-(3-fluoro- 1H-indazol-5-yl)-2- phenylbut-1-en-1- yl)pyridin-2- yl)oxy)ethyl)amino) pent-2-enamide |
| 79 | 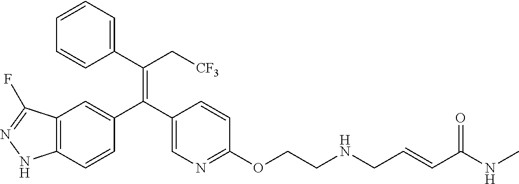 [SMILES: CNC(=O)/C=C/CNCCOc1ccc(/C(=C(\CC(F)(F)F)c2ccccc2)c2ccc3[nH]nc(F)c3c2)cn1] | (E)-N-methyl-4-((2-((5- ((E)-4,4,4-trifluoro-1- (3-fluoro-1H-indazol-5- yl)-2-phenylbut-1-en-1- yl)pyridin-2- yl)oxy)ethyl)amino)but- 2-enamide |
| 80 | 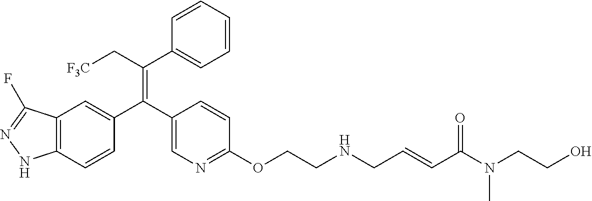 [SMILES: CN(CCO)C(=O)/C=C/CNCCOc1ccc(/C(=C(/CC(F)(F)F)c2ccccc2)c2ccc3[nH]nc(F)c3c2)cn1] | (E)-N-(2-hydroxyethyl)- N-methyl-4-((2-((5-((Z)- 4,4,4-trifluoro-1-(3- fluoro-1H-indazol-5- yl)-2-phenylbut-1-en-1- yl)pyridin-2- yl)oxy)ethyl)amino)but- 2-enamide |
| 81 | 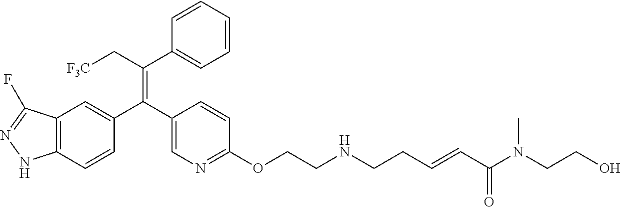 [SMILES: CN(CCO)C(=O)/C=C/CCNCCOc1ccc(/C(=C(/CC(F)(F)F)c2ccccc2)c2ccc3[nH]nc(F)c3c2)cn1] | (E)-N-(2-hydroxyethyl)- N-methyl-5-((2-((5-((Z)- 4,4,4-trifluoro-1-(3- fluoro-1H-indazol-5- yl)-2-phenylbut-1-en-1- yl)pyridin-2- yl)oxy)ethyl)amino) pent-2-enamide |
| 82 | 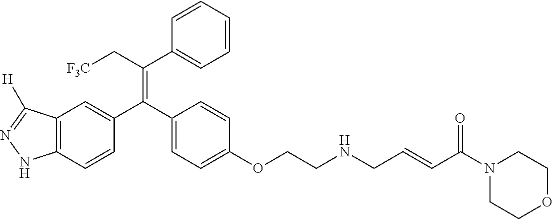 [SMILES: O=C(/C=C/CNCCOc1ccc(/C(=C(/CC(F)(F)F)c2ccccc2)c2ccc3[nH]ncc3c2)cc1)N1CCOCC1] | (E)-1-morpholino-4-((2- (4-((E)-4,4,4-trifluoro- 1-(1H-indazol-5-yl)-2- phenylbut-1-en-1- yl)phenoxy)ethyl) amino)but-2-en-1-one |
| 83 | 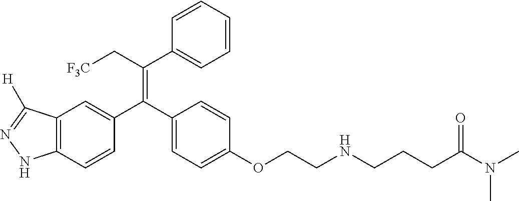 [SMILES: CN(C)C(=O)CCCNCCOc1ccc(/C(=C(/CC(F)(F)F)c2ccccc2)c2ccc3[nH]ncc3c2)cc1] | (E)-N-dimethyl-4-((2- (4-(4,4,4-trifluoro-1- (1H-indazol-5-yl)-2- phenylbut-1-en-1- yl)phenoxy)ethyl) amino)butanamide |
| 84 | 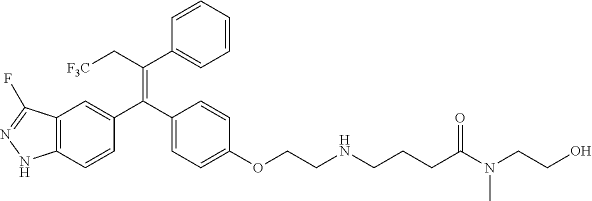 [SMILES: CN(CCO)C(=O)CCCNCCOc1ccc(/C(=C(/CC(F)(F)F)c2ccccc2)c2ccc3[nH]nc(F)c3c2)cc1] | (E)-N-(2-hydroxyethyl)- N-methyl-4-((2-(4- (4,4,4-trifluoro-1-(3- fluoro-1H-indazol-5- yl)-2-phenylbut-1-en-1- yl)phenoxy)ethyl) amino)butanamide |
| 85 | 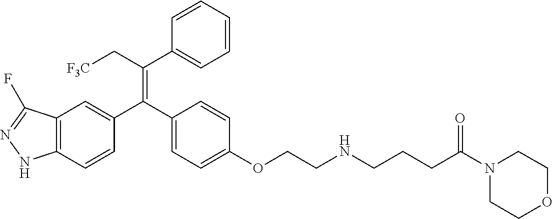 [SMILES: O=C(CCCNCCOc1ccc(/C(=C(/CC(F)(F)F)c2ccccc2)c2ccc3[nH]nc(F)c3c2)cc1)N1CCOCC1] | (E)-1-morpholino-4-((2- (4-(4,4,4-trifluoro-1-(3- fluoro-1H-indazol-5- yl)-2-phenylbut-1-en-1- yl)phenoxy)ethyl) amino)butan-1-one |
| 86 | 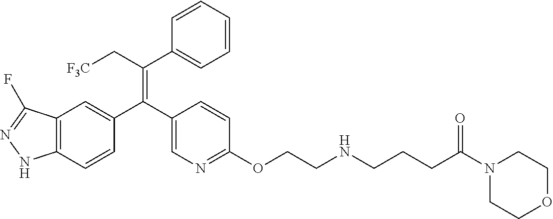 [SMILES: O=C(CCCNCCOc1ccc(/C(=C(/CC(F)(F)F)c2ccccc2)c2ccc3[nH]nc(F)c3c2)cn1)N1CCOCC1] | (Z)-1-morpholino-4-((2- ((5-(4,4,4-trifluoro-1-(3- fluoro-1H-indazol-5- yl)-2-phenylbut-1-en-1- yl)pyridin-2- yl)oxy)ethyl)amino) butan-1-one |
| 87 | 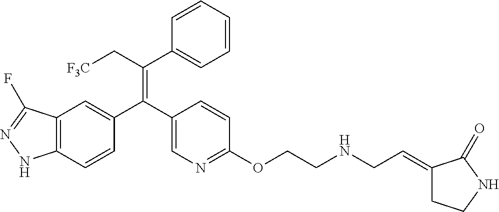 [SMILES: O=C1NCC/C1=C\CNCCOc1ccc(/C(=C(/CC(F)(F)F)c2ccccc2)c2ccc3[nH]nc(F)c3c2)cn1] | (E)-3-(2-((2-((5-((Z)- 4,4,4-trifluoro-1-(3- fluoro-1H-indazol-5- yl)-2-phenylbut-1-en-1- yl)pyridin-2- yl)oxy)ethyl)amino) ethylidene)pyrrolidin- 2-one |
| 88 | 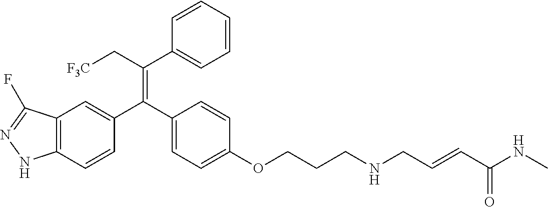 [SMILES: CNC(=O)/C=C/CNCCCOc1ccc(/C(=C(/CC(F)(F)F)c2ccccc2)c2ccc3[nH]nc(F)c3c2)cc1] | (E)-N-methyl-4-((3-((5- ((Z)-4,4,4-trifluoro-1- (3-fluoro-1H-indazol-5- yl)-2-phenylbut-1-en-1- yl)pyridin-2- yl)oxy)propyl)amino) but-2-enamide |
| 89 | 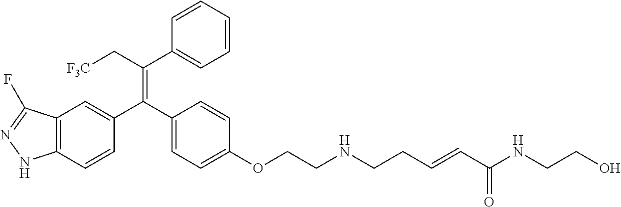 [SMILES: O=C(/C=C/CCNCCOc1ccc(/C(=C(/CC(F)(F)F)c2ccccc2)c2ccc3[nH]nc(F)c3c2)cc1)NCCO] | (E)-N-(2-hydroxyethyl)- 5-((2-(4-((E)-4,4,4- trifluoro-1-(3-fluoro- 1H-indazol-5-yl)-2- phenylbut-1-en-1- yl)phenoxy)ethyl) amino)pent-2-enamide |

### General Procedures

The following abbreviations may be used herein:
ACN: Acetonitrile
Boc: tert-butyloxycarbonyl
CAN: ceric ammonium nitrate
Conc.: concentrated
Cs₂CO₃: Cesium carbonate
DABCO: 1,4-Diazabicyclo[2.2.2]octane
DCM: Dichloromethane
DHP: Dihydropyran
DIPEA: N,N-diisopropylethylamine, Hunig's base
DMA: Dimethylacetamide
DMF: Dimethylformamide
DMSO: dimethylsulfoxide
DPEphos: (Oxydi-2,1-phenylene)bis(diphenylphosphine)
EDCI.HCl: N-(3-Dimethylaminopropyl)-N-ethylcarbodiimide hydrochloride
EtOH: Ethanol
EtOAc: Ethyl acetate
Et₃N: Triethylamine
Ex.: Example
h: Hours
HATU: 1-[Bis(dimethylamino)methylene]-1H-1,2,3-triazolo[4,5-b]pyridinium 3-oxid hexafluorophosphate
HCl: Hydrochloric acid
HMPA: Hexamethylphosphoramide
HPLC: High-performance liquid chromatography
H₂SO₄: Sulfuric acid
IPA: Isopropyl alcohol
K₂CO₃: Potassium carbonate
KOH: Potassium hydroxide
LCMS: Liquid chromatography—mass spectrometry
MeOH: Methanol
Na₂CO₃: Sodium carbonate
NBS: n-bromosuccinimide
nBuLi: n-Butyllithium
NH₄Cl: Ammonium chloride
NH₄OH: Ammonium hydroxide
NMR: nuclear magnetic resonance
on or o.n.: overnight
Pd/C: Palladium (0) on carbon
Pd₂(dba)₃: Tris(dibenzylideneacetone)dipalladium(0)
PPTS: pyridinium p-toluenesulfonate
PTSA: p-toluenesulfonic acid
R.T. or r.t.: room temperature
TBAF: Tetrabutylammonium fluoride
TEA: Triethylamine
TFA: Trifluoroacetic acid
THF: Tetrahydrofuran
TLC: Thin-layer chromatography
Pt/C: Platinum (0) on carbon

Unless indicated otherwise, ¹H NMR spectra were taken on a Bruker 300 MHz or 400 MHz NMR.

### Examples

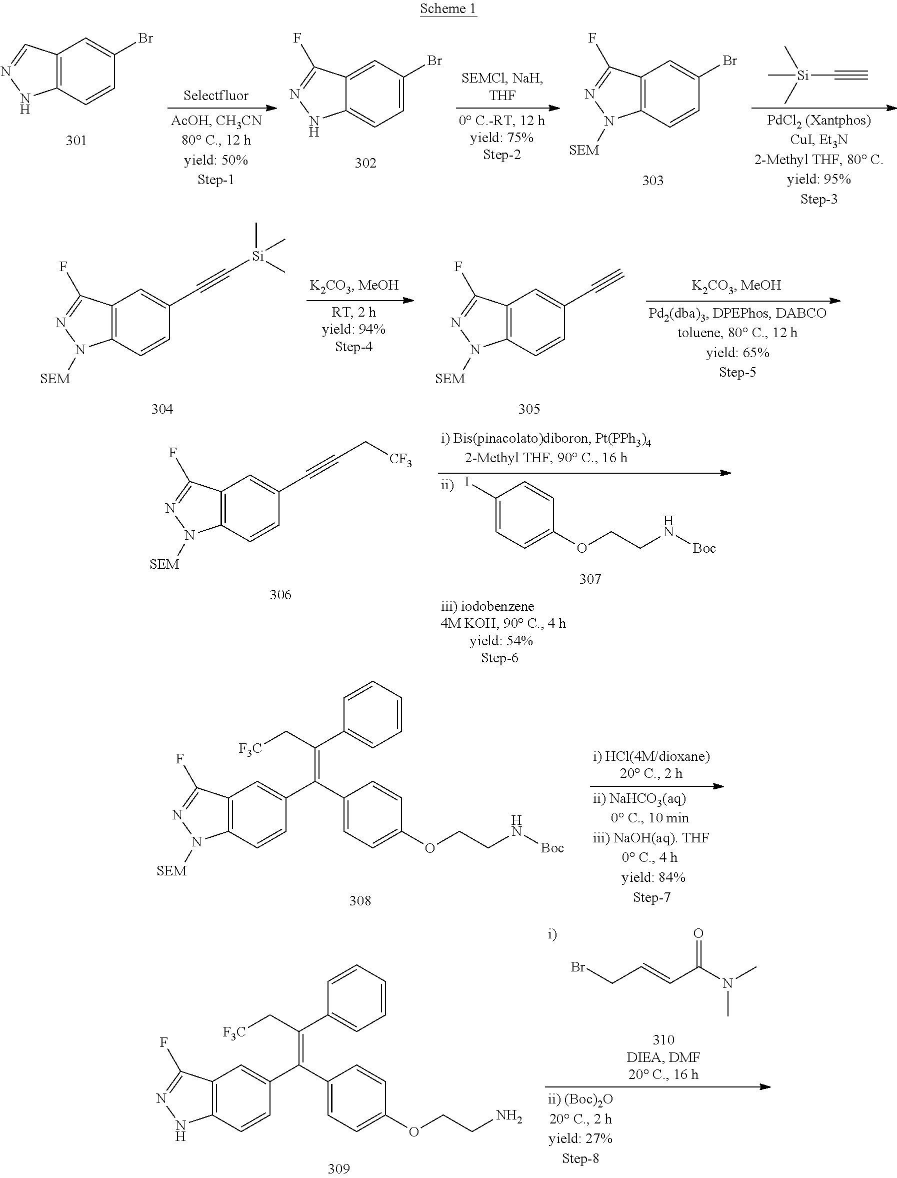

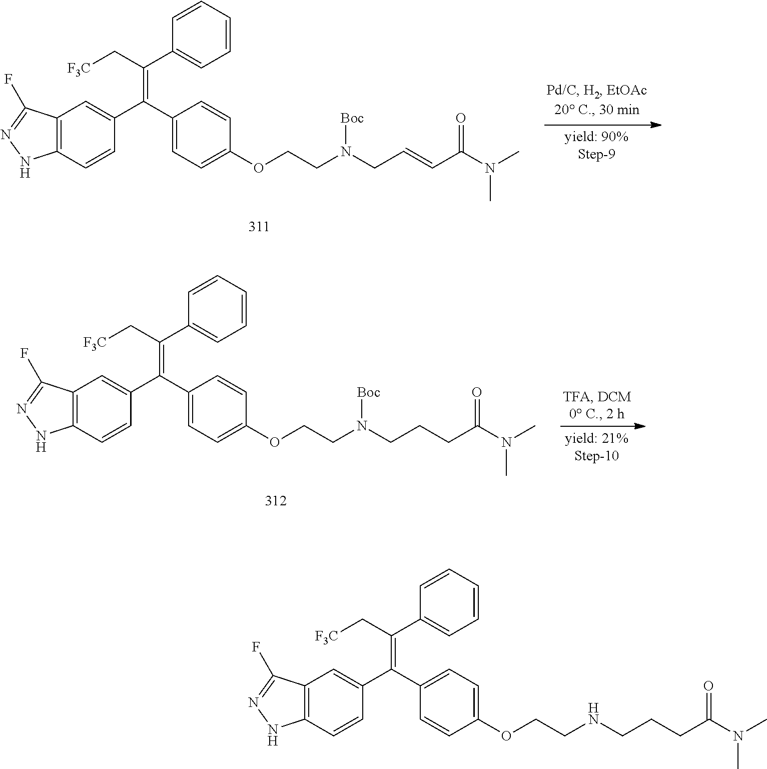 [SMILES: CN(C)C(=O)/C=C/CN(CCOc1ccc(/C(=C(/CC(F)(F)F)c2ccccc2)c2ccc3[nH]nc(F)c3c2)cc1)C(=O)OC(C)(C)C ; CN(C)C(=O)CCCN(CCOc1ccc(/C(=C(/CC(F)(F)F)c2ccccc2)c2ccc3[nH]nc(F)c3c2)cc1)C(=O)OC(C)(C)C ; CN(C)C(=O)CCCNCCOc1ccc(/C(=C(/CC(F)(F)F)c2ccccc2)c2ccc3[nH]nc(F)c3c2)cc1]

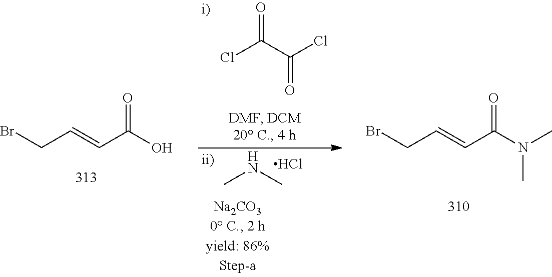

### Example 1: Synthesis of (E)-N,N-dimethyl-((2-(4-(4,4-trifluoro-1-(3-fluoro-1H-indazol-5-yl)-2-phenylbut-1-en-1-yl)phenoxy)ethyl)amino)butanamide (Compound 1)

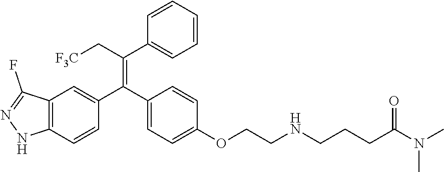 [SMILES: CN(C)C(=O)CCCNCCOc1ccc(/C(=C(/CC(F)(F)F)c2ccccc2)c2ccc3[nH]nc(F)c3c2)cc1]

### Step-1: Synthesis of 5-bromo-3-fluoro-1H-indazole

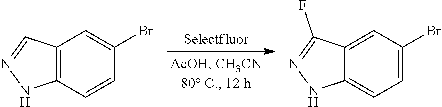 [SMILES: Brc1ccc2[nH]ncc2c1 ; Fc1n[nH]c2ccc(Br)cc12]

Into a 500-mL round-bottom flask was placed 5-bromo-1H-indazole (20 g, 101.51 mmol, 1.00 equiv), selectfluor (71.6 g, 2.00 equiv), AcOH (30 mL), and CH₃CN (300 mL). The resulting solution was stirred at 80° C. in an oil bath until completion. The reaction was then quenched by the addition of 100 mL of water. The resulting solution was extracted with 3×100 mL of ethyl acetate and the organic layers combined. The residue was applied onto a silica gel column with ethyl acetate/petroleum ether (1:4). The collected fractions were combined and concentrated under vacuum to deliver the title compound in 11 g (50%) as a white solid. LCMS: 215.1 [M+H]⁺.

### Step-2: Synthesis of 5-bromo-3-fluoro-1-((2-(trimethylsilyl)ethoxy)methyl)-1H-indazole

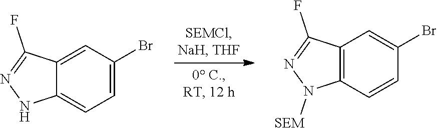

Into a 500-mL round-bottom flask was placed 5-bromo-3-fluoro-1H-indazole (10 g, 46.51 mmol, 1.00 equiv) and THF (250 mL). This was followed by the addition of sodium hydride (2.4 g, 100.00 mmol, 1.30 equiv) in portions at 0° C. The resulting solution was stirred for 30 min at 0° C. in a water/ice bath. To this was added SEMCl (8.5 g, 1.10 equiv) dropwise with stirring at 0° C. The resulting solution was allowed to react, with stirring, at room temperature until completion. The reaction was then quenched by the addition of 50 mL of NH₄Cl (sat. aq.). The resulting solution was extracted with 3×50 mL of ethyl acetate and the organic layers combined and concentrated under vaccum. The residue was applied onto a silica gel column with ethyl acetate/petroleum ether (1:20). The solid was dried in an oven under reduced pressure to deliver the title compound in 12 g (75%) as brown oil. LCMS: 345, 347 [M+H]⁺.

### Step-3: Synthesis of 3-fluoro-1-((2-(trimethylsilyl)ethoxy)methyl)-5-(2-(trimethylsilyl)ethynyl)-1H-indazole

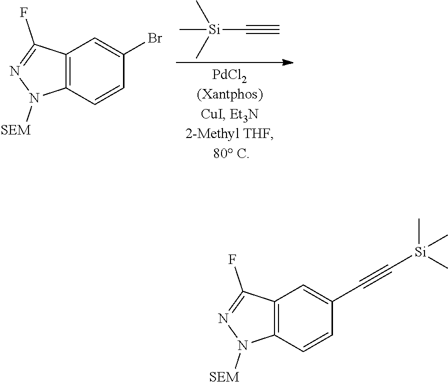

Into a 250-mL round-bottom flask purged and maintained with an inert atmosphere of nitrogen was placed 5-bromo-3-fluoro-1-((2-(trimethylsilyl)ethoxy)methyl)-1H-indazole (8.0 g, 23.17 mmol, 1.00 equiv), CuI (1.36 g, 7.14 mmol, 0.30 equiv), triethylamine (12 g, 118.59 mmol, 5.00 equiv). PdCl₂ (0.4 g, 0.10 equiv), Xantphos (2.72 g, 4.70 mmol, 0.20 equiv), ethynyltrimethylsilane (11.4 g, 116.07 mmol, 5.00 equiv), and 2-Methyl THF (20 mL). The resulting solution was stirred at 80° C. in an oil bath until completion. The resulting mixture was concentrated under vacuum and the residue was applied onto a silica gel column with ethyl acetate/petroleum ether (1:10). The solid was dried in an oven under reduced pressure to deliver the title compound in 8 g (95%) as brown oil.

### Step-4: Synthesis of 5-ethynyl-3-fluoro-1-((2-(trimethylsilyl)ethoxy)methyl)-1H-indazole

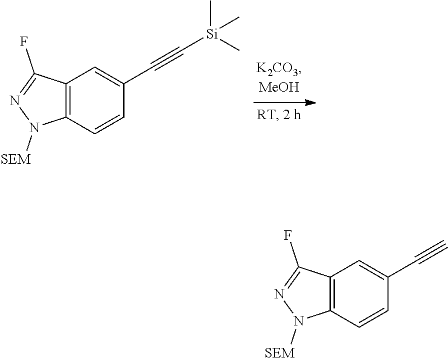

Into a 50-mL round-bottom flask was placed 3-fluoro-1-((2-(trimethylsilyl)ethoxy)methyl)-5-(2-(trimethylsilyl)ethynyl)-1H-indazole (8 g, 22.06 mmol, 1.00 equiv), potassium carbonate (6.1 g, 44.14 mmol, 2.00 equiv), and methanol (20 mL). The resulting solution was stirred at room temperature until completion. The reaction was then quenched by the addition of 40 mL of water. The resulting solution was extracted with 3×20 mL of ethyl acetate and the organic layers combined and concentrated under vacuum. The residue was applied onto a silica gel column with ethyl acetate/petroleum ether (1:20). The solid was dried in an oven under reduced pressure to deliver the title compound in 6.0 g (94%) as a brown oil. LCMS: 291 [M+H]⁺.

### Step-5: Synthesis of 3-fluoro-1-(4,4,4-trifluorobut-1-yn-1-yl)-1-((2-(trimethylsilyl)ethoxy)methyl)-1H-indazole

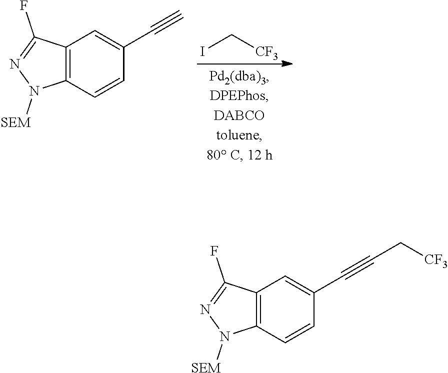

Into a 100-mL round-bottom flask purged and maintained with an inert atmosphere of nitrogen was placed 5-ethynyl-3-fluoro-1-((2-(trimethylsilyl)ethoxy)methyl)-1H-indazole (6 g, 20.66 mmol, 1.00 equiv), 1,1,1-trifluoro-2-iodoethane (8.69 g, 41.39 mmol, 2.00 equiv), toluene (50 mL), Pd₂(dba)₃CHCl₃ (1.08 g, 0.05 equiv), DPEPhos (2.22 g, 0.20 equiv), and DABCO (4.62 g, 2.00 equiv). The resulting solution was stirred at 80° C. in an oil bath until completion. The resulting mixture was concentrated under vacuum. The residue was applied onto a silica gel column with ethyl acetate/petroleum ether (1:10). The solid was dried in an oven under reduced pressure to deliver the title compound in 5 g (65%) as a brown oil. LCMS: 373 [M+H]⁺.

### Step-6: Synthesis of tert-butyl (E)-(2-(4-(4,4,4-trifluoro-1-(3-fluoro-1-((2-(trimethylsilyl)ethoxy)methyl)-1H-indazol-5-yl)-2-phenylbut-1-en-1-yl)phenoxy)ethyl)carbamate

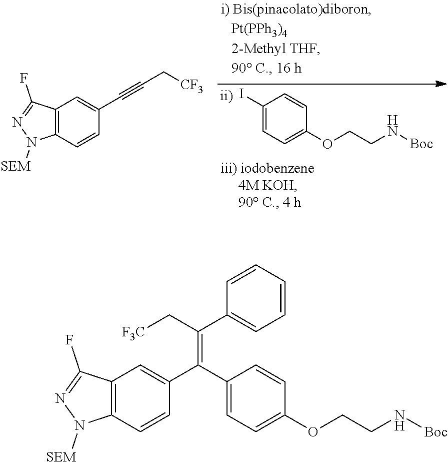

Into a 40-mL vial purged and maintained with an inert atmosphere of nitrogen was placed 3-fluoro-5-(4,4,4-trifluorobut-1-yn-1-yl)-1-((2-(trimethylsilyl)ethoxy)methyl)-1H-indazole (1.0 g, 2.69 mmol, 1.00 equiv), 2-methyl THF (20 mL), 4,4,5,5-tetramethyl-2-(tetramethyl-1,3,2-dioxaborolan-2-yl)-1,3,2-dioxaborolane (680 mg, 2.68 mmol, 1.00 equiv), and Pt(PPh₃)₄ (33 mg, 0.03 mmol, 0.01 equiv). The resulting solution was stirred at 90° C. until completion. The reaction progress was monitored by LCMS. The solution was allowed to cool to room temperature and tert-butyl (2-(4-iodophenoxy)ethyl)carbamate (976 mg, 2.69 mmol, 1.00 equiv) (Scheme 5. Step-1), PdCl₂(PPh₃)₂ (95 mg, 0.14 mmol, 0.05 equiv), Cs₂CO₃ (2.2 g, 6.73 mmol, 2.51 equiv), and water (5 mL) were added. This mixture was degassed with nitrogen and then stirred at room temperature until completion. To the above reaction mixture, iodobenzene (1.23 g, 6.03 mmol, 2.25 equiv) and KOH (1.05 g, 18.71 mmol, 6.98 equiv) were added. Reaction mixture was stirred at 90° C. until completion, and then cooled to room temperature. The resulting solution was diluted with 30 mL of H₂O and was extracted with 3×30 mL of ethyl acetate. The organic layers were combined and dried over anhydrous sodium sulfate. The residue was applied onto a silica gel column with ethyl acetate/petroleum ether (0:100-10:90). The collected fractions were combined and concentrated under vacuum to deliver the title compound in 1.0 g (54%) as a yellow oil. LCMS: 708 [M+Na]⁺.

### Step-7: Synthesis of (E)-2-(4-(4,4,4-trifluoro-1-(3-fluoro-1H-indazol-1-yl)-2-phenylbut-1-en-1-yl)phenoxy)ethan-1-amine

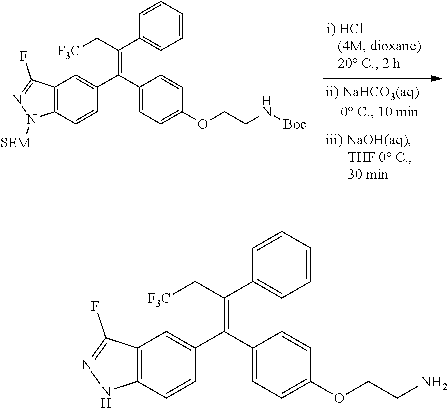

Into a 100-mL round-bottom flask was placed tert-butyl (E)-(2-(4-(4,4,4-trifluoro-1-(3-fluoro-1-((2-(trimethyl silyl)ethoxy)methyl)-1H-indazol-5-yl)-2-phenylbut-1-en-1-yl)phenoxy)ethyl)carbamate (900 mg, 1.31 mmol, 1.00 equiv), and saturated hydrogen chloride in dioxane (4M, 5 mL). The reaction was stirred at 20° C. until completion, then sodium bicarbonate (sat. aq.) (10 mL) was added. The reaction was stirred for 10 min at 0° C., then sodium hydroxide (sat. aq.) (10 mL), and THF (20 mL) were added. The resulting solution was stirred at 0° C. until completion. The reaction progress was monitored by LCMS. The resulting solution was diluted with 20 mL of H₂O, then extracted with 3×40 mL of ethyl acetate. The organic layers combined and dried over anhydrous sodium sulfate, then concentrated under vacuum to deliver the title compound in 500 mg (84%) as a yellow solid. LCMS: 456 [M+H]⁺.

### Step-8: Synthesis of tert-butyl ((E)-4-(dimethylamino)-4-oxobut-2-en-1-yl)(2-(4-((E)-4,4,4-trifluoro-1-(3-fluoro-1H-indazol-5-yl)-2-phenylbut-1-en-1-yl)phenoxy)ethyl)carbamate

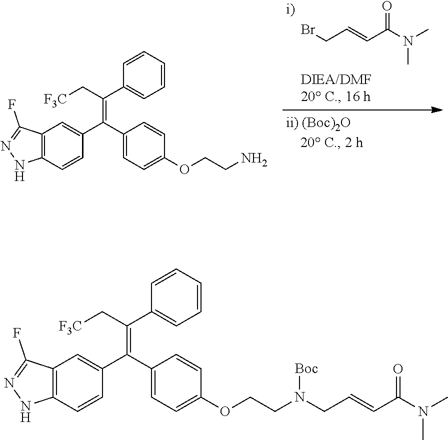

Into a 50-mL round-bottom flask was placed (E)-2-(4-(4,4,4-trifluoro-1-(3-fluoro-1H-indazol-5-yl)-2-phenylbut-1-en-1-yl)phenoxy)ethan-1-amine (500 mg, 1.10 mmol, 1.00 equiv), N,N-dimethylformamide (10 mL), DIEA (284 mg, 2.20 mmol, 2.00 equiv), and (E)-4-bromo-N,N-dimethylbut-2-enamide (148 mg, 0.77 mmol, 0.70 equiv) (preparation shown below, Step-a). The resulting solution was stirred for at 20° C. until completion. The reaction progress was monitored by LCMS. To the above reaction solution, (Boc)₂O (300 mg, 1.37 mmol, 1.50 equiv) was added. The resulting solution was stirred at 20° C. until completion, then the solution was concentrated under vacuum. The residue was applied onto a silica gel column with ethyl acetate/petroleum ether (0:100-100:0). The collected fractions were combined and concentrated under vacuum to deliver the title compound in 200 mg (27%) as a yellow solid. LCMS: 667 [M+H]⁺.

### Step-9: Synthesis of tert-butyl (E)-(4-(dimethylamino)-4-oxobutyl)(2-(4-(4,4,4-trifluoro-1-(3-fluoro-1H-Indazol-5-yl)-2-phenylbut-1-en-1-yl)phenoxy)ethyl)carbamate

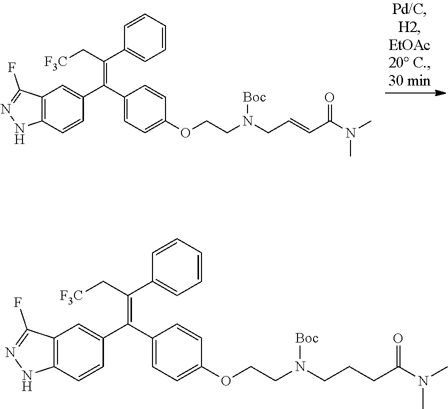 [SMILES: CN(C)C(=O)/C=C/CN(CCOc1ccc(/C(=C(/CC(F)(F)F)c2ccccc2)c2ccc3[nH]nc(F)c3c2)cc1)C(=O)OC(C)(C)C ; CN(C)C(=O)CCCN(CCOc1ccc(/C(=C(/CC(F)(F)F)c2ccccc2)c2ccc3[nH]nc(F)c3c2)cc1)C(=O)OC(C)(C)C]

Into a 50-mL round-bottom flask was placed tert-butyl ((E)-4-(dimethylamino)-4-oxobut-2-en-1-yl)(2-(4-((E)-4,4,4-trifluoro-1-(3-fluoro-1H-indazol-5-yl)-2-phenylbut-1-en-1-yl)phenoxy)ethyl)carbamate (200 mg, 0.30 mmol, 1.00 equiv), ethyl acetate (10 mL), and 10% Pd/C (50 mg). To the above solution H₂(g) was introduced in. The resulting solution was stirred at 20° C. until completion. The reaction progress was monitored by LCMS. The solids were filtered out upon completion. The resulting mixture was concentrated under vacuum to deliver the title compound in 180 mg (90%) as a yellow oil. LCMS: 669 [M+H]⁺.

### Step-10: Synthesis of (E)-N,N-dimethyl-4-((2-(4-(4,4,4-trifluoro-1-(3-fluoro-1H-indazol-5-yl)-2-phenylbut-1-en-1-yl)phenoxy)ethyl)amino)butanamide

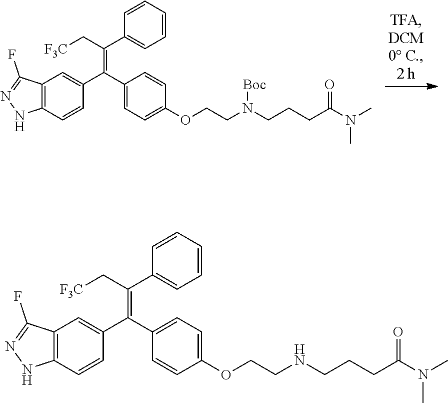 [SMILES: CN(C)C(=O)CCCN(CCOc1ccc(/C(=C(/CC(F)(F)F)c2ccccc2)c2ccc3[nH]nc(F)c3c2)cc1)C(=O)OC(C)(C)C ; CN(C)C(=O)CCCNCCOc1ccc(/C(=C(/CC(F)(F)F)c2ccccc2)c2ccc3[nH]nc(F)c3c2)cc1]

Into a 50-mL round-bottom flask was placed tert-butyl (E)-(4-(dimethylamino)-4-oxobutyl)(2-(4-(4,4,4-trifluoro-1-(3-fluoro-1H-indazol-5-yl)-2-phenylbut-1-en-1-yl)phenoxy)ethyl)carbamate- (160 mg, 0.24 mmol, 1.00 equiv), DCM (10 mL), and trifluoroacetic acid (5 mL). The resulting solution was stirred at 0° C. until completion. The reaction progress was monitored by LCMS. The resulting mixture was concentrated under vacuum. The crude product was purified by Prep-HPLC with the following conditions (2#-AnalyseHPLC-SHIMADZU(HPLC-10)): Column, XSelect CSH Prep C18 OBD Column, 5 um, 19*150 mm; mobile phase, Water (0.05% TFA) and ACN (25.0% ACN up to 52.0% in 12 min); Detector, uv 254/220 nm. 100 mL product was obtained and concentrated under vacuum to deliver the title compound in 14.4 mg, 0.5% overall yield. ¹H NMR (400 MHz, CD3OD): δ 7.63 (s, 1H), 7.51-7.43 (m, 1H), 7.31-7.12 (m, 6H), 6.90-6.81 (m, 2H), 6.70-6.61 (m, 2H), 3.98 (t, J=5.3 Hz, 2H), 3.45-3.35 (m, 2H), 3.05 (s, 3H), 2.94-2.88 (m, 5H), 2.68 (t, J=7.2 Hz, 2H), 2.43 (t, J=7.4 Hz, 2H), 1.81 (p, J=7.4 Hz, 2H). LCMS: 569.6 [M+H]⁺.

### Step-a: Synthesis of (E)-4-bromo-N,N-dimethylbut-2-enamide

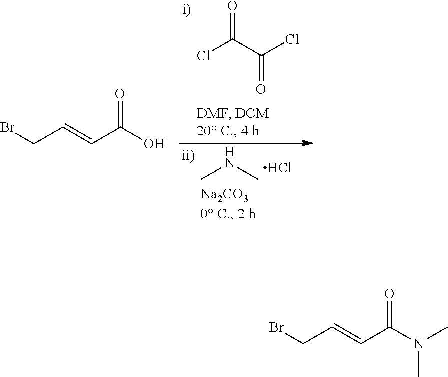

Into a 250-mL round-bottom flask purged and maintained with an inert atmosphere of nitrogen was placed (E)-4-bromobut-2-enoic acid (5 g, 30.31 mmol, 1.00 equiv), DCM (50 mL), and N,N-dimethylformamide (0.5 mL). This was followed by the addition of oxalyl dichloride (3.8 g, 29.94 mmol, 0.99 equiv) dropwise with stirring at 0° C. over 30 min. The resulting solution was stirred at 20° C. until completion. The reaction progress was monitored by LCMS. To the above reaction solution dimethylamine hydrochloride (2.5 g, 30.66 mmol, 1.02 equiv), and sodium carbonate (9.6 g, 90.57 mmol, 3.02 equiv) were added. The resulting solution was stirred at 0° C. until completion. The reaction was then quenched by the addition of 100 mL of water, extracted with 3×100 mL of DCM. The organic layers were combined, dried over anhydrous sodium sulfate and concentrated under vacuum to deliver the title compound in 5.0 g (86%) as a off-white solid.

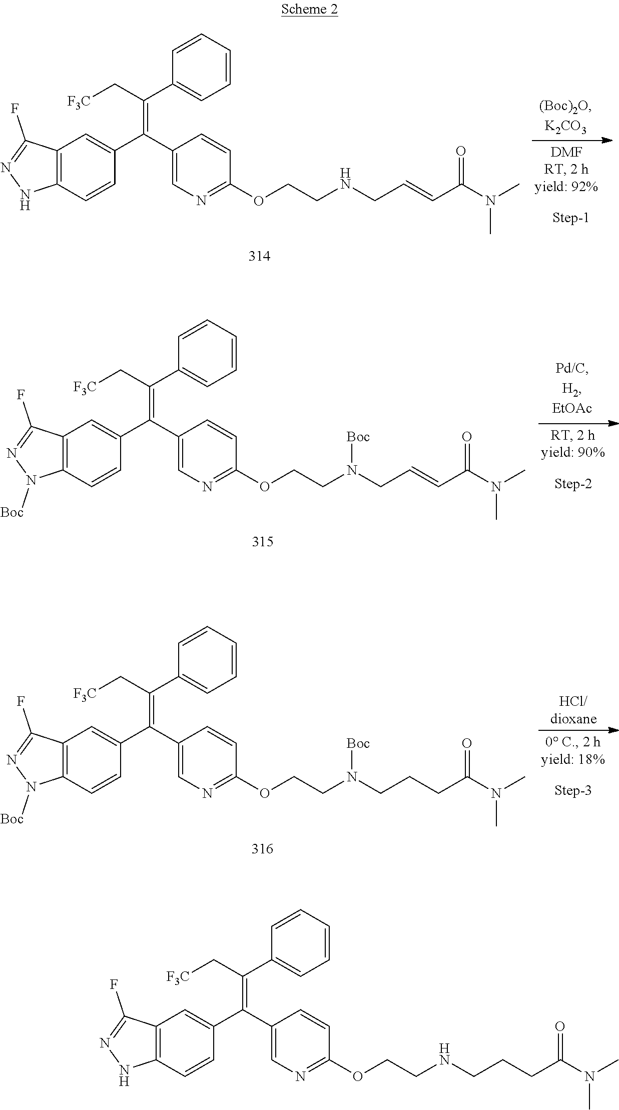

### Example 2: Synthesis of (Z)—N,N-dimethyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide (Compound 2)

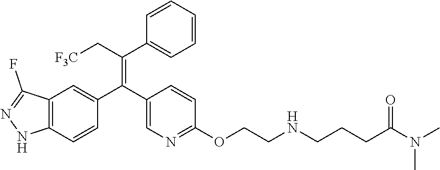 [SMILES: CN(C)C(=O)CCCNCCOc1ccc(/C(=C(/CC(F)(F)F)c2ccccc2)c2ccc3[nH]nc(F)c3c2)cn1]

### Step-1: Synthesis of tert-butyl 5-((Z)-1-(6-(2-((tert-butoxycarbonyl)((E)-4-(dimethylamino)-4-oxobut-2-en-1-yl)amino)ethoxy)pyridin-3-yl)-4,4,4-trifluoro-2-phenylbut-1-en-1-yl)-3-fluoro-1H-indazole-1-carboxylate

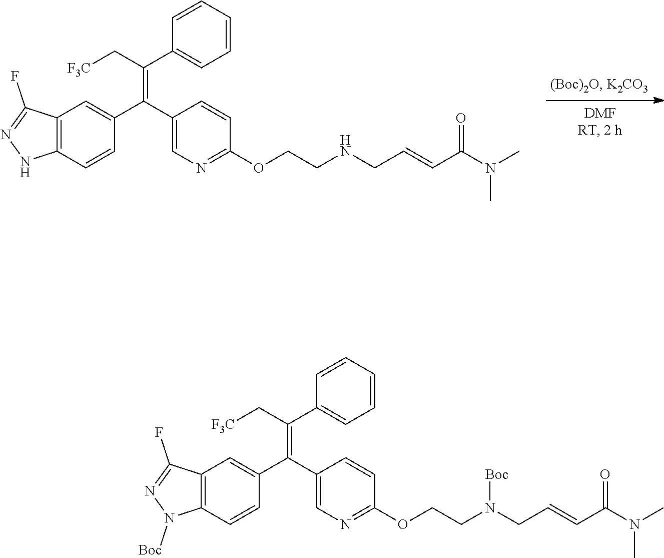 [SMILES: CN(C)C(=O)/C=C/CNCCOc1ccc(/C(=C(/CC(F)(F)F)c2ccccc2)c2ccc3[nH]nc(F)c3c2)cn1 ; CN(C)C(=O)/C=C/CN(CCOc1ccc(/C(=C(/CC(F)(F)F)c2ccccc2)c2ccc3c(c2)c(F)nn3C(=O)OC(C)(C)C)cn1)C(=O)OC(C)(C)C]

Into a 8-mL vial was placed (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (80 mg, 0.14 mmol, 1.00 equiv) (synthesized following the approach outlined in Patent Application Publication No. US 2016347717 A1), N,N-dimethylformamide (2 mL), potassium carbonate (58 mg, 0.42 mmol, 2.98 equiv), and (Boc)₂O (61 mg, 0.28 mmol, 1.98 equiv). The resulting solution was stirred at 20° C. until completion. The reaction progress was monitored by LCMS. The resulting solution was diluted with 10 mL of H₂O and extracted with 2×10 mL of ethyl acetate. The organic layers were combined and concentrated under vacuum. The residue was applied onto a silica gel column with ethyl acetate:petroleum ether (0:100-20:80). The collected fractions were combined and concentrated under vacuum to deliver the title compound in 100 mg (92%) as a yellow solid. LCMS: 768.3 [M+H]⁺.

### Step-2: Synthesis of tert-butyl (Z)-5-(1-(6-(2-((tert-butoxycarbonyl)(4-(diethylamino)-4-oxobutyl)amino)ethoxy)pyridin-3-yl)-4,4,4-trifluoro-2-phenylbut-1-en-1-yl)-3-fluoro-1H-indazole-1-carboxylate

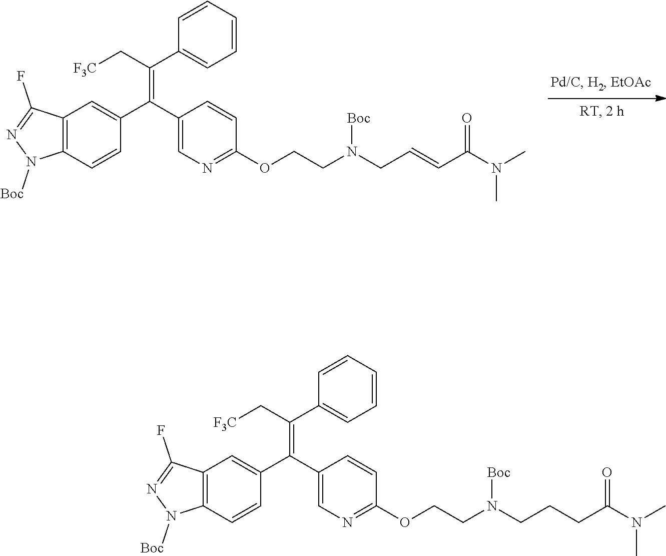 [SMILES: CN(C)C(=O)/C=C/CN(CCOc1ccc(/C(=C(/CC(F)(F)F)c2ccccc2)c2ccc3c(c2)c(F)nn3C(=O)OC(C)(C)C)cn1)C(=O)OC(C)(C)C ; CN(C)C(=O)CCCN(CCOc1ccc(/C(=C(/CC(F)(F)F)c2ccccc2)c2ccc3c(c2)c(F)nn3C(=O)OC(C)(C)C)cn1)C(=O)OC(C)(C)C]

Into a 50-mL round-bottom flask was placed tert-butyl 5-((Z)-1-(6-(2-((tert-butoxycarbonyl)((E)-4-(dimethylamino)-4-oxobut-2-en-1-yl)amino)ethoxy)pyridin-3-yl)-4,4,4-trifluoro-2-phenylbut-1-en-1-yl)-3-fluoro-1H-indazole-1-carboxylate (100 mg, 0.13 mmol, 1.00 equiv), ethyl acetate (10 mL), and 10% Pd/C (20 mg). To the above solution, H₂(g) was introduced in. The resulting solution was stirred at 20° C. until completion. The reaction progress was monitored by LCMS. The solids were filtered out upon completion. The resulting mixture was concentrated under vacuum to deliver the title compound in 90 mg (90%) as a yellow solid. LCMS: 770 [M+H]⁺.

### Step-3: Synthesis of (Z)—N,N-dimethyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide

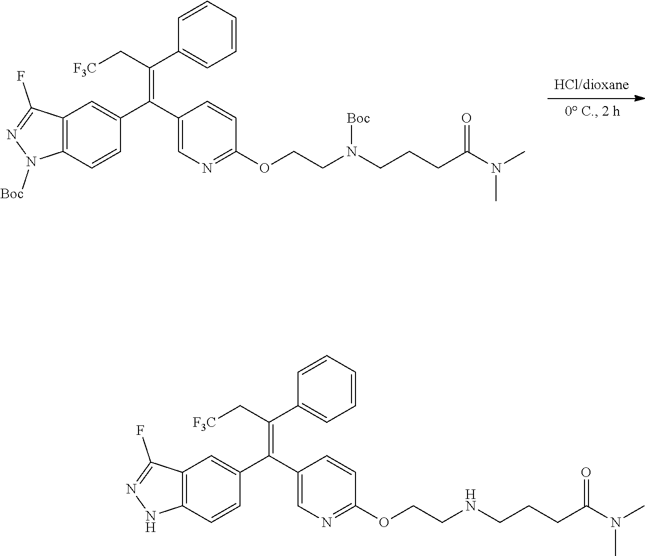 [SMILES: CN(C)C(=O)CCCN(CCOc1ccc(/C(=C(/CC(F)(F)F)c2ccccc2)c2ccc3c(c2)c(F)nn3C(=O)OC(C)(C)C)cn1)C(=O)OC(C)(C)C ; CN(C)C(=O)CCCNCCOc1ccc(/C(=C(/CC(F)(F)F)c2ccccc2)c2ccc3[nH]nc(F)c3c2)cn1]

Into a 25-mL round-bottom flask was placed tert-butyl (Z)-5-(1-(6-(2-((tert-butoxycarbonyl)(4-(dimethylamino)-4-oxobutyl)amino)ethoxy)pyridin-3-yl)-4,4,4-trifluoro-2-phenylbut-1-en-1-yl)-3-fluoro-1H-indazole-1-carboxylate (90 mg, 0.12 mmol, 1.00 equiv), and hydrogen chloride/dioxane (5 mL). The resulting solution was stirred at 0° C. until completion. The reaction progress was monitored by LCMS. The resulting mixture was concentrated under vacuum. The crude product (5 mL) was purified by Prep-HPLC with the following conditions: Column, XBridge Shield RP18 OBD Column, 5 um, 19*150 mm; mobile phase, Mobile Phase A: water (10 MMOL/L NH4HCO3). Mobile Phase B: ACN; Detector, 254/220 nm. 100 mL product was obtained and concentrated under vacuum to deliver the title compound in 12.1 mg, 14.9% overall yield. ¹H NMR (300 MHz, CD3OD): δ 7.64-7.63 (m, 2H), 7.47-7.46 (m, 1H), 7.29-7.20 (m, 7H), 6.57-6.54 (d, J=8.1 Hz, 1H). 4.26-4.23 (t, J=5.1 Hz, 2H), 3.46-3.39 (m, 2H), 3.08 (s, 3H), 2.94-2.89 (m, 5H), 2.69-2.64 (t, J=6.9 Hz, 2H), 2.43-2.38 (t, J=7.2 Hz, 2H), 1.80-1.75 (t, J=7.2 Hz, 2H) ppm. LCMS: 570.0 [M+H]⁺.

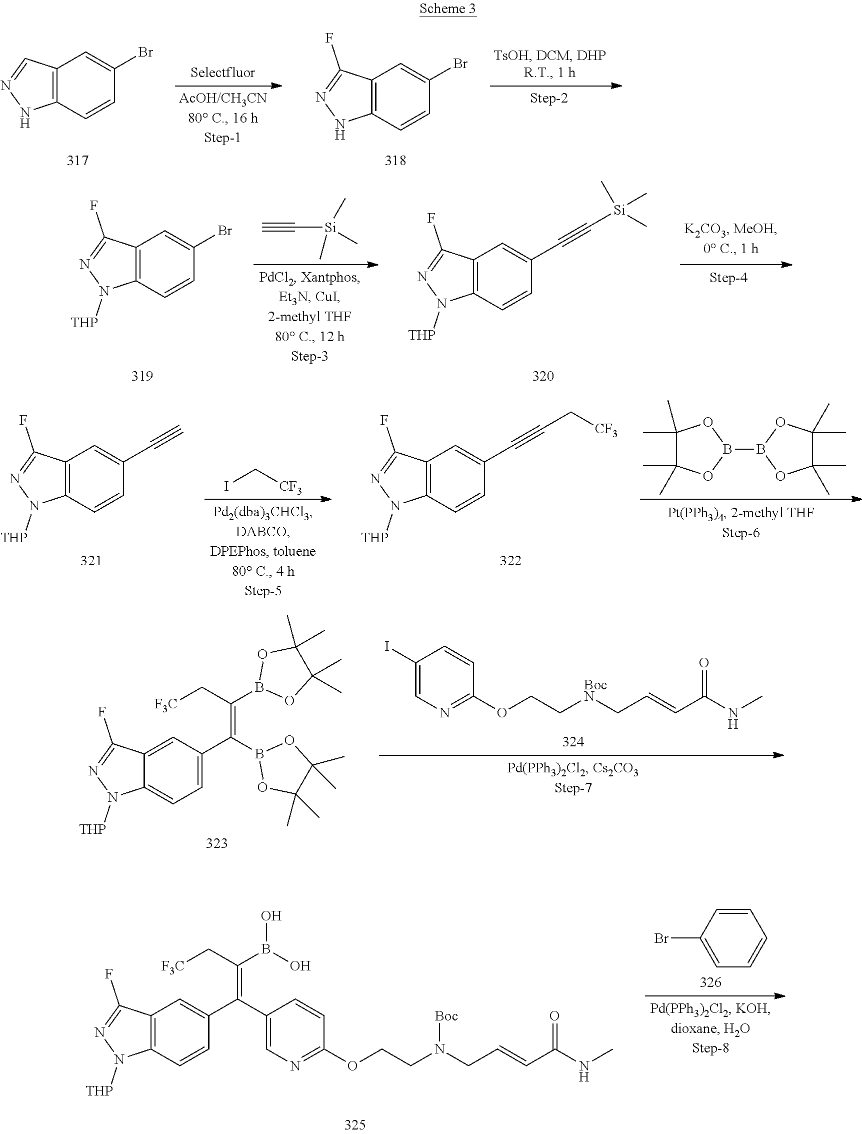

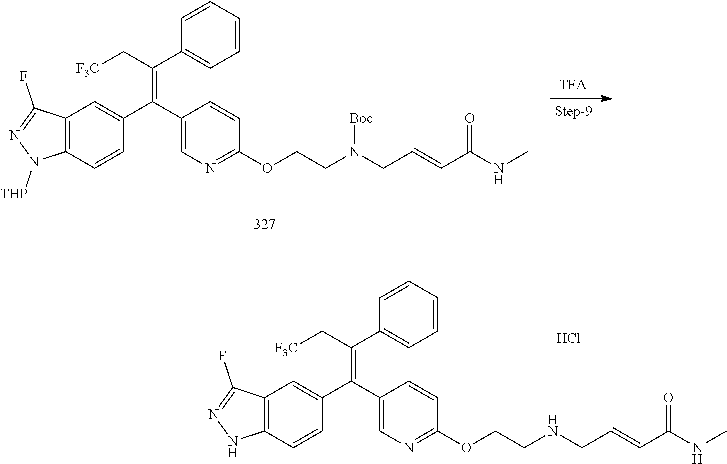

### Example 3: Synthesis of (E)-N-methyl-4-(2-(5-((Z)-4,4,4-trifluoro-1-(3-fluoro-1H-indazol-5-yl)-2-phenylbut-1-enyl)pyridin-2-yloxy)ethylamino)but-2-enamide (Compound 3)

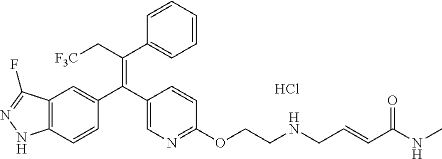 [SMILES: CNC(=O)/C=C/CNCCOc1ccc(/C(=C(/CC(F)(F)F)c2ccccc2)c2ccc3[nH]nc(F)c3c2)cn1 ; Cl]

### Step-1: Synthesis of 5-bromo-3-fluoro-1H-indazole

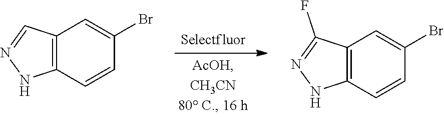 [SMILES: Brc1ccc2[nH]ncc2c1 ; Fc1n[nH]c2ccc(Br)cc12]

Into a 5-L 3-necked round-bottom flask purged and maintained with an inert atmosphere of nitrogen was placed 5-bromo-1H-indazole (200 g, 1.0204 mol, 1.00 equiv), CH₃CN (3.5 L), acetic acid (120 mL), and selectfluoro (544 g, 1.5367 mol, 1.51 equiv). The resulting solution was stirred at 80° C. until completion. The reaction progress was monitored by LCMS. The resulting solution was diluted with 8 L of ethyl acetate and washed with 3×4000 mL of H₂O. The organic layer was dried over anhydrous sodium sulfate and concentrated under vacuum. The residue was applied onto a silica gel column with ethyl acetate/petroleum ether (0:100-15:85). The collected fractions were combined and concentrated under vacuum to deliver the title compound in 72 g (33%) as a yellow solid. ¹H NMR (400 MHz, DMSO-d₆) δ 12.77 (s, 1H), 8.03-7.90 (m, 1H), 7.59-7.48 (m, 2H). LCMS: 215 [M+H]⁺.

### Step-2: Synthesis of 5-bromo-3-fluoro-1-(tetrahydro-2H-pyran-2-yl)-1H-indazole

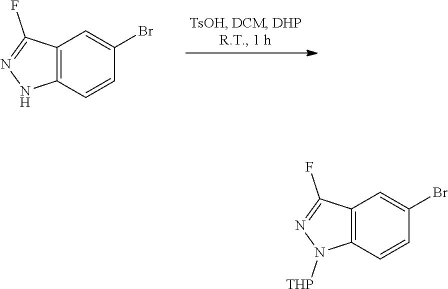

Into a 2-L 3-necked round-bottom flask was placed 5-bromo-3-fluoro-1H-indazole (70 g, 325.55 mmol, 1.00 equiv), DCM (700 mL), and TsOH (5.6 g, 32.52 mmol, 0.10 equiv). This was followed by the drop-wise addition of DHP (82.4 g, 979.55 mmol, 3.01 equiv) while stirring at 0° C. The resulting solution was stirred at 0° C. until completion. The reaction was monitored by LCMS. The resulting mixture was washed with 2×500 mL of H₂O. and the organic layer was dried over anhydrous sodium sulfate and concentrated under vacuum. The residue was applied onto a silica gel column with ethyl acetate/petroleum ether (0:100-10:90). The collected fractions were combined and concentrated under vacuum to deliver the title compound in 96.3 g (99%) as yellow oil. ¹H NMR (300 MHz, DMSO-d₆) δ 8.02 (d, J=1.8 Hz, 1H), 7.78-7.74 (m, 1H), 7.67-7.63 (m, 1H), 5.88-5.71 (m, 1H), 3.95-3.79 (m, 1H), 3.75-3.71 (m, 1H), 2.31-2.13 (m, 1H), 2.11-1.86 (m, 2H), 1.74-1.70 (m, 1H), 1.58-1.50 (m, 2H). LCMS: 299 [M+H]⁺.

### Step-3: Synthesis of 3-fluoro-1-(tetrahydro-2H-pyran-2-yl)-5-((trimethylsilyl)ethynyl)-1H-indazole

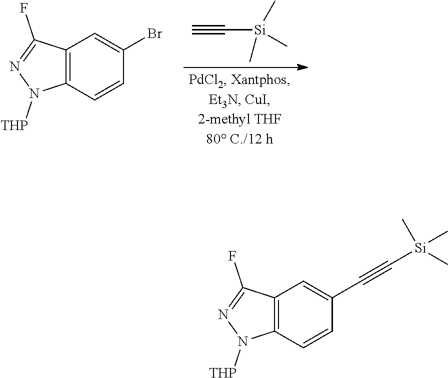

Into a 2-L round-bottom flask purged and maintained with an inert atmosphere of nitrogen was placed 5-bromo-3-fluoro-1-(tetrahydro-2H-pyran-2-yl)-1H-indazole (94.3 g, 315.24 mmol, 1.00 equiv), 2-Methyl THF (950 mL), TEA (95.6 g, 944.76 mmol, 3.00 equiv), ethynyltrimethylsilane (154.5 g, 1.57 mol, 4.99 equiv), PdCl₂ (5.6 g, 31.64 mmol, 0.10 equiv), Xantphos (36.5 g, 63.08 mmol, 0.20 equiv), and CuI (12 g, 63.01 mmol, 0.20 equiv). The resulting solution was stirred at 80° C. until completion. The reaction progress was monitored by LCMS. The resulting solution was diluted with 1 L of 2-Methyl THF and was washed with 1×1 L of brine. The mixture was dried over anhydrous sodium sulfate and concentrated under vacuum to deliver the title compound in 134 g (crude) as a black oil. The crude product was used directly to the next step.

### Step-4: Synthesis of 5-ethynyl-3-fluoro-1-(tetrahydro-2H-pyran-2-yl)-1H-indazole

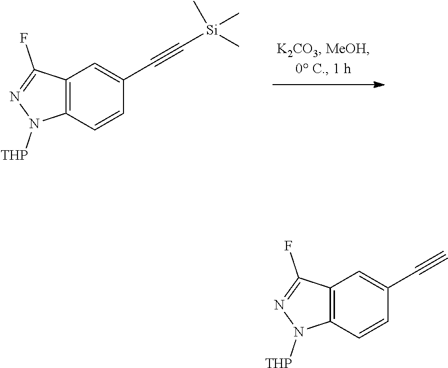

Into a 2-L round-bottom flask was placed 3-fluoro-1-(tetrahydro-2H-pyran-2-yl)-5-((trimethylsilyl)ethynyl)-1H-indazole (131.3 g, 414.92 mmol, 1.00 equiv), methanol (950 mL), and potassium carbonate (114.7 g, 829.90 mmol, 2.00 equiv). The resulting solution was stirred at 0° C. until completion. The reaction progress was monitored by LCMS. The resulting mixture was concentrated under vacuum and then diluted with 1 L of H₂O. The solution was extracted with 3×1 L of ethyl acetate and the organic layers combined, dried over anhydrous sodium sulfate, and concentrated under vacuum to deliver the title compound in 77 g (76%/o) as black oil. LCMS: 245 [M+H]⁺.

### Step-5: Synthesis of 3-fluoro-1-(tetrahydro-2H-pyran-2-yl)-5-(4,4,4-trifluorobut-1-yn-1-yl)-1H-indazole

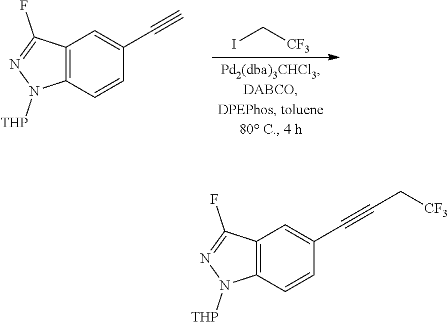

Into a 2-L round-bottom flask purged and maintained with an inert atmosphere of nitrogen was placed 5-ethynyl-3-fluoro-1-(tetrahydro-2H-pyran-2-yl)-1H-indazole (72 g, 294.76 mmol. 1.00 equiv), toluene (900 mL), 1,1,1-trifluoro-2-iodoethane (186 g, 885.98 mmol, 3.01 equiv), DABCO (99 g, 883.93 mmol, 3.00 equiv), DPEPhos (31.8 g, 59.00 mmol, 0.20 equiv), and Pd₂(dba)₃CHCl₃ (15.3 g, 14.78 mmol, 0.05 equiv). The resulting solution was stirred at 80° C. until completion. The reaction progress was monitored by LCMS. The resulting solution was diluted with 1 L of H₂O and extracted with 2×1 L of ethyl acetate. The organic layers were combined, dried over anhydrous sodium sulfate, and concentrated under vacuum. The residue was applied onto a silica gel column with ethyl acetate/petroleum ether (0:100-15:85). The collected fractions were combined and concentrated under vacuum to deliver the title compound in 45 g (47%) as a yellow solid. ¹H NMR (300 MHz. DMSO-d₆) δ 7.89 (d, J=1.4 Hz, 1H), 7.81-7.77 (m, 1H), 7.58-7.54 (m, 1H), 5.84-5.80 (m, 1H), 3.94-3.60 (m, 4H), 2.35-2.12 (m, 1H), 2.06-1.89 (m, 2H), 1.86-1.64 (m, 1H), 1.58-1.54 (m, 2H). LCMS: 327 [M+H]⁺.

### Step-6: Synthesis of (Z)-3-fluoro-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole

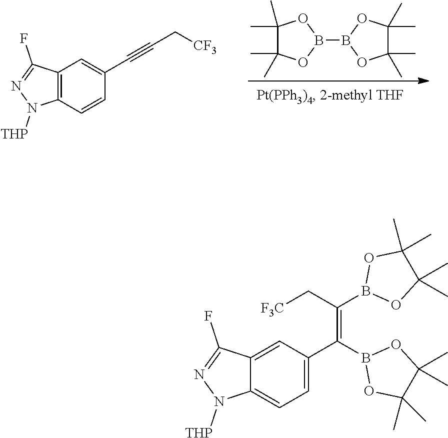

Into a 500-mL round-bottom flask purged and maintained with an inert atmosphere of nitrogen was placed 4,4,5,5-tetramethyl-2-(tetramethyl-1,3,2-dioxaborolan-2-yl)-1,3,2-dioxaborolane (57 g, 219.74 mmol, 2.00 equiv), 3-fluoro-1-(tetrahydro-2H-pyran-2-yl)-5-(4,4,4-trifluorobut-1-en-1-yl)-1H-indazole (36 g, 110.34 mmol, 1.00 equiv). Pt(PPh₃)₄ (6.84 g, 0.05 equiv), and 2-Methyl THF (450 mL). The solution was stirred at 90° C. until completion to deliver the title compound (crude) that was used directly to the next step.

### Step-7: Synthesis of ((E)-1-(6-(2-((tert-butoxycarbonyl)((E)-4-(methylamino)-4-oxobut-2-en-1-yl)amino)ethoxy)pyridin-3-yl)-4,4,4-trifluoro-1-(3-fluoro-1-(tetrahydro-2H-pyran-2-yl)-1H-indazol-5-yl)but-1-en-2-yl)boronic acid

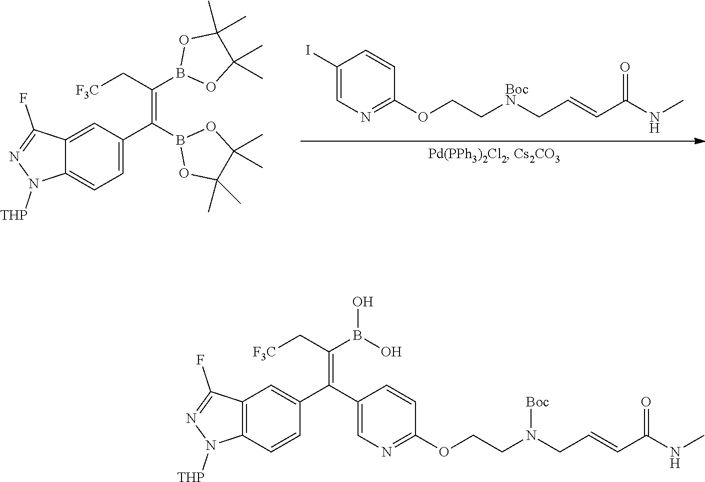

Into a 40-mL vial purged and maintained with an inert atmosphere of nitrogen was placed (Z)-3-fluoro-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (101.4 g, 180.6 mmol, 1.00 equiv), tert-butyl (E)-(2-((5-iodopyridin-2-yl)oxy)ethyl)(4-(methylamino)-4-oxobut-2-en-1-yl)carbamate (83.44 g, 180.6 mmol, 1.00 equiv) (Scheme 4, Steps-1-3), Pd(PPh₃)₂Cl₂ (6.38 g, 8.94 mmol, 0.05 equiv), Cs₂CO₃ (2.0 g, 119.2 mmol, 2.00 equiv), 2-Methyl THF (600 mL), and water (60 mL). The solution was stirred at 25° C. until completion. The resulting mixture was concentrated under vacuum. The residue was applied onto a silica gel column with DCM/methanol (10:1) to deliver the title compound in 59.6 g (crude) as a yellow solid.

### Step-8: Synthesis of tert-butyl ((E)-4-(methylamino)-4-oxobut-2-en-1-yl)(2-((5-((Z)-4,4,4-trifluoro-1-(3-fluoro-1-(tetrahydro-2H-pyran-2-yl)-1H-indazol-5-yl)-2-phenylbut-1-en-1-yl)pyridin-2-yl)oxy)ethyl)carbamate

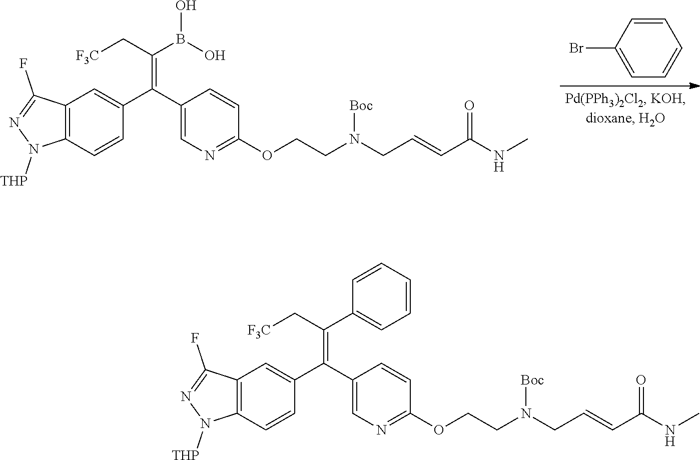

Into a 1000-mL round-bottom flask vial purged and maintained with an inert atmosphere of nitrogen was placed ((E)-1-(6-(2-((tert-butoxycarbonyl)((E)-4-(methylamino)-4-oxobut-2-en-1-yl)amino)ethoxy)pyridin-3-yl)-4,4,4-trifluoro-1-(3-fluoro-1-(tetrahydro-2H-pyran-2-yl)-1H-indazol-5-yl)but-1-en-2-yl)boronic acid (59.6 g, 75.66 mmol, 1.00 equiv), bromobenzene (14.30 g, 90.79 mmol, 1.20 equiv), KOH (8.34 g, 148.70 mmol, 2.00 equiv), Pd(PPh₃)₂Cl₂ (2.59 g, 3.70 mmol, 0.05 equiv), dioxane (1000 mL), and water (200 mL). The solution was stirred at 80° C. in an oil bath until completion. The reaction was then quenched by the addition of 800 mL of water. The resulting solution was extracted with 3×1000 mL of ethyl acetate and the organic layers combined and concentrated under vacuum. The residue was applied onto a silica gel column with DCM/methanol (10:1) to deliver the title compound in 25.0 g (crude) as a yellow solid.

### Step-9: Synthesis of (E)-N-methyl-4-(2-(5-((Z)-4,4,4-trifluoro-1-(3-fluoro-1H-indazol-5-yl)-2-phenylbut-1-enyl)pyridin-2-yloxy)ethylamino)but-2-enamide

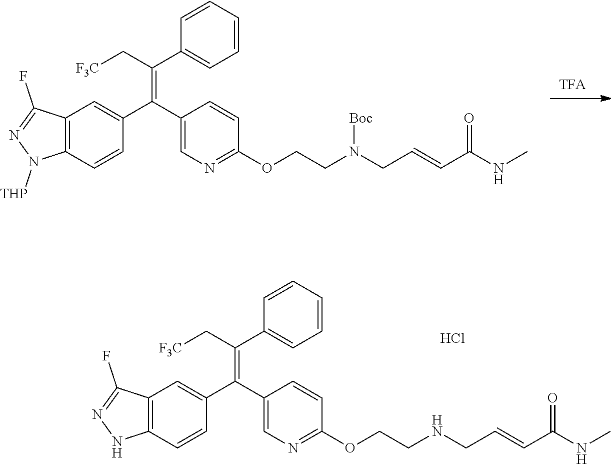

Into a 500-mL round-bottom flask was placed tert-butyl ((E)-4-(methylamino)-4-oxobut-2-en-1-yl)(2-((5-((Z)-4,4,4-trifluoro-1-(3-fluoro-1-(tetrahydro-2H-pyran-2-yl)-1H-indazol-5-yl)-2-phenylbut-1-en-1-yl)pyridin-2-yl)oxy)ethyl)carbamate (25 g, 33.88 mmol, 1.00 equiv), TFA (50 mL), and DCM (250 mL). The resulting solution was stirred at 25° C. until completion. The resulting mixture was concentrated under vacuum. The crude product was purified by Prep-HPLC with the following conditions: Column: X-bridge Prep phenyl 5 um. 19*150 mmh Prep C012 (T)186003581138241113.01; mobile phase, Phase A:water with 0.5% NH₄HCO₃, Phase B:CH₃CN. (20% CH₃CN up to 65% in 60 min, hold 95% in 10 min, down to 20% in 2 min); Detector, UV 254 nm. This resulted in 4.9 g (24%) of (E)-N-methyl-4-(2-(5-((Z)-4,4,4-trifluoro-1-(3-fluoro-1H-indazol-5-yl)-2-phenylbut-1-enyl)pyridin-2-yloxy)ethylamino)but-2-enamide as a yellow solid. ¹H NMR (300 MHz. DMSO-d6) δ 12.6 (s, 1H), 7.92-7.81 (m, 2H), 7.61-7.57 (m, 2H), 7.54 (dd, J=8.7, 2.3 Hz, 1H), 7.23 (dd, J=8.8, 1.5 Hz, 1H), 7.14-7.10 (m, 3H), 7.00-6.97 (m, 2H), 6.69-6.66 (dd, J=8.5, 0.7 Hz, 1H), 6.64-6.54 (m, 1H), 6.02-5.94 (m, 1H), 4.18 (t, J=5.7 Hz, 2H), 3.50-3.46 (t, J=10.9 Hz, 2H), 3.32 (d, J=6.3 Hz, 2H), 2.79 (t, J=5.7 Hz, 2H), 2.63-2.61 (d, J=4.6 Hz, 3H). The 4.9 g solid was dissolved in 80 mL CH₃CN and acidified with 9.75 mL HCl (1N) (1 mL 12N HCl(aq) dissolved in 1 mL CH₃CN) at 0° C. and stirred for 30 min at R.T., then evaporated at 30° C. to remove the excess HCl. The product was then dissolved in 150 mL H₂O and lyophilized for 48 h to deliver the title compound in 5.2 g, 0.82% overall yield, as a yellow solid. ¹H NMR (400 MHz, Methanol-d4) δ 7.75 (dd, J=2.5, 0.8 Hz, 1H), 7.67 (t, J=1.2 Hz, 1H), 7.54-7.50 (m, 1H), 7.43-7.39 (dd, J=8.7, 2.5 Hz, 1H), 7.34 (dd, J=8.8, 1.6 Hz, 1H), 7.31-7.18 (m, 5H), 6.75-6.64 (m, 2H), 6.33-6.28 (m, 1H), 4.52-4.48 (m, 2H), 3.87 (dd, J=7.0, 1.4 Hz, 2H), 3.49-3.39 (m, 4H), 2.82 (s, 3H). LCMS: 554.69 [M+H]⁺.

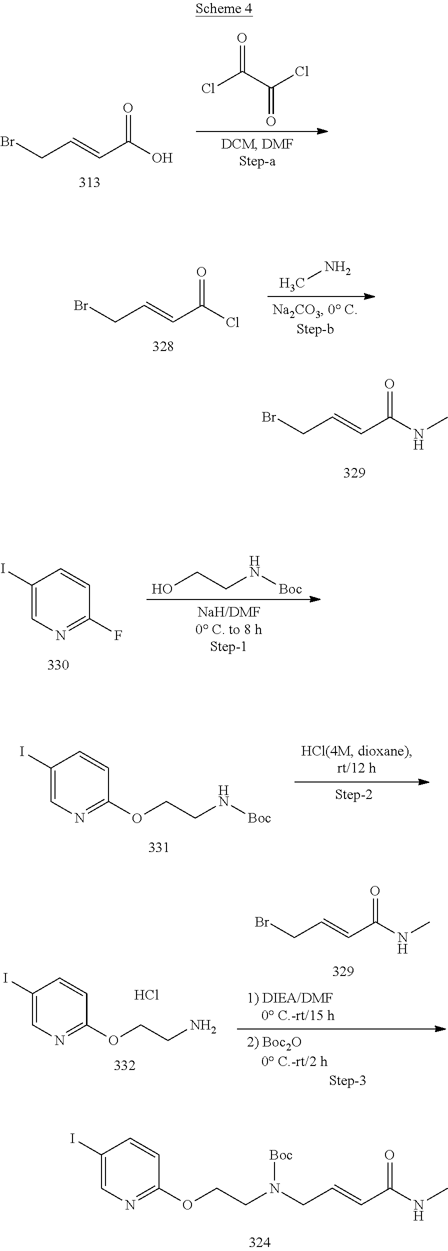

### Step-a: Synthesis of (E)-4-bromobut-2-enoyl chloride

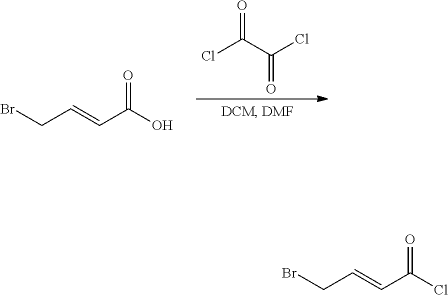 [SMILES: O=C(O)/C=C/CBr ; O=C(Cl)C(=O)Cl ; O=C(Cl)/C=C/CBr]

Into a 500-mL round-bottom flask, was placed (E)-4-bromobut-2-enoic acid (10 g, 60.61 mmol, 1.00 equiv), DCM (200 mL), and N, N-dimethylformamide (0.5 mL). Oxalyl dichloride (7.7 g, 1.00 equiv) was added at 0° C. in dropwise. The resulting solution was stirred until completion at 0° C. The mixture was used directly to the next step without isolation of the product.

### Step-b: Synthesis of (E)-4-bromo-N-methylbut-2-enamide

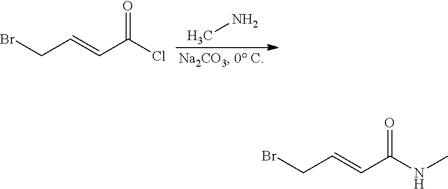

Into a 250-mL round-bottom flask was placed CH₃NH₂.HCl (1.005 g, 1.00 equiv), sodium carbonate (3.18 g, 30.00 mmol, 2.00 equiv), and DCM (100 mL). Then, (E)-4-bromobut-2-enoyl chloride (15.00 mmol, 1.00 equiv) was added dropwise at 0° C. The resulting solution was stirred at 0° C. in a water/ice bath until completion. The mixture was then washed with 2×100 mL of water. The organic layer was concentrated under vacuum to deliver the title compound in 3 g (62%) as a yellow solid. LCMS: 178, 180[M+H]⁺.

### Step-1: Synthesis of tert-butyl (2-((5-iodopyridin-2-yl)ox)ethyl)carbamate

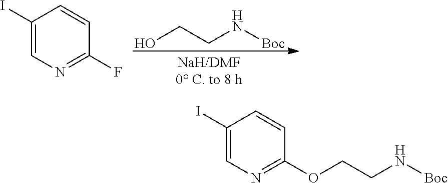

To a stirred solution of 2-fluoro-5-iodopyridine (250 g, 1.12 mmol) in DMF (2.5 L) was added sodium hydride (67.2 g, 1.68 mol), and the solution was stirred for 10 min at 0° C. Then, tert-butyl (2-hydroxyethyl)carbamate (180.4 g, 1.12 mol) was added. The contents were stirred at R.T. until completion. The reaction mixture was poured onto ice cold water, the solid separated was filtered and dried under reduced pressure to deliver the title compound in 301 g as an off-white solid.

### Step-2: Synthesis of 2-((5-iodopyridin-2-yl)oxy)ethan-1-amine hydrochloride

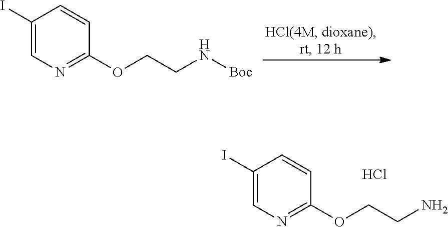 [SMILES: CC(C)(C)OC(=O)NCCOc1ccc(I)cn1 ; NCCOc1ccc(I)cn1 ; Cl]

Into a 100-mL round-bottom flask was placed tert-butyl tert-butyl (2-((5-iodopyridin-2-yl)oxy)ethyl)carbamate (5.6 g, 15.38 mmol, 1.00 equiv), and hydrogen chloride (4M, dioxane) (20 mL). The resulting solution was stirred at room temperature until completion, then concentrated under vacuum to deliver the title compound in 4.0 g (87%) as a white solid.

### Step-3: Synthesis of tert-butyl (E)-2-(S-iodopyridin-2-yloxy)ethyl(4-(methylamino)-4-oxobut-2-enyl)carbamate

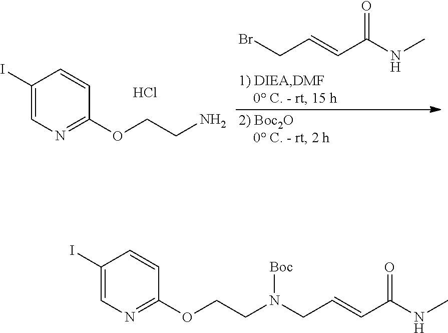

Into a 100-mL round-bottom flask was placed 2-((5-iodopyridin-2-yl)oxy)ethan-1-amine hydrochloride (2 g, 7.57 mmol, 1.00 equiv). DIEA (1.95 g, 2.00 equiv), and N, N-dimethylformamide (10 mL). This was followed by the addition of (E)-4-bromo-N-methylbut-2-enamide (1.02 g, 5.31 mmol, 0.70 equiv) (Scheme 4, Steps-a-b) dropwise with stirring at 0° C. The resulting solution was allowed to react with stirring at room temperature until completion. To this was added (Boc)₂O (1.8 g, 1.20 equiv). The resulting solution was stirred at room temperature until completion. The reaction mixture was diluted with ice cold water (100 mL) and extracted with 3×100 mL ethyl acetate. The organic layers were combined, washed with brine (100 mL), dried over anhydrous sodium sulfate and concentrated under reduced pressure. The crude material was purified by column chromatography with silica gel column using 80% ethyl acetate in n-hexane as an eluent and further purified with C18 column (MeOH/H₂O=7:3) to deliver the title compound in 620 mg (20%) as colorless oil. ¹H NMR (400 MHz, Methanol-d4) δ 8.32 (dd, J=2.4, 0.7 Hz, 1H), 7.96-7.88 (m, 1H), 6.69 (t, J=9.6 Hz, 2H), 5.93 (t, J=13.1 Hz, 1H), 4.42 (d, J=5.4 Hz, 2H), 4.07 (dd, J=5.4, 1.8 Hz, 2H), 3.62 (t, J=5.4 Hz, 2H), 2.78 (s, 3H), 1.44 (s, 9H). LCMS: 462 [M+H]⁺.

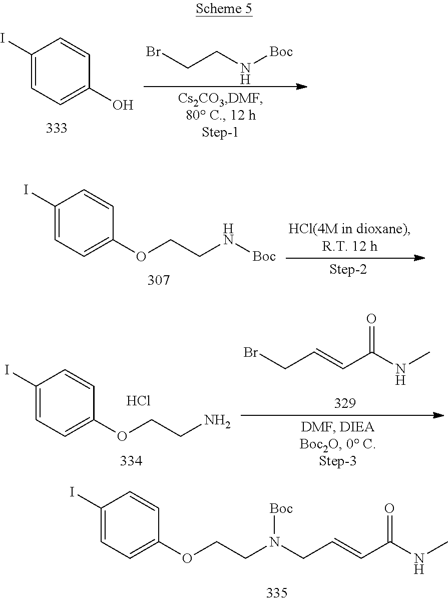

### Step-1: Synthesis of tert-butyl (2-(4-iodophenoxy)ethyl)carbamate

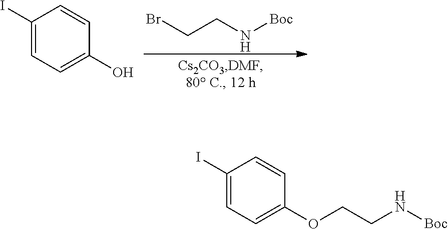 [SMILES: Oc1ccc(I)cc1 ; CC(C)(C)OC(=O)NCCBr ; CC(C)(C)OC(=O)NCCOc1ccc(I)cc1]

To a stirred solution of 4-iodophenol (50 g, 0.227 mol) in DMF (750 mL) was added Cs₂CO₃ (493 g, 1.363 mol). The mixture was stirred for 30 min at room temperature and then tert-butyl (2-bromoethyl)carbamate (71.27 g, 0.318 mol) was added. The solution was stirred at 80° C. until completion. The reaction mixture was then poured onto ice water, solid separated was filtered and dried under reduced pressure to deliver the title compound in 80 g (97%) as an off-white solid. LCMS: 264 [M-Boc+H]⁺.

### Step-2: Synthesis of 2-(4-iodophenoxy)ethan-1-amine hydrochloride

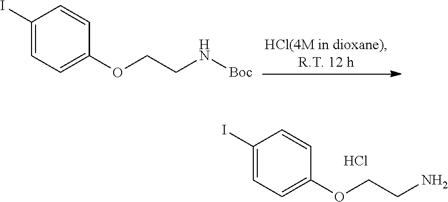 [SMILES: CC(C)(C)OC(=O)NCCOc1ccc(I)cc1 ; NCCOc1ccc(I)cc1 ; Cl]

To a stirred solution of tert-butyl (2-(4-iodophenoxy)ethyl)carbamate (25 g, 68.6 mmol) in dioxane (50 mL) at 0° C. was added 4M HCl in dioxane (250 mL). The reaction mixture was stirred at room temperature until completion. The reaction mixture was then concentrated under reduced pressure to deliver the title compound in 16 g (88%) as crude material, used in next step without further purification.

### Step-3: Synthesis of tert-butyl (E)-2-(4-iodophenoxy)ethyl(4-(methylamino)-4-oxobut-2-enyl)carbamate

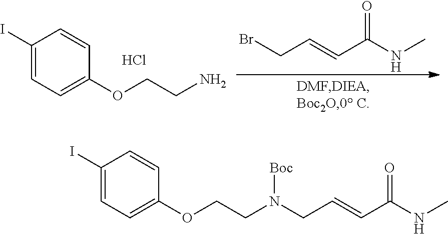

Into a 40-mL vial was placed 2-(4-iodophenoxy)ethan-1-amine hydrochloride (2.26 g, 7.57 mmol, 1.00 equiv), DIEA (1.9 g, 14.70 mmol, 2.00 equiv), and N,N-dimethylformamide (20 mL). (E)-4-bromo-N-methylbut-2-enamide (1.08 g, 6.07 mmol, 0.80 equiv) (Scheme 4. Steps-a-b) was then added to the solution at 0° C., which was then stirred at R.T. until completion. Boc₂O (2.62 g, 12 mmol) was then added and the resulting mixture was stirred at room temperature until completion. Upon completion by TLC, the reaction mixture was cooled to 0° C., quenched with ice cold water (100 mL) and extracted with 3×250 mL of DCM. The combined organic extracts were washed with brine (250 mL), dried over anhydrous sodium sulfate and concentrated under reduced pressure. The crude material was purified by column chromatography over 100-200 mesh silica using 50-80% ethyl acetate in n-hexane as an eluent, then further purified with C18 column (MeOH/H2O=7:3) to deliver the title compound in 690 mg (20%). ¹H NMR (400 MHz, DMSO-d6) δ 7.97-7.91 (m, 1H), 7.63-7.55 (m, 2H), 6.84-6.76 (m, 2H), 6.52 (d, J=12.9 Hz, 1H), 5.89 (d, J=15.5 Hz, 1H), 4.08-4.02 (m, 2H), 4.01-3.94 (m, 2H), 3.49 (d, J=4.8 Hz, 2H), 2.63 (d, J=4.6 Hz, 3H). 1.37 (s, 9H). LCMS: 461 [M+H]⁺.

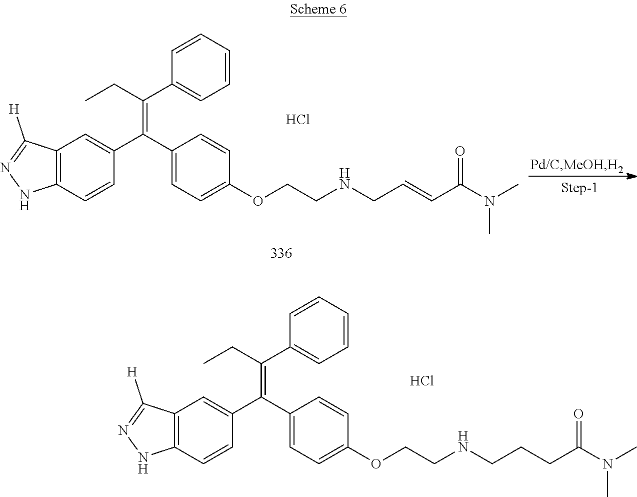 [SMILES: CC/C(=C(/c1ccc(OCCNC/C=C/C(=O)N(C)C)cc1)c1ccc2[nH]ncc2c1)c1ccccc1 ; Cl ; CC/C(=C(/c1ccc(OCCNCCCC(=O)N(C)C)cc1)c1ccc2[nH]ncc2c1)c1ccccc1 ; Cl]

### Example 4: Synthesis of (E)-4-(2-(4-(1-(1H-Indazol-5-yl)-2-phenylbut-1-enyl)phenoxy)ethylamino)-N,N-dimethylbutanamide (Compound 4)

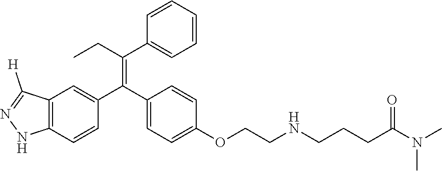

### Step-1: Synthesis of (E)-4-(2-(4-(1-(1H-indazol-5-yl)-2-phenylbut-1-enyl)phenoxy)ethylamino)-N,N-dimethylbutanamide

Into a 250-mL round-bottom flask was placed (E)-4-((2-(4-((E)-1-(1H-indazol-5-yl)-2-phenylbut-1-en-1-yl)phenoxy)ethyl)amino)-N,N-dimethylbut-2-enamide (3 g, 6.07 mmol, 1.00 equiv) (synthesized following the approach outlined in patent US 2016347717 A1), methanol (100 mL), and palladium/carbon (300 mg, 0.10 equiv). To the above, H₂(g) was introduced in. The resulting solution was stirred in a water/ice bath until completion, and then the solids were filtered out. The resulting mixture was concentrated under vacuum to deliver the title compound in 2.07 g, 69% overall yield, as a hydrochloride salt. ¹H NMR (400 MHz, DMSO-d6) δ 13.11 (d, J=2.6 Hz, 1H), 8.98 (brs, 2H), 8.07 (s, 1H), 7.61 (s, 1H), 7.53-7.51 (d, J=8.6 Hz, 1H), 7.23-7.19 (m, 6H), 6.81-6.79 (m, 2H), 6.66-6.64 (m, 2H), 4.10 (d, J=5.0 Hz, 2H), 3.32-3.24 (d, J=6.5 Hz, 2H), 2.93 (s, 5H), 2.80 (s, 3H), 2.43-2.40 (m, 4H), 1.85-1.78 (m, 2H), 0.90-0.86 (t, J=7.4 Hz, 3H). LCMS: 497.4 [M+H]⁺.

### Example 5: Synthesis of (E)-N-methyl-4-((2-((5-((E)-4,4,4-trifluoro-1-(3-fluoro-1H-indazol-5-yl)-1-phenylbut-1-en-2-yl)pyridin-2-yl)oxy)ethyl)amino)but-2-enamide (Compound 5)

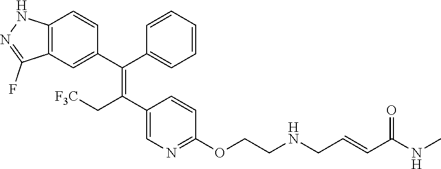 [SMILES: CNC(=O)/C=C/CNCCOc1ccc(/C(CC(F)(F)F)=C(\c2ccccc2)c2ccc3[nH]nc(F)c3c2)cn1]

Compound 5 was synthesized following the approach outlined in Scheme 3, Example 3. Its precursor was formed in Step 7 as a minor product, which was carried through the remaining steps outlined in the Scheme to deliver the title compound in 4.9 g (2.69%) as a yellow solid. ¹H-NMR (300 MHz, DMSO-d6) δ 12.70 (s, 1H), 7.97-7.85 (m, 2H), 7.65-7.49 (m, 3H), 7.23 (dd, J=8.8, 1.5 Hz, 1H), 7.21-7.03 (m, 3H), 7.04-6.94 (m, 2H), 6.69 (dd, J=8.5, 0.7 Hz, 1H), 6.07-5.94 (m, 1H), 4.19 (t, J=5.7 Hz, 2H), 3.47 (t, J=10.9 Hz, 2H), 3.30 (d, J=6.2 Hz, 2H), 2.80 (t, J=5.7 Hz, 2H), 2.63 (d, J=4.6 Hz, 3H). LCMS: 554.69 [M+H]⁺.

### Example 6: Synthesis of (E)-N-methyl-5-((2-((5-((Z)-4,4,4-trifluoro-1-(3-fluoro-1H-indazol-5-yl)-2-phenylbut-1-en-1-yl)pyridin-2-yl)oxy)ethyl)amino)pent-2-enamide (Compound 6)

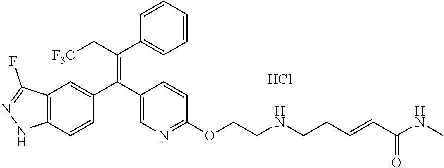 [SMILES: CNC(=O)/C=C/CCNCCOc1ccc(/C(=C(/CC(F)(F)F)c2ccccc2)c2ccc3[nH]nc(F)c3c2)cn1 ; Cl]

Compound 6 was synthesized following the approach outlined in Scheme 3 by modifying: a) Step-7 by substituting tert-butyl (E)-(2-((5-iodopyridin-2-yl)oxy)ethyl)(5-(methylamino)-5-oxopent-3-en-1-yl)carbamate (preparation shown below, Steps-a-g) for compound 324, 0.1 equiv Pd(dppf)Cl₂ for Pd(PPh₃)₂Cl₂, dioxane for 2-Methyl THF, using 2.5 equiv of Cs₂CO₃, and stirring the reaction at 50° C. until completion without purification, and b) Step-8 by substituting 0.1 equiv Pd(dppf)Cl₂ for Pd(PPh₃)₂Cl₂ to deliver the title compound in 58.5 mg, 0.90% overall yield. ¹H NMR (400 MHz, Methanol-d₄) δ 7.70 (dd, J=2.4, 0.8 Hz, 1H), 7.64 (t, J=1.2 Hz, 1H), 7.50-7.48 (m, 1H), 7.41-7.16 (m, 7H), 6.68-6.60 (m, 2H), 6.06-6.02 (d, J=15.2 Hz, 1H), 4.46-4.44 (m, 2H), 3.45-3.37 (m, 4H), 3.30-3.15 (t, J=7.6 Hz, 2H), 2.79 (s, 3H), 2.60-2.55 (m, 2H). LCMS: 568 [M+H]⁺.

### Step-a: Synthesis of 3-((tert-butyldimethylsilyl)oxy)propanal

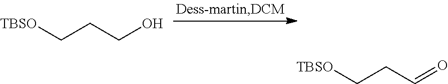 [SMILES: CC(C)(C)[Si](C)(C)OCCCO ; CC(C)(C)[Si](C)(C)OCCC=O]

Into a 500-mL round-bottom flask was placed 3-((tert-butyldimethylsilyl)oxy)propan-1-ol (20 g, 105.07 mmol, 1.00 equiv), DCM (200 mL), and Dess-Martin Periodinane (53 g, 1.00 equiv). The resulting solution was stirred at 25° C. until completion. The solids were filtered out via a Buchner funnel to deliver the title compound in 20 g (crude) as yellow oil. The material was used directly in the next step without purification.

### Step-b: Synthesis of ethyl (E)-5-((tert-butyldimethylsilyl)oxy)pent-2-enoate

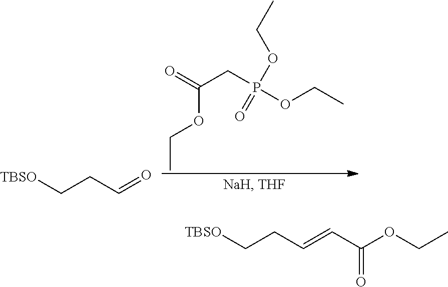

Into a 500-mL round-bottom flask was placed ethyl 2-(diethoxyphosphoryl)acetate (24 g. 107.05 mmol, 1.00 equiv), THF (200 mL), and sodium hydride (4.24 g, 176.67 mmol, 1.00 equiv). The resulting solution was stirred for 2 h at 0° C. in a water/ice bath. Then 3-((tert-butyldimethylsilyl)oxy)propanal (20 g, 106.19 mmol, 1.00 equiv) was added. The resulting solution was allowed to react, with stirring, at 25° C. until completion. The reaction was then quenched by the addition of 100 mL of water. The resulting solution was extracted with 2×200 mL of ethyl acetate and the organic layers were combined and concentrated under vacuum. The residue was applied onto a silica gel column with ethyl acetate/petroleum ether (1:10) to deliver the title compound in 10 g (36%) as yellow oil.

### Step-c: Synthesis of (E)-5-((tert-butyldimethylsilyl)oxy)pent-2-enoic acid

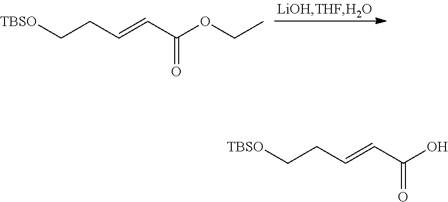 [SMILES: CCOC(=O)/C=C/CCO[Si](C)(C)C(C)(C)C ; CC(C)(C)[Si](C)(C)OCC/C=C/C(=O)O]

Into a 100-mL round-bottom flask was placed ethyl (E)-5-((tert-butyldimethylsilyl)oxy)pent-2-enoate (1 g, 3.87 mmol, 1.00 equiv), methanol (5 mL), LiOH (500 mg, 20.88 mmol, 5.00 equiv), and water (5 mL). The resulting solution was stirred at 0° C. in a water/ice bath until completion. The pH value of the solution was adjusted to 7 with hydrogen chloride (1M) (3 mL). The resulting solution was extracted with 3×50 mL of DCM and the organic layers were combined. The organic layers were dried over anhydrous sodium sulfate and concentrated under vacuum to deliver the title compound in 0.5 g (56%) as yellow oil. LCMS: 231 [M+H]⁺.

### Step-d Synthesis of (E)-5-((tert-butyldimethylsilyl)oxy)-N-methylpent-2-enamide

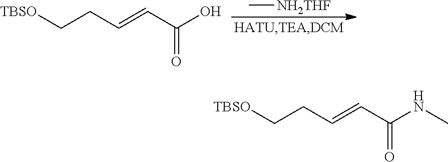

Into a 250-mL round-bottom flask was placed (E)-5-((tert-butyldimethylsilyl)oxy)pent-2-enoic acid (3.0 g, 13.02 mmol, 1.00 equiv), DCM (50 mL), HATU (7.4 g, 19.46 mmol, 1.50 equiv), TEA (2.6 g, 25.69 mmol, 2.00 equiv), CH₃NH₂-THF (13 mL). The resulting solution was stirred at room temperature until completion. The reaction was then quenched by the addition of water. The resulting solution was extracted with 3×100 mL of DCM and the organic layers combined and the organic layer was washed with brine (100 mL), dried over anhydrous sodium sulfate and concentrated under vacuum to deliver the title compound in 3.0 g (95%) as brown oil. LCMS: 244 [M+H]⁺.

### Step-e: Synthesis of (E)-5-hydroxy-N-methylpent-2-enamide

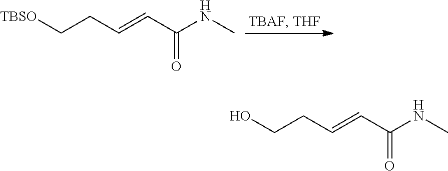

Into an 8-mL round-bottom flask purged and maintained with an inert atmosphere of nitrogen was placed (E)-5-((tert-butyldimethylsilyl)oxy)-N-methylpent-2-enamide (100 mg, 0.41 mmol, 1.00 equiv), TBAF (215 mg, 0.82 mmol, 2.00 equiv), and THF (2 mL). The resulting solution was stirred at room temperature until completion to deliver the title compound as crude material, used in next step without further purification, considering 100% yield.

### Step-f: Synthesis of (E)-5-(methylamino)-5-oxopent-3-en-1-yl methanesulfonate

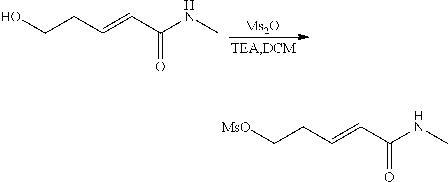

Into a 50-mL round-bottom flask purged and maintained with an inert atmosphere of nitrogen was placed (E)-5-hydroxy-N-methylpent-2-enamide (2.7 g, 20.90 mmol, 1.00 equiv), TEA (4.14 g, 40.91 mmol, 2.00 equiv), DCM (20 mL), and methanesulfonylmethanesulfonate (7.14 g, 40.99 mmol, 2.00 equiv). The resulting solution was stirred at room temperature until completion. The reaction was then quenched by the addition of water. The resulting mixture was concentrated under vacuum. The crude product (10 mL) was purified by Flash-Prep-HPLC with Column C18 using (50%-60%) CH₃CN in water to deliver the title compound in 4.0 g (92%) as a brown syrup. LCMS: 208 [M+H]⁺.

### Step-g: Synthesis of tert-butyl (E)-(2-((5-iodopyridin-2-yl)oxy)ethyl)(5-(methylamino)-5-oxopent-3-en-1-yl)carbamate

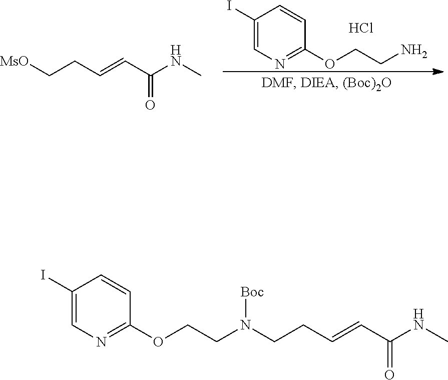 [SMILES: CNC(=O)/C=C/CCOS(C)(=O)=O ; NCCOc1ccc(I)cn1 ; Cl ; CNC(=O)/C=C/CCN(CCOc1ccc(I)cn1)C(=O)OC(C)(C)C]

Into a 500-mL round-bottom flask was placed 2-(2-aminoethoxy)-5-iodopyridine hydrochloride (6.5 g, 19.29 mmol, 1.00 equiv), and N,N-dimethylformamide (50 mL). This was followed by the addition of DIEA (10 g, 77.38 mmol, 4.00 equiv) dropwise with stirring at 0° C. To this was added (E)-5-(methylamino)-5-oxopent-3-en-1-yl methanesulfonate (4.0 g, 19.30 mmol, 1.00 equiv), in portions at 0° C. The resulting solution was stirred at 40° C. in an oil bath until completion. To the mixture was added (Boc)₂O (8.4 g, 38.49 mmol, 2.00 equiv). The resulting solution was allowed to react at room temperature until completion. The reaction was then quenched by the addition of water. The resulting solution was extracted with 3×100 mL of ethyl acetate and the organic layers combined and the organic layer was washed with brine (100 mL), dried over anhydrous sodium sulfate and concentrated under vacuum. The residue was applied onto a silica gel column with ethyl acetate/petroleum ether (20:1). The solid was dried in an oven under reduced pressure to deliver the title compound in 1.5 g (16%) as a yellow syrup. Product isolated was still not clean, taken forward to the next step without further purification. LCMS: 476 [M+H]⁺.

### Example 7: Synthesis of (E)-N-(2-hydroxyethyl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (Compound 7)

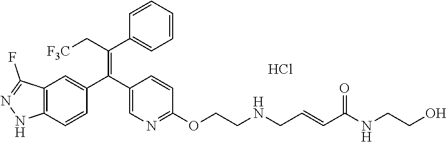 [SMILES: O=C(/C=C/CNCCOc1ccc(/C(=C(/CC(F)(F)F)c2ccccc2)c2ccc3[nH]nc(F)c3c2)cn1)NCCO ; Cl]

Compound 7 was synthesized following the approach outlined in Scheme 3 by modifying: a) Step-7 by substituting tert-butyl (E)-(4-((2-hydroxyethyl)amino)-4-oxobut-2-en-1-yl)-2-((5-iodopyridin-2-yl)oxy)ethyl)carbamate (preparation shown below, Steps-a-b) for compound 324, dioxane for 2-Methyl THF, using 3.0 equiv of Cs₂CO₃, and stirring at 40° C. until completion, b) Step-8 by substituting 3.0 equiv potassium carbonate for KOH, and c) Step-9 by substituting concentrated HCl (to make a 0.03M solution) for TFA to deliver the title compound in 19.5 mg, 0.13% overall yield. ¹H NMR (400 MHz, Methanol-d4) δ 7.73 (m, 1H), 7.72 (m, 1H), 7.66-7.50 (m, 1H), 7.35-7.31 (m, 2H), 7.26-7.20 (m, 5H), 6.73-6.66 (m, 2H), 6.35-6.31 (m, 1H), 4.49-4.46 (m, 2H), 3.87-3.85 (m, 2H), 3.65-3.63 (t, J=5.7 Hz, 2H), 3.48-3.38 (m, 6H). LCMS: 584.2 [M+H]⁺.

### Step-a. Synthesis of (E)-4-bromo-N-(2-(tert-butyldimethylsilyloxy)ethyl)but-2-enamide

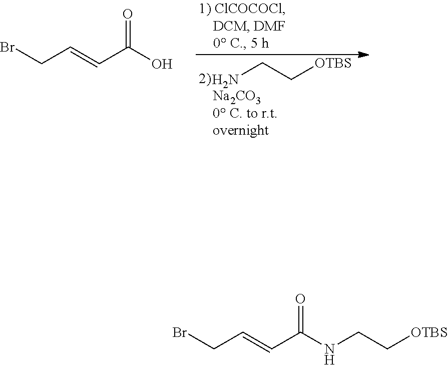 [SMILES: O=C(O)/C=C/CBr ; CC(C)(C)[Si](C)(C)OCCN ; CC(C)(C)[Si](C)(C)OCCNC(=O)/C=C/CBr]

Into a 3000-mL round-bottom flask was placed (E)-4-bromobut-2-enoic acid (105 g, 636.42 mmol, 1.00 equiv), DCM (1000 mL), and N,N-dimethylformamide (5 mL), then oxalyl dichloride (88.7 g, 698.83 mmol, 1.10 equiv) was added in dropwise. The resulting solution was stirred at 0° C. until completion to obtain the corresponding acid chloride. Into a 5000-mL round-bottom flask, was placed (2-aminoethoxy)(tert-butyl)dimethylsilane (111.4 g, 636.42 mmol, 1.00 equiv), DCM (1000 mL), and sodium carbonate (203.5 g, 1.92 mol, 3.00 equiv). The acid chloride solution was then added in dropwise to the solution with stirring at 0° C. The resulting solution was stirred overnight at room temperature. The reaction was then quenched by the addition of 1000 mL of water and extracted with 3×1000 mL of DCM. The organic layers are combined, dried over anhydrous sodium sulfate and concentrated under vacuum to deliver the title compound in 206 g (crude) as a yellow oil. LCMS: 322 [M+H]⁺.

### Step-b: Synthesis of tert-butyl (E)-(4-((2-hydroxyethyl)amino)-4-oxobut-2-en-1-yl)(2-((5-iodopyridin-2-yl)oxy)ethyl)carbamate

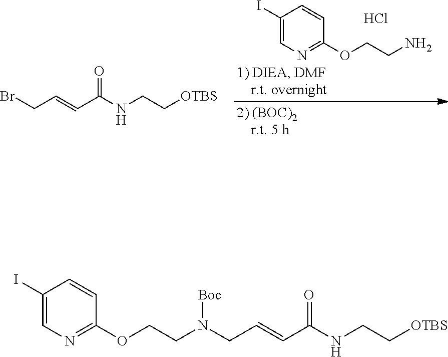 [SMILES: NCCOc1ccc(I)cn1 ; Cl ; CC(C)(C)[Si](C)(C)OCCNC(=O)/C=C/CBr ; CC(C)(C)OC(=O)N(C/C=C/C(=O)NCCO[Si](C)(C)C(C)(C)C)CCOc1ccc(I)cn1]

Into a 5000-mL round-bottom flask was placed 2-((5-iodopyridin-2-yl)oxy)ethan-1-amine hydrochloride (172 g, 510 mmol, 1.0 equiv), DIEA (263 g, 2.03 mol, 4.00 equiv), and N,N-dimethylformamide (800 mL), and the solution was cooled to 0° C. (E)-4-bromo-N-(2-(tert-butyldimethylsilyloxy)ethyl)but-2-enamide (206 g, 640 mmol, 1.25 equiv) was dissolved into 200 mL DMF. This solution was then added dropwise to the flask with stirring at 0° C. The resulting solution was stirred overnight at room temperature. After that, di-tert-butyl dicarbonate (222 g, 1.02 mol, 2.00 equiv) was added. The resulting solution was stirred at room temperature until completion. The resulting solution was diluted with 2 L of ethyl acetate, then washed with 3×2000 mL of H₂O. The mixture was dried over anhydrous sodium sulfate and concentrated under vacuum. The residue was applied onto a silica gel column with ethyl acetate/petroleum ether (1:5) to deliver the title compound in 22 g (6%) as a yellow oil. ¹H NMR (300 MHz, Chloroform-d) δ 8.33 (s, 1H), 7.81 (d, J=8.7 Hz, 1H), 6.81-6.75 (m, 1H), 6.62-6.59 (m, 1H), 5.95-5.70 (m, 1H), 4.40 (s, 2H), 4.08 (s, 2H), 3.73 (t, J=5.1 Hz, 2H), 3.59 (s, 2H), 3.49-3.43 (m, 2H), 1.46 (s, 9H), 0.92 (s, 9H), 0.09 (s, 6H). LCMS: 606 [M+H]⁺.

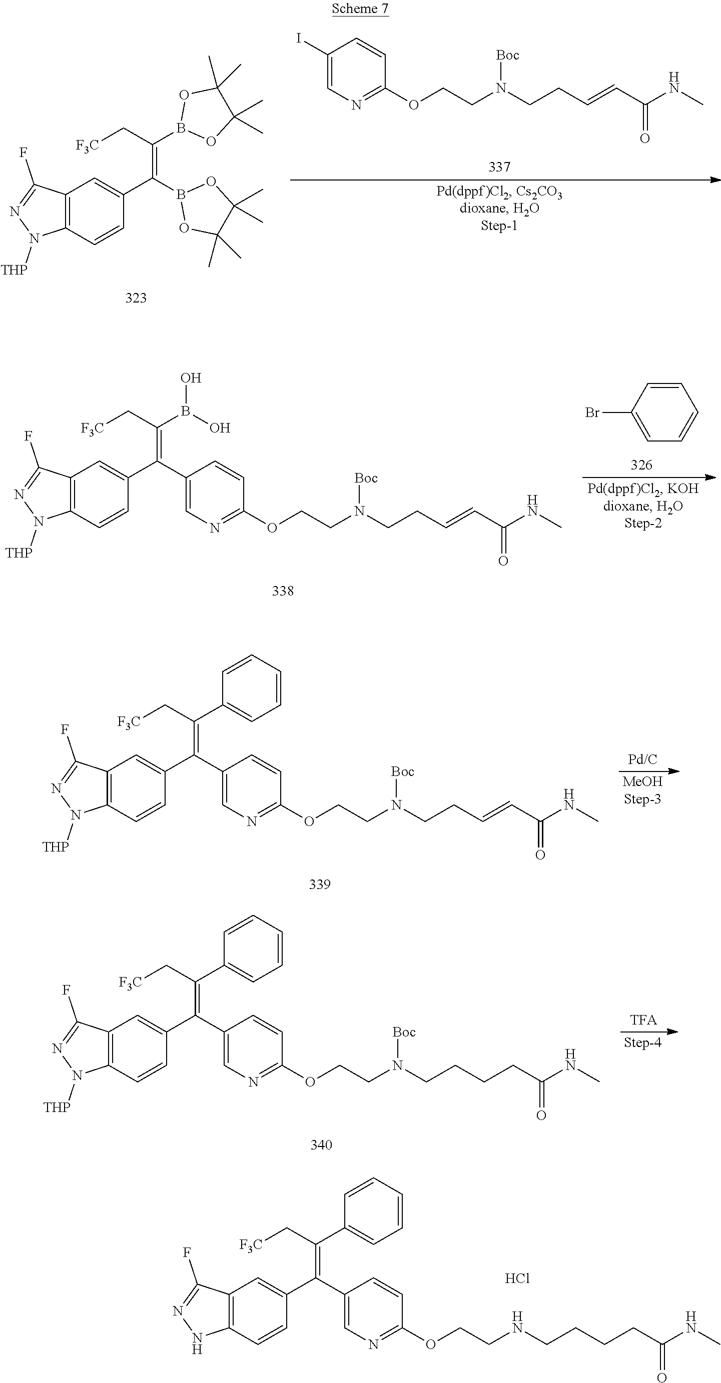

### Example 8: Synthesis of (Z)—N-methyl-5-((2-((5-(4,4,4-trifluoro-1-(3-fluoro-1H-Indazol-5-yl)-2-phenylbut-1-en-1-yl)pyridin-2-yl)oxy)ethylamino)pentanamide (Compound 8)

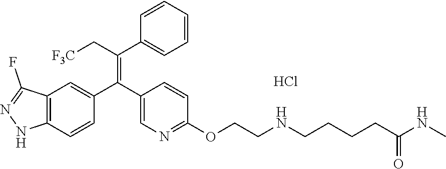 [SMILES: CNC(=O)CCCCNCCOc1ccc(/C(=C(/CC(F)(F)F)c2ccccc2)c2ccc3[nH]nc(F)c3c2)cn1 ; Cl]

### Step-1: Synthesis of ((E)-1-(6-(2-((tert-butoxycarbonyl)((E)-5-(methylamino)-5-oxopent-3-en-1-yl)amino)ethoxy)pyridin-3-yl)-4,4,4-trifluoro-1-(3-fluoro-1-(tetrahydro-2H-pyran-2-yl)-1H-indazol-5-yl)but-1-en-2-yl)boronic acid

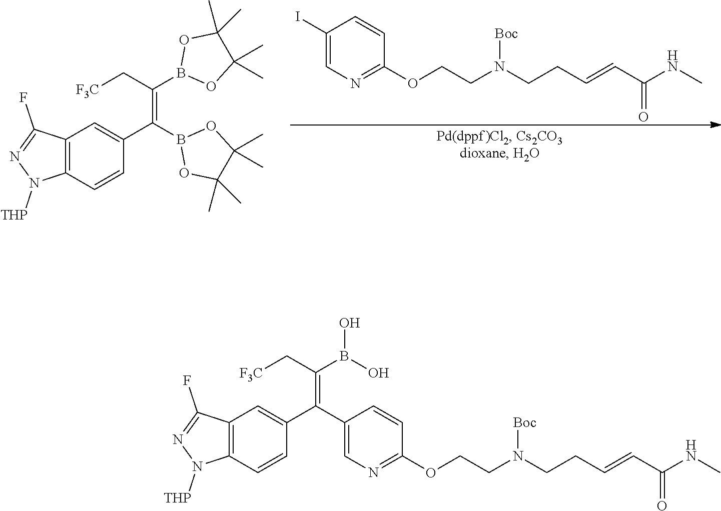

Into a 40-mL round-bottom flask purged and maintained with an inert atmosphere of nitrogen was placed (Z)-3-fluoro-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (1.6 g, 2.76 mmol, 1.00 equiv) (Scheme 3, steps 1-6), tert-butyl (E)-(2-((5-iodopyridin-2-yl)oxy)ethyl)(5-(methylamino)-5-oxopent-3-en-1-yl)carbamate (1.3 g, 2.73 mmol, 1.00 equiv) (preparation shown in example 6, Steps-a-d). Pd(dppf)Cl₂ (190 mg, 0.26 mmol, 0.10 equiv), Cs₂CO₃ (2.2 g, 6.75 mmol, 2.50 equiv), dioxane (10 mL), and water (2 mL). The resulting solution was stirred at 50° C. in an oil bath until completion. The crude material was used in next step without further purification

### Step-2: Synthesis of ter-butyl ((E)-5-(methylamino)-5-oxopent-3-en-1-yl)(2-((5-((Z)-4,4,4-trifluoro-1-(3-fluoro-1-(tetrahydro-2H-pyran-2-yl)-1H-indazol-5-yl)-2-phenylbut-1-en-1-yl)pyridin-2-yl)oxy)ethyl)carbamate

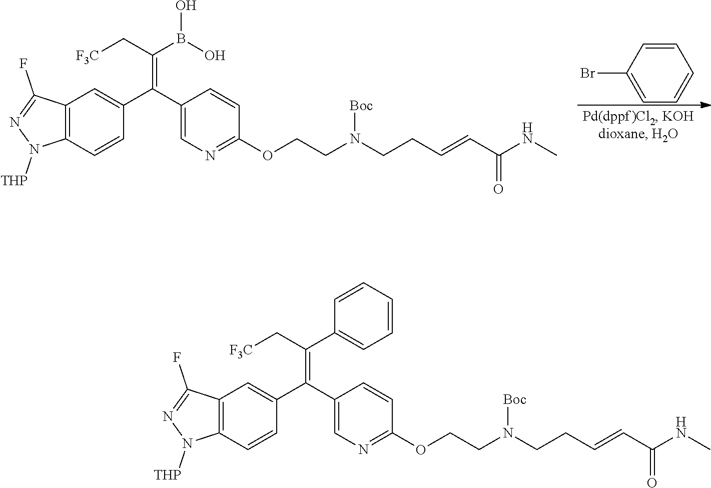

Into a 40-mL round-bottom flask purged and maintained with an inert atmosphere of nitrogen was placed ((E)-1-(6-(2-((tert-butoxycarbonyl)((E)-5-(methylamino)-5-oxopent-3-en-1-yl)amino)ethoxy)pyridin-3-yl)-4,4,4-trifluoro-1-(3-fluoro-1-(tetrahydro-2H-pyran-2-yl)-1H-indazol-5-yl)but-1-en-2-yl)boronic acid (2.0 g, 2.49 mmol, 1.00 equiv), bromobenzene (430 mg, 2.74 mmol, 1.10 equiv), Pd(dppf)Cl₂ (180 mg, 0.25 mmol, 0.10 equiv), KOH (980 mg, 17.47 mmol, 7.00 equiv), dioxane (10 mL), and water (2 mL). The resulting solution was stirred at 80° C. in an oil bath until completion. The reaction was then quenched by the addition of water. The resulting solution was extracted with 3×50 mL of ethyl acetate and the organic layer was washed with brine (100 mL), dried over anhydrous sodium sulfate and concentrated under vacuum. The residue was applied onto a silica gel column with ethyl acetate/petroleum ether (1:1). The solid was dried in an oven under reduced pressure to deliver the title compound in 1.0 g (53%) as an off-white solid.

### Step-3: Synthesis of tert-butyl (Z)-(5-(methylamino)-5-oxopentyl)(2-((5-(4,4,4-trifluoro-1-(3-fluoro-1-(tetrahydr-2H-pyran-2-yl)-1H-Indazol-5-yl)-2-phenylbut-1-en-1-yl)pyridin-2-yl)oxy)ethyl)carbamate

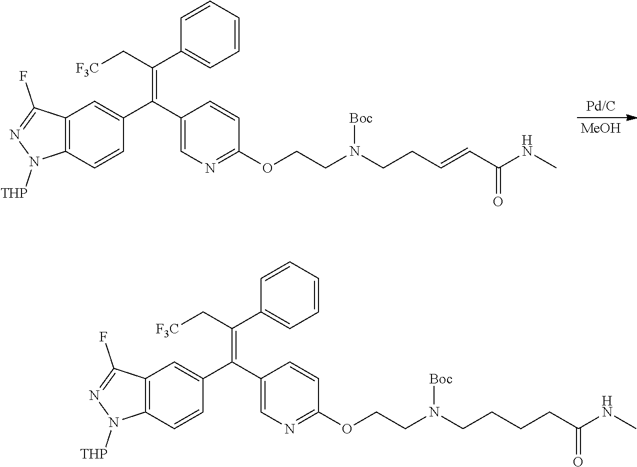

Into a 50-mL round-bottom flask purged and maintained with an inert atmosphere of nitrogen was placed tert-butyl ((E)-5-(methylamino)-5-oxopent-3-en-1-yl)(2-((5-((Z)-4,4,4-trifluoro-1-(3-fluoro-1-(tetrahydro-2H-pyran-2-yl)-1H-indazol-5-yl)-2-phenylbut-1-en-1-yl)pyridin-2-yl)oxy)ethyl)carbamate (500 mg, 0.67 mmol, 1.00 equiv), Pd/C (200 mg), and methanol (30 mL). The resulting solution was stirred at room temperature until completion. The solids were filtered out and the resulting mixture was concentrated under vacuum to deliver the title compound in 0.4 g (80%) as a brown solid.

### Step-4: Synthesis of (Z)—N-methyl-5-((2-((5-(4,4,4-trifluoro-1-(3-fluoro-1H-indazol-5-yl)-2-phenylbut-1-en-1-yl)pyridin-2-yl)oxy)ethyl)amino)pentanamide

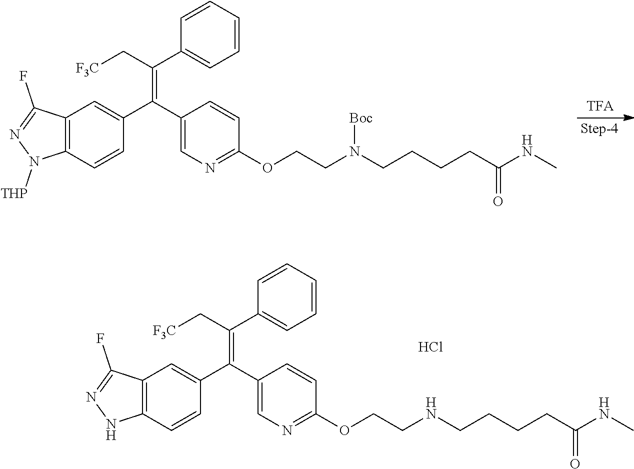

Into a 40-mL round-bottom flask was placed tert-butyl (Z)-(5-(methylamino)-5-oxopentyl)(2-((5-(4,4,4-trifluoro-1-(3-fluoro-1-(tetrahydro-2H-pyran-2-yl)-1H-indazol-5-yl)-2-phenylbut-1-en-1-yl)pyridin-2-yl)oxy)ethyl)carbamate (400 mg, 0.53 mmol, 1.00 equiv), and trifluoroacetic acid (5 mL). The resulting solution was stirred at room temperature until completion. The crude product (10 mL) was purified by Flash-Prep-HPLC with Column C18 using (15%-45%) CH₃CN in water (HCl 0.05%) to deliver the title compound in 78.4 mg, 1.09% overall yield. ¹H NMR (300 MHz, Methanol-d₄) δ 7.88-7.85 (m, 2H), 7.75 (s, 1H), 7.59-7.55 (dd, J=8.4, 2.1 Hz, 1H), 7.41-7.38 (dd, J=8.7, 1.5 Hz, 1H), 7.33-7.27 (m, 5H), 7.201-7.18 (d, J=9.6 Hz, 1H), 4.67-7.64 (t, J=4.8 Hz, 2H), 3.54-3.43 (m, 4H), 3.15-3.10 (t, J=7.2 Hz, 2H), 2.76 (s, 3H), 2.36-2.31 (t, J=6.6 Hz, 2H), 1.79-1.69 (m, 4H). LCMS: 570 [M+H]⁺.

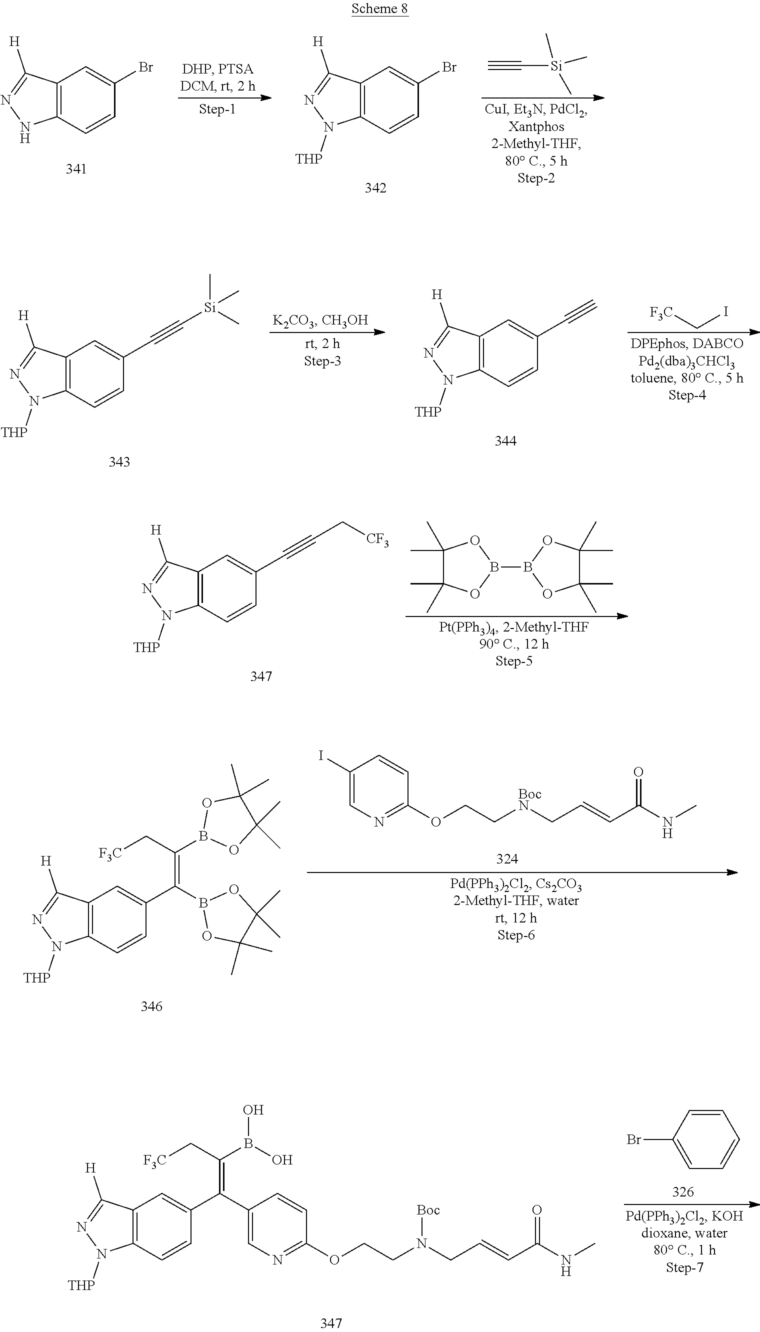

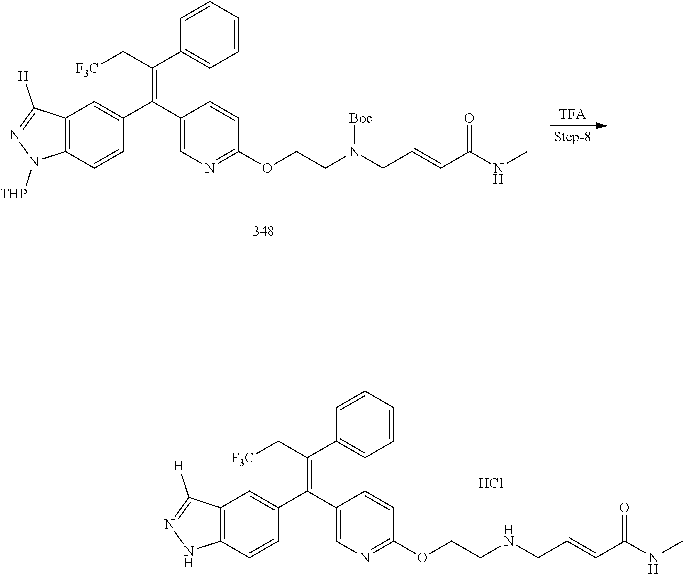

### Example 9: Synthesis of (E)-N-methyl-4-((2-((5-((Z)-4,4,4-trifluoro-1-(1H-indazol-5-yl)-2-phenylbut-1-en-1-yl)pyridin-2-yl)phenoxy)ethyl)amino)but-2-enamide (Compound 9)

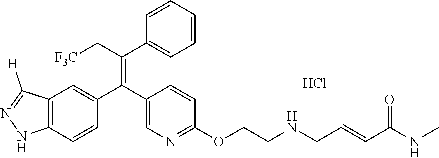 [SMILES: CNC(=O)/C=C/CNCCOc1ccc(/C(=C(/CC(F)(F)F)c2ccccc2)c2ccc3[nH]ncc3c2)cn1 ; Cl]

### Step-1: Synthesis of 5-bromo-1-(tetrahydro-2H-pyran-2 yl)-1H-indazole

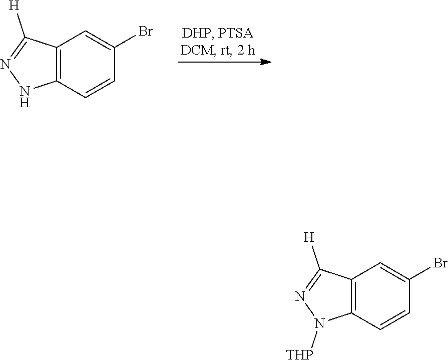

Into a 5000-mL round-bottom flask was placed 5-bromo-1H-indazole (200 g, 1.015 mol, 1.0 eq), DHP (170.6 g, 2.03 mmol, 2.00 equiv), DCM (3000 mL), and PTSA (19.3 g, 0.10 equiv). The resulting solution was stirred at room temperature until completion. The reaction was then quenched by adding saturated NaHCO₃ (aq). The resulting solution was extracted with 3×1000 mL of DCM and the organic layers were combined and concentrated under vacuum. The residue was applied onto a silica gel column with ethyl acetate/petroleum ether (1:10). The collected fractions were combined and concentrated under vacuum to deliver the title compound in 200 g (70%) as a light brown oil. ¹H NMR (300 MHz, DMSO-d₆) δ 8.11 (s, 1H), 8.04 (s, 1H), 7.74 (d, J=9.0 Hz, 1H), 7.55 (dd, J=9.0, 1.8 Hz, 1H), 5.87 (dd, J=9.6, 2.4 Hz, 1H), 3.95-3.65 (m, 2H), 2.50-2.30 (m, 1H), 2.01-1.94 (m, 1H), 1.86-1.41 (m, 4H). LCMS: 281.0 [M+H]⁺.

### Step-2: Synthesis of 1-(tetrahydro-2H-pyran-2-yl)-5-((trimethylsilyl)ethynyl)-1H-indazole

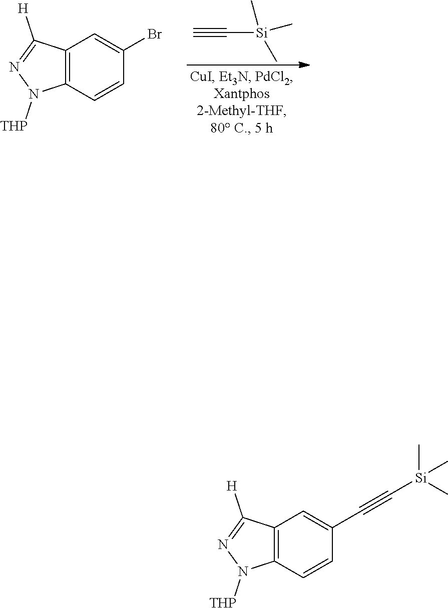

Into a 5000-mL round-bottom flask purged and maintained with an inert atmosphere of nitrogen was placed 5-bromo-1-(tetrahydro-2H-pyran-2-yl)-1H-indazole (200 g, 711.35 mmol, 1.00 equiv), ethynyltrimethylsilane (700 g, 7.15 mol, 10.00 equiv), CuI (40 g, 210.05 mmol, 0.30 equiv), triethylamine (360 g, 3.56 mol, 500 equiv), PdCl₂ (13 g, 0.10 equiv), Xantphos (80 g, 138.25 mmol, 0.20 equiv), and 2-Methyl THF (2000 mL). The resulting solution was stirred at 80° C. in an oil bath until completion. The reaction was then quenched by water. The resulting solution was extracted with 3×2000 mL of ethyl acetate, the organic layers were combined and concentrated under vacuum. The residue was applied onto a silica gel column with ethyl acetate/petroleum ether (1:10). The solid was dried in an oven under reduced pressure to deliver the title compound in 100 g (47%) as a brown oil. ¹H NMR (400 MHz, DMSO-d₆) δ 8.12 (s, 1H), 7.94 (s, 1H), 7.74 (d, J=9.0 Hz, 1H), 7.46 (dd, J=9.0, 1.8 Hz, 1H), 5.87 (dd, J=9.6, 2.4 Hz, 1H), 3.91-3.86 (m, 1H), 3.77-3.71 (m, 1H), 2.43-2.34 (m, 1H), 2.06-1.95 (m, 1H), 1.77-1.41 (m, 4H), 0.24 (s, 9H). LCMS: 299.0 [M+H]⁺.

### Step-3: Synthesis of 5-ethynyl-1-(tetrahydro-2H-pyran-2-yl)-1H-indazole

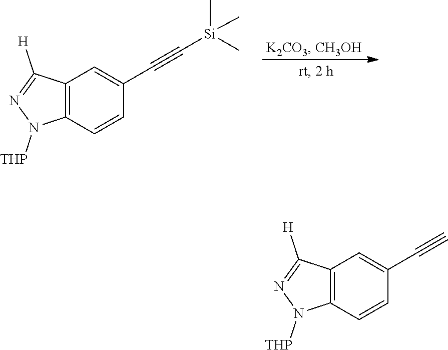

Into a 1000-mL round-bottom flask was placed 1-(tetrahydro-2H-pyran-2-yl)-5-((trimethylsilyl)ethynyl)-1H-indazole (60 g, 201.04 mmol, 1.00 equiv), potassium carbonate (55 g, 397.95 mmol, 2.00 equiv), and methanol (600 mL). The resulting solution was stirred at room temperature until completion. The reaction was then quenched by the addition of water (500 mL). The resulting solution was extracted with 3×500 mL of ethyl acetate and the organic layers combined and concentrated under vacuum to deliver the title compound in 43 g (95%) as a brown oil. ¹H NMR (400 MHz, DMSO-d6) δ 8.14 (s, 1H), 7.96 (s, 1H), 7.75 (d, J=8.0 Hz, 1H), 7.48 (dd, J=8.8, 1.6 Hz, 1H), 5.86 (dd, J=9.8, 2.4 Hz, 1H), 4.08 (s, 1H), 3.90-3.86 (m, 1H), 3.79-3.71 (m, 1H), 2.43-2.34 (m, 1H), 2.07-1.94 (m, 1H), 1.80-1.40 (m, 4H). LCMS: 227.0 [M+H]⁺.

### Step-4: Synthesis of 1-(tetrahydro-2H-pyran-2-yl)-5-(4,4,4-trifluorobut-1-yn-1-yl)-1H-indazole

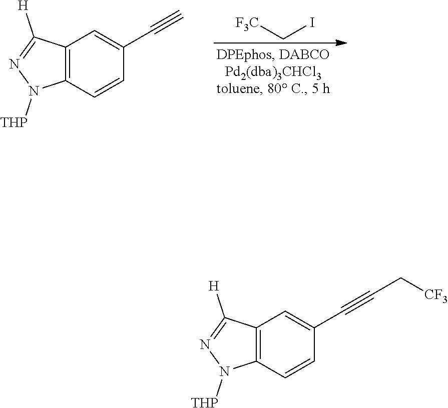

Into a 1000-mL round-bottom flask purged and maintained with an inert atmosphere of nitrogen was placed 5-ethynyl-1-(tetrahydro-2H-pyran-2-yl)-1H-indazole (30 g, 132.58 mmol, 1.00 equiv), 1,1,1-trifluoro-2-iodoethane (55.5 g, 264.37 mmol, 2.00 equiv), DPEPhos (14.1 g, 0.20 equiv). DABOC (29.7 g, 2.00 equiv), Pd₂(dba)₃CHCl₃ (6.84 g, 0.05 equiv), and toluene (300 mL). The resulting solution was stirred at 80° C. in an oil bath until completion. The reaction was then quenched by water (300 mL). The resulting solution was extracted with 3×300 mL of ethyl acetate and the organic layers combined and concentrated under vacuum. The residue was applied onto a silica gel column with ethyl acetate/petroleum ether (1:4) to deliver the title compound in 30 g (73%) as a yellow solid. ¹H NMR (400 MHz. Methanol-d₄) δ 8.05 (s, 1H), 7.88 (s, 1H), 7.66 (d, J=8.8 Hz, 1H), 7.45 (dd, J=8.8, 1.6 Hz, 1H), 5.79 (dd, J=9.6, 2.4 Hz, 1H), 4.00-3.79 (m, 1H), 3.83-3.79 (m, 1H), 3.47-3.51 (m, 2H), 2.50-2.47 (m, 1H), 2.19-1.96 (m, 2H), 1.92-1.51 (m, 3H). LCMS: 309.0 [M+H]⁺.

### Step-5: Synthesis of (Z)-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole

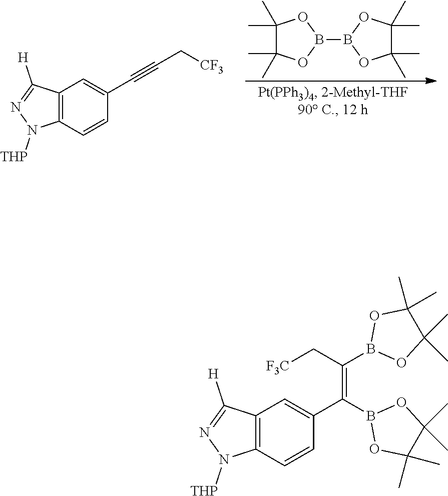

Into a 500-mL round-bottom flask purged and maintained with an inert atmosphere of nitrogen was placed 1-(tetrahydro-2H-pyran-2-yl)-5-(4,4,4-trifluorobut-1-yn-1-yl)-1H-indazole (24 g, 77.85 mmol, 1.00 equiv). 4,4,5,5-tetramethyl-2-(tetramethyl-1,3,2-dioxaborolan-2-yl)-1,3,2-dioxaborolane (19.7 g, 77.58 mmol, 1.00 equiv), Pt(PPh₃)₄ (4.8 g, 0.05 equiv), and 2-Methyl THF (200 mL). The resulting solution was stirred at 90° C. in an oil bath until completion. The resulting solution was used to the next step without further purification.

### Step-6: Synthesis of ((E)-1-(6-(2-((tert-butoxycarbonyl)((E)-4-(methylamino)-4-oxobut-2-en-1-yl)amino)ethoxy)pyridin-3-yl)-4,4,4-trifluoro-1-(1-(tetrahydro-2H-pyran-2-yl)-1H-indazol-5-yl)but-1-en-2-yl)boronic acid

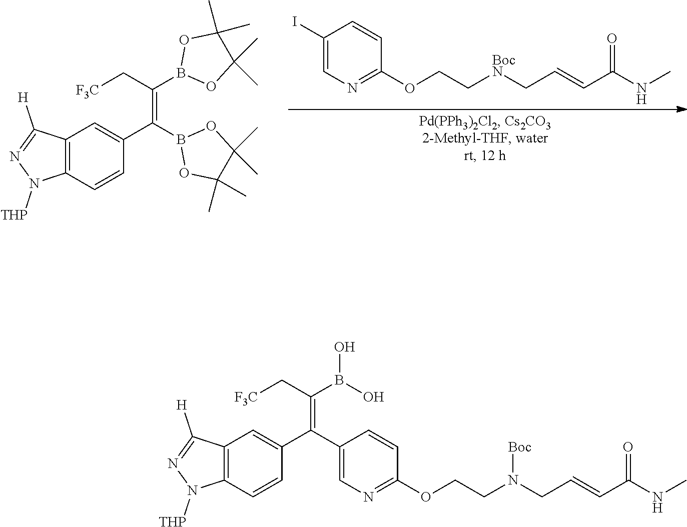

Into a 1000-mL round-bottom flask purged and maintained with an inert atmosphere of nitrogen was placed (Z)-1-(tetrahydro-2H-pyran-2-yl)-5-(4,4-trifluoro-1,2-bis(4,4,5,5-tetramethyl-1,3,2-dioxaborolan-2-yl)but-1-en-1-yl)-1H-indazole (44 g, 78.26 mmol, 1.00 equiv), tert-butyl (E)-(2-((5-iodopyridin-2-yl)oxy)ethyl)-4-(methylamino)-4-oxobut-2-en-1-yl)carbamate (36 g, 78.04 mmol, 1.00 equiv) (Scheme 4, Steps-1-3). Cs₂CO₃ (63 g, 193.36 mmol, 2.50 equiv), Pd(PPh₃)₂Cl₂ (5.5 g, 7.84 mmol, 0.10 equiv), 2-Methyl THF (400 mL), and water (80 mL). The resulting solution was stirred at room temperature until completion. The reaction was then quenched with ice water (500 mL). The resulting solution was extracted with 3×500 mL of ethyl acetate and the organic layers combined and concentrated under vacuum. The residue was applied onto a silica gel column with DCM/methanol (10:1) to deliver the title compound in 40 g (74%) as a brown solid.

### Step-7: Synthesis of tert-butyl ((E)-4-(methylamino)-4-oxobut-2-en-1-yl)(2-(4-((E)-4,4,4-trifluoro-2-phenyl-1-(1-(tetrahydro-2H-pyran-2-yl)-1H-indazol-5-yl)but-1-en-1-yl)phenoxy)ethyl)carbamate

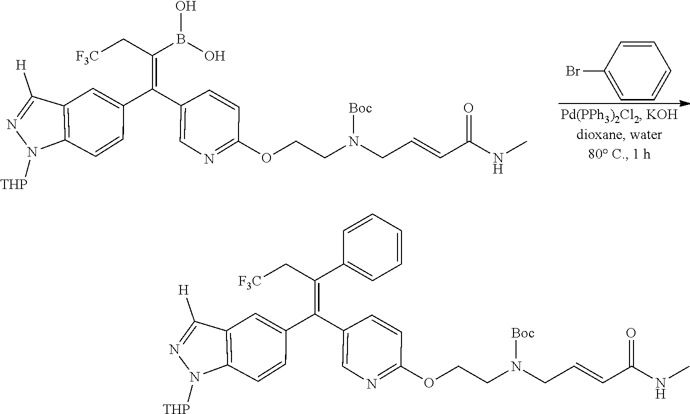

Into a 1000-mL round-bottom flask purged and maintained with an inert atmosphere of nitrogen was placed ((E)-1-(6-(2-((tert-butoxycarbonyl)((E)-4-(methylamino)-4-oxobut-2-en-1-yl)amino)ethoxy)pyridin-3-yl)-4,4,4-trifluoro-1-(1-(tetrahydro-2H-pyran-2-yl)-1H-indazol-5-yl)but-1-en-2-yl)boronic acid (20 g, 25.99 mmol, 1.00 equiv), bromobenzene (5.0 g, 31.85 mmol, 1.10 equiv), Pd(PPh₃)Cl₂ (2.0 g, 0.10 equiv), KOH (4.9 g, 87.33 mmol, 3.00 equiv), dioxane (250 mL), and water (50 mL). The resulting solution was stirred at 80° C. until completion. The reaction was concentrated under vacuum and the residue was applied onto a silica gel column with ethyl acetate/petroleum ether (10:1) to deliver the title compound in 12 g (64%) as an off-white solid. LCMS: 720.0 [M+H]⁺.

### Step-8: Synthesis of (E)-N-methyl-4-((2-((5-((Z)-4,4,4-trifluoro-1-(1H-indazol-5-yl)-2-phenylbut-1-en-1-ylpyridin-2-yl)oxy)ethyl)amino)but-2-enamide

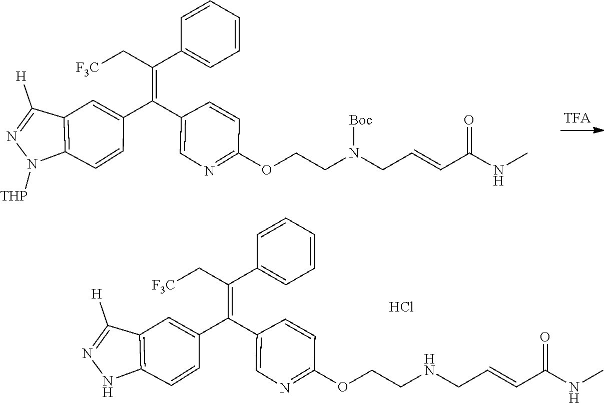

Into a 250-mL round-bottom flask was placed tert-butyl ((E)-4-(methylamino)-4-oxobut-2-en-1-yl)(2-(4-((E)-4,4,4-trifluoro-2-phenyl-1-(1-(tetrahydro-2H-pyran-2-yl)-1H-indazol-5-yl)but-1-en-1-yl)phenoxy)ethyl)carbamate (20 g, 27.79 mmol, 1.00 equiv), trifluoroacetic acid (50 mL), and DCM (50 mL). The resulting solution was stirred at room temperature until completion, then concentrated under vacuum. The residue was dissolved in 20 mL of CH₃CN and purified by Flash-Prep-HPLC with the following conditions (IntelFlash-1): Column, silica gel; mobile phase, water (NH₄HCO₃ 10 mmol/L)/CH₃CN=35%, water (NH₄HCO₃ 10 mmol/L) increased to CH₃CN=45% within 10 min; Detector, UV 254 nm. The residue was dissolved in 20 mL of CH₃CN. The freebase product was converted to HCl salt with hydrogen chloride (1.1 equiv), lyophilized for 48 h to deliver the title compound in 5.2094 g, 3.53% overall yield, as a yellow solid. ¹H NMR (400 MHz, Methanol-d₄) δ 8.30 (s, 1H), 7.86 (s, 1H), 7.74 (d, J=2.4 Hz, 1H), 7.67-7.64 (d, J=2.4 Hz, 1H), 7.47-7.43 (dd, J=8.8, 2.4 Hz, 1H), 7.37-7.36 (dd, J=8.8, 1.6 Hz, 1H), 7.34-7.20 (m, 5H), 6.79-6.76 (d, J=8.8 Hz, 1H), 6.72-6.63 (m, 1H), 6.32-6.27 (d, J=16 Hz, 1H), 4.51-4.48 (m, 2H), 3.86-3.84 (dd, J=6.8, 1.6 Hz, 2H), 3.44-3.37 (m, 4H), 2.79 (s, 3H). LCMS: 536.2 [M+H]⁺.

### Example 10: Synthesis of (E)-N-methyl-4-((2-(4-((E)-4,4,4-trifluoro-1-(1H-indazol-5-yl)-2-phenylbut-1-en-1-yl)phenoxy)ethyl)amino)but-2-enamide (Compound 10)

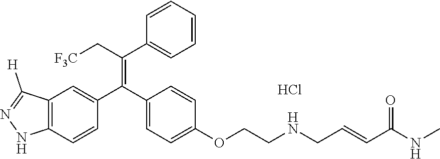

Compound 10 was synthesized following the approach outlined in Scheme 8 by modifying: a) Step-6 by substituting tert-butyl (E)-(2-(4-iodophenoxy)ethyl)(4-(methylamino)-4-oxobut-2-en-1-yl)carbamate (Scheme 5, Steps-1-3) for compound 324, 2-Methyl THF:H₂O (5:1) for dioxane:H₂O, and stirring at 40° C. until completion, and b) Step-7 by using 1.0 equiv of bromobenzene and stirring at 40° C. until completion to deliver the title compound in 175 mg. 3.76% overall yield. ¹H NMR (400 MHz, Methanol-d4) δ 8.70-8.65 (s, 1H), 7.96-7.94 (s, 1H), 7.75-7.71 (dt, J=8.8, 0.9 Hz, 1H), 7.50-7.46 (dd, J=8.8, 1.5 Hz 1H), 7.22-7.12 (m, 5H), 6.91-6.89 (m, 2H), 6.73-6.67 (m, 3H), 6.32-6.29 (m, 1H), 4.17-4.15 (m, 2H), 3.87-3.85 (m, 2H), 3.41-3.34 (m, 4H), 2.79 (s, 3H). LCMS: 535.30 [M+H]⁺.

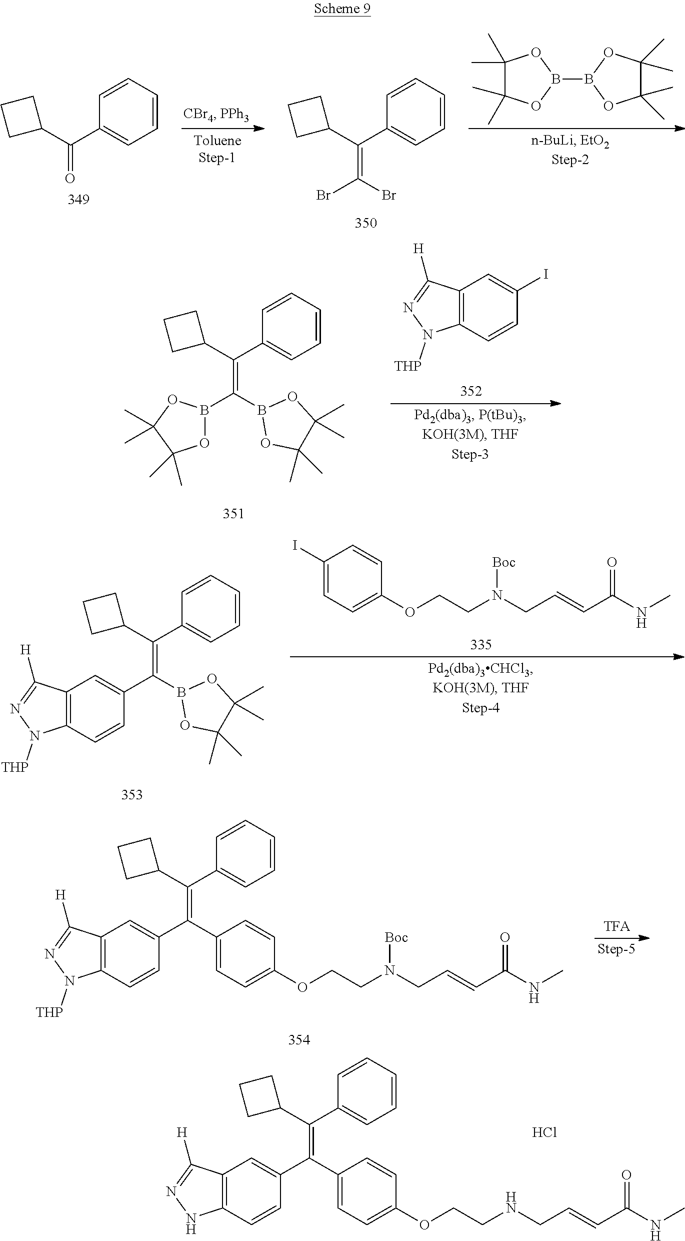

### Example 11: Synthesis of (E)-4-((2-(4-((E)-2-cyclobutyl-1-(1H-indazol-5-yl)-2-phenylvinyl))phenoxy)ethyl)amino)-N-methylbut-2-enamide (Compound 11)

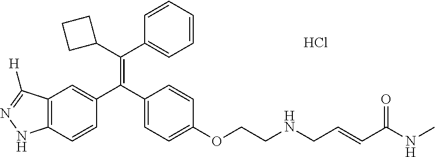 [SMILES: CNC(=O)/C=C/CNCCOc1ccc(/C(=C(\c2ccccc2)C2CCC2)c2ccc3[nH]ncc3c2)cc1 ; Cl]

### Step-1: Synthesis of (2,2-dibromo-1-cyclobutylvinyl) benzene

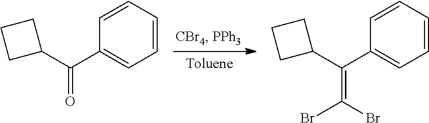 [SMILES: O=C(c1ccccc1)C1CCC1 ; BrC(Br)=C(c1ccccc1)C1CCC1]

Into a 1000-mL round-bottom flask purged and maintained with an inert atmosphere of nitrogen was placed PPh₃ (65.5 g, 249.72 mmol, 4.00 equiv) and toluene (300 mL). This was followed by the addition of a solution of CBr₄ (41 g, 125.00 mmol, 2.00 equiv) in toluene (100 mL) dropwise at 0° C. while stirring. Then, a solution of cyclobutyl(phenyl)methanone (10 g, 62.42 mmol, 1.00 equiv) in toluene (100 mL) was added in dropwise. The resulting solution was stirred until completion at 120° C. in an oil bath. The solution was then diluted with H₂O (400 mL) and extracted with 3×400 mL of ethyl acetate. The organic layers were combined, dried over Na₂SO₄, and the resulting mixture was concentrated under vacuum. The residue was applied onto a silica gel column with ethyl acetate/petroleum ether (0:10) to deliver the title compound in 7.5 g (38%) as yellow oil.

### Step-2: Synthesis of 2,2′-(2-cyclobutyl-2-phenylethene-1,1-diyl)bis(4,4,5,5-tetramethyl-1,3,2-dioxaborolane)

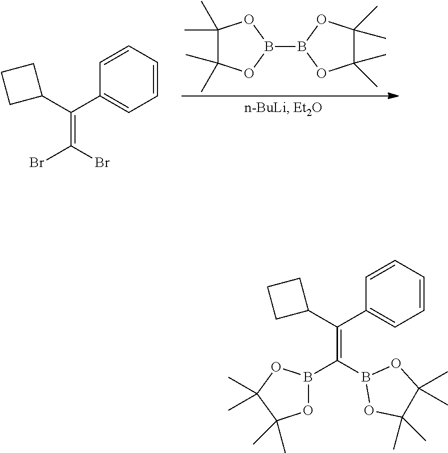

Into a 500-mL round-bottom flask purged and maintained with an inert atmosphere of nitrogen was placed (2,2-dibromo-1-cyclobutylvinyl)benzene (3 g, 9.49 mmol, 1.00 equiv). Et₂O (200 mL), and a solution of 4,4,5,5-tetramethyl-2-(tetramethyl-1,3,2-dioxaborolan-2-yl)-1,3,2-dioxaborolane (2.41 g, 9.49 mmol, 1.00 equiv) in ether (100 mL). The reaction was then cooled to −78° C., and n-BuLi (2.5M in hexane, 4.2 mL) was added dropwise. The resulting solution was stirred until completion at −110° C. in a liquid nitrogen bath. The reaction was then quenched by the addition of methanol (100 mL). The mixture was dried over anhydrous sodium sulfate and concentrated under vacuum. The residue was applied onto a silica gel column with ethyl acetate/petroleum ether (1:9) to deliver the title compound in 600 mg (15%) as a yellow solid.

### Step-3: Synthesis of (E)-5-(2-cyclobutyl-2-phenyl-1-(4,4,5,5-tetramethyl-1,3,2-dioxaborolan-2-yl)vinyl)-1-(tetrahydro-2H-pyran-2-yl)-1H-indazole

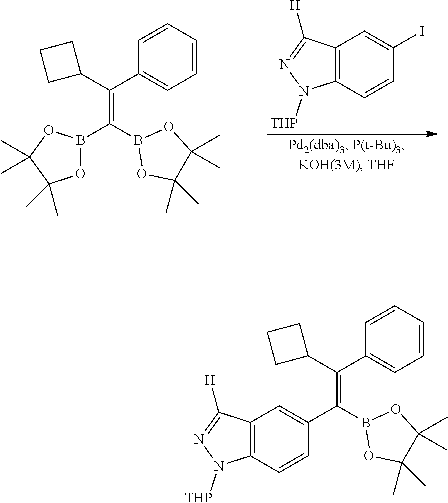

Into a 40-mL round-bottom flask purged and maintained with an inert atmosphere of nitrogen was placed a solution of 2,2′-(2-cyclobutyl-2-phenylethene-1,1-diyl)bis(4,4,5,5-tetramethyl-1,3,2-dioxaborolane) (410 mg, 1.00 mmol, 1.00 equiv) in THF (30 mL), 5-iodo-1-(oxan-2-yl)-1H-indazole (328 mg, 1.00 mmol, 1.00 equiv), Pd₂(dba)₃ (110 mg, 0.12 mmol, 0.10 equiv), P(t-Bu)₃.HBF (60 mg, 0.21 mmol, 0.20 equiv), and KOH (3M) (3.5 mL). The resulting solution was stirred at 25° C. until completion. The reaction was then quenched by the addition of 50 mL of water, extracted with 2×100 mL of DCM, and the organic layers were combined. The resulting mixture was washed with 1×100 mL of brine, dried over anhydrous sodium sulfate and concentrated under vacuum. The residue was applied onto a silica gel column with ethyl acetate:petroleum ether (7:3) to deliver the title compound in 320 mg (66%) as a yellow solid. LCMS: 485.5 [M+H]⁺.

### Step-4: Synthesis of tert-butyl (E)-(2-(4-(2-cyclobutyl-2-phenyl-1-(1-(tetrahydro-2H-pyran-2-yl)-1H-indazol-5-yl)vinyl)phenoxy)ethyl)(4-(methylamino)-4-oxobutyl)carbamate

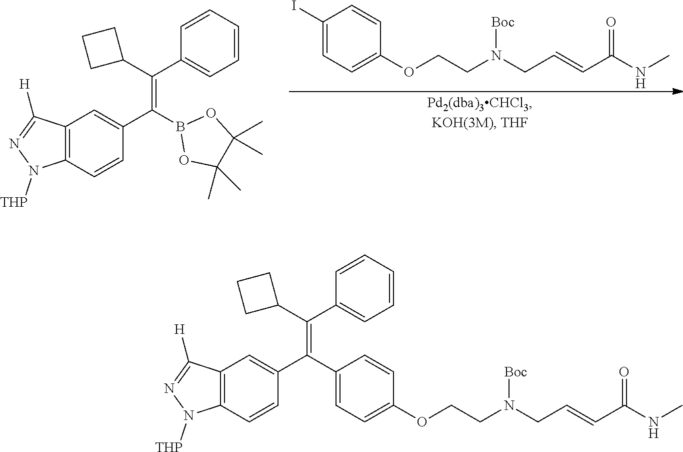

Into a 100-mL round-bottom flask purged and maintained with an inert atmosphere of nitrogen was placed a solution of (E)-5-(2-cyclobutyl-2-phenyl-1-(4,4,5,5-tetramethyl-1,3,2-dioxaborolan-2-yl)vinyl)-1-(tetrahydro-2H-pyran-2-yl)-1H-indazole (320 mg, 0.66 mmol, 1.00 equiv), Pd₂(dba)₃CHCl (68 mg, 0.066 mmol, 0.10 equiv), KOH (3M) (3.5 mL), THF (25 mL), and tert-butyl (E)-(2-(4-iodophenoxy)ethyl)(4-(methylamino)-4-oxobut-2-en-1-yl)carbamate (304 mg, 0.66 mmol, 1.00 equiv) (Scheme 5, Steps-1-3). The resulting solution was stirred at 80° C. in an oil bath until completion. The reaction was then quenched by the addition of 50 mL of water and extracted with 2×100 mL of DCM. The organic layers were combined, then washed with 1×100 mL of brine and dried over anhydrous sodium sulfate. The solution was concentrated under vacuum and the residue was applied onto a silica gel column with EA: PE (7:3) to deliver the title compound in 180 mg (39%) as a yellow solid. LCMS: 691.4 [M+H]⁺.

### Step-S: Synthesis of (E)-4-((2-(4-((E)-2-cyclobutyl-1-(1H-indazol-5-yl)-2-phenylvinyl)phenoxy)ethylamino)-N-methylbut-2-enamide

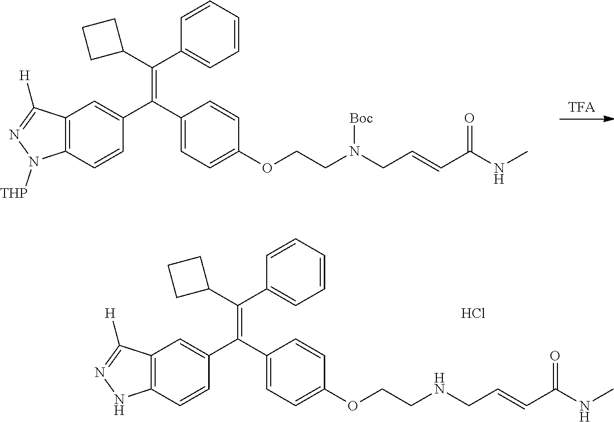

Into a 40-mL round-bottom flask purged and maintained with an inert atmosphere of nitrogen was placed tert-butyl (E)-(2-(4-(2-cyclobutyl-2-phenyl-1-(1-(tetrahydro-2H-pyran-2-yl)-1H-indazol-5-yl)vinyl)phenoxy)ethyl)(4-(methylamino)-4-oxobutyl)carbamate (250 mg, 0.36 mmol, 1.00 equiv), DCM (3 mL), and trifluoroacetic acid (30 mL). The resulting solution was stirred at 25° C. in a water bath until completion, then the mixture was concentrated under vacuum. The crude product (150 mg) was purified by Prep-HPLC with the following conditions (2#-AnalyseHPLC-SHIMADZU(HPLC-10)): Column, X-Select CSH Prep C18 OBD Column, 19*250 mm, 5 um; mobile phase, Water (0.05% NH4CO₃) and ACN (30.0% ACN up to 48.0% in 10 min); Detector, UV 254/220 nm. This resulted in 25.6 mg (14%) of (E)-4-[(2-[4-[(E)-2-cyclobutyl-1-(1H-indazol-5-yl)-2-phenylethenyl]phenoxy]ethyl)amino]-N-methylbut-2-enamide as a white solid. Then, into a 50-mL round-bottom flask was placed (E)-4-((2-(4-((E)-2-cyclobutyl-1-(1H-indazol-5-yl)-2-phenylvinyl)phenoxy)ethyl)amino)-N-methylbut-2-enamide (25.6 mg, 0.05 mmol, 1.00 equiv), acetonitrile (5 mL), water (10 mL), and 0.045 mL hydrochloric acid (1M). The solution was then lyophilized for 12 h to deliver the title compound in 26.0 mg, 0.21% overall yield. ¹H NMR (300 MHz, Methanol-d4) δ 8.34 (s, 1H), 7.74 (s, 1H), 7.62-7.59 (d, J=8.7 Hz, 1H), 7.37-7.34 (m, 1H), 7.25-7.10 (m, 5H), 6.91-6.88 (m, 2H), 6.71-6.64 (m, 3H), 6.31-6.26 (m, 1H), 4.14-4.11 (m, 2H), 3.86-3.84 (dd, J=7.0, 1.4 Hz, 2H), 3.40-3.37 (m, 1H), 3.36-3.32 (m, 2H), 2.81 (s, 3H), 1.94-1.83 (m, 4H), 1.75-1.56 (m, 1H), 1.46-1.40 (m, 1H). LCMS: 507.2 [M+H]⁺.

### Example 12: Synthesis of (Z)-1-(2-((5-(4,4,4-trifluoro-1-(3-fluoro-1H-indazol-5-yl)-2-phenylbut-1-en-1-yl)pyridin-2-yl)oxy)ethyl)pyrrolidin-2-one (Compound 12)

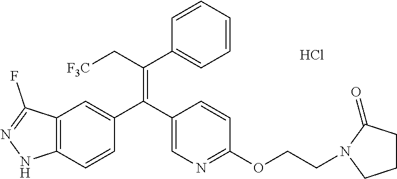 [SMILES: O=C1CCCN1CCOc1ccc(/C(=C(/CC(F)(F)F)c2ccccc2)c2ccc3[nH]nc(F)c3c2)cn1 ; Cl]

Compound 12 was synthesized following the approach outlined in Scheme 3 by modifying: a) Step-7 by substituting 1-(2-(5-iodopyridin-2-yl)oxy)ethyl)pyrrolidin-2-one (preparation shown below in Step-a) for compound 324 and stirring at 50° C., and b) Step-8 by substituting 2-Methyl THF (to make a 0.9M solution) for dioxane and water to deliver the title compound in 216.7 mg, 2.18% overall yield. ¹H NMR (400 MHz, Methanol-d4) δ 7.90-7.88 (d, J=8.4 Hz, 1H), 7.80 (d, J=2.0 Hz, 1H), 7.74 (s, 1H), 7.56-7.54 (m, 1H), 7.38-7.36 (d, J=8.6 Hz, 1H), 7.33-7.25 (m, 5H), 7.21-7.19 (d, J=8.9 Hz, 1H), 4.49-4.46 (t, J=5.0 Hz, 2H), 3.68-3.66 (m, 2H), 3.57-3.39 (m, 4H), 2.37-2.30 (t, J=8.0 Hz, 2H), 2.04-1.99 (m, 2H). LCMS: 525 [M+H]⁺.

### Step-a: Synthesis of 1-(2-((5-iodopyridin-2-yl)oxy)ethyl)pyrrolidin-2-one

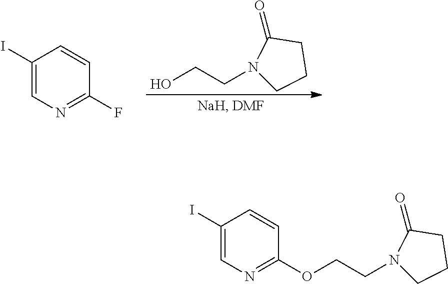 [SMILES: Fc1ccc(I)cn1 ; O=C1CCCN1CCO ; O=C1CCCN1CCOc1ccc(I)cn1]

Into a 250-mL round-bottom flask was placed 1-(2-hydroxyethyl)pyrrolidin-2-one (8.6 g, 66.59 mmol, 1.00 equiv). N,N-dimethylformamide (50 mL), and sodium hydride (1.1 g, 45.83 mmol, 1.20 equiv). The resulting solution was stirred at 0° C. in a water/ice bath until completion. 2-fluoro-5-iodopyridine (5 g, 22.42 mmol, 1.00 equiv) was then added. The resulting solution was allowed to react, with stirring, at 25° C. until completion. The solution was diluted with H₂O (100 mL), extracted with 3×100 mL of ethyl acetate, dried over Na₂SO₄ and the organic layers combined. The crude product was purified by Flash-Prep-HPLC with the following conditions (IntelFlash-1): Column, silica gel; mobile phase, ethyl acetate/petroleum ether (1:9); Detector, UV 254 nm to deliver the title compound in 5.0 g (67%) as light yellow oil. ¹H NMR (400 MHz. Methanol-d4) δ 8.34 (dd, J=2.4, 0.7 Hz, 1H), 7.94-7.91 (dd, J=8.7, 2.4 Hz, 1H), 6.69-6.67 (dd, J=8.7, 0.7 Hz, 1H), 4.46-4.43 (m, 2H), 3.68-3.65 (t, J=5.3 Hz, 2H), 3.60-3.56 (m, 2H), 2.382.34 (t, J=8.1 Hz, 2H), 2.07-2.00 (m, 2H). LCMS: 333 [M+H]⁺.

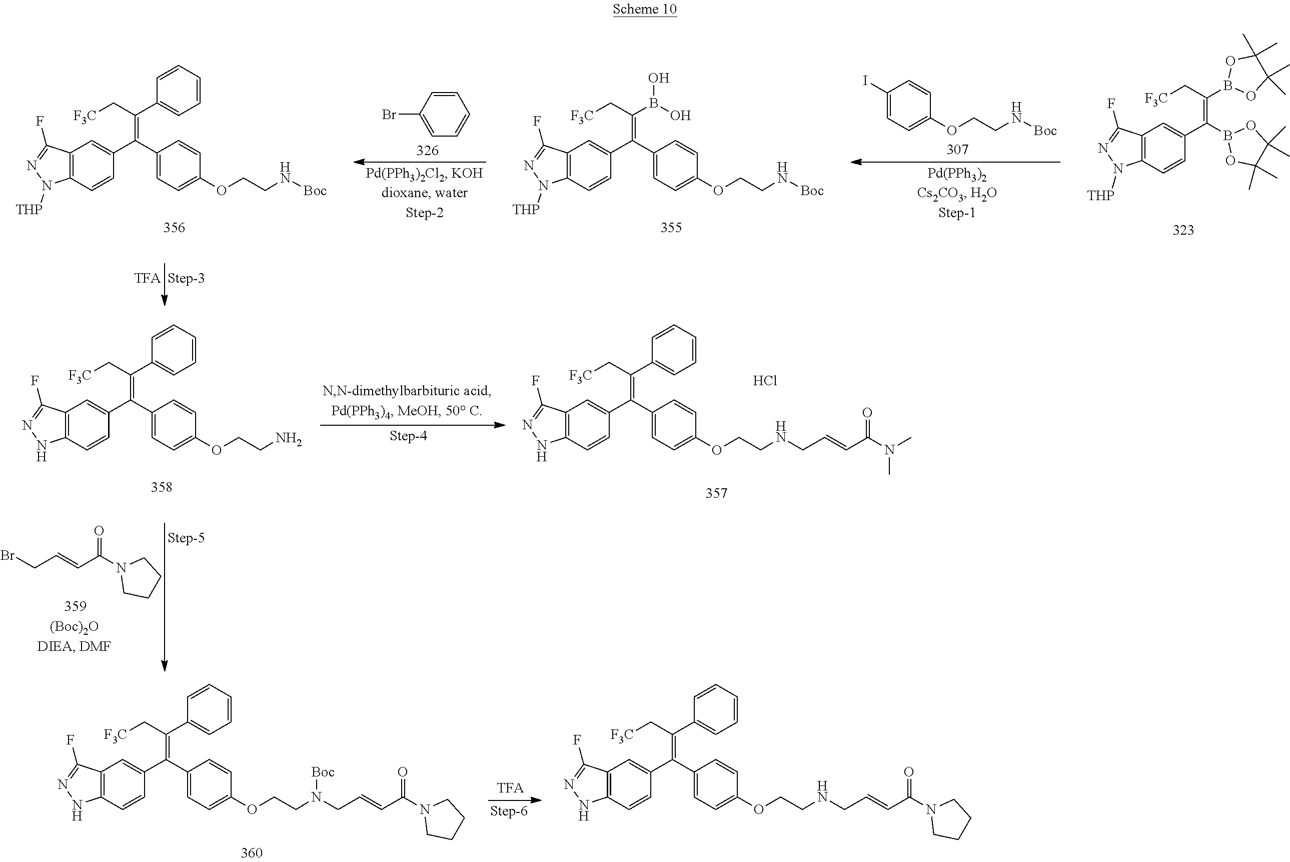

### Example 13: Synthesis of (E)-1-(pyrrolidin-1-yl)-4-((2-(4-((E)-4,4,4-trifluoro-1-(3-fluoro-1H-indazol-5-yl)-2-phenylbut-1-en-1-yl)phenoxy)ethyl)amino)but-2-en-1-one (Compound 13)

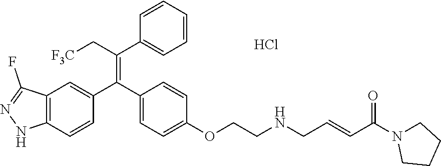 [SMILES: O=C(/C=C/CNCCOc1ccc(/C(=C(/CC(F)(F)F)c2ccccc2)c2ccc3[nH]nc(F)c3c2)cc1)N1CCCC1 ; Cl]

### Step-1: Synthesis of (Z)-(1-(4-(2-((ten-butoxycarbonyl)amino)ethoxy)phenyl)-4,4,4-trifluoro-1-(3-fluoro-1-(tetrahydro-2H-pyran-2-yl)-1H-indazol-5-yl)but-1-en-2-yl)boronic acid

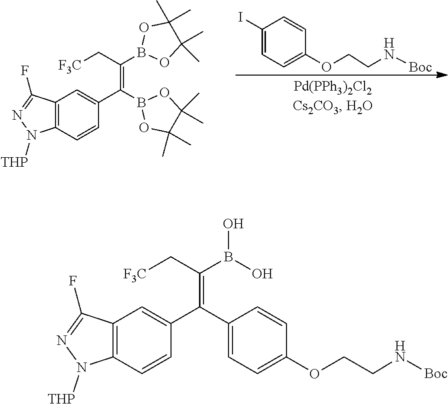

Into a 250-mL round-bottom flask purged and maintained with an inert atmosphere of nitrogen was placed (Z)-3-fluoro-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (6.4 g, 11.31 mmol, 1.00 equiv) (Scheme 3, Steps-1-6), Cs₂CO₃ (12.6488 g, 38.82 mmol, 2.00 equiv), tert-butyl (2-(4-iodophenoxy)ethyl)carbamate (7.08 g, 19.44 mmol, 1.00 equiv) (Scheme 5, Step-1), water (2 mL), Pd(PPh₃)Cl₂ (1.36188 g, 1.94 mmol, 0.10 equiv), and 2-Methyl-THF (20 mL). The resulting solution was stirred at 50° C. until completion and used directly to the next step.

### Step-2: Synthesis of tert-butyl (E)-(2-(4-(4,4,4-trifluoro-1-(3-fluoro-1-(tetrahydro-2H-pyran-2-yl)-1H-Indazol-5-yl)-2-phenylbut-1-en-1-yl)phenoxy)ethyl)carbamate

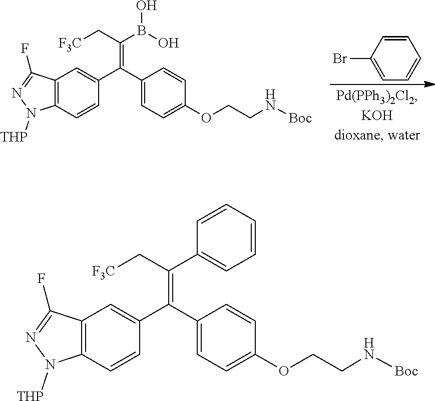

Into a 250-mL round-bottom flask purged and maintained with an inert atmosphere of nitrogen was placed (Z)-1-(4-(2-(tert-butoxycarbonylamino)ethoxy)phenyl)-4,4,4-trifluoro-1-(3-fluoro-1-(tetrahydro-2H-pyran-2-yl)-1H-indazol-5-yl)but-1-en-2-ylboronic acid (5.15 g, 8.47 mmol, 1.00 equiv), dioxane (30 mL), water (5 mL), KOH (3.25 g, 57.92 mmol, 3.00 equiv), Pd(PPh₃)₂Cl₂ (1.36188 g, 1.94 mmol, 0.10 equiv), and bromobenzene (3.0259 g, 19.27 mmol, 1.00 equiv). The resulting solution was stirred at 80° C. until completion. The solution was then diluted with 30 mL of water and extracted with 3×50 mL of ethyl acetate. Then the organic layers were combined and washed with 3×50 mL of brine. The mixture was dried over anhydrous sodium sulfate and concentrated under vacuum. The residue was applied onto a silica gel column eluting with DCM/methanol (14:1) to deliver the title compound in 2.3 g (43%) as a yellow oil.

### Step-3: Synthesis of (E)-2-(4-(4,4,4-trifluoro-1-(3-fluoro-1H-indazol-5-yl-2-phenylbut-1-en-1-yl)phenoxy)ethan-1-amine hydrochloride

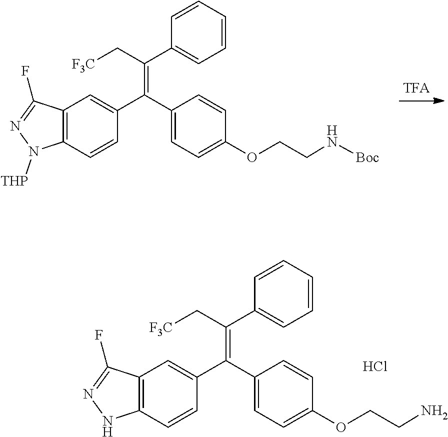

Into a 8-mL round-bottom flask was placed tert-butyl (E)-(2-(4-(4,4,4-trifluoro-1-(3-fluoro-1-(tetrahydro-2H-pyran-2-yl)-1H-indazol-5-yl)-2-phenylbut-1-en-1-yl)phenoxy)ethyl)carbamate (510 mg, 0.80 mmol, 1.00 equiv), DCM (2 mL), and TFA (4 mL). The resulting solution was stirred at 25° C. until completion. The resulted solution was concentrated under vacuum. The crude product was purified by Prep-HPLC with the following conditions (1#-Waters 2767-1): Column, Sun-Fire Prep C18, 5 um, 19*100 mm; mobile phase: water in 0.5% HCl and CH₃CN (12% CH₃CN up to 29% in 20 min, up to 100% in 1 min, down to 6% in 1 min); Detector, UV 254 nm. The product fractions were combined and concentrated under vacuum to deliver the title compound in 169 mg (37%) as a white solid.

### Step-4: Alternative Method for the Synthesis of (E)-2-(4-(4,4,4-trifluoro-1-(3-fluoro-1H-indazol-5-yl)-2-phenylbut-1-en-1-yl)phenoxy)ethan-1-amine

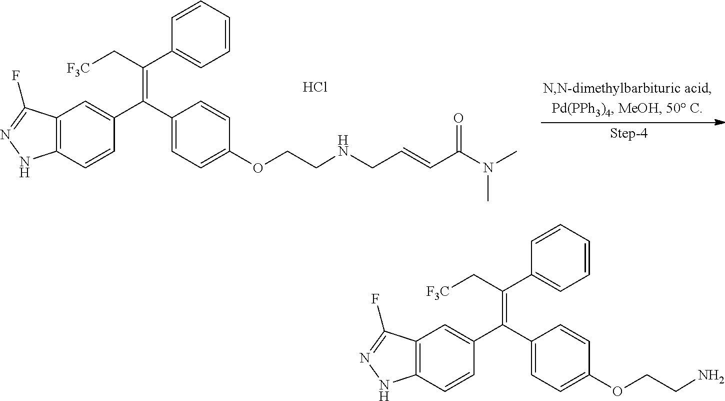 [SMILES: CN(C)C(=O)/C=C/CNCCOc1ccc(/C(=C(/CC(F)(F)F)c2ccccc2)c2ccc3[nH]nc(F)c3c2)cc1 ; Cl ; NCCOc1ccc(/C(=C(/CC(F)(F)F)c2ccccc2)c2ccc3[nH]nc(F)c3c2)cc1]

Into a 2-L round-bottom flask was placed (E)-N,N-dimethyl-4-((2-(4-((E)-4,4,4-trifluoro-1-(3-fluoro-1H-indazol-5-yl)-2-phenylbut-1-en-1-yl)phenoxy)ethyl)amino)but-2-enamide hydrochloride (25 g, 44.12 mmol, 1.00 equiv) (synthesized following the approach outlined in patent US 2016347717 A1), methanol (750 mL), N,N-dimethylbarbituric acid (17.2 g, 110.16 mmol, 2.50 equiv), and Pd(PPh₃)₄ (12.8 g, 11.08 mmol, 0.25 equiv). The resulting solution was stirred at 50° C. until completion. The reaction progress was monitored by LCMS. The resulting mixture was concentrated under vacuum, then diluted with 500 mL of DCM and washed with 3×200 mL of aqueous sodium carbonate. The mixture was then dried over anhydrous sodium sulfate. The residue was applied onto a silica gel column with DCM/methanol (100:0-90:10). The collected fractions were combined and concentrated under vacuum to deliver the title compound in 15 g (75%) as a yellow solid. LCMS: 456.1 [M+H]⁺.

### Step-5: Synthesis of tert-butyl ((E)-4-oxo-4-(pyrrolidin-1-yl)but-2-en-1-yl)(2-(4-((E)-4,4,4-trifluoro-1-(3-fluoro-1H-indazol-5-yl)-2-phenylbut-1-en-1-yl)phenoxy)ethyl)carbamate

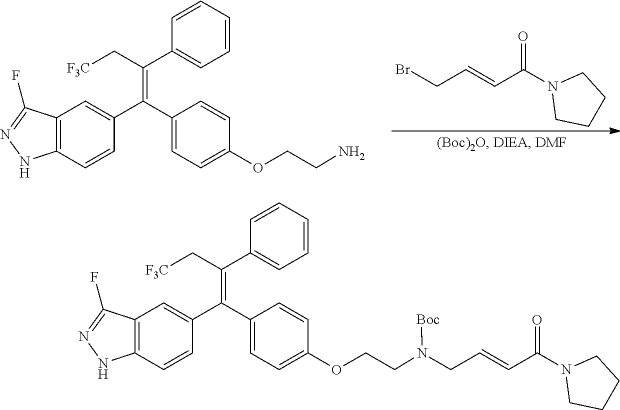

Into a 8-mL vial, was placed (E)-2-(4-(4,4,4-trifluoro-1-(3-fluoro-1H-indazol-5-yl)-2-phenylbut-1-en-1-yl)phenoxy)ethan-1-amine (62 mg, 0.14 mmol, 1.00 equiv), N,N-dimethylformamide (1 mL), DIEA (46 mg, 0.36 mmol, 2.62 equiv), and (E)-4-bromo-1-(pyrrolidin-1-yl)but-2-en-1-one (20.4 mg, 0.09 mmol, 0.69 equiv) (Scheme 4, steps-a-b, substituting pyrrolidine for methylamine). The resulting solution was stirred at 25° C. until completion, then Boc₂O (51.3 mg, 0.24 mmol, 1.73 equiv) was added. The resulting solution was allowed to react, with stirring, at 25° C. until completion. The reaction progress was monitored by LCMS. The resulting solution was diluted with 20 mL of ethyl acetate, washed with 3×20 mL of brine, and then the mixture was dried over anhydrous sodium sulfate. The residue was applied onto a silica gel column with ethyl acetate/petroleum ether (0:100-100:0). The collected fractions were combined and concentrated under vacuum to deliver the title compound in 15 mg (16%) as a yellow solid. LCMS: 593 [M-Boc+H]⁺.

### Step-6: Synthesis of (E)-1-(pyrrolidin-1-yl-4-((2-(4-((E)-4,4,4-trifluoro-1-(3-fluoro-1H-Indazol-5-yl)-2-phenylbut-1-en-1-yl)phenoxy)ethyl)amino)but-2-en-1-one

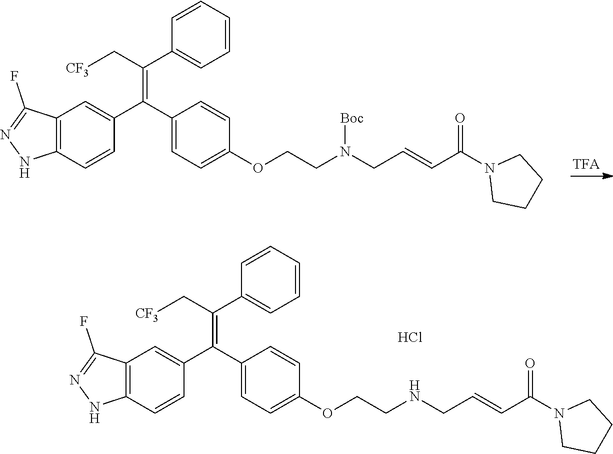 [SMILES: CC(C)(C)OC(=O)N(C/C=C/C(=O)N1CCCC1)CCOc1ccc(/C(=C(/CC(F)(F)F)c2ccccc2)c2ccc3[nH]nc(F)c3c2)cc1 ; O=C(/C=C/CNCCOc1ccc(/C(=C(/CC(F)(F)F)c2ccccc2)c2ccc3[nH]nc(F)c3c2)cc1)N1CCCC1 ; Cl]

Into a 250-mL round-bottom flask was placed tert-butyl ((E)-4-oxo-4-(pyrrolidin-1-yl)but-2-en-1-yl)(2-(4-((E)-4,4,4-trifluoro-1-(3-fluoro-1H-indazol-5-yl)-2-phenylbut-1-en-1-yl)phenoxy)ethyl)carbamate (5 g, 8.42 mmol, 1.00 equiv), and TFA (30 mL). The resulting solution was stirred at 25° C. until completion, then concentrated under vacuum. The crude product was purified by Prep-HPLC with the following conditions (1#-Waters 2767-1): Column, X-bridge; mobile phase, Phase A:water with 0.5% NH₄HCO₃ Phase B:CH₃CN. Water with 0.5% NH₄HCO₃ and CH₃CN (25% CH₃CN up to 55% in 60 min); Detector, uv 254 nm to give the freebase product. ¹H NMR (400 MHz, Methanol-d4) δ 7.63 (s, 1H), 7.47-7.45 (m, 1H), 7.28 (dd, J=8.8, 1.5 Hz, 1H), 7.26-7.15 (m, 5H), 6.87-6.80 (m, 3H), 6.67-6.64 (m, 2H), 6.45-6.41 (m, 1H), 4.00-3.98 (m, 2H), 3.60-3.56 (d, J=5.4 Hz, 2H), 3.49-3.35 (m, 6H), 2.95-2.93 (t, J=5.2 Hz, 2H), 1.99-1.88 (m, 4H).

The freebase product was converted to HCl salt with 1.1 equiv of HCl (1M) to deliver the title compound in 3.0952 g, 1.73% overall yield, as a yellow solid. ¹H NMR (400 MHz, Methanol-d4) δ 7.62 (d, J=1.3 Hz, 1H), 7.46-7.44 (m, 1H), 7.28 (dd, J=8.8, 1.5 Hz, 1H), 7.26-7.12 (m, 5H), 6.90-6.88 (m, 2H), 6.72-6.69 (m, 4H), 4.17-4.15 (m, 2H), 3.90-3.89 (d, J=5.4 Hz, 2H), 3.61-3.58 (t, J=6.8 Hz, 2H), 3.49-3.34 (t, J=6.9 Hz, 2H), 2.00-1.96 (m, 2H), 1.93-1.88 (m, 2H). LCMS: 593 [M+H]⁺.

### Example 14: Synthesis of (E)-1-(pyrrolidin-1-yl)-4-((2-(4-((E)-4,4,4-trifluoro-1-(1H-indazol-5-yl)-2-phenylbut-1-en-1-yl)phenoxy)ethyl)amino)but-2-en-1-one (Compound 14)

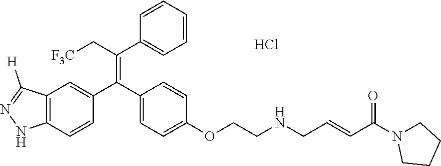 [SMILES: O=C(/C=C/CNCCOc1ccc(/C(=C(/CC(F)(F)F)c2ccccc2)c2ccc3[nH]ncc3c2)cc1)N1CCCC1 ; Cl]

Compound 14 was synthesized following the approach outlined in Scheme 8 by modifying: a) Step-6 by substituting tert-butyl (E)-(2-(4-iodophenoxy)ethyl)(4-oxo-4-(pyrrolidin-1-yl)but-2-en-1-yl)carbamate (preparation shown below, Step-a) for compound 324 and 2-Methyl THF:H₂O (4:1) for dioxane:H₂O, using 2.0 equiv of Cs₂CO₃ and stirring at 60° C. until completion, and b) Step-7 by using 4.0 equiv of KOH and 1.2 equiv of bromobenzene to deliver the title compound in 59.9 mg, 0.47% overall yield. ¹H NMR (400 MHz, Methanol-d4) δ 8.29 (s, 1H), 7.81 (s, 1H), 7.62-7.60 (m, J=8.7, 1.0 Hz, 1H), 7.32-7.30 (dd, J=8.7, 1.6 Hz, 1H), 7.22-7.12 (m, 5H), 6.90-6.88 (m, 2H), 6.72-6.70 (m, 4H), 4.18-4.15 (m, 2H), 3.90-3.89 (m, 2H), 3.62-3.58 (m, 2H), 3.49-3.34 (m, 6H), 2.00-1.88 (m, 4H). LCMS: 575.20 [M+H]⁺.

### Step-a: Synthesis of tert-butyl (E)-(2-(4-iodophenoxy)ethyl)(4-oxo-4-(pyrolidin-1-yl)but-2-en-1-yl)carbamate

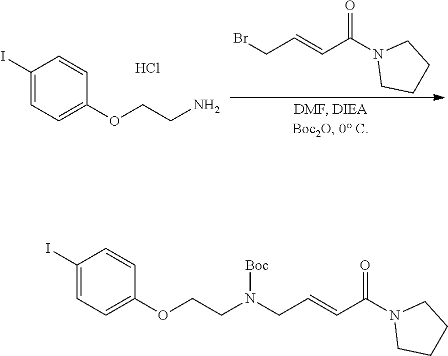 [SMILES: NCCOc1ccc(I)cc1 ; Cl ; O=C(/C=C/CBr)N1CCCC1 ; CC(C)(C)OC(=O)N(C/C=C/C(=O)N1CCCC1)CCOc1ccc(I)cc1]

The title compound was synthesized following the approach outlined in Scheme 5. Step-3, substituting (E)-4-bromo-1-(pyrrolidin-1-yl)but-2-en-1-one for compound 329. ¹H NMR (400 MHz, Methanol-d4) δ 7.58-7.56 (m, 2H), 6.81-6.74 (m, 3H), 6.24-6.17 (t, J=13.3 Hz, 1H), 4.14-4.09 (m, 4H), 3.68-3.65 (t, J=5.2 Hz, 2H), 3.46-3.35 (m, 4H), 1.95-1.84 (m, 4H), 1.48 (d, J=2.2 Hz, 9H). LCMS: 501.2 [M+H]⁺.

### Example 15: Synthesis of (E)-1-(pyrolidin-1-yl)-4-((2-((5-((Z)-4,4,4-trifluoro-1-(3-fluoro-1H-indazol-1-yl)-2-phenylbut-1-en-1-yl)pyridin-2-yl)oxy)ethyl)amino)but-2-en-1-one (Compound 15)

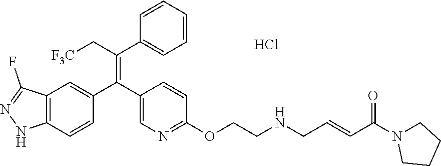 [SMILES: O=C(/C=C/CNCCOc1ccc(/C(=C(/CC(F)(F)F)c2ccccc2)c2ccc3[nH]nc(F)c3c2)cn1)N1CCCC1 ; Cl]

Compound 15 was synthesized following the approach outlined in Scheme 3, modifying Step-7 by a) substituting (E)-tert-butyl 2-(5-iodopyridin-2-yloxy)ethyl(4-oxo-4-(pyrrolidin-1-yl)but-2-enyl)carbamate (preparation shown below, Step-a) for compound 324 and b) stirring at 50° C. until completion to deliver the title compound in 1.8 g, 0.61% overall yield. ¹H NMR (400 MHz, Methanol-d₄) δ 7.72 (dd, J=2.4, 0.7 Hz, 1H), 7.64 (t, J=1.2 Hz, 1H), 7.51-7.49 (m, 1H), 7.38-7.31 (m, 1H), 7.29-7.22 (m, 1H), 7.21-7.17 (m, 5H), 6.71-6.69 (m, 3H), 4.50-4.47 (m, 2H), 3.89-3.88 (m, 2H), 3.61-3.58 (t, J=6.8 Hz, 2H), 3.49-3.29 (m, 6H), 2.00-1.88 (m, 4H). LCMS: 594.30 [M+H]⁺.

### Step-a: Synthesis of tert-butyl (E)-2-(5-iodopyridin-2-yloxy)ethyl(4-oxo-4-(pyrrolidin-1-yl)but-2-enyl)carbamate

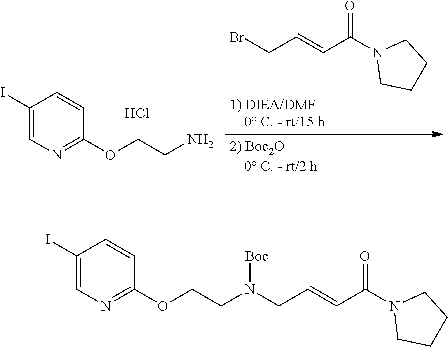 [SMILES: O=C(/C=C/CBr)N1CCCC1 ; NCCOc1ccc(I)cn1 ; Cl ; CC(C)(C)OC(=O)N(C/C=C/C(=O)N1CCCC1)CCOc1ccc(I)cn1]

The title compound was synthesized following the approach outlined in Scheme 4, Step-3, substituting (E)-4-bromo-1-(pyrrolidin-1-yl)but-2-en-1-one for compound 329. ¹H NMR (400 MHz, DMSO-d6) δ 8.35 (d, J=2.3 Hz, 1H), 8.01-7.97 (t, J=6.8 Hz, 1H), 6.72-6.67 (dd, J=12.6, 8.5 Hz, 1H), 6.59-6.51 (m, 1H), 6.22-6.15 (m, 1H), 4.34-4.30 (q, J=5.7 Hz, 2H), 4.06-3.98 (dd, J=7.8, 4.5 Hz, 2H), 3.55-3.52 (t, J=5.4 Hz, 2H), 3.40-3.29 (m, 4H), 1.90-1.83 (m, 2H), 1.80-1.73 (m, 2H), 1.37-1.24 (d, J=19.2 Hz, 9H).

### Example 16: Synthesis of (E)-1-(pyrrolidin-1-yl)-4-((2-((5-((Z)-4,4,4-trifluoro-1-(1H-indazol-5-yl)-2-phenylbut-1-en-1-yl)pyridin-2-yl)oxy)ethyl)amino)but-2-en-1-one (Compound 16)

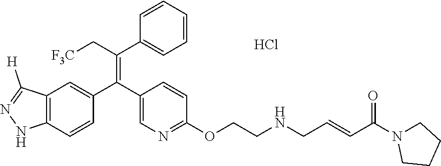 [SMILES: O=C(/C=C/CNCCOc1ccc(/C(=C(/CC(F)(F)F)c2ccccc2)c2ccc3[nH]ncc3c2)cn1)N1CCCC1 ; Cl]

Compound 16 was synthesized following the approach outlined in Scheme 8 by modifying: a) Step-6 by substituting tert-butyl (E)-(2-((5-iodopyridin-2-yl)oxy)ethyl)(4-oxo-4-(pyrrolidin-1-yl)but-2-en-1-yl)carbamate (preparation shown in Example 15. Step-a) for compound 324 and 2-Methyl THF:H₂O (5:1) for dioxane:H₂O and isolating the final product as a pinacol boronic ester instead of a boronic acid, and b) Step-7 by using a 4:1 ratio of dioxane:H₂O to deliver the title compound in 3.53 g, 1.53% overall yield. ¹H NMR (300 MHz. Methanol-d₄) δ 8.87 (s, 1H), 8.16 (s, 1H), 8.05-8.02 (dd, J=9.0, 2.4 Hz, 1H), 7.96 (s, 1H), 7.90-7.87 (J=9.0, 2.4 Hz, 1H), 7.70-7.67 (dd, J=8.8, 1.4 Hz, 1H), 7.38-7.28 (m, 6H), 6.82-6.79 (d, J=2.4 Hz, 2H), 4.75-4.73 (t, J=4.8 Hz, 2H), 4.01 (m, 2H), 3.70-3.66 (m, 2H), 3.61-3.58 (m, 2H), 3.54-3.34 (m, 4H), 2.04-1.91 (m, 4H). LCMS: 576 [M+H]⁺.

### Example 17: Synthesis of (E)-1-morpholino-((2-(4-((2-(((E)-4,4,4-trifluoro-1-(3-fluoro-1H-indazol-yl)-2-yl)-2-phenylbut-1-en-1-yl)phenoxy)ethyl)amino)but-2-en-1-one (Compound 17)

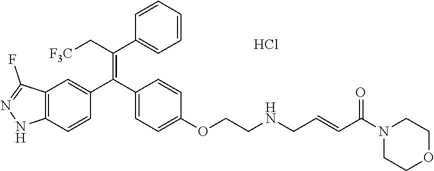 [SMILES: O=C(/C=C/CNCCOc1ccc(/C(=C(/CC(F)(F)F)c2ccccc2)c2ccc3[nH]nc(F)c3c2)cc1)N1CCOCC1 ; Cl]

Compound 17 was synthesized following the approach outlined in Scheme 3, modifying Step-7 by a) substituting tert-butyl (E)-(2-(4-iodophenoxy)ethyl)(4-morpholino-4-oxobut-2-en-1-yl)carbamate (preparation shown below in Step-a) for compound 324 and b) stirring at 50° C. to deliver the title compound in 98.4 mg, 0.57% overall yield. ¹H NMR (400 MHz, Methanol-d4) δ 7.59 (s, 1H), 7.46-7.44 (m, 1H), 7.28-7.27 (dd, J=8.7, 1.5 Hz, 1H), 7.25-7.12 (m, 5H), 6.90-6.83 (m, 3H), 6.73-6.65 (m, 3H), 4.17-4.15 (m, 2H), 3.89-3.88 (dd, J=6.7, 1.3 Hz, 2H), 3.65-3.63 (m, 8H), 3.42-3.34 (m, 4H). LCMS: 608.6 [M+H]⁺.

### Step-a: Synthesis of ter-butyl (E)-(2-(4-iodophenoxy)ethyl)(4-morpholino-4-oxobut-2-en-1-yl)carbamate

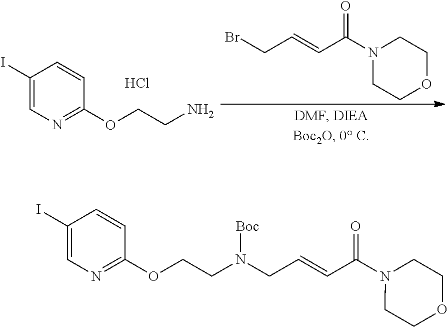 [SMILES: NCCOc1ccc(I)cn1 ; Cl ; O=C(/C=C/CBr)N1CCOCC1 ; CC(C)(C)OC(=O)N(C/C=C/C(=O)N1CCOCC1)CCOc1ccc(I)cn1]

The title compound was synthesized following the approach outlined in Scheme 5, Step-3, substituting (E)-4-bromo-1-morpholinobut-2-en-1-one for compound 329. ¹H NMR (400 MHz, Chloroform-d) δ 7.57-7.55 (m, 2H), 6.86-6.78 (t, J=15.3 Hz, 1H), 6.69-6.65 (m, 2H), 6.28-6.19 (t, J=17.7 Hz, 1H), 4.11-4.03 (m, 4H), 3.68-3.58 (m, 8H), 3.49-3.47 (d, J=8.3 Hz, 2H), 1.49-1.45 (m, 9H).

### Example 18: Synthesis of (E)-1-morpholino-4-(2-(5-((Z)-4,4,4-trifluoro-1-(3-fluoro-1H-indazol-5-yl)-2-phenylbut-1-enyl)pyridin-2-yloxy)ethylamino)but-2-en-1-one (Compound

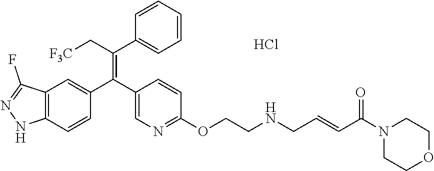 [SMILES: O=C(/C=C/CNCCOc1ccc(/C(=C(/CC(F)(F)F)c2ccccc2)c2ccc3[nH]nc(F)c3c2)cn1)N1CCOCC1 ; Cl]

Compound 18 was synthesized following the approach outlined in Scheme 3, modifying Step-7 by substituting tert-butyl (E)-(2-((5-iodopyridin-2-yl)oxy)ethyl)(4-morpholino-4-oxobut-2-en-1-yl)carbamate (preparation show below in Step-a) for compound 324 to deliver the title compound in 48.0 mg, 3.82% overall yield. ¹H NMR (400 MHz, DMSO-d6) δ 12.74 (s, 1H), 9.17 (s, 2H), 7.69-7.64 (m, 2H), 7.57-7.55 (m, 1H), 7.33-7.18 (m, 7H), 6.85-6.81 (dd, J=15.3, 1.3 Hz, 1H), 6.64-6.57 (m, 2H), 4.38-4.35 (t, J=5.0 Hz, 2H), 3.56-3.44 (m, 10H), 3.25 (d, J=6.1 Hz, 2H), 2.51-2.50 (m, 2H). LCMS: 609.63 [M+H]⁺.

### Step-a: Synthesis of tert-butyl (E)-(2-((5-iodopyridin-2-yl)oxy)ethyl)(4-morpholino-4-oxobut-2-en-1-yl)carbamate

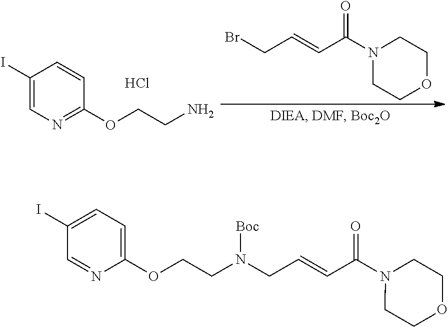 [SMILES: NCCOc1ccc(I)cn1 ; Cl ; O=C(/C=C/CBr)N1CCOCC1 ; CC(C)(C)OC(=O)N(C/C=C/C(=O)N1CCOCC1)CCOc1ccc(I)cn1]

The title compound was synthesized following the approach outlined in Scheme 4, Step-3, substituting (E)-4-bromo-1-morpholinobut-2-en-1-one for compound 329. ¹H NMR (400 MHz, DMSO-d6) δ 8.36 (d, J=2.3 Hz, 1H), 8.01-7.99 (d, J=7.9 Hz, 1H), 6.73-6.68 (m, 1H), 6.60-6.55 (dd, J=15.1, 5.9 Hz, 1H), 6.47-6.41 (m, 1H), 4.34-4.32 (t, J=5.3 Hz, 2H), 3.99-3.98 (m, 2H), 3.55-3.34 (dt, J=19.7, 5.1 Hz, 10H), 1.37-1.32 (d, J=17.6 Hz, 9H). LCMS: 518 [M+H]⁺.

### Example 19: Synthesis of (E)-1-morpholino-4-((2-((5-((Z)-4,4,4-trifluoro-1-(1H-indazol-5-yl)-2-phenylbut-1-en-1-yl)pyridin-2-yl)oxy)ethylamino)but-2-en-1-one (Compound 19)

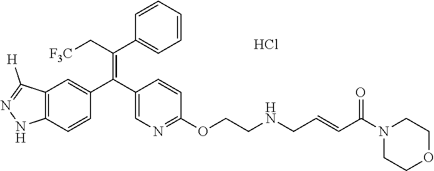 [SMILES: O=C(/C=C/CNCCOc1ccc(/C(=C(/CC(F)(F)F)c2ccccc2)c2ccc3[nH]ncc3c2)cn1)N1CCOCC1 ; Cl]

Compound 19 was synthesized following the approach outlined in Scheme 8 by modifying: a) Step-6 by substituting tert-butyl (E)-(2-((5-iodopyridin-2-yl)oxy)ethyl)(4-morpholino-4-oxobut-2-en-1-yl)carbamate (preparation shown in Example 18, Step-a) for compound 324, 2-Methyl THF:H₂O (4:1) for dioxane:H₂O, using 2.0 equiv of Cs₂CO₃, and stirring at 60° C. until completion, and b) Step-7 by using 4.0 equiv of KOH and 1.2 equiv of bromobenzene to deliver the title compound in 59.9 mg, 1.16% overall yield. ¹H NMR (400 MHz, Methanol-d4) δ 8.44 (d, J=1.0 Hz, 1H), 7.93 (m, J=1.2 Hz, 1H), 7.79 (dd, J=2.4, 0.7 Hz, 1H), 7.72-7.70 (m, J=8.7, 0.9 Hz, 1H), 7.61-7.58 (dd, J=8.8, 2.4 Hz, 1H), 7.44-7.42 (dd, J=8.8, 1.6 Hz, 1H), 7.28-7.22 (m, 5H), 6.93-6.87 (m, 2H), 6.72-6.64 (m, J=15.2, 6.7 Hz, 1H), 4.57-4.54 (m, 2H), 3.91-3.89 (dd, J=6.7, 1.3 Hz, 2H), 3.67-3.62 (m, 8H), 3.48-3.40 (m, 4H). LCMS: 592.3 [M+H]⁺.

### Example 20: Synthesis of (E)-N-(2-methoxyethyl)-4-((2-((5-((Z)-4,4,4-trifluoro-(3-fluoro-1H-indazol-5-yl)-2-phenylbut-1-en-1-yl)pyridin-2-yl)oxy)ethyl)amino)but-2-enamide (Compound 20)

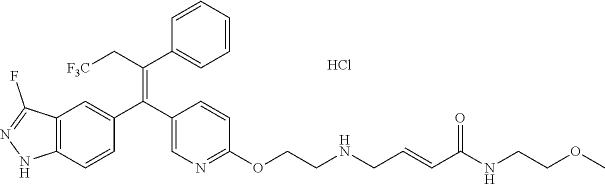 [SMILES: COCCNC(=O)/C=C/CNCCOc1ccc(/C(=C(/CC(F)(F)F)c2ccccc2)c2ccc3[nH]nc(F)c3c2)cn1 ; Cl]

Compound 20 was synthesized following the approach outlined in Scheme 3 by modifying: a) Step-7 by substituting tert-butyl (E)-(2-((5-iodopyridin-2-yl)oxy)ethyl)(4-((2-methoxyethyl)amino)-4-oxobut-2-en-1-yl)carbamate (preparation shown below in Steps-a-b) for compound 324, and b) Step-8 by stirring at 90° C. until completion to deliver the title compound in 32.9 mg, 3.50% overall yield. ¹H NMR (400 MHz, Methanol-d4) δ 7.70-7.69 (m, 1H), 7.63 (s, 1H), 7.50-7.48 (m, 1H), 7.31-7.29 (m, 2H), 7.24-7.18 (m, 5H), 6.71-6.62 (m, 2H), 6.32-6.28 (m, 1.2 Hz, 1H), 4.46-4.43 (m, 2H), 3.84-3.82 (m, 2H), 3.48-3.33 (m, 11H). LCMS: 620 [M+Na]⁺.

### Step-a: Synthesis of (E)-4-bromo-N-(2-methoxyethyl)but-2-enamide

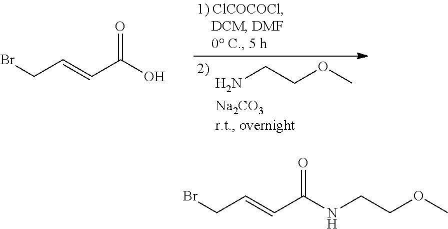

Into a 250-mL round-bottom flask was placed (E)-4-bromobut-2-enoic acid (10 g, 60.61 mmol, 1.00 equiv), DCM (200 mL, 1.00 equiv), and N,N-dimethylformamide (1.0 mL), then oxalyl dichloride (8.45 g, 66.57 mmol, 1.10 equiv) was added in dropwise at 0° C. The resulting solution was stirred at 25° C. until completion. Then, into a 250-mL round-bottom flask was placed 2-methoxyethan-1-amine (5.49 g, 73.09 mmol, 1.20 equiv), sodium carbonate (25.86 g, 243.99 mmol, 4.00 equiv), followed by the addition of the above acetyl chloride. The resulting solution was stirred at R.T until completion. Then the solution was diluted with 300 mL of water and extracted with 3×300 mL of DCM. Then the organic layers was combined and washed with 300 mL of brine. The mixture was dried over anhydrous sodium sulfate and concentrated under vacuum to deliver the title compound in 12.5 g (85%) as an oil.

### Step-b: Synthesis of tert-butyl (E)-(2-((5-iodopyridin-2-yl)oxy)ethyl(4-((2-methoxyethyl)amino)-4-oxobut-2-en-1-yl)carbamate

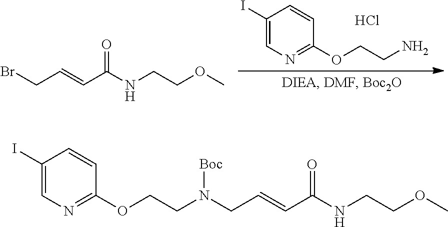 [SMILES: COCCNC(=O)/C=C/CBr ; NCCOc1ccc(I)cn1 ; Cl ; COCCNC(=O)/C=C/CN(CCOc1ccc(I)cn1)C(=O)OC(C)(C)C]

Into a 250-mL round-bottom flask was placed (E)-4-bromo-N-(2-methoxyethyl)but-2-enamide (10 g, 45.03 mmol, 1.00 equiv), 2-((5-iodopyridin-2-yl)oxy)ethan-1-amine hydrochloride (12.3 g, 40.93 mmol, 0.90 equiv), DIEA (17.4 g, 134.63 mmol, 3.00 equiv), and N,N-dimethylformamide (100 mL). The resulting solution was stirred at RT until completion. Then Boc₂O (26.8 g, 134.63 mmol, 3.00 equiv) was added and the resulting solution was stirred at R.T until completion. The solution was diluted with 250 mL of water and extracted with 3×300 mL of ethyl acetate, then the organic layers were combined and washed with 300 mL of brine. The mixture was dried over anhydrous sodium sulfate and concentrated under vacuum. The residue was applied onto a silica gel column eluting with petroleum ether/ethyl acetate (1:1) to deliver the title compound in 11.0 g (96%) as an oil. The product isolated was still not clean, taken forward to the next step without further purification. LCMS: 506 [M+H]⁺.

### Example 21: Synthesis of (E)-N-methyl-4-((2-(4-((E)-4,4,4-trifluoro-1-(3-fluoro-1H-indazol-5-yl)-2-phenylbut-1-en-1-yl)phenoxy)ethyl)amino)but-2-enamide (Compound 21)

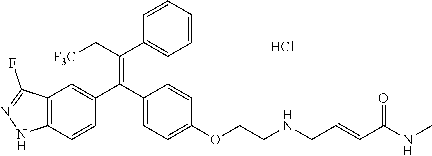 [SMILES: CNC(=O)/C=C/CNCCOc1ccc(/C(=C(/CC(F)(F)F)c2ccccc2)c2ccc3[nH]nc(F)c3c2)cc1 ; Cl]

Compound 21 was synthesized beginning with Step-1 through Step-6 in Example 3, resulting in the preparation of (Z)-3-fluoro-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole. Preparation of Compound 21 then continued with Step-7 as set forth below.

### Step-7: Synthesis of ((Z)-1-(4-(2-((tert-butoxycarbonyl((E)-4-(methylamino)-4-oxobut-2-en-1-yl)amino)ethoxy)phenyl)-4,4,4-trifluoro-1-(3-fluoro-1-(tetrahydr-2H-pyran-2-yl)-1H-indazol-5-yl)but-1-en-2-yl)boronic acid

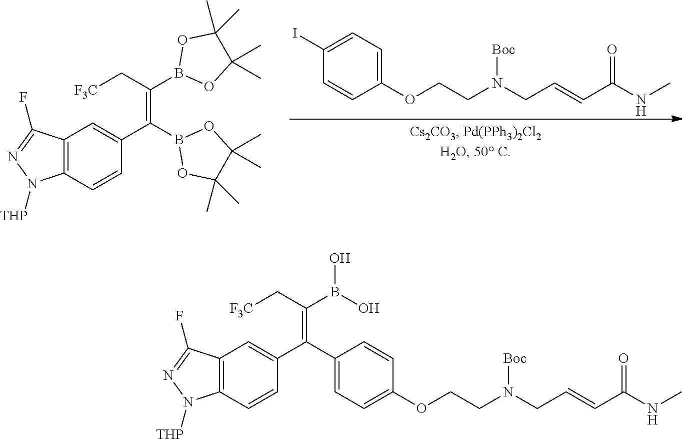

Into a 1000-mL round-bottom flask purged and maintained with an inert atmosphere of nitrogen, was placed (Z)-3-fluoro-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (53 g, 91.94 mmol, 1.00 equiv), 2-Methyl THF (500 mL), tert-butyl (E)-(2-(4-iodophenoxy)ethyl)(4-(methylamino)-4-oxobut-2-en-1-yl)carbamate (42.3 g, 91.90 mmol, 1.00 equiv), Cs₂CO₃ (90 g, 276.23 mmol, 3.00 equiv), Pd(PPh₃)₂Cl₂ (6.46 g, 9.20 mmol, 0.10 equiv), and water (100 mL) were added. The resulting solution was stirred at 50° C. until completion. The reaction progress was monitored by LCMS. The solution was diluted with 500 mL of H₂O, extracted with 2×600 mL of ethyl acetate, then the organic layers were combined, dried over anhydrous sodium sulfate, and concentrated under vacuum. The residue was applied onto a silica gel column with ethyl acetate/petroleum ether (1:2). The collected fractions were combined and concentrated under vacuum to deliver the title compound in 36 g (54%) as yellow oil.

### Step-8: Synthesis of tert-butyl ((E)-4-(methylamino)-4-oxobut-2-en-1-yl)(2-(4-((E)-4,4,4-trifluoro-1-(3-fluoro-1-(tetrahydro-2H-pyran-2-yl)-1H-indazol-5-yl)-2-phenylbut-1-en-1-yl)phenoxy)ethyl)carbamate

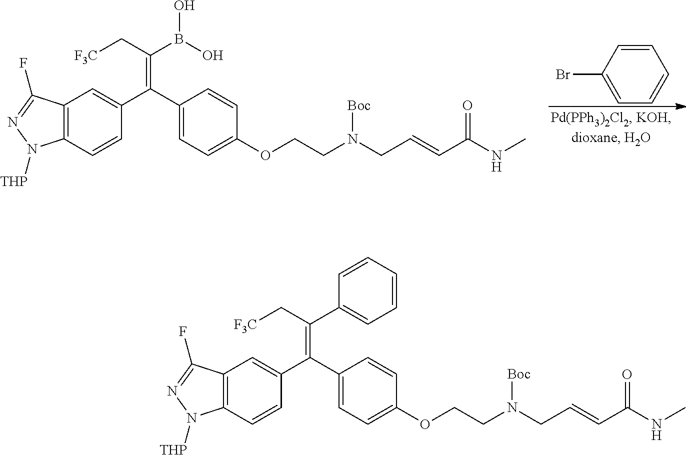

Into a 1-L round-bottom flask purged and maintained with an inert atmosphere of nitrogen, was placed ((Z)-1-(4-(2-((tert-butoxycarbonyl)((E)-4-(methylamino)-4-oxobut-2-en-1-yl)amino)ethoxy)phenyl)-4,4,4-trifluoro-1-(3-fluoro-1-(tetrahydro-2H-pyran-2-yl)-1H-indazol-5-yl)but-1-en-2-yl)boronic acid (36 g, 51.10 mmol, 1.00 equiv), Pd(PPh)₂Cl₂ (3.5 g, 4.99 mmol, 0.10 equiv), potassium hydroxide (8.4 g, 149.71 mmol, 3.00 equiv), dioxane (200 mL), water (40 mL), and bromobenzene (8.4 g, 53.50 mmol, 1.00 equiv). The resulting solution was stirred at 80° C. until completion. The reaction progress was monitored by LCMS. The resulting solution was diluted with 200 ml of H₂O, extracted with 3×500 ml of ethyl acetate, then the organic layers combined, washed with brine (200 ml) and dried over anhydrous sodium sulfate. The residue was applied onto a silica gel column with ethyl acetate/petroleum ether (1:3). The collected fractions were combined and concentrated under vacuum to deliver the title compound in 16 g (42%) as a yellow solid.

### Step-9: Synthesis of (E)-N-methyl-4-((2-(4-((E)-4,4,4-trifluoro-1-(3-fluoro-1H-indazol-5-yl)-2-phenylbut-1-en-1-yl)phenoxy)ethyl)amino)but-2-enamide

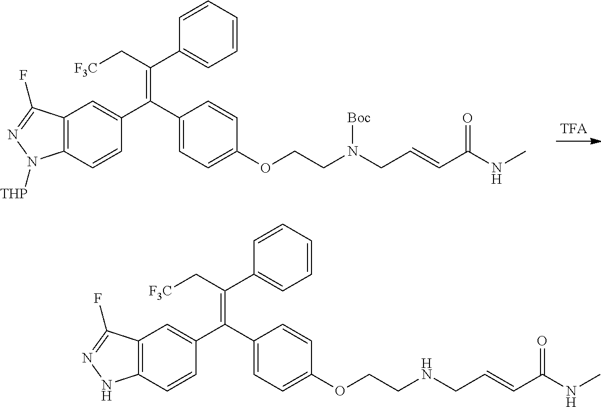

Into a 250-mL round-bottom flask was placed tert-butyl ((E)-4-(methylamino)-4-oxobut-2-en-1-yl)(2-(4-((E)-4,4,4-trifluoro-1-(3-fluoro-1-(tetrahydro-2H-pyran-2-yl)-1H-indazol-5-yl)-2-phenylbut-1-en-1-yl)phenoxy)ethyl)carbamate (16 g, 21.72 mmol, 1.00 equiv) and TFA (100 mL). The resulting solution was stirred at 25° C. until completion. The reaction progress was monitored by LCMS. The resulting solution was concentrated under vacuum and the crude product was purified by Prep-HPLC with the following conditions: Column: X-Bridge Prep OBD C18 Column 30×150 mm Sum; Mobile Phase A:Water (10 mmol/L NH4HCO₃), Mobile Phase B: ACN; Flow rate: 60 mL/min; Gradient: 40% B to 55% B in 60 min; 254,220 nm, to deliver the title compound as a free base in 4.05 g as a yellow solid. ¹H NMR (400 MHz, Methanol-d4) δ 7.60 (s, 1H), 7.52-7.42 (m, 1H), 7.25-7.11 (m, 6H), 6.83-6.81 (m, 2H), 6.78-6.71 (m, 1H), 6.63-6.61 (m, 2H), 6.05-6.01 (m, 1H), 3.97-3.94 (t, J=5.3 Hz, 2H), 3.41-3.33 (m, 4H), 2.98-2.88 (t, J=5.2 Hz, 2H), 2.76 (s, 3H).

The solid was then dissolved in 100 mL CH₃CN and acidified with 8.07 mL HCl (1N) (1 mL 12N HCl(aq) dissolved in 11 mL CH₃CN) at 0° C., and stirred for 30 min at R.T., then evaporated at 30° C. to remove the excess HCl. Then the product was dissolved in 150 mL H₂O and lyophilized for 48 h to deliver the title compound in 4.4 g, 0.96% overall yield, as a yellow solid. ¹H NMR (400 MHz, Methanol-d4) δ 7.59 (s, 1H), 7.46-7.44 (m, 1H), 7.27 (m, 1H) 7.25-7.12 (m, 5H), 6.91-6.87 (m, 2H), 6.72-6.65 (m, 3H), 6.30-6.26 (m, 1H), 4.16-4.14 (t, J=4.9 Hz, 2H), 3.86-3.84 (m, 2H), 3.42-3.34 (m, 4H), 2.79 (s, 3H). LCMS: 553 [M+H]⁺.

Compound 21 was also synthesized following the approach outlined in Scheme 10, omitting Steps 1-3, by modifying Step-5 by substituting (E)-4-bromo-N-methylbut-2-enamide (Scheme 4, Steps-a-b) for compound 359 to deliver the title compound in 675 mg, 21.9% overall yield. ¹H NMR (400 MHz, Methanol-d4) δ 7.61 (d, J=1.5 Hz, 1H), 7.49-7.46 (m, 1H), 7.30-7.28 (m, 1H), 7.24-7.15 (m, 5H), 6.92-6.89 (m, 2H), 6.74-6.67 (m, 3H), 6.31-6.27 (m, 1H), 4.18-4.15 (t, J=4.8 Hz, 2H), 3.88-3.86 (m, 2H), 3.45-3.37 (m, 4H), 2.82 (s, 3H

### Example 22: Synthesis of (E)-N,N-di(²H₃)methyl-4-((2-(4-((E)-4,4,4-trifluoro-1-(3-fluoro-1H-indazol-5-yl)-2-phenylbut-1-en-1-yl)phenoxy)ethyl)amino)but-2-enamide (Compound 22)

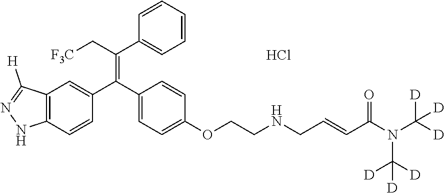 [SMILES: [2H]C([2H])([2H])N(C(=O)/C=C/CNCCOc1ccc(/C(=C(/CC(F)(F)F)c2ccccc2)c2ccc3[nH]ncc3c2)cc1)C([2H])([2H])[2H] ; Cl]

Compound 22 was synthesized following the approach outlined in Scheme 10, omitting Step 4, modifying Step-5 by substituting (E)-4-bromo-N,N-bis(methyl-d3)but-2-enamide (Scheme 4, Steps-a-b, substituting bis(methyl-d3)amine hydrochloride for methylamine) for compound 359 to deliver the title compound in 96.0 mg, 2.08% overall yield, as a white solid. ¹H NMR (300 MHz, DMSO-d6) δ12.72 (s, 1H), 9.18 (s, 2H), 7.58-7.52 (m, 2H), 7.26-7.14 (m, 6H), 6.87-6.79 (m, 3H), 6.62-6.60 (m, 2H), 6.58-6.53 (s, 1H), 4.14-4.11 (t, J=9.0 Hz, 2H), 3.80-3.74 (m, 2H), 3.51-3.32 (m, 2H), 3.26-3.13 (m, 2H). LCMS: 610.1 [M+H]⁺.

### Example 23: Synthesis of (E)-N,N-di(²H₃)methyl-4-((E)-4,4,4-trifluoro-1-(1H-indazol-5-yl)-2-phenylbut-1-en-1-yl)phenoxy)ethyl)amino)but-2-enamide (Compound 23)

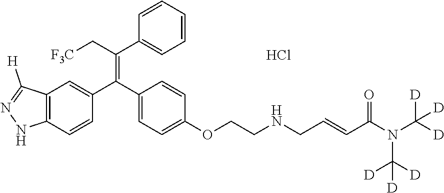 [SMILES: [2H]C([2H])([2H])N(C(=O)/C=C/CNCCOc1ccc(/C(=C(/CC(F)(F)F)c2ccccc2)c2ccc3[nH]ncc3c2)cc1)C([2H])([2H])[2H] ; Cl]

Compound 23 was synthesized following the approach outlined in Scheme 10, omitting Step 4, by modifying: a) Step-1 by substituting (Z)-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (Scheme 8, Steps-1-5) for compound 323, and b) Step-5 by substituting (E)-4-bromo-N,N-bis(methyl-d3)but-2-enamide (Scheme 4, Steps-a-b, substituting bis(methyl-d3)amine hydrochloride for methylamine) for compound 359 to deliver the title compound in 30.0 mg, 0.18% overall yield, as a yellow solid. ¹H NMR (400 MHz, Methanol-d4) δ 8.32 (s, 1H), 7.81 (s, 1H), 7.62-7.60 (m, 1H), 7.33-7.30 (dd, J=8.7, 1.5 Hz, 1H), 7.22-7.10 (m, 5H), 6.91-6.83 (m, 3H), 6.72-6.62 (m, 3H), 4.17-4.15 (m, 2H), 3.89-3.88 (dd, J=6.6, 1.3 Hz, 2H), 3.47-3.34 (m, 4H). LCMS: 555.51 [M+H]⁺.

### Example 24: Synthesis of (E)-N,N-di(²H₃)methyl-4-((2-((5-((Z)-4,4,4-trifluoro-1-(3-fluoro-1H-indazol-1-yl)-2-phenylbut-1-en-1-yl)pyridin-2-yl)oxy)ethyl)amino)but-2-enamide (Compound 24)

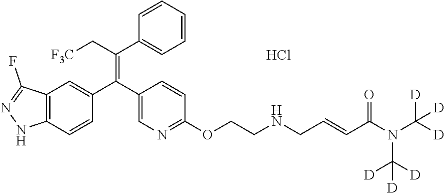 [SMILES: [2H]C([2H])([2H])N(C(=O)/C=C/CNCCOc1ccc(/C(=C(/CC(F)(F)F)c2ccccc2)c2ccc3[nH]nc(F)c3c2)cn1)C([2H])([2H])[2H] ; Cl]

Compound 24 was synthesized following the approach outlined in Scheme 10, omitting Step-4, by modifying: a) Step-1 by substituting tert-butyl (2-((5-iodopyridin-2-yl)oxy)ethyl)carbamate (Scheme 4, Step-1) for compound 307, and b) modifying Step-5 by substituting (E)-4-bromo-N,N-bis(methyl-d3)but-2-enamide (Scheme 4, Steps-a-b, substituting bis(methyl-d3)amine hydrochloride for methylamine) for compound 359 to deliver the title compound in 59.9 mg, 0.35% overall yield. ¹H NMR (400 MHz, Methanol-d4) δ 7.77-7.64 (m, 2H), 7.51-7.29 (d, J=9.1 Hz, 3H), 7.24-7.19 (d, J=8.5 Hz, 5H), 6.87-6.61 (m, 3H), 4.50-4.45 (dd, J=3.1, 1.7 Hz, 2H), 3.87-3.86 (s, 2H), 3.48-3.46 (s, 4H). LCMS: 574 [M+H]⁺.

### Example 25: Synthesis of (E)-N,N-di(²H₃)methyl-4-((2-((5-((Z)-4,4,4-trifluoro-1-(1H-indazol-5-yl)-2-phen ylbut-1-en-1-yl)pyridin-2-yl)oxy)ethyl)amino)but-2-enamide (Compound 25)

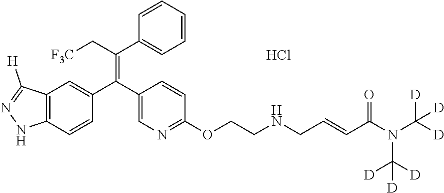 [SMILES: [2H]C([2H])([2H])N(C(=O)/C=C/CNCCOc1ccc(/C(=C(/CC(F)(F)F)c2ccccc2)c2ccc3[nH]ncc3c2)cn1)C([2H])([2H])[2H] ; Cl]

Compound 25 was synthesized following the approach outlined in Scheme 10, omitting Step-4, by modifying: a) Step-1 by substituting (Z)-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (Scheme 8, Steps-1-5) for compound 323, and tert-butyl (2-((5-iodopyridin-2-yl)oxy)ethyl)carbamate (Scheme 4, Step-1) for compound 307 and b) modifying Step-5 by substituting (E)-4-bromo-N,N-bis(methyl-d3)but-2-enamide (Scheme 4, Steps-a-b, substituting bis(methyl-d3)amine hydrochloride for methylamine) for compound 359 to deliver the title compound in 70.5 mg, 0.83% overall yield. ¹H NMR (300 MHz, DMSO-d6) δ 9.32 (s, 2H), 8.14 (d, J=1.0 Hz, 1H), 7.70-7.59 (m, 3H), 7.33-7.16 (m, 8H), 6.84-6.79 (m, 1H), 6.63-6.52 (m, 2H), 4.39-4.36 (t, J=5.0 Hz, 2H), 3.78-3.76 (d, J=6.1 Hz, 2H), 3.54-3.47 (m, 2H), 3.23 (s, 2H). LCMS: 556 [M+H]⁺.

### Example 26: Synthesis of (E)-4-((2-(4-((E)-1-(3-fluoro-1H-indazol-5-yl)-2-phenylbut-1-en-1-yl)phenoxy)ethyl)amino)-N-methylbut-2-enamide (Compound 26)

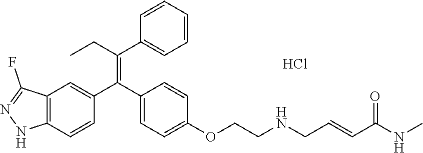 [SMILES: CC/C(=C(/c1ccc(OCCNC/C=C/C(=O)NC)cc1)c1ccc2[nH]nc(F)c2c1)c1ccccc1 ; Cl]

Compound 26 was synthesized following the approach outlined in Scheme 9 by modifying: a) Step-1 by substituting 1-phenylpropan-1-one for compound 349, DCM for toluene, and stirring at room temperature until completion, b) Step-2 by substituting THF (to make a 0.43M solution) for ether, adding the n-BuLi at −78° C., using 1.25 equiv of 4,4,5,5-tetramethyl-2-(tetramethyl-1,3,2-dioxaborolan-2-yl)-1,3,2-dioxaborolane, and stirring at room temperature once all reagents were added, c) Step-3 by substituting 5-bromo-3-fluoro-1-(tetrahydro-2H-pyran-2-yl)-1H-indazole (Scheme 3, Steps-1-2) for compound 352, Pd(dppf)Cl₂ for Pd₂(dba)₃, 4.0 equiv of Cs₂CO₃ for KOH, a 10:1 ratio of dioxane:H₂O for THF, and removing P(t-Bu)₃.HBF, and d) Step-4 by substituting Pd(dppf)Cl₂ for Pd₂(dba)₃.CHCl₃, and dioxane (to male a 0.2M solution) for THF to deliver the title compound in 76.9 mg, 1.23% overall yield. ¹H NMR (400 MHz, Methanol-d4) δ 7.51 (d, J=1.3 Hz, 1H), 7.45-7.42 (m, 1H), 7.30-7.27 (dd, J=8.8, 1.6 Hz, 1H), 7.20-7.10 (m, 5H), 6.89-6.87 (d, J=8.8 Hz, 2H), 6.74-6.67 (dd, J=8.7, 7.2 Hz, 3H), 6.31-6.27 (m, 1H), 4.17-4.15 (m, 2H), 3.88-3.86 (m, 2H), 3.50-3.49 (t, J=4.9 Hz, 2H), 2.82 (s, 3H), 2.53-2.47 (q, J=7.5 Hz, 2H), 0.98-0.94 (t, J=7.4 Hz, 3H). LCMS: 499.0 [M+H]⁺.

### Example 27: Synthesis of (E)-4-((2-((5-((Z)-1-(3-fluoro-1H-indazol-5-yl)-2-phenylbut-1-en-1-yl)pyridin-2-yl)oxy)ethyl)amino)-N-methylbut-2-enamide (Compound 27)

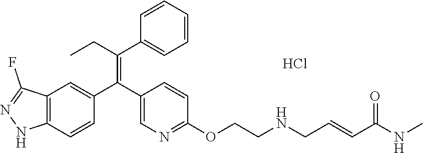 [SMILES: CC/C(=C(/c1ccc(OCCNC/C=C/C(=O)NC)nc1)c1ccc2[nH]nc(F)c2c1)c1ccccc1 ; Cl]

Compound 27 was synthesized following the approach outlined in Scheme 9 by modifying: a) Step-1 by substituting 1-phenylpropan-1-one for compound 349, DCM for toluene, and stirring at room temperature until completion, b) Step-2 by substituting THF (to make a 0.43M solution) for ether, adding the n-BuLi at −78° C., using 1.25 equiv of 4,4,5,5-tetramethyl-2-(tetramethyl-1,3,2-dioxaborolan-2-yl)-1,3,2-dioxaborolane, and stirring at room temperature once all reagents were added until completion, c) Step-3 by substituting 5-bromo-3-fluoro-1-(tetrahydro-2H-pyran-2-yl)-1H-indazole (Scheme 3, Steps-1-2) for compound 352. Pd(dppf)Cl₂ for Pd₂(dba)₃, 4.0 equiv of Cs₂CO₃ for KOH, a 10:1 ratio of dioxane:HzO for THF, and removing P(t-Bu)₃.HBF, d) Step-4 by substituting tert-butyl (E)-(2-((5-iodopyridin-2-yl)oxy)ethyl)(4-(methylamino)-4-oxobut-2-en-1-yl)carbamate (Scheme 4, Steps-1-3) for compound 335, dioxane (to make a 0.4M solution) for THF, Pd(PPh₃)₂Cl₂ for Pd₂(dba)₃.CHCl₃, and stirring at 60° C., and e) Step-5 by using a 2:1 ratio of TFA:DCM to deliver the title compound in 41.2 mg, 0.28% overall yield. ¹H NMR (400 MHz, Methanol-d4) δ 7.71 (dd, J=2.5, 0.7 Hz, 1H), 7.58 (m, J=1.2 Hz, 1H), 7.50-7.47 (m, J=8.7, 2.4, 0.9 Hz, 1H), 7.41-7.39 (dd, J=8.7, 2.4 Hz, 1H), 7.34-7.31 (dd, J=8.8, 1.6 Hz, 1H), 7.27-7.18 (m, 5H), 6.75-6.67 (m, 2H), 6.32-6.28 (m, J=15.4, 1.4 Hz, 1H), 4.50-4.48 (m, 2H), 3.88-3.86 (dd, J=6.9, 1.4 Hz, 2H), 3.44-3.41 (m, 2H), 2.82 (s, 3H), 2.56-2.50 (m, J=7.4 Hz, 2H), 1.00-0.96 (m, J=7.4 Hz, 3H). LCMS: 500.3 [M+H]⁺.

### Example 28: Synthesis of (E)-4-((2-((5-((Z)-1-(1H-indazol-5-yl)-2-phenylbut-1-en-1-yl)pyridin-2-yl)oxy)ethyl)amino)-N-methylbut-2-enamide (Compound 28)

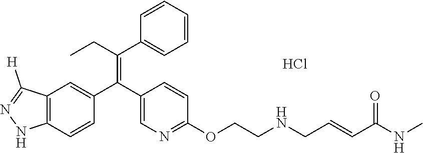 [SMILES: CC/C(=C(/c1ccc(OCCNC/C=C/C(=O)NC)nc1)c1ccc2[nH]ncc2c1)c1ccccc1 ; Cl]

Compound 28 was synthesized following the approach outliend in Scheme 9 by modifying: a) Step-1 by substituting 1-phenylpropan-1-one for compound 349, DCM for toluene, and stirring at room temperature until completion, b) Step-2 by substituting THF (to make a 0.43M solution) for ether, adding the n-BuLi at −78° C., using 1.25 equiv of 4,4,5,5-tetramethyl-2-(tetramethyl-1,3,2-dioxaborolan-2-yl)-1,3,2-dioxaborolane, and stirring at room temperature once all reagents were added, c) Step-3 by substituting Pd(dppf)Cl₂ for Pd₂(dba)₃, e equiv of Cs₂CO₃ for KOH, dioxane:H₂O (4:1) for THF, removing P(t-Bu)₃.HBF, and stirring at 80° C., d) Step-4 by substituting tert-butyl (E)-(2-((5-iodopyridin-2-yl)oxy)ethyl)(4-(methylamino)-4-oxobut-2-en-1-yl)carbamate (Scheme 4. Steps-1-3) for compound 335, Pd(dppf)Cl₂ for Pd₂(dba)₃.CHCl₃, a 4:1 ratio of dioxane:H₂O for THF, and stirring at 60° C., and e) Step-5 by using a 1:1 ratio of TFA:DCM to deliver the title compound in 117.6 mg, 1.20% overall yield, as an off-white solid. ¹H NMR (400 MHz, Methanol-d4) δ 8.12 (m, 1H), 7.70-7.66 (m, 2H), 7.57-7.55 (m, 1H), 7.31-7.15 (m, 7H), 6.68-6.61 (m, 2H), 6.29-6.25 (m, 1H), 4.46-4.43 (m, 2H), 3.84-3.82 (dd, J=6.9, 1.4 Hz, 2H), 3.40-3.37 (m, 2H), 2.79 (s, 3H), 2.52-2.48 (m, 2H), 0.97-0.93 (t, J=7.4 Hz, 3H). LCMS: 482.21 [M+H]⁺.

### Example 29: Synthesis of (E)-4-((2-(4-((E)-1-(3-fluoro-1H-indazol-5-yl)-2-phenylbut-1-en-1-yl)phenoxy)ethyl)amino)-1-(pyrrolidin-1-yl)but-2-en-1-one (Compound 29)

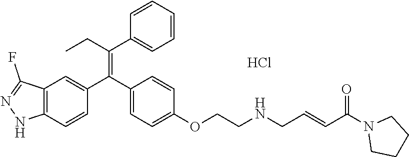 [SMILES: CC/C(=C(/c1ccc(OCCNC/C=C/C(=O)N2CCCC2)cc1)c1ccc2[nH]nc(F)c2c1)c1ccccc1 ; Cl]

Compound 29 was synthesized following the approach outlined in Scheme 9 by modifying: a) Step-1 by substituting 1-phenylpropan-1-one for compound 349, DCM for toluene, and stirring at room temperature until completion, b) Step-2 by substituting THF (to make a 0.43M solution) for ether, adding the n-BuLi at −78° C., using 1.25 equiv of 4,4,5,5-tetramethyl-2-(tetramethyl-1,3,2-dioxaborolan-2-yl)-1,3,2-dioxaborolane, and stirring at room temperature once all reagents were added until completion, c) Step-3 by substituting 5-bromo-3-fluoro-1-(tetrahydro-2H-pyran-2-yl)-1H-indazole (Scheme 3, Steps-1-2) for compound 352, Pd(dppf)Cl₂ for Pd₂(dba)₃, 4.0 equiv of Cs₂CO₃ for KOH, a 10:1 ratio of dioxane:H₂O for THF, and removing P(t-Bu)₃.HBF, and d) Step-4 by substituting tert-butyl (E)-(2-(4-iodophenoxy)ethyl)(4-oxo-4-(pyrrolidin-1-yl)but-2-en-1-yl)carbamate (preparation shown in Example 14, Step-a) for compound 335, Pd(dppf)Cl₂.CH₂Cl₂ for Pd₂(dba)₃.CHCl₃, dioxane (to make a 0.3M solution) for THF, and stirring at 60° C. to deliver the title compound in 40 mg, 0.22% overall yield, as a white solid. ¹H NMR (400 MHz, Methanol-d4) δ 7.51 (t, J=1.2 Hz, 1H), 7.44-7.42 (m, 1H), 7.23-7.27 (dd, J=8.7, 1.5 Hz, 1H), 7.18-7.11 (m, 5H), 6.89-6.87 (m, 2H), 6.74-6.69 (m, 4H), 4.19-4.16 (m, 2H), 3.92-3.91 (d, J=5.1 Hz, 2H), 3.64-3.60 (t, J=6.8 Hz, 2H), 3.51-3.48 (m, 2H), 3.44-3.41 (m, 2H) 2.51-2.49 (q, J=7.4 Hz, 2H), 2.05-1.91 (m, 4H), 0.98-0.94 (t, J=7.4 Hz, 3H). LCMS: 539.3 [M+H]⁺.

### Example 30: Synthesis of (E)-4-((2-((5-((Z)-1-(3-fluoro-1H-indazol-5-yl)-2-phenylbut-1-en-1-yl)pyridin-2-yloxy)ethyl)amino)-1-(pyrrolidin-1-yl)but-2-en-1-one (Compound 30)

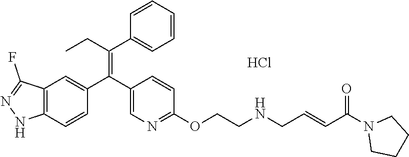 [SMILES: CC/C(=C(/c1ccc(OCCNC/C=C/C(=O)N2CCCC2)nc1)c1ccc2[nH]nc(F)c2c1)c1ccccc1 ; Cl]

Compound 30 was synthesized following the approach outlined in Scheme 9 by modifying: a) Step-1 by substituting 1-phenylpropan-1-one for compound 349, DCM for toluene, and stirring at room temperature until completion, b) Step-2 by substituting THF (to make a 0.43M solution) for ether, adding the n-BuLi at −78° C., using 1.25 equiv of 4,4,5,5-tetramethyl-2-(tetramethyl-1,3,2-dioxaborolan-2-yl)-1,3,2-dioxaborolane, and stirring at room temperature once all reagents were added until completion, c) Step-3 by substituting 5-bromo-3-fluoro-1-(tetrahydro-2H-pyran-2-yl)-1H-indazole (Scheme 3, Steps-1-2) for compound 352, Pd(dppf)Cl₂ for Pd₂(dba)₃, 4.0 equiv of Cs₂CO₃ for KOH, a 10:1 ratio of dioxane:H₂O for THF, and removing P(t-Bu)₃.HBF, and d) Step-4 by substituting tert-butyl (E)-(2-((5-iodopyridin-2-yl)oxy)ethyl)(4-oxo-4-(pyrrolidin-1-yl)but-2-en-1-yl)carbamate (preparation shown in Example 15, Step-a) for compound 335, Pd(dppf)Cl₂ for Pd₂(dba)₃.CHCl₃, dioxane (to make a 0.3M solution) for THF, and stirring at 60° C. to deliver the title compound in 50 mg, 1.38% overall yield, as a yellow solid. ¹H NMR (400 MHz, Methanol-d4) δ 7.75 (d, J=2.3 Hz, 1H), 7.67-7.60 (m, 2H), 7.50-7.48 (m, 1H), 7.35 (dd, J=8.7, 1.5 Hz, 1H), 7.33-7.20 (m, 5H), 7.00-6.98 (d, J=8.9 Hz, 1H), 6.72-6.70 (d, J=2.7 Hz, 2H), 4.58-4.56 (t, J=4.8 Hz, 2H), 3.92-3.91 (m, 2H), 3.63-3.59 (t, J=6.8 Hz, 2H), 3.49-3.46 (dd, J=8.6, 5.4 Hz, 4H), 2.53-2.50 (q, J=7.4 Hz, 2H), 2.01-1.89 (m, 4H), 0.98-0.94 (t, J=7.4 Hz, 3H). LCMS: 540.15 [M+H]⁺.

### Example 31: Synthesis of (E)-4-((2-(4-((E)-2-cyclobutyl-1-(3-fluoro-1H-indazol-5-yl)-2-phenylvinyl)phenoxy)ethyl)amino)-N-methylbut-2-enamide (Compound 31)

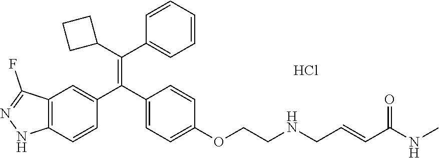 [SMILES: CNC(=O)/C=C/CNCCOc1ccc(/C(=C(\c2ccccc2)C2CCC2)c2ccc3[nH]nc(F)c3c2)cc1 ; Cl]

Compound 31 was synthesized following the approach outlined in Scheme 9 by modifying: a) Step-3 by substituting 1.2 equiv of 5-bromo-3-fluoro-1-(tetrahydro-2H-pyran-2-yl)-1H-indazole (Scheme 3, Steps-1-2) for compound 352 and stirring at 20° C., b) Step-4 by substituting Pd(dppf)Cl₂ for Pd₂(dba)₃.CHCl₃, and c) Step-5 by removing DCM to deliver the title compound in 41.4 mg, 0.42% overall yield. ¹H NMR (400 MHz, Methanol-d4) δ 7.50 (s, 1H), 7.49-7.39 (m, 1H), 7.30-7.29 (m, 1H), 7.23-7.21 (m, 2H), 7.20-7.15 (m, 1H), 7.14-7.10 (m, 2H), 6.91-6.88 (m, 2H), 6.68-6.65 (m, 3H), 6.31-6.27 (m, 1H), 4.13-4.11 (m, 2H), 3.86-3.85 (m, 2H), 3.56-3.44 (m, 1H), 3.33 (t, J=4.9 Hz, 2H), 2.81 (s, 3H), 1.97-1.92 (m, 2H), 1.90-1.82 (m, 2H), 1.70-1.66 (m, 1H), 1.52-1.32 (m, 1H). LCMS: 547.2 [M+Na]⁺.

### Example 32: Synthesis of (E)-4-((2-((Z)-2-cyclobutyl-1-(3-fluoro-1H-indazol-1-yl)-2-phenylvinyl)pyridin-2-yl)oxy)ethyl)amino)-N-methylbut-2-enamide (Compound 32)

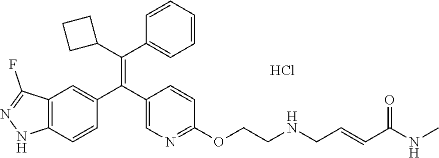 [SMILES: CNC(=O)/C=C/CNCCOc1ccc(/C(=C(\c2ccccc2)C2CCC2)c2ccc3[nH]nc(F)c3c2)cn1 ; Cl]

Compound 32 was synthesized following the approach outlined in Scheme 9 by modifying: a) Step-3 by substituting 5-bromo-3-fluoro-1-(tetrahydro-2H-pyran-2-yl)-1H-indazole (Scheme 3, Steps-1-2) for compound 352 and stirring at 20° C. until completion, b) Step-4 by substituting tert-butyl (E)-(2-((5-iodopyridin-2-yl)oxy)ethyl)(4-(methylamino)-4-oxobut-2-en-1-yl)carbamate (Scheme 4, Steps-1-3) for compound 335, 0.2 equiv of Pd(dppf)Cl₂ for Pd₂(dba)₃.CHCl₃, and c) using a 25:2 ratio of TFA:DCM to deliver the title compound in 39.9 mg, 0.36% overall yield. ¹H NMR (300 MHz, Methanol-d4) δ 7.78 (s, 1H), 7.57 (s, 1H), 7.51-7.45 (m, 2H), 7.34-7.27 (m, 3H), 7.23-7.15 (m, 3H), 6.75-6.66 (m, 2H), 6.32-6.26 (m, 1H), 4.49-4.46 (m, 2H), 3.87-3.84 (dd, J=6.9, 1.4 Hz, 2H), 3.53 (m, 1H), 3.43-3.40) (m, 2H), 2.81 (s, 3H), 1.96-1.86 (m, 4H), 1.79-1.58 (m, 1H), 1.52-1.35 (m, 1H). LCMS: 526.3 [M+H]⁺.

### Example 33: Synthesis of (E)-4-((2-((5-((Z)-2-cyclobutyl-1-(1H-indazol-5-yl)-2-phenylvinyl)pyridin-2-yl)oxy)ethyl)amino)-N-methylbut-2-enamide (Compound 33)

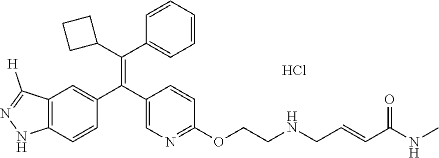 [SMILES: CNC(=O)/C=C/CNCCOc1ccc(/C(=C(\c2ccccc2)C2CCC2)c2ccc3[nH]ncc3c2)cn1 ; Cl]

Compound 33 was synthesized following the approach outlined in Scheme 9 by modifying: a) Step-4 by substituting tert-butyl (E)-(2-((5-iodopyridin-2-yl)oxy)ethyl)(4-(methylamino)-4-oxobut-2-en-1-yl)carbamate (Scheme 4, Steps-1-3) for compound 335 and 0.2 equiv of Pd(dppf)Cl₂ for Pd₂(dba)₃.CHCl₃ (in a 0.04M solution), and b) Step-5 by using a 35:1 ratio of TFA:DCM to deliver the title compound in 44.4 mg, 1.02% overall yield. ¹H NMR (300 MHz, Methanol-d4) δ 8.41 (d, J=1.0 Hz, 1H), 7.83 (m, 2H), 7.69-7.64 (m, 2H), 7.45-7.41 (m, 1H), 7.33-7.28 (m, 2H), 7.24-7.16 (m, 3H), 6.94-6.91 (d, J=8.8 Hz, 1H), 6.70-6.65 (m, 1H), 6.33-6.27 (m, 1H), 4.54-4.50 (m, 2H), 3.87-3.84 (m, 2H), 3.52-3.42 (m, 3H), 2.79 (s, 3H), 1.95-1.85 (m, 4H), 1.79-1.55 (m, 1H), 1.48-1.28 (m, 1H). LCMS: 508.3 [M+H]⁺.

### Example 34: Synthesis of (E)-4-((2-(4-((E)-2-cyclobutyl-1-(3-fluoro-1H-indazol-1-yl)-2-phenylvinyl)phenoxy)ethyl)amino)-1-(pyrrolidin-1-yl)but-2-en-1-one (Compound 34)

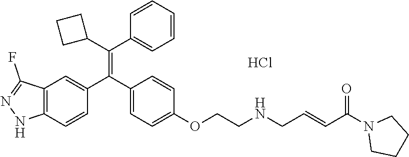 [SMILES: O=C(/C=C/CNCCOc1ccc(/C(=C(\c2ccccc2)C2CCC2)c2ccc3[nH]nc(F)c3c2)cc1)N1CCCC1 ; Cl]

Compound 34 was synthesized following the approach outlined in Scheme 9 by modifying: a) Step-3 by substituting 5-bromo-3-fluoro-1-(tetrahydro-2H-pyran-2-yl)-1H-indazole (Scheme 3, Steps-1-2) for compound 352 and stirring at 20° C. until completion, b) Step-4 by substituting tert-butyl (E)-(2-(4-iodophenoxy)ethyl)(4-oxo-4-(pyrrolidin-1-yl)but-2-en-1-yl)carbamate (preparation shown in Example 14, Step-a) for compound 335. Pd(dppf)Cl₂ for Pd₂(dba)₃.CHCl₃ (in a 0.05M solution), and c) Step-5 by using a 40:3 ratio of TFA:DCM to deliver the title compound in 45.0 mg, 0.49% overall yield. ¹H NMR (300 MHz, Methanol-d4) δ 7.47 (s, 1H), 7.42-7.39 (m, 1H), 7.27-7.07 (m, 6H), 6.89-6.86 (d, J=8.6 Hz, 2H), 6.70-6.68 (m, 4H), 4.13-4.10 (t, J=4.9 Hz, 2H), 3.88-3.86 (d, J=4.9 Hz, 2H), 3.61-3.56 (t, J=6.7 Hz, 2H), 3.49-3.45 (m, 3H), 3.39-3.36 (m, 2H), 2.01-1.81 (m, 8H), 1.75-1.50 (m, 1H), 1.48-1.30 (m, 1H). LCMS: 587.1 [M+Na]⁺.

### Example 35: Synthesis of (E)-4-((2-(4-((E)-2-cyclobutyl-1-(1H-indazol-5-yl)-2-phenylvinyl)phenoxy)ethyl)amino-1-(pyrrolidin-1-yl)but-2-en-1-one (Compound 35)

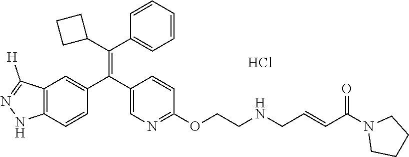 [SMILES: O=C(/C=C/CNCCOc1ccc(/C(=C(\c2ccccc2)C2CCC2)c2ccc3[nH]ncc3c2)cn1)N1CCCC1 ; Cl]

Compound 35 was synthesized following the approach outlined in Scheme 9 by modifying: a) Step-4 by substituting tert-butyl (E)-(2-(4-iodophenoxy)ethyl)(4-oxo-4-(pyrrolidin-1-yl)but-2-en-1-yl)carbamate (preparation shown in Example 14, Step-a) for compound 335, and 0.2 equiv of Pd(dppf)Cl₂ for Pd₂(dba)₃.CHCl₃ to deliver the title compound in 43.0 mg, 0.65% overall yield. ¹H NMR (300 MHz, Methanol-d4) δ 8.36 (s, 1H), 7.73 (s, 1H), 7.61-7.58 (d, J=8.7 Hz, 1H), 7.36-7.33 (m, 1H), 7.24-7.19 (m, 2H), 7.15-7.08 (m, 3H), 6.90-6.87 (m, 2H), 6.70-6.62 (m, 4H), 4.13-4.10 (m, 2H), 3.88-3.86 (m, 2H), 3.61-3.57 (m, 2H), 3.49-3.45 (m, 3H), 3.39-3.36 (m, 2H), 1.98-1.81 (m, 8H), 1.75-1.56 (m, 1H), 1.46-1.32 (m, 1H). LCMS: 547.25 [M+H]⁺.

### Example 36: Synthesis of (E)-4-((2-((5-((Z)-2-cyclobutyl-1-(3-fluoro-1H-indazol-5-yl)-2-phenylvinyl)pyridin-2-yl)oxy)ethyl)amino)-1-(pyrrolidin-1-yl)but-2-en-1-one (Compound 36)

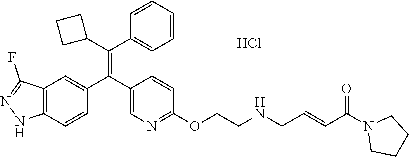 [SMILES: O=C(/C=C/CNCCOc1ccc(/C(=C(\c2ccccc2)C2CCC2)c2ccc3[nH]nc(F)c3c2)cn1)N1CCCC1 ; Cl]

Compound 36 was synthesized following the approach outlined in Scheme 9 by modifying: a) Step-3 by substituting 5-bromo-3-fluoro-1-(tetrahydro-2H-pyran-2-yl)-1H-indazole (Scheme 3, Steps-1-2) for compound 352 and stirring at 20° C. until completion, b) Step-4 by substituting tert-butyl (E)-(2-((5-iodopyridin-2-yl)oxy)ethyl)(4-oxo-4-(pyrrolidin-1-yl)but-2-en-1-yl)carbamate (preparation shown in Example 15, Step-a) for compound 335, 0.2 equiv of Pd(dppf)Cl₂ for Pd₂(dba)₃.CHCl₃, and c) Step-5 by using a 5:1 ratio of TFA:DCM to deliver the title compound in 190.0 mg, 0.66% overall yield. ¹H NMR (300 MHz, Methanol-d4) δ 7.73 (d, J=2.3 Hz, 1H), 7.52 (s, 1H), 7.44 (d, 1H), 7.36-7.12 (m, 7H), 6.68-6.60 (m, 3H), 4.45-4.42 (m, 2H), 3.86 (d, J=5.0 Hz, 2H), 3.60-3.56 (m, 2H), 3.49-3.45 (m, 3H), 3.40-3.36 (m, 2H), 2.01-1.86 (m, 8H), 1.72-1.68 (m, 1H), 1.44 (d, J=9.0 Hz, 1H). LCMS: 566.1 [M+H]⁺.

### Example 37: Synthesis of (E)-4-((2-((5-((Z)-2-cyclobutyl-1-(1H-indazol-5-yl)-2-phenylvinyl)pyridin-2-yl)oxy)ethyl)amino)-1-(pyridin-1-yl)but-2-en-1-one (Compound 37)

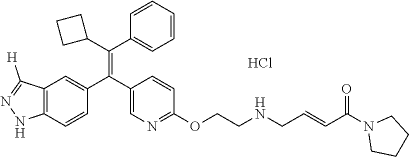 [SMILES: O=C(/C=C/CNCCOc1ccc(/C(=C(\c2ccccc2)C2CCC2)c2ccc3[nH]ncc3c2)cn1)N1CCCC1 ; Cl]

Compound 37 was synthesized following the approach outlined in Scheme 9 by modifying: a) Step-4 by substituting tert-butyl (E)-(2-((5-iodopyridin-2-yl)oxy)ethyl)(4-oxo-4-(pyrrolidin-1-yl)but-2-en-1-yl)carbamate (preparation shown in Example 15, Step-a) for compound 335, 0.2 equiv of Pd(dppf)Cl₂ for Pd₂(dba)₃.CHCl₃, and c) Step-5 by using a 4:1 ratio of TFA:DCM to deliver the title compound in 60.5 mg, 0.92% overall yield. ¹H NMR (300 MHz, Methanol-d4) δ 8.52 (m, 1H), 7.94-7.86 (m, 2H), 7.79-7.70 (m, 2H), 7.50-7.47 (m, 1H), 7.34-7.29 (m, 2H), 7.25-7.18 (m, 3H), 7.06-7.03 (d, J=8.9 Hz, 1H), 6.72-6.70 (m, 2H), 4.58-4.55 (m, 2H), 3.92-3.90 (m, 2H), 3.63-3.59 (m, 2H), 3.52-3.45 (m, 5H), 2.03-1.85 (m, 8H), 1.79-1.55 (m, 1H), 1.50-1.35 (m, 1H). LCMS: 549.3 [M+H]⁺.

### Example 38: Synthesis of (E)-N-methyl-4-((2-(4-(4,4,4-trifluoro-1-(1H-indazol-5-yl)-2-phenylbut-1-en-1-yl)phenoxy)ethyl)amino)butanamide (Compound 38)

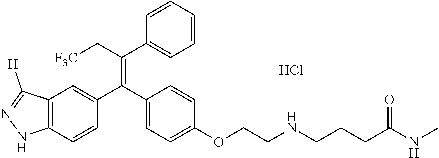 [SMILES: CNC(=O)CCCNCCOc1ccc(/C(=C(/CC(F)(F)F)c2ccccc2)c2ccc3[nH]ncc3c2)cc1 ; Cl]

Compound 38 was synthesized following the approach outlined in Scheme 6, modifying Step-1 by substituting (E)-N-methyl-4-((2-(4-((E)-4,4,4-trifluoro-1-(1H-indazol-5-yl)-2-phenylbut-1-en-1-yl)phenoxy)ethyl)amino)but-2-enamide (preparation shown in Example 10) for compound 336 to deliver the title compound in 54.3 mg, 17% overall yield. ¹H NMR (400 MHz, Methanol-d4) δ 8.49 (s, 1H), 7.90 (s, 1H), 7.70-7.68 (d, J=8.7 Hz, 1H), 7.42-7.40 (m, 1H), 7.25-7.14 (m, 5H), 6.93-6.90 (m, 2H), 6.74-6.72 (m, 2H), 4.18-4.16 (t, J=4.9 Hz, 2H), 3.45-3.37 (m, 4H), 3.13-3.09 (t, J=7.3 Hz, 2H), 2.74 (s, 3H), 2.40-2.37 (t, J=6.8 Hz, 2H), 2.05-1.93 (p, J=7.0 Hz, 2H). LCMS: 537.3 [M+H]⁺.

### Example 39: Synthesis of (E)-N-methyl-4-((2-(4-(4,4,4-trifluoro-1-(3-fluoro-1H-Indazol-5-yl)-2-phenylbut-1-en-1-yl)phenoxy)ethyl)amino)butanamide (Compound 39)

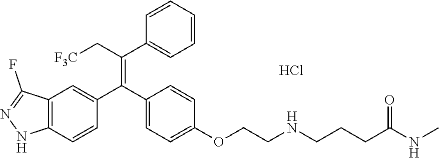 [SMILES: CNC(=O)CCCNCCOc1ccc(/C(=C(/CC(F)(F)F)c2ccccc2)c2ccc3[nH]nc(F)c3c2)cc1 ; Cl]

Compound 39 was synthesized following the approach outlined in Scheme 6, modifying step 1 by substituting (E)-N-methyl-4-((2-(4-((E)-4,4,4-trifluoro-1-(3-fluoro-1H-indazol-5-yl)-2-phenylbut-1-en-1-yl)phenoxy)ethyl)amino)but-2-enamide (preparation shown in Example 21) for compound 336 to deliver the title compound in 49.0 mg, 23% overall yield. ¹H NMR (400 MHz, Methanol-d₄) δ 7.61 (m, J=1.1 Hz; 1H), 7.49-7.46 (m, J=8.8, 2.3, 0.9 Hz, 1H), 7.30-7.27 (dd, J=8.8, 1.6 Hz, 1H), 7.24-7.15 (m, 5H), 6.92-6.89 (m, 2H), 6.75-6.72 (m, 2H), 4.18-4.15 (m, 2H), 3.44-3.36 (m, 4H), 3.13-3.09 (m, 2H), 2.71 (s, 3H), 2.40-2.37 (m, 2H), 1.99-1.93 (m, 2H). LCMS: 555.2 [M+H]⁺.

### Example 40: Synthesis of (Z)—N-methyl-4-((2-((5-(4,4,4-trifluoro-1-(1H-indazol-5-yl)-2-phenylbut-1-en-1-yl)pyridin-2-yl)oxy)ethyl)amino)butanamide (Compound 40)

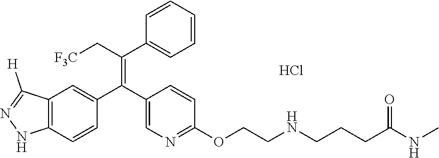 [SMILES: CNC(=O)CCCNCCOc1ccc(/C(=C(/CC(F)(F)F)c2ccccc2)c2ccc3[nH]ncc3c2)cn1 ; Cl]

Compound 40 was synthesized following the approach outlined in Scheme 6, modifying step 1 by substituting (E)-N-methyl-4-((2-((5-((Z)-4,4,4-trifluoro-1-(1H-indazol-5-yl)-2-phenylbut-1-en-1-yl)pyridin-2-yl)oxy)ethyl)amino)but-2-enamide (preparation shown in Example 9) for compound 336 to deliver the title compound in 62.4 mg, 29% overall yield. ¹H NMR (300 MHz, Methanol-d4) δ 8.41 (d, J=1.0 Hz, 1H), 7.93 (d, J=1.3 Hz, 1H), 7.80 (dd, J=2.4, 0.7 Hz, 1H), 7.72-7.70 (m, 1H), 7.64-7.60 (m, 1H), 7.45-7.41 (m, 1H), 7.28-7.21 (m, 5H), 6.96-6.63 (dd, J=8.8, 0.7 Hz, 1H), 4.56-4.53 (m, 2H), 3.49-3.39 (m, 4H), 3.13-3.08 (t, J=7.1 Hz, 2H), 2.70 (s, 3H), 2.40-2.36 (t, J=6.7 Hz, 2H), 2.03-1.89 (m, 2H). LCMS: 538.2 [M+H]⁺.

### Example 41: Synthesis of (E)-1-(pyrrolidin-1-yl)-4-((2-(4-(4,4,4-trifluoro-1-(3-fluoro-1H-indazol-5-yl-2-phenylbut-1-en-1-yl)phenoxy)ethyl)amino)butan-1-one (Compound 41)

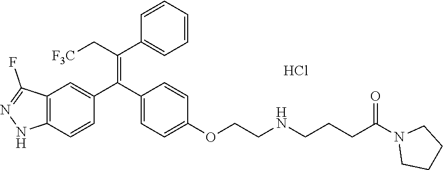 [SMILES: O=C(CCCNCCOc1ccc(/C(=C(/CC(F)(F)F)c2ccccc2)c2ccc3[nH]nc(F)c3c2)cc1)N1CCCC1 ; Cl]

Compound 41 was synthesized following the approach outlined in Scheme 6, modifying step 1 by substituting (E)-1-(pyrrolidin-1-yl)-4-((2-(4-((E)-4,4,4-trifluoro-1-(3-fluoro-1H-indazol-5-yl)-2-phenylbut-1-en-1-yl)phenoxy)ethyl)amino)but-2-en-1-one (preparation shown in Example 13) for compound 336 to deliver the title compound in 54.2 mg, 11.94% overall yield. ¹H NMR (300 MHz, Methanol-d4) δ 7.60 (s, 1H), 7.47-7.43 (m, 1H), 7.28-7.25 (dd, J=8.8, 1.5 Hz, 1H), 7.21-7.12 (m, 5H), 6.90-6.87 (m, 2H), 6.72-6.69 (m, 2H), 4.16-4.13 (m, 2H), 3.45-3.34 (m, 8H), 3.13-3.08 (t, J=7.0 Hz, 2H), 2.54-2.50 (t, J=6.5 Hz, 2H), 1.97-1.90 (m, 4H), 1.86-1.81 (m, 2H). LCMS: 595 [M+H]⁺.

### Example 42: Synthesis of (E)-1-(pyrrolidin-1-yl)-4-((2-(4-(4,4,4-trifluoro-1-(1H-indazol-5-yl)-2-phenylbut-1-en-1-yl)phenoxy)ethyl)amino)butan-1-one (Compound 42)

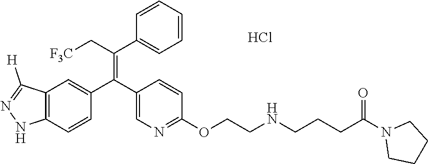 [SMILES: O=C(CCCNCCOc1ccc(/C(=C(/CC(F)(F)F)c2ccccc2)c2ccc3[nH]ncc3c2)cn1)N1CCCC1 ; Cl]

Example 42 was synthesized following the approach outlined in Scheme 7 by modifying: a) Step-1 by substituting (Z)-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (Scheme 8, Steps-1-5) for compound 323 and tert-butyl (E)-(2-((5-iodopyridin-2-yl)oxy)ethyl)(4-oxo-4-(pyrrolidin-1-yl)but-2-en-1-yl)carbamate (preparation shown in Example 15, Step-a) for compound 337 to deliver the title compound in 108.7 mg, 1.91% overall yield, as a yellow solid. ¹H NMR (300 MHz, DMSO-d6) δ 9.06 (s, 2H), 8.12 (d, J=1.0 Hz, 1H), 7.65 9 s, 1H), 7.59-7.56 (m, 1H). 7.25-7.11 (m, 6H), 6.84-6.81 (m, 2H), 6.69-6.66 (m, 2H), 4.13-4.10 (t, J=4.8 Hz, 2H), 3.51-3.40 (m, 2H), 3.37-3.32 (m, 4H), 3.27-3.22 (m, 4H), 2.97-2.95 (m, 2H), 2.39-2.34 (t, J=6.9 Hz, 2H), 1.86-1.68 (m, 6H). LCMS: 577 [M+H]⁺.

### Example 43: Synthesis of (Z)-1-(pyrrolidin-1-yl)-4-((2-((5-(4,4,4-trifluoro-1-(3-fluoro-1H-indazol-5-yl)-2-phenylbut-1-en-1-ylpyridin-2-yl)oxy)ethyl)amino)butan-1-one (Compound 43)

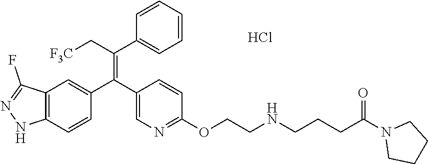 [SMILES: O=C(CCCNCCOc1ccc(/C(=C(/CC(F)(F)F)c2ccccc2)c2ccc3[nH]nc(F)c3c2)cn1)N1CCCC1 ; Cl]

Compound 43 was synthesized following the approach outlined in Scheme 7, modifying Step-1 by substituting tert-butyl (E)-(2-((5-iodopyridin-2-yl)oxy)ethyl)(4-oxo-4-(pyrrolidin-1-yl)but-2-en-1-yl)carbamate (preparation shown in Example 15, Step-a) for compound 337 to deliver the title compound in 29.6 mg, 1.12% overall yield, as a white solid. ¹H NMR (300 MHz, Methanol-d4) δ 7.77 (d, J=2.3 Hz, 1H), 7.66 (s, 1H), 7.53-7.49 (m, 2H), 7.34-7.22 (m, 6H), 6.86-6.83 (m, 1H), 4.53-4.50 (m, 2H), 3.46-3.34 (m, 8H), 3.13-3.08 (t, J=6.9 Hz, 2H), 2.55-2.51 (t, J=6.5 Hz, 2H), 1.98-1.92 (m, 4H), 1.86-1.84 (m, 2H). LCMS: 596.3 [M+H]⁺.

### Example 44: Synthesis of (E)-N-methyl-4-((2 methyl-1-((methyl-((Z)-4,4,4-trifluoro-1-(3-fluoro-1H-indazol-5-yl)-2-phenylbut-1-en-1-yl)pyridin-2-yl(oxy)ethyl(amino)but-2-enamide (Compound 44)

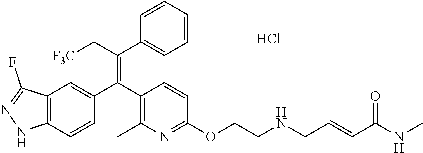 [SMILES: CNC(=O)/C=C/CNCCOc1ccc(/C(=C(/CC(F)(F)F)c2ccccc2)c2ccc3[nH]nc(F)c3c2)c(C)n1 ; Cl]

Compound 44 was synthesized following the approach outlined in Scheme 3, modifying Step-7 by substituting tert-butyl (E)-(2-((5-iodo-6-methylpyridin-2-yl)oxy)ethyl)(4-(methylamino)-4-oxobut-2-en-1-yl)carbamate (preparation shown below in Steps-a-d) for compound 324 to deliver the title compound in 91.1 mg, 0.04% overall yield. ¹H NMR (400 MHz, Methanol-d4) δ 7.98-7.96 (s, 1H), 7.69 (s, 1H), 7.55-7.53 (dd, J=8.8, 2.1 Hz, 1H), 7.43-7.40 (dd, J=8.8, 1.6 Hz, 1H), 7.28-7.19 (m, 5H), 7.05-7.03 (m, J=9.0 Hz, 1H), 6.76-6.69 (m, J=15.4, 6.8 Hz, 1H), 6.37-6.33 (m, 1H), 4.62 (t, J=4.6 Hz, 2H), 3.93-3.91 (dd, J=6.9, 1.3 Hz, 2H), 3.58-3.49 (m, 4H), 2.83-2.81 (s, 3H), 2.37 (s, 3H). LCMS: 568.1 [M+H]⁺.

### Step-a: Synthesis of 2-((5-iodo-6-methylpyridin-2-yl)oxy)ethan-1-ol

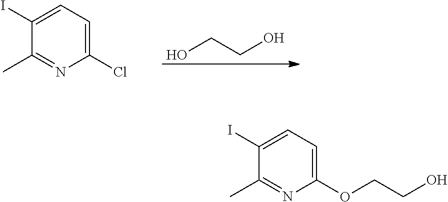

Into a 8-mL round-bottom flask was placed 6-chloro-3-iodo-2-methylpyridine (100 mg, 0.39 mmol, 1.00 equiv), sodium hydroxide (31.49 mg, 0.79 mmol, 2.00 equiv), and ethane-1,2-diol (244.09 mg, 3.93 mmol, 10.00 equiv). The resulting solution was stirred at 110° C. until completion. The solution was diluted with 30 mL of water and extracted with 3×50 mL of ethyl acetate. Then the organic layers were combined and washed with 3×50 mL of brine. The mixture was dried over anhydrous sodium sulfate and concentrated under vacuum. The residue was applied onto a silica gel column eluting with DCM/methanol (14:1) to deliver the title compound in 60 mg (54.5%) as a white solid. LCMS: 279.9 [M+H]⁺.

### Step-b: Synthesis of 2-(2-((5-iodo-6-methylpyridin-2-yl)oxy)ethyl)isoindoline-1,3-done

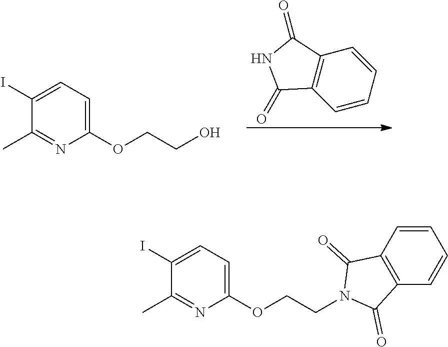 [SMILES: Cc1nc(OCCO)ccc1I ; O=C1NC(=O)c2ccccc21 ; Cc1nc(OCCN2C(=O)c3ccccc3C2=O)ccc1I]

Into a 8-mL round-bottom flask purged and maintained with an inert atmosphere of nitrogen was placed 2-((5-iodo-6-methylpyridin-2-yl)oxy)ethan-1-ol (100 mg, 0.36 mmol, 1.00 equiv), PPh3 (188.0215 g, 716.85 mmol, 2.00 equiv), THF (20 mL), 2,3-dihydro-1H-isoindole-1,3-dione (52 mg, 0.35 mmol, 1.00 equiv), and DIAD (145.08 mg, 0.72 mmol, 2.00 equiv). The resulting solution was stirred at 25° C. until completion. The resulting solution was diluted with H₂O (50 mL), extracted with 3×50 mL of ethyl acetate and the organic layers combined, dried over Na₂SO₄, and concentrated under vacuum. The residue was applied onto a silica gel column with ethyl acetate/petroleum ether (1; 99). The collected fractions were combined and concentrated under vacuum to deliver the title compound in 102 mg (70%) as a yellow solid.

### Step-c: Synthesis of 2-((5-iodo-6-methylpyridin-2-yl)oxy)ethan-1-amine

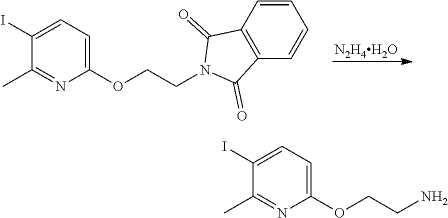

Into a 8-mL round-bottom flask was placed 2-(2-((5-iodo-6-methylpyridin-2-yl)oxy)ethyl)isoindoline-1,3-dione (100 mg, 0.24 mmol, 1.00 equiv), THF (1 mL), and hydrogen diazene hydrate (2 mL, 2.00 equiv). The resulting solution was stirred at 25° C. until completion. The resulting solution was extracted with of ethyl acetate and the organic layers combined and concentrated under vacuum. The residue was applied onto a silica gel column with ethyl acetate/petroleum ether (1:99). The crude product was purified by Flash-Prep-HPLC with the following conditions (IntelFlash-1): Column, silica gel; Detector, UV 254 nm to deliver the title compound in 47 mg (70%) as a yellow solid.

### Step-d Synthesis of tert-butyl (E)-(2-((5-iodo-6-methylpyridin-2-yl)oxy)ethyl)(4-(methylamino)-4-oxobut-2-en-1-yl)carbamate

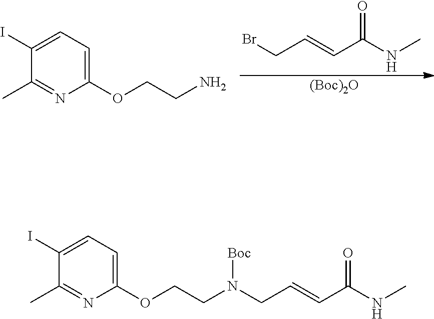

Into a 500-mL round-bottom flask was placed 2-((5-iodo-6-methylpyridin-2-yl)oxy)ethan-1-amine (5.3 g, 19.06 mmol, 1.00) equiv), DIEA (7.35 g, 56.87 mmol, 3.00 equiv), (E)-4-bromo-N-methylbut-2-enamide (2.18 g, 13.29 mmol, 0.70 equiv) (Scheme 4, steps a-b). The solution was then stirred for 2 h at 25° C., and then di-tert-butyl dicarbonate (8.6 g, 38 mmol, 2.0 eq) was added. The resulting solution was stirred at 25° C. until completion. The solution was diluted with 30 mL of water and extracted with 3×50 mL of ethyl acetate. Then the organic layers were combined and washed with 1×60 mL of brine. The mixture was dried over anhydrous sodium sulfate and concentrated under vacuum. The residue was applied onto a silica gel column eluting with DCM/methanol (14:1) to deliver the title compound in 1.8 g (20%) as a white solid. ¹H NMR (400 MHz, DMSO-d6) δ 8.00-7.95 (dd, J=13.6, 6.6 Hz, 2H), 6.54-6.44 (dq, J=16.1, 7.4, 6.9 Hz, 2H), 5.91-5.80 (dd, J=15.4, 2.3 Hz, 1H), 4.35-4.30 (q, J=8.6, 6.6 Hz, 2H), 3.96-3.95 (m, 2H), 3.52-3.49 (t, J=5.6 Hz, 2H), 2.63 (d, J=4.6 Hz, 3H), 2.53 (s, 3H), 1.35-1.32 (d, J=9.5 Hz, 9H).

### Example 45: Synthesis of (E)-N-methyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (Compound 45)

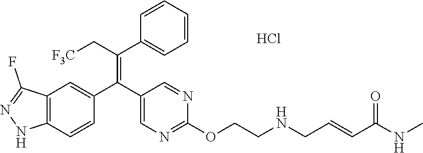 [SMILES: CNC(=O)/C=C/CNCCOc1ncc(/C(=C(/CC(F)(F)F)c2ccccc2)c2ccc3[nH]nc(F)c3c2)cn1 ; Cl]

Compound 45 was synthesized following the approach outlined in Scheme 3, modifying Step-7 by substituting tert-butyl (E)-(2-((5-iodopyrimidin-2-yl)oxy)ethyl)(4-(methylamino)-4-oxobut-2-en-1-yl)carbamate (preparation shown below in Steps-a-c) for compound 324 to deliver the title compound in 20.0 mg, 6.53% overall yield. ¹H NMR (400 MHz, Methanol-d4) δ 8.15 (s, 2H), 7.72 (s, 1H), 7.57-7.54 (m, 1H), 7.38-7.36 (m, 1H), 7.32-7.25 (m, 5H), 6.72-6.65 (m, 6.9 Hz, 1H), 6.30-6.26 (m, 1H), 4.56-4.53 (m, 2H), 3.88-3.86 (m, 1.4 Hz, 2H), 3.50-3.42 (m, 4H), 2.83-2.81 (s, 3H).

### Step-a: Synthesis of tert-butyl (2-((5-iodopyridin-2-yl)oxy)ethyl)carbamate

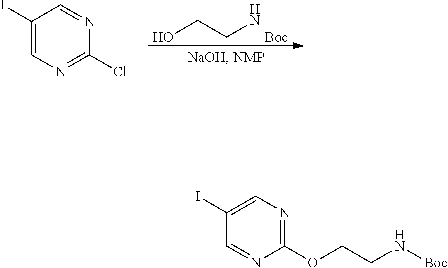

Into a 500-mL round-bottom flask was placed 2-chloro-5-iodopyrimidine (10 g, 41.59 mmol, 1.00 equiv), NMP (200 mL), sodium hydroxide (3.3 g, 82.50 mmol, 2.00 equiv), and tert-butyl (2-hydroxyethyl)carbamate (6.7 g, 41.56 mmol, 1.00 equiv). The resulting solution was stirred at 100° C. until completion. The resulting solution was diluted with H₂O (100 mL), extracted with of ethyl acetate (3×100 mL) and the organic layers combined, washed with brine (100 mL), dried over Na₂SO₄ and concentrated under vacuum. The residue was applied onto a silica gel column with ethyl acetate/petroleum ether (1:3) to deliver the title compound in 7.6 g (50%) as a brown solid.

### Step-b: Synthesis of 2-((5-iodopyridin-2-yl)oxy)ethan-1-amine

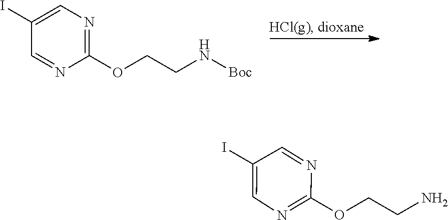 [SMILES: CC(C)(C)OC(=O)NCCOc1ncc(I)cn1 ; NCCOc1ncc(I)cn1]

Into a 50-mL round-bottom flask was placed tert-butyl (2-((5-iodopyrimidin-2-yl)oxy)ethyl)carbamate (2.4 g, 6.57 mmol, 1.00 equiv). To the above, hydrogen chloride (g) in dioxane (24 mL, 1.00 equiv) was added. The resulting solution was stirred at 24° C. until completion, and then the resulting mixture was concentrated under vacuum to deliver the title compound in 1.8 g (91%) as a yellow solid. LCMS: 265.8 [M+H]⁺.

### Step-c: Synthesis of tert-butyl (E)-(2-((5-iodopyrimidin-2-yl)oxy)ethyl)(4-(methylamino)-4-oxobut-2-en-1-yl)carbamate

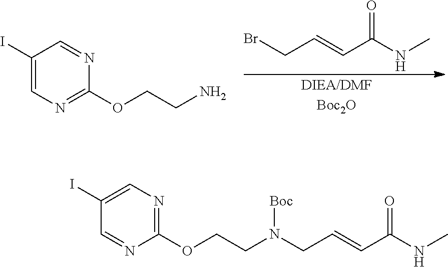

Into a 40-mL round-bottom flask was placed 2-((5-iodopyrimidin-2-yl)oxy)ethan-1-amine (1.8 g, 6.79 mmol, 1.0 equiv), N,N-dimethylformamide (30 mL. 1.00 equiv), DIEA (4.63 g, 35.82 mmol, 6.00 equiv), and (E)-4-bromo-N-methylbut-2-enamide (1.38 g, 7.75 mmol, 1.30 equiv) (Scheme 4, steps a-b). The resulting solution was stirred at 25° C. until completion. Then di-tert-butyl dicarbonate (2.6 g, 11.91 mmol, 2.00 equiv) was added. The resulting solution was stirred at 25° C. until completion. The solution was diluted with 200 mL of water and extracted with 3×200 mL of ethyl acetate. Then the organic layers were combined and washed with 3×200 mL of brine. The mixture was dried over anhydrous sodium sulfate and concentrated under vacuum. The residue was applied onto a silica gel column eluting with petroleum ether/ethyl acetate (3:2) to deliver the title compound in 1.1 g (40%) as a brown solid. ¹H NMR (400 MHz. DMSO-d6) δ 8.80 (s, 2H), 7.95 (d, J=5.4 Hz, 1H), 6.50 (s, 1H), 5.90-5.86 (d, J=15.8 Hz, 1H), 4.38 (s, 2H), 3.95 (s, 2H), 3.55-3.52 (t, J=5.6 Hz, 2H), 2.63 (d, J=4.7 Hz, 3H), 1.35-1.32 (d, J=14.7 Hz, 9H). LCMS: 463 [M+H]⁺.

### Example 46: Synthesis of (E)-4-((2-(4-((E)-2-(2-chloro-4-fluorophenyl)-4,4,4-trifluo-1-(1H-indazol-5-yl)but-1-en-1-yl)phenoxy)ethyl)amino)-N-methylbut-2-enamide (Compound 46)

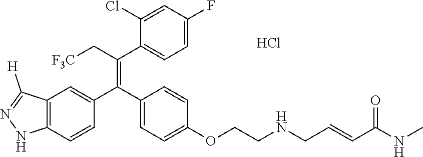 [SMILES: CNC(=O)/C=C/CNCCOc1ccc(/C(=C(/CC(F)(F)F)c2ccc(F)cc2Cl)c2ccc3[nH]ncc3c2)cc1 ; Cl]

Compound 46 was synthesized following the approach outlined in Scheme 3 by modifying: a) Step-7 by substituting (Z)-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (Scheme 8, Steps-1-5) for compound 323 and tert-butyl (E)-(2-(4-iodophenoxy)ethyl)(4-(methylamino)-4-oxobut-2-en-1-yl)carbamate (Scheme 5, Steps-1-3) for compound 324, and b) Step-8 by substituting 2.0 equiv 2-chloro-4-fluoro-1-iodobenzene for compound 326 and using 7.0 equiv of KOH to deliver the title compound in 32.7 mg, 0.19% overall yield, as a white solid. ¹H NMR (400 MHz, Methanol-d4) δ 8.14 (s, 1H), 7.77 (s, 1H), 7.59-7.56 (d, J=8.6 Hz, 1H), 7.28-7.23 (m, 2H), 7.15-7.12 (dd, J=8.8, 2.6 Hz, 1H), 6.98-6.90 (m, 3H), 6.75-6.64 (m, 3H), 6.29-6.25 (d, J=15.4 Hz, 1H), 4.16-4.14 (m, 2H), 3.86-3.84 (m, 2H), 3.47-3.35 (m, 4H), 2.79 (s, 3H). LCMS: 587.10 [M+H]⁺, 609.10 [M+Na]⁺.

### Example 47: Synthesis of (E)-4-((2-(4-((E)-2-(2-chloro-4-fluorophenyl)-4,4,4-trifluoro-1-(3-fluoro-1H-indazol-5-yl)but-1-en-1-yl)phenoxy)ethyl)amine)-N-methylbut-2-enamide (Compound 47)

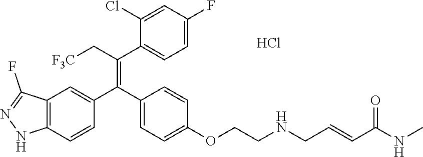 [SMILES: CNC(=O)/C=C/CNCCOc1ccc(/C(=C(/CC(F)(F)F)c2ccc(F)cc2Cl)c2ccc3[nH]nc(F)c3c2)cc1 ; Cl]

Compound 47 was synthesized following the approach outlined in Scheme 3 by modifying: a) Step-7 by substituting tert-butyl (E)-(2-(4-iodophenoxy)ethyl)(4-(methylamino)-4-oxobut-2-en-1-yl)carbamate (Scheme 5, Steps-1-3) for compound 324, and b) Step-8 by substituting 2.0 equiv 2-chloro-4-fluoro-1-iodobenzene for compound 326 and using 7.0 equiv of KOH to deliver the title compound in 50 mg, 0.13% overall yield, as a light brown solid. ¹H NMR (400 MHz, Methanol-d₄) δ 7.64 (s, 1H), 7.49-7.46 (dd, J=8.8, 2.1 Hz, 1H), 7.31-7.24 (m, 2H), 7.15-7.12 (dd, J=8.7, 2.6 Hz, 1H), 6.98-6.91 (m, 3H), 6.76-6.64 (m, 3H), 6.28-6.24 (d, J=15.6 Hz, 1H), 4.16-4.14 (t, J=4.9 Hz, 2H), 3.86-3.84 (m, 2H), 3.47-3.34 (m, 4H), 2.79 (s, 3H). LCMS: 605.10 [M+H]⁺, 627.10 [M+Na]⁺.

### Example 48: Synthesis of (E)-4-((2-(4-((E)-2-(2-chloro-4-fluorophenyl)-1-(3-fluoro-1H-indazol-5-yl)but-1-en-1-yl)phenoxy)ethyl)amino)-N-methylbut-2-enamide (Compound 48)

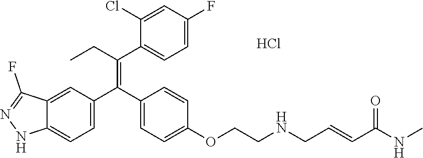 [SMILES: CC/C(=C(/c1ccc(OCCNC/C=C/C(=O)NC)cc1)c1ccc2[nH]nc(F)c2c1)c1ccc(F)cc1Cl ; Cl]

Compound 48 was synthesized following the approach outlined in Scheme 3 by modifying: a) Step-7 by substituting (Z)-5-(1,2-bis(4,4,5,5-tetramethyl-1,3,2-dioxaborolan-2-yl)but-1-en-1-yl)-3-fluoro-1-(tetrahydro-2H-pyran-2-yl)-1H-indazole (preparation shown below in Steps-a-b) for compound 323 and tert-butyl (E)-(2-(4-iodophenoxy)ethyl)(4-(methylamino)-4-oxobut-2-en-1-yl)carbamate (Scheme 5, Steps-1-3) for compound 324, and b) Step-8 by substituting 2-chloro-4-fluoro-1-iodobenzene for compound 326 and using 3.0 equiv of KOH to deliver the title compound in 60.6 mg, 0.72% overall yield, as an off-white solid. ¹H NMR (400 MHz, Methanol-d) δ 7.56 (t, J=1.2 Hz, 1H), 7.47-7.44 (m, 1H), 7.34-7.31 (dd, J=8.7, 1.5 Hz, 1H), 7.28-7.24 (dd, J=8.5, 6.2 Hz, 1H), 7.12-7.09 (dd, J=8.8, 2.6 Hz, 1H), 6.98-6.94 (m, 3H), 6.74-6.69 (m, 3H), 6.33-6.29 (m, 1H), 4.18-4.16 (m, 2H), 3.89-3.87 (dd, J=6.9, 1.4 Hz, 2H), 3.43-3.33 (t, J=4.9 Hz, 2H), 2.81 (s, 3H), 2.49-2.45 (m, 2H), 1.00-0.96 (t, J=7.5 Hz, 3H). LCMS: 551.21 [M+H]⁺.

### Step-a Syntheses of 5-(but-1-yn-1-yl)-3-fluoro-1-(tetrahydro-2H-pyran-2-yl)-1H-Indazole

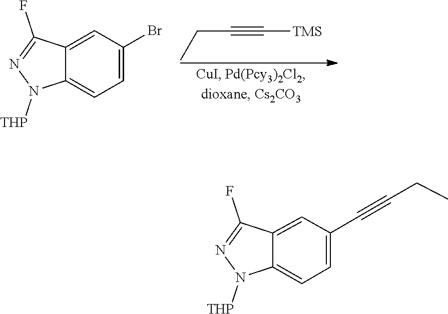

Into a 100-mL round-bottom flask purged and maintained with an inert atmosphere of nitrogen was placed 5-bromo-3-fluoro-1-(tetrahydro-2H-pyran-2-yl)-1H-indazole (4 g, 13.37 mmol, 1.00 equiv) (Scheme 3, Steps-1-2), dioxane (40 mL), Cs₂CO₃ (8.68 g, 26.64 mmol, 2.00 equiv), Pd(Pcy₃)₂Cl₂ (984 mg, 0.10 equiv), CuI (760 mg, 3.99 mmol, 0.30 equiv), and but-1-yn-1-yltrimethylsilane (16.84 g, 133.36 mmol, 10.00 equiv). The resulting solution was stirred 80° C. in an oil bath until completion, then cooled to room temperature. The reaction progress was monitored by LCMS. The resulting solution was extracted with of 3×50 mL ethyl acetate and the organic layers were combined, then washed with 1×50 mL of brine, dried over anhydrous Na₂SO₄, and concentrated under vacuum. The residue was applied onto a silica gel column eluting with petroleum ether/ethyl acetate (10:1) to deliver the title compound in 3.4 g (93%) as a yellow liquid.

### Step-b: Synthesis of (Z)-5-(1,2-bis(4,4,5,5-tetraethyl-1,3,2-dioxaborolan-2-yl)but-1-en-1-yl)-3-fluoro-1-(tetrahydro-2H-pyran-2-yl)-1H-indazole

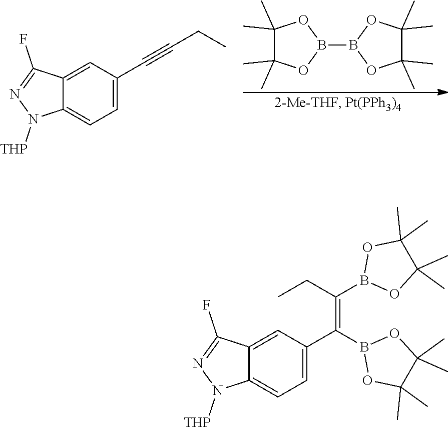

Into a 100-mL round-bottom flask purged and maintained with an inert atmosphere of nitrogen was placed 5-(but-1-yn-1-yl)-3-fluoro-1-(tetrahydro-2H-pyran-2-yl)-1H-indazole (4 g, 14.69 mmol, 1.00 equiv), 2-Me-THF (40 mL), Pt(PPh₃)₄ (912 mg, 0.05 equiv), and 4,4,5,5-tetramethyl-2-(tetramethyl-1,3,2-dioxaborolan-2-yl)-1,3,2-dioxaborolane (3.72 g, 14.65 mmol, 1.00 equiv). The resulting solution was stirred at 90° C. in an oil bath until completion, then cooled to room temperature. The reaction progress was monitored by LCMS. The solution was then extracted with of 3×50 mL ethyl acetate, the organic layers were combined, washed with 1×50 mL of brine, dried over anhydrous Na₂SO₄, and concentrated under vacuum. The residue was applied onto a silica gel column eluting with petroleum ether/ethyl acetate (10:1) to deliver the title compound in 2.0 g (27%) as a yellow solid.

### Example 49: Synthesis of (E)-N-methyl-4-((2-((5-((Z)-1-(3-methyl-1H-indazol-5-yl)-2-phenylbut-1-en-1-yl)pyridin-2-yl)oxy)ethyl)amino)but-2-enamide (Compound 49)

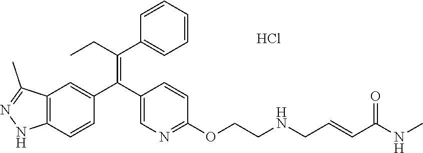 [SMILES: CC/C(=C(/c1ccc(OCCNC/C=C/C(=O)NC)nc1)c1ccc2[nH]nc(C)c2c1)c1ccccc1 ; Cl]

Compound 49 was synthesized following the approach outlined in Scheme 9 by modifying: a) Step-3 by substituting 5-bromo-3-methyl-1-(tetrahydro-2H-pyran-2-yl)-1H-indazole (preparation show below in Step-a) for compound 352, Pd(dppf)Cl₂ for Pd₂(dba)₃, 2.0 equiv of Cs₂CO₃ for KOH, dioxane:water (6:1) for THF (to make a 0.2M solution), and removing P(t-Bu)₃, b) Step-4 by substituting tert-butyl (E)-(2-((5-iodopyridin-2-yl)oxy)ethyl)(4-(methylamino)-4-oxobut-2-en-1-yl)carbamate (Scheme 4, Steps-1-3) for compound 335, Pd(PPh₃)₂Cl₂ for Pd₂(dba)₃.CHCl₃, using 1.0 equiv of KOH, dioxane:HzO (10:3) for THF (to make a 0.5M solution), and stirring at 60° C., and c) Step-5 by using a 5:1 ratio of TFA:DCM to deliver the title compound in 140 mg, 2.96% overall yield, as an off-white solid. ¹H NMR (400 MHz, Methanol-d4) δ 8.04 (s, 1H), 7.83-7.70 (d, J=8.7 Hz, 4H), 7.31-7.27 (m, 5H), 7.17 (s, 1H), 6.75-6.70 (dd, J=14.7, 7.4 Hz, 1H), 6.39-6.35 (d, J=15.0 Hz, 1H), 4.64 (s, 2H), 3.96-3.92 (m, 2H), 3.52 (s, 2H), 2.85-2.81 (d, J=15.0 Hz, 6H), 2.57-2.51 (m, J=7.5 Hz, 2H), 1.02-0.98 (s, J=7.3 Hz, 3H). LCMS: 469.3 [M+H]⁺.

### Step-a: Synthesis of 5-bromo-3-methyl-1-(tetrahydro-2H-pyran-2-yl)-1H-indazole

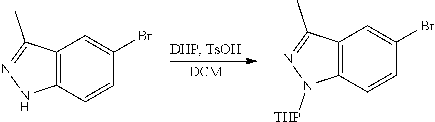

Into a 8-mL round-bottom flask was placed 5-bromo-3-methyl-1H-indazole (50 mg, 0.24 mmol, 1.00 equiv), DCM (2 mL), 3,4-dihydro-2H-pyran (60.06 g, 714.01 mmol, 3.0) equiv), and 4-methylbenzene-1-sulfonic acid (4.09 mg, 0.02 mmol, 0.10 equiv). The resulting solution was stirred at 25° C. until completion. The solution was then diluted with 30 mL of water and extracted with 3×50 mL of ethyl acetate. Then the organic layers were combined, washed with 3×50 mL of brine, dried over anhydrous sodium sulfate, and concentrated under vacuum. The residue was applied onto a silica gel column eluting with DCM/methanol (14:1) to deliver the title compound in 40 mg (16%) as a white solid.

### Example 50: Synthesis of (E)-4-((2-(4-((E)-1-(1H-indazol-5-yl)-2-phenyl)but-1-en-1-yl)phenoxy)ethyl)amino)-N-methylbut-2-enamide (Compound 50)

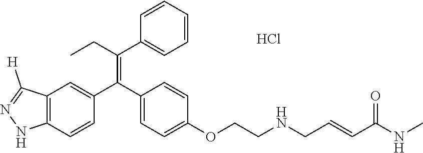 [SMILES: CC/C(=C(/c1ccc(OCCNC/C=C/C(=O)NC)cc1)c1ccc2[nH]ncc2c1)c1ccccc1 ; Cl]

Compound 50 was synthesized following the approach outlined in Scheme 10, omitting Steps-1-3, by modifying: a) Step-4 by substituting (E)-4-((2-(4-((E)-1-(1H-indazol-5-yl)-2-phenylbut-1-en-1-yl)phenoxy)ethyl)amino)-N,N-dimethylbut-2-enamide hydrochloride (synthesized following the approach outlined in patent US 2016347717 A1) for compound 357, ethanol for methanol, and heating at 70° C., and b) Step-5 by substituting 1.0 equiv of (E)-4-bromo-N-methylbut-2-enamide (Scheme 4, Step-a-b) for compound 359 to deliver the title compound in 2.7 g, 56.1% overall yield, as a yellow solid. ¹H NMR (300 MHz, Methanol-d4) δ 8.12 (s, 1H), 7.68 (s, 1H), 7.55-7.52 (d, J=8.7 Hz, 1H), 7.26-7.23 (d, J=8.8 Hz, 1H), 7.16-7.09 (m, 5H), 6.87-6.84 (m, 2H), 6.73-6.64 (m, 3H), 6.30-6.25 (d, J=15.4 Hz, 1H), 4.16-4.12 (t, J=4.9 Hz, 2H), 3.86-3.84 (m, 2H), 3.40-3.37 (t, J=4.9 Hz, 2H), 2.79 (s, 3H), 2.52-2.44 (q. J=7.4 Hz, 2H), 0.96-0.91 (1, J=7.4 Hz, 3H). LCMS: 481.3 [M+H]⁺.

### Example 51: Synthesis of (E)-4-((2-(4-(1-(1H-indazol-5-yl)-2-phenylbut-1-en-1-yl)phenoxy)ethyl)amino)-N-methylbutanamide (Compound 51)

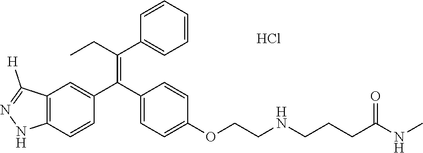 [SMILES: CC/C(=C(/c1ccc(OCCNCCCC(=O)NC)cc1)c1ccc2[nH]ncc2c1)c1ccccc1 ; Cl]

Compound 51 was synthesized following the approach outlined in Scheme 6, modifying step 1 by substituting (E)-4-((2-(4-((E)-1-(1H-indazol-5-yl)-2-phenylbut-1-en-1-yl)phenoxy)ethyl)amino)-N-methylbut-2-enamide (preparation shown in Example 50) for compound 336 to deliver the title compound in 50.9 mg, 31% overall yield, as an off-white solid. ¹H NMR (300 MHz. Methanol-d4) δ 8.62 (s, 1H), 7.92-7.84 (m, 1H), 7.70 (d, J=8.8 Hz, 1H), 7.48 (dd, J=8.8, 1.5 Hz, 1H), 7.25-7.07 (m, 4H), 6.87 (d, J=8.7 Hz, 2H), 6.70 (d, J=8.7 Hz, 2H), 4.16 (t, J=4.9 Hz, 2H), 3.39 (t, J=4.9 Hz, 2H), 3.11 (t, J=7.3 Hz, 2H), 2.70 (s, 3H), 2.56-2.33 (m, 4H), 2.07-1.87 (m, 2H), 0.96 (t, J=7.4 Hz, 3H). LCMS: 483.3 [M+H]⁺.

### Example 52: Synthesis of (E)-1-(piperidin-1-yl)-4-((2-(4-((E)-4,4,4-trifluoro-1-(3-fluoro-1H-indazol-5-yl)-2-phenylbut-1-en-1-yl)phenoxy)ethyl)amino)but-2-en-1-one (Compound 52)

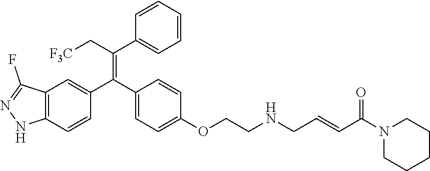 [SMILES: O=C(/C=C/CNCCOc1ccc(/C(=C(/CC(F)(F)F)c2ccccc2)c2ccc3[nH]nc(F)c3c2)cc1)N1CCCCC1]

Compound 52 was synthesized following the approach outlined in Scheme 10, omitting Steps-1-3 and Step-6, by modifying: a) Step-4 by substituting 76 equiv of KOH for N,N-dimethylbarbituric acid, 0.2 equiv of Pd(OH)₂ for Pd(PPh₃)₄, and stirring at room temperature, and b) Step-5 by substituting 0.8 equiv of (E)-4-bromo-1-(piperidin-1-yl)but-2-en-1-one (Scheme 4, Steps-a-b, substituting piperidine for methylamine in Step-b) for compound 359, and not adding (Boc)₂O to deliver the title compound in 17.0 mg, 10.2% overall yield. ¹H NMR (400 MHz, METHANOL-d₄) δ 7.63 (s, 1H), 7.46 (dd, J=8.78, 1.63 Hz; 1H), 7.23-7.36 (m, 1H), 7.12-7.23 (m, 5H), 6.81-6.88 (m, 2H), 6.72-6.81 (m, 1H), 6.57-6.68 (m, 3H), 3.99 (t, J=5.27 Hz, 2H), 3.53-3.61 (m, 4H), 3.43-3.48 (m, 2H), 3.40 (d, J=10.54 Hz, 2H), 3.37 (s, 2H), 2.94 (t, J=5.33 Hz, 2H), 1.63-1.72 (m, 2H), 1.50-1.63 (m, 4H). LCMS: 606.6 [M+H]⁺.

### Example 53: Synthesis of (2)-3-(2-((2-((5-(4,4,4-trifluoro-1-(3-fluoro-1H-indazol-5-yl)-2-phenylbut-1-en-1-ylpyridin-2-yl)oxy)ethyl)amino)ethyl)pyrrolidin-2-one (Compound 53)

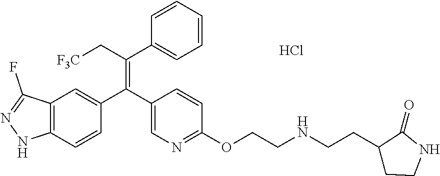 [SMILES: O=C1NCCC1CCNCCOc1ccc(/C(=C(/CC(F)(F)F)c2ccccc2)c2ccc3[nH]nc(F)c3c2)cn1 ; Cl]

Compound 53 was synthesized following the approach outlined in Scheme 10, omitting Step-4 and Step-6, by modifying: a) Step-1 by substituting tert-butyl (2-((5-iodopyridin-2-yl)oxy)ethyl)carbamate (Scheme 4, Step-1) for compound 307, and b) Step-5 by substituting 2.0 equiv of 2-(2-oxopyrrolidin-3-yl)acetaldehyde (preparation shown below in Steps-a-c) for compound 359 and reacting it with (Z)-2-((5-(4,4,4-trifluoro-1-(3-fluoro-1H-indazol-5-yl)-2-phenylbut-1-en-1-yl)pyridin-2-yl)oxy)ethan-1-amine in DCM (0.2M) for 1 hour at room temperature, then adding 2.0 equiv of NaBH₄ batchwise and stirring until completion. The HCl salt was formed by HPLC purification using CH₃CN in water (HCl 0.05%) to deliver the title compound in 10.3 mg, 0.32% overall yield, as a light brown solid. ¹H NMR (400 MHz. Methanol-d₄) δ 7.77 (d, J=2.4 Hz, 1H), 7.68 (s, 1H), 7.59-7.56 (m, 1H), 7.53-7.51 (m, 1H), 7.34 (dd, J=8.7, 1.6 Hz, 1H), 7.32-7.21 (m, 5H), 6.93-6.91 (d, J=8.8 Hz, 1H), 4.56-4.52 (m, 2H), 3.47-3.40 (m, 4H), 3.37-3.34 (m, 2H), 3.24-3.21 (t, J=6.0 Hz, 2H), 2.60 (m, 1H), 2.35 (m, 1H), 1.93-1.80 (m, 3H). LCMS: 568 [M+H]⁺.

### Step-a: Synthesis of 3-(2-nitroethyl)dihydrofuran-2(3H)-one

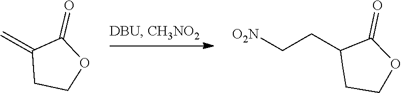

Into a 250-mL round-bottom flask was placed 3-methylideneoxolan-2-one (5 g, 50.97 mmol, 1.00 equiv), DBU (1 g, 6.57 mmol, 0.13 equiv), and CH₃NO₂ (100 mL). The resulting solution was stirred at 25° C. until completion. The resulting mixture was concentrated under vacuum, then taken up in 100 mL of DCM and washed with 2×100 mL of 3.0 M HCl, 1×100 mL of water, 1×100 mL of saturated aqueous NaHCO₃, and 1×100 mL of brine. The solution was dried over anhydrous sodium sulfate, then concentrated under vacuum to deliver the title compound in 5.1 g (63%) as a brown oil. The product was carried forward to the next step without further purification.

### Step-b: Synthesis of 3-(2-hydroxyethyl)pyrrolidin-2-one

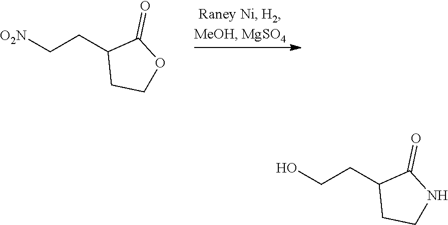 [SMILES: O=C1OCCC1CC[N+](=O)[O-] ; O=C1NCCC1CCO]

Into a 500-mL round-bottom flask was placed 3-(2-nitroethyl)dihydrofuran-2(3H)-one (15 g. 94.26 mmol, 1.00 equiv), Raney Ni (11.89 g, 2.00 equiv), methanol (200 mL), and magnesium sulfate (10.7 g, 3.00 equiv). The resulting solution was stirred at 25° C. until completion. The solids were filtered out, and the solution was concentrated under vacuum to deliver the title compound in 9.6 g (79%) as a light yellow oil. LCMS: 130 [M+H]⁺.

### Step-c: Synthesis of 2-(2-oxopyrrolidin-3-yl)acetaldehyde

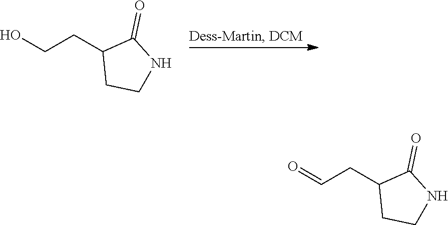 [SMILES: O=C1NCCC1CCO ; O=CCC1CCNC1=O]

Into a 40-mL round-bottom flask was placed 3-(2-hydroxyethyl)pyrrolidin-2-one (130 mg, 1.01 mmol, 1.00 equiv), Dess-Martin (2.1 g, 5.00 equiv), and DCM (5 mL). The resulting solution was stirred at room temperature until completion. The reaction was filtered and the solution was used directly in the next step without further purification, considering 100% yield.

### Example 54: Synthesis of (E)-N-methyl-4-((2-((6-((Z)-4,4,4-trifluoro-1-(3-fluoro-1H-indazol-5-yl)-2-phenylbut-1-en-1-yl)pyridazin-3-yl)oxy)ethyl)amino)but-2-enamide (Compound 54)

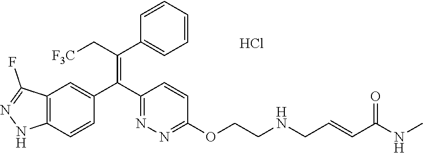 [SMILES: CNC(=O)/C=C/CNCCOc1ccc(/C(=C(/CC(F)(F)F)c2ccccc2)c2ccc3[nH]nc(F)c3c2)nn1 ; Cl]

Compound 54 was synthesized following the approach outlined in Scheme 10, omitting Step-4, by modifying: a) Step-1 by substituting tert-butyl (2-((6-iodopyridazin-3-yl)oxy)ethyl)carbamate (preparation shown below in Step-a) for compound 307 and using 3.0 equiv of Cs₂CO₃, b) Step-2 by substituting 1.1 equiv of iodobenzene for bromobenzene, Pd(dppf)Cl₂ for Pd(PPh₃)₂Cl₂, and K₂CO₃ for KOH, and c) Step-5 by substituting 1.0 equiv of (E)-4-bromo-N-methylbut-2-enamide for compound 359, using 4.0 equiv of DIEA and 2.0 equiv of (Boc)₂O to deliver the title compound in 11.5 mg, 0.30% overall yield, as a yellow solid. ¹H NMR (400 MHz, Methanol-d₄) δ 7.63 (d, J=1.2 Hz, 1H), 7.56 (d, J=9.2 Hz, 1H), 7.54 (m, 1H), 7.44-7.41 (dd, J=8.8, 1.6 Hz, 1H), 7.33-7.24 (m, 6H), 6.72-6.65 (d, J=15.8 Hz, 1H), 6.32-6.28 (m, 1H), 4.85-4.66 (m, 2H), 3.88-3.86 (dd, J=7.2, 1.2 Hz, 2H), 3.60-3.58 (d, J=10.4 Hz, 2H), 3.55-3.47 (m, 2H), 2.80 (s, 3H). LCMS: 555 [M+H]⁺.

### Step-a: Synthesis of tert-butyl (2-((6-iodopyridazin-3-yl)oxy)ethyl)carbamate

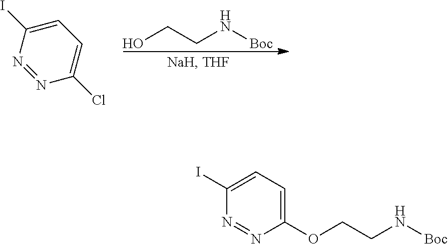 [SMILES: Clc1ccc(I)nn1 ; CC(C)(C)OC(=O)NCCO ; CC(C)(C)OC(=O)NCCOc1ccc(I)nn1]

Into a 500-mL 3-necked round-bottom flask was placed 3-chloro-6-iodopyridazine (10 g, 41.59 mmol, 1.00 equiv) and THF (300 mL). This was followed by the addition of sodium hydride (2.23 g, 92.92 mmol, 1.30 equiv) in portions at 0° C. The resulting solution was stirred at 0° C. in an ice/salt bath until completion. To this was added tert-butyl (2-hydroxyethyl)carbamate (10.1 g, 62.66 mmol, 1.50 equiv) and the solution was stirred until completion. The reaction was then quenched by the addition of water (200 mL), extracted with 3×200 mL of ethyl acetate, and the organic layers were combined and concentrated under vacuum. The residue was applied onto a silica gel column with ethyl acetate/petroleum ether (1:10). The fractions were combined and concentrated under vacuum to deliver the title compound in 13 g (85.5%) as a brown solid.

### Example 55: Synthesis of (E)-1-(piperidin-1-yl)-4-((2-((4,4,4-trifluoro-1-(3-fluoro-1H-indazol-1-yl)-2-phenylbut-1-en-1-yl)pyridin-2-yl)oxy)ethyl)amino)but-2-en-1-one (Compound 55)

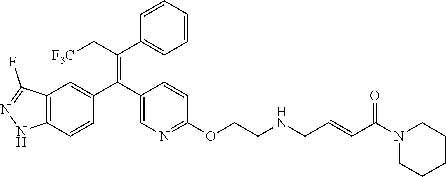 [SMILES: O=C(/C=C/CNCCOc1ccc(/C(=C(/CC(F)(F)F)c2ccccc2)c2ccc3[nH]nc(F)c3c2)cn1)N1CCCCC1]

Compound 55 was synthesized following the approach outlined in Scheme 3, by modifying: a) Step-7 by substituting tert-butyl (E)-(2-((5-iodopyridin-2-yl)oxy)ethyl)(4-oxo-4-(piperidin-1-yl)but-2-en-1-yl)carbamate (preparation shown below in Steps-a-b) for compound 324, using 0.1 equiv of Pd(PPh₃)₂Cl₂ and a 6:1 ratio of 2-Methyl THF:H₂O, and stirring at 50° C. until completion, b) Step-8 by using 0.1 equiv of Pd(PPh₃)₂Cl₂ and a 4:1 ratio of dioxane:H₂O. and c) Step-9 by using a 5:2 ratio of TFA:DCM to deliver the title compound in 88.0 mg, 1.23% overall yield, as an off-white solid. ¹H NMR (400 MHz, Methanol-d4) δ 7.72 (dd, J=2.4, 0.7 Hz, 1H), 7.65 (s, 1H), 7.53-7.50 (m, 1H), 7.35-7.31 (m, 2H), 7.26-7.20 (m, 5H), 6.89-6.85 (m, 1H), 6.67-6.60 (m, 2H), 4.49-4.47 (m, 2H), 3.88-3.86 (dd, J=6.8, 1.4 Hz, 2H), 3.63-3.56 (m, 4H), 3.45-3.40 (m, 4H), 1.72-1.70 (m, 2H), 1.60-1.57 (d, J=5.2 Hz, 4H). LCMS: 608.3 [M+H]⁺.

### Step-a. Synthesis of (E)-4-bromo-1-(piperidin-1-yl)but-2-en-1-one

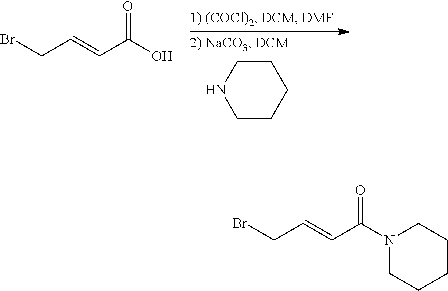 [SMILES: O=C(O)/C=C/CBr ; C1CCNCC1 ; O=C(/C=C/CBr)N1CCCCC1]

Into a 250-mL 3-necked round-bottom flask was placed (E)-4-bromobut-2-enoic acid (5.0 g, 30.31 mmol, 1.00 equiv), DCM (100 mL), and N,N-dimethylformamide (0.1 mL), and then oxalyl dichloride (4.23 g, 33.33 mmol, 1.10 equiv) was added dropwise with stirring at 0° C. The reaction was then stirred at room temperature until completion, and then a mixture of piperidine (2.6 g, 30.54 mmol, 1.00 equiv), sodium carbonate (9.6 g, 90.57 mmol, 3.00 equiv) and DCM (50 mL) were added at 0° C. The resulting solution was stirred at room temperature until completion. The reaction was then quenched by the addition of water (100 mL), extracted with 3×100 mL ethyl acetate, and washed with 100 mL brine. The organic layers were combined, dried over anhydrous sodium sulfate, and concentrated under vacuum to deliver the title compound in 6.0 g (85%) as brown oil. The product was taken forward without any further purification.

### Step-b: Synthesis of tert-butyl (E)-(2-((5-iodopyridin-2-yl)oxy)ethyl)(4-oxo-1-(piperidin-1-yl)but-2-en-1-yl)carbamate

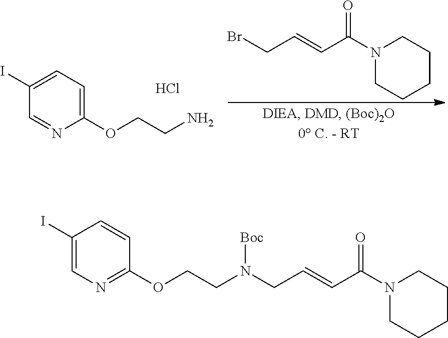

Into a 250-mL round-bottom flask was placed 2-((5-iodopyridin-2-yl)oxy)ethan-1-amine hydrochloride (9.7 g, 28.78 mmol, 1.00 equiv) (Scheme 4, Steps-1-2) and N,N-dimethylformamide (50 mL), and then DIEA (11 g, 85.11 mmol, 3.00 equiv) was added dropwise with stirring at 0° C. To this solution was added (E)-4-bromo-1-(piperidin-1-yl)but-2-en-1-one (6 g, 25.85 mmol, 0.90 equiv) dropwise. The resulting solution was then stirred at room temperature until completion. To the mixture was then added Boc₂O (12.5 g, 57.27 mmol, 2.00 equiv). The resulting solution was allowed to react, with stirring, at room temperature until completion. The reaction was then quenched by the addition of water, extracted with 3×100 mL ethyl acetate, and washed with 100 mL brine. The organic layers were combined, dried over anhydrous sodium sulfate, and concentrated under vacuum. The residue was applied onto a silica gel column with ethyl acetate/petroleum ether (1:1) to deliver the title compound in 2.6 g (18%) as yellow oil. ¹H NMR (400 MHz, Chloroform-d) δ 8.31 (d, J=2.3 Hz, 1H), 7.81-7.78 (dd, J=8.7, 2.4 Hz, 1H), 6.75-6.71 (m, 1H), 6.60-6.58 (d, J=8.6 Hz, 1H), 6.32-6.23 (t, J=16.6 Hz, 1H), 4.41-4.37 (m, 2H), 4.07-4.04 (m, 2H), 3.60-3.55 (dq, J=11.0, 5.7 Hz, 4H), 3.44 (s, 2H), 1.67-1.53 (m, 6H), 1.44 (s, 9H). LCMS: 516 [M+H]⁺.

### Example 56: Synthesis of (E)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (Compound 56)

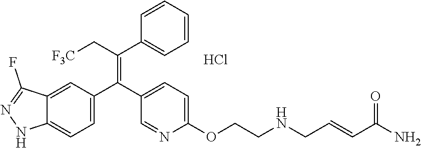 [SMILES: NC(=O)/C=C/CNCCOc1ccc(/C(=C(/CC(F)(F)F)c2ccccc2)c2ccc3[nH]nc(F)c3c2)cn1 ; Cl]

Compound 56 was synthesized following the approach outlined in Scheme 3 by modifying: a) Step-7 by substituting tert-butyl (E)-(4-amino-4-oxobut-2-en-1-yl)(2-((5-iodopyridin-2-yl)oxy)ethyl)carbamate (preparation shown below in Step-a) for compound 324, using 1.2 equiv of (Z)-3-fluoro-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole. 0.1 equiv of Pd(PPh₃)₂Cl₂, 3.0 equiv of Cs₂CO₃, and stirring at 50° C. until completion, b) Step-8 by using 1.5 equiv of bromobenzene, 0.1 equiv of Pd(PPh₃)₂Cl₂, and 3.0 equiv of KOH, and c) Step-9 by using a 1:1 ratio of TFA:DCM to deliver the title compound in 172.2 mg, 2.99% overall yield, as an off-white solid. ¹H NMR (400 MHz. Methanol-d4) δ 7.72 (s, 1H), 7.66 (s, 1H), 7.53-7.50 (m, 1H), 7.35-7.31 (m, 2H), 7.26-7.19 (m, 5H), 6.75-6.65 (m, 2H), 6.36-6.32 (m, 1H), 4.49-4.46 (m, 2H), 3.87-3.85 (dd, J=6.8, 1.4 Hz, 2H), 3.50-3.40 (m, 4H). LCMS: 540 [M+H]⁺.

### Step-a: Synthesis of tert-butyl (E)-(4-amino-4-oxobut-2-en-1-yl)(2-((5-iodopyridin-2-yl)oxy)ethyl)carbamate

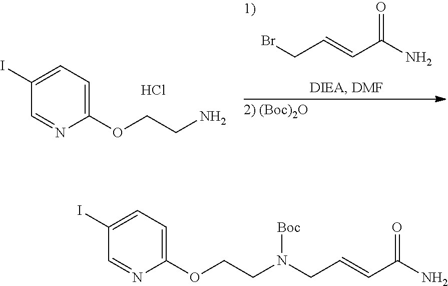 [SMILES: NC(=O)/C=C/CBr ; NCCOc1ccc(I)cn1 ; Cl ; CC(C)(C)OC(=O)N(C/C=C/C(N)=O)CCOc1ccc(I)cn1]

Into a 100-mL round-bottom flask was placed 2-((5-iodopyridin-2-yl)oxy)ethan-1-amine hydrochloride (9.2 g, 30.61 mmol, 1.00 equiv) and DMF (30 mL), and then DIEA (21 g, 162.49 mmol, 3.00 equiv) was added in dropwise with stirring at 0° C. To this solution was added (E)-4-bromobut-2-enamide (9 g, 62.76 mmol, 2.00 equiv) (Scheme 4, Steps-a-b, substituting 1M NH₃ in THF for methylamine in Step-b) dropwise. The resulting solution was stirred at room temperature until completion. Then (Boc)₂O (1.9 g, 8.71 mmol, 2.00 equiv) was added. The resulting solution was stirred at room temperature until completion. The reaction was then quenched by the addition of 500 mL of water, extracted with 3×100 mL of ethyl acetate, and washed with brine (100 mL). The organic layers were combined, dried over anhydrous sodium sulfate, and concentrated under vacuum. The crude product was purified by C18 chromatography (methanol/H₂O=7/3), and the fractions were concentrated under vacuum to deliver the title compound in 1.0 g (52%) as yellow oil. LCMS: 448 [M+H]⁺.

### Example 57: Synthesis of (E)-4-((2-(4-((E)-4,4,4-trifluoro-1-(3-fluoro-1H-indazol-5-yl)-2-phenylbut-1-en-1-yl)phenoxy)ethyl)amino)but-2-enamide (Compound 57)

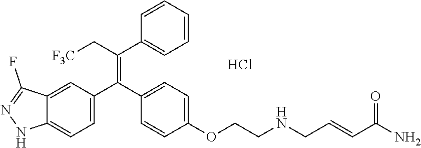 [SMILES: NC(=O)/C=C/CNCCOc1ccc(/C(=C(/CC(F)(F)F)c2ccccc2)c2ccc3[nH]nc(F)c3c2)cc1 ; Cl]

Compound 57 was synthesized following the approach outlined in Scheme 3 by modifying: a) Step-7 by substituting 0.8 equiv tert-butyl (E)-(4-amino-4-oxobut-2-en-1-yl)(2-(4-iodophenoxy)ethyl)carbamate (preparation shown in Example 56, Step-a, substituting 2-(4-iodophenoxy)ethan-1-amine hydrochloride (Scheme 5, Steps-1-2) for 2-((5-iodopyridin-2-yl)oxy)ethan-1-amine hydrochloride), 0.1 equiv of Pd(PPh₃)₂Cl₂, and stirring at 60° C. until completion, b) Step-8 by using 1.5 equiv of bromobenzene, 7.0 equiv of KOH, and 0.1 equiv of Pd(PPh₃)₂Cl₂, and c) Step-9 by using a 5:1 ratio of TFA:DCM to deliver the title compound in 103.0 mg, 0.27% overall yield, as a white solid. ¹H NMR (400 MHz, Methanol-d4) δ 7.61 (d, J=1.3 Hz, 1H), 7.49-7.46 (m, 1H), 7.30-7.27 (m, 1H), 7.24-7.13 (m, 5H), 6.92-6.89 (m, 2H), 6.77-6.70 (m, 3H), 6.38-6.34 (dt, J=15.5, 1.4 Hz, 1H), 4.19-4.16 (m, 2H), 3.90-3.88 (m, 2H), 3.44-3.35 (m, 4H). LCMS: 539, 1 [M+H]⁺.

### Example 58: Synthesis of (E)-4-((2-((5-((Z)-2-(2-chloro-4-fluorophenyl)-4,4,4-trifluoro-1-(1H-indazol-5-yl)but-1-en-1-yl)pyridin-2-yl)oxy)ethyl)amino)-N-methylbut-2-enamide (Compound 58)

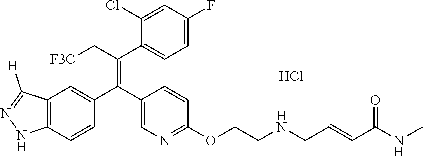 [SMILES: CNC(=O)/C=C/CNCCOc1ccc(/C(=C(/CC(F)(F)F)c2ccc(F)cc2Cl)c2ccc3[nH]ncc3c2)cn1 ; Cl]

Compound 58 was synthesized following the approach outlined in Scheme 3 by modifying: a) Step-7 by substituting (Z)-1-(tetrahydro-2H-pyran-2-yl)-5-(4,4,4-trifluoro-1,2-bis(4,4,5,5-tetramethyl-1,3,2-dioxaborolan-2-yl)but-1-en-1-yl)-1H-indazole (Scheme 8, Steps-1-5) for compound 323, using 2.5 equiv of Cs₂CO₃, 0.1 equiv of Pd(PPh₃)₂Cl₂, and a 10:2 ratio of 2-Methyl THF:H₂O. and stirring at 50° C. until completion, b) Step-8 by substituting 3.0 equiv of 2-chloro-4-fluoro-1-iodobenzene for bromobenzene, using 0.1 equiv of Pd(PPh₃)₂Cl₂ and 7.0 equiv of KOH, and c) Step-9 by using a 1:1 ratio of TFA:DCM to deliver the title compound in 70.9 mg, 0.82% overall yield, as a white solid. ¹H NMR (400 MHz, Methanol-d₄) δ 8.25 (d, J=1.0 Hz, 1H), 7.86-7.83 (m, 2H), 7.68-7.66 (m, 1H), 7.44-7.35 (m, 3H). 7.21-7.18 (dd, J=8.7, 2.6 Hz, 1H), 7.07-7.03 (m, 1H), 6.73-6.65 (m, 2H), 6.32-6.28 (m, 1H), 4.51-4.48 (m, 2H), 3.87-3.85 (dd, J=6.9, 1.4 Hz, 2H), 3.47-3.39 (m, 4H), 2.81 (s, 3H). LCMS: 588.2 [M+H]⁺.

### Example 59: Synthesis of (E)-4-((2-((5-((Z)-2-(2-chloro-4-fluorophenyl)-1-(1H-indazol-5-yl)but-1-en-1-yl)pyridin-2-yl)oxy)ethyl)amino)-N-methylbut-2-enamide (Compound 59)

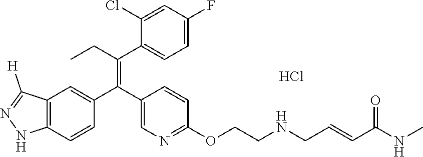 [SMILES: CC/C(=C(/c1ccc(OCCNC/C=C/C(=O)NC)nc1)c1ccc2[nH]ncc2c1)c1ccc(F)cc1Cl ; Cl]

Compound 59 was synthesized following the approach outlined in Scheme 3 by modifying: a) Step-7 by substituting (Z)-5-(1,2-bis(4,4,5,5-tetramethyl-1,3,2-dioxaborolan-2-yl)but-1-en-1-yl)-1-(tetrahydro-2H-pyran-2-yl)-1H-indazole (following the preparation shown in Example 48, Steps-a-b, substituting 5-bromo-1-(tetrahydro-2H-pyran-2-yl)-1H-indazole for 5-bromo-3-fluoro-1-(tetrahydro-2H-pyran-2-yl)-1H-indazole) for compound 323, using 0.1 equiv of Pd(PPh₃)₂Cl₂, 3.0 equiv of Cs₂CO₃, and a 4:1 ratio of 2-Methyl THF:H₂O, and b) Step-8 by substituting 1.5 equiv of 2-chloro-4-fluoro-1-iodobenzene for bromobenzene, using 0.1 equiv of Pd(PPh₃)₂Cl₂ and 3.0 equiv of KOH to deliver the title compound in 192.6 mg, 1.13% overall yield, as an off-white solid. ¹H NMR (300 MHz, Methanol-d4) δ 8.77 (d, J=1.0 Hz, 1H), 8.07 (s, 1H), 8.12-7.98 (m, 2H), 7.84-7.81 (m, 1H), 7.71-7.67 (m, 1H), 7.30 (m, 1H), 7.20 (m, 1H), 7.17-7.13 (m, 2H), 6.70 (m, 1H), 6.39 (m, 1H), 4.69-4.66 (t, J=4.8 Hz, 2H), 3.93-3.91 (dd, J=6.9, 1.4 Hz; 2H), 3.55-3.52 (m, 2H), 2.79 (s, 3H), 2.65-2.45 (m, 2H), 1.03-0.98 (t, J=7.5 Hz, 3H). LCMS: 534.1 [M+H]⁺.

### Example 60: Synthesis of (E)-1-(azetidin-1-yl)-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-en-1-one (Compound 60)

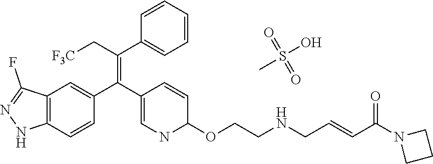

Compound 60 was synthesized following the approach outlined in Scheme 3 by modifying: a) Step-7 by substituting 0.7 equiv of tert-butyl (E)-(4-(azetidin-1-yl)-4-oxobut-2-en-1-yl)(2-((5-iodopyridin-2-yl)oxy)ethyl)carbamate (preparation shown below in Steps-a-b) for compound 324, using 0.1 equiv of Pd(PPh₃)₂Cl₂ and a ratio of 10:2 2-MethylTHF:H₂O, and stirring at 60° C. until completion, b) Step-8 by using 0.1 equiv of Pd(PPh)₂Cl₂, 3.0 equiv of KOH, 1.0 equiv of bromobenzene, and a 3:1 ratio of dioxane:H₂O, and c) Step-9 by using a 4:1 ratio of TFA:DCM to deliver the title compound as a free base. The free base compound was then converted to the methanesulfonic acid salt with CH₃SO₃H (1.1 eq, 1N in CH₃CN) to deliver the title compound in 142.0 mg, 1.12%6 overall yield, as an off-white solid. ¹H NMR (400 MHz, Methanol-d4) δ 7.70 (dd, J=2.4, 0.7 Hz, 1H), 7.69-7.63 (m, 1H), 7.50-7.48 (m, 1H), 7.37-7.30 (m, 2H), 7.29-7.19 (m, 5H), 6.68-6.62 (m, 2H), 6.39-6.35 (m, 1H), 4.46-4.44 (m, 2H), 4.32-4.28 (m, 2H), 4.09-4.05 (m, 2H), 3.86-3.84 (dd, J=6.8, 1.5 Hz, 2H), 3.42-3.37 (m, 4H), 2.71 (s, 3H), 2.36-2.32 (m, 2H). LCMS: 580.3 [M+H]⁺.

### Step-a: Synthesis of (E)-1-(azetidin-1-yl)-4-bromobut-2-en-1-one

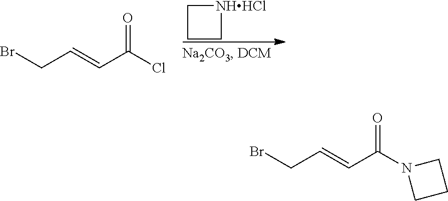

Into a 500-mL round-bottom flask was placed azetidine hydrochloride (20 g, 0.2162 mol, 1.00 equiv), DCM (200 mL), and sodium carbonate (68.75 g, 0.6486 mol, 3.00 equiv), followed by the dropwise addition of (E)-4-bromobut-2-enoyl chloride (39.135 g, 0.2162 mol, 1.00 equiv) (Scheme 4. Step-a) at 0° C. The resulting solution was stirred at 25° C. until completion, then the solution was diluted with 500 mL of water and extracted with 3×500 mL of ethyl acetate. The organic layers were combined, washed with 500 mL of brine, dried over anhydrous sodium sulfate, and concentrated under vacuum to deliver the title compound in 18 g (81%) as a yellow oil.

### Step-b: Synthesis of tert-butyl (E)-(4-(azetidin-1-yl)-4-oxobut-2-en-1-yl)(2-((5-iodopyridin-2-yl)oxy)ethyl)carbamate

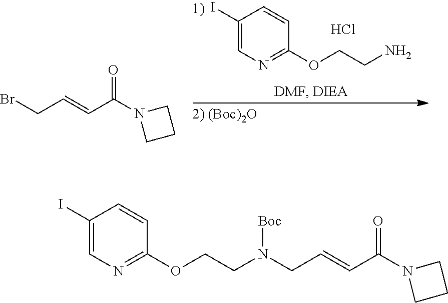 [SMILES: NCCOc1ccc(I)cn1 ; Cl ; O=C(/C=C/CBr)N1CCC1 ; CC(C)(C)OC(=O)N(C/C=C/C(=O)N1CCCC1)CCOc1ccc(I)cn1]

Into a 20-mL round-bottom flask, was placed 2-(5-iodopyridin-2-yloxy)ethanamine hydrochloride (1.66098 g, 6.29 mmol, 1.00 equiv), N,N-dimethylformamide (10 mL), DIEA (1.94 g, 15.01 mmol, 3.00 equiv), followed by the added of (2E)-1-(azetidin-1-yl)-4-bromobut-2-en-1-one (1 g, 4.90 mmol, 1.00 equiv) in batchwise. The resulting solution was stirred at 25° C. until completion. Then Boc₂O (2.15 g, 12.4 mmol, 2 equiv) was added and the solution was stirred at 25° C. until completion. The solution was then diluted with 100 mL of water and extracted with 3×100 mL of ethyl acetate. The organic layers were combined, washed with 100 mL of brine, dried over anhydrous sodium sulfate and concentrated under vacuum. The residue was applied onto a silica gel column eluting with DCM/methanol (10:1) to deliver the title compound in 460 mg (20%) as a yellow oil. ¹H NMR (300 MHz, Chloroform-d) δ 8.34 (d, J=2.3 Hz, 1H), 7.83 (dd, J=8.6, 2.3 Hz, 1H), 6.82 (m, 1H), 6.63 (d, J=8.7 Hz, 1H), 5.90 (d, J=16.3 Hz, 1H), 4.42 (d, J=6.9 Hz, 2H), 4.11 (m, 6H), 3.65-3.53 (m, 2H), 2.45-2.25 (m, 2H), 1.46 (s, 9H).

### Example 61: Synthesis of (E)-N-methyl-4-((3-((5-((Z)-4,4,4-trifluoro-1-(3-fluoro-1H-indazol-5-yl)-2-phenylbut-1-en-1-yl)pyridin-2-yl)oxy)propylamino)but-2-enamide (Compound 61)

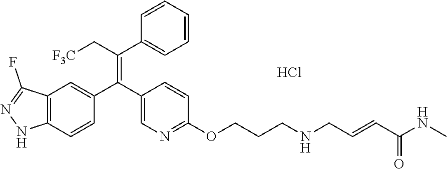 [SMILES: CNC(=O)/C=C/CNCCCOc1ccc(/C(=C(/CC(F)(F)F)c2ccccc2)c2ccc3[nH]nc(F)c3c2)cn1 ; Cl]

Compound 61 was synthesized following the approach outlined in Scheme 3 by modifying: a) Step-7 by substituting tert-butyl (E)-(3-((5-iodopyridin-2-yl)oxy)propyl)(4-(methylamino)-4-oxobut-2-en-1-yl)carbamate (preparation shown below in Steps-a-c) for compound 324, using 2.5 equiv of Cs₂CO₃ and 0.1 equiv of Pd(PPh₃)₂Cl₂, and stirring at 50° C. until completion, b) Step-8 by using 0.1 equiv of Pd(PPh₃)₂Cl₂, 7.0 equiv of KOH, and 1.1 equiv of bromobenzene, and c) Step-9 by using a 1:1 ratio of TFA:DCM to deliver the title compound in 56.0 mg, 1.39% overall yield, as a white solid. ¹H NMR (300 MHz, Methanol-d₄) δ 7.68-7.65 (m, 2H), 7.53-7.49 (dd, J=8.6, 2.2 Hz, 1H), 7.33-7.20 (m, 7H), 6.71-6.56 (m, 2H), 6.29-6.24 (d, J=15.3 Hz, 1H), 4.30-4.26 (t, 0.1=5.8 Hz, 2H), 3.80-3.77 (d, J=6.8 Hz, 2H), 3.45-3.38 (t, J=10.5 Hz, 2H), 3.18-3.09 (t, J=7.4 Hz, 2H), 2.82 (s, 3H), 2.13-2.08 (m, 2H). LCMS: 590.15 [M+Na]⁺.

### Step-a: Synthesis of tert-butyl (3-((5-iodopyridin-2-yl)oxy)propyl)carbamate

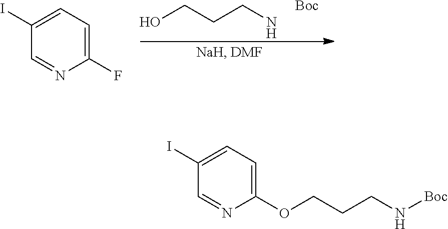 [SMILES: Fc1ccc(I)cn1 ; NCCCO ; CC(C)(C)OC(=O)NCCCOc1ccc(I)cn1 ; "Boc"]

Into a 250-mL round-bottom flask was placed 2-fluoro-5-iodopyridine (10 g, 44.85 mmol, 1.00 equiv) and N,N-dimethylformamide (100 mL). This was followed by the addition of sodium hydride (4.48 g, 186.67 mmol, 1.50 equiv) in batches with stirring at 0° C. The resulting solution was stirred at 0° C. in an ice/salt bath until completion. Then tert-butyl N-(3-hydroxypropyl) carbamate (7.85 g, 44.80 mmol, 1.00 equiv) was added. The resulting solution was stirred at room temperature until completion. The reaction progress was monitored by LCMS. The resulting solution was diluted with 300 mL of water, extracted with 3×300 mL of ethyl acetate and the organic layers were combined and dried over anhydrous sodium sulfate. The residue was applied onto a silica gel column with ethyl acetate/petroleum ether (0:100-10:90). The collected fractions were combined and concentrated under vacuum to deliver the title compound in 13.8 g (81%) as a white solid. LCMS: 379.05 [M+H]⁺.

### Step-b: Synthesis of 3-((5-iodopyridin-2-yl)oxy)propan-1-amine hydrochloride

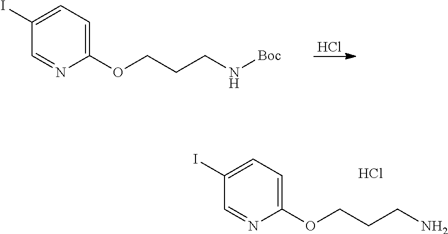 [SMILES: CC(C)(C)OC(=O)NCCCOc1ccc(I)cn1 ; Cl ; NCCCOc1ccc(I)cn1 ; Cl]

Into a 250-mL round-bottom flask was placed tert-butyl (3-((5-iodopyridin-2-yl)oxy)propyl)carbamate (13.8 g, 36.49 mmol, 1.00 equiv) and hydrogen chloride (4M in dioxane, 60 mL). The resulting solution was stirred at room temperature until completion. The reaction mixture was concentrated under vacuum to deliver the title compound in 10 g (87%) as a yellow solid. The material was taken forward without any further purification.

### Step-c: Synthesis of tert-butyl (E)-(3-((5-iodopyridin-2-yl)oxy)propyl)(4-(methylamino)-4-oxobut-2-en-1-yl)carbamate

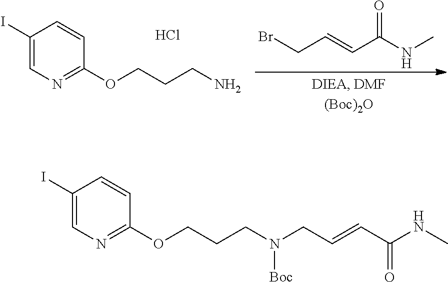

Into a 500-mL round-bottom flask was placed 3-((5-iodopyridin-2-yl)oxy)propan-1-amine hydrochloride (12.6 g, 40.06 mmol, 1.00 equiv), N,N-dimethylformamide (150 mL), and DIEA (46.45 g, 359.41 mmol, 10.00 equiv). This was followed by the addition of (E)-4-bromo-N-methylbut-2-enamide (6.37 g, 35.78 mmol, 1.00 equiv) (Scheme 4, Steps-a-b) in 3 portions at 0° C. over 30 min. The resulting solution was stirred at room temperature until completion. To this was added (Boc)₂O (15.7 g, 71.94 mmol, 2.00 equiv) with stirring. The resulting solution was stirred at room temperature until completion. The reaction progress was monitored by LCMS. The reaction was quenched by the addition of 300 mL of water/ice, extracted with 3×300 mL of ethyl acetate, and washed with 100 mL of brine. The mixture was then dried over anhydrous sodium sulfate and concentrated under vacuum. The residue was applied onto a silica gel column with ethyl acetate/petroleum ether (0:100-10:90). The collected fractions were combined and concentrated under vacuum to deliver the title compound in 1.9 g (90%) as an oil. LCMS: 498.05 [M+Na]⁺.

### Example 62: Synthesis of (Z)-4-((2-((5-(1-(1H-indazol-5-yl)-2-phenylbut-1-en-1-yl)pyridin-2-yl)oxy)ethyl)amino)-N-methylbutanamide (Compound 62)

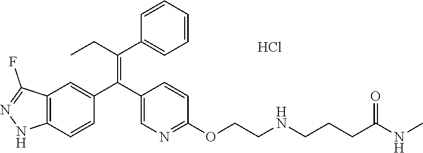 [SMILES: CC/C(=C(/c1ccc(OCCNCCCC(=O)NC)nc1)c1ccc2[nH]nc(F)c2c1)c1ccccc1 ; Cl]

Compound 62 was synthesized following the approach outlined in Scheme 9 by modifying: a) Step-1 by substituting 1-phenylpropan-1-one for compound 349, DCM for toluene, and stirring at room temperature until completion, b) Step-2 by substituting THF (to make a 0.43M solution) for ether, adding the n-BuLi at −78° C., using 1.25 equiv of 4,4,5,5-tetramethyl-2-(tetramethyl-1,3,2-dioxaborolan-2-yl)-1,3,2-dioxaborolane, and stirring at room temperature once all reagents were added until completion, c) Step-3 by substituting Pd(dppf)Cl₂ for Pd₂(dba)₃, 4.0 equiv of Cs₂CO₃ for KOH, a 10:1 ratio of dioxane:H₂O for THF, and removing P(t-Bu)₃.HBF, and d) Step-4 by substituting tert-butyl (E)-(2-((5-iodopyridin-2-yl)oxy)ethyl)(4-(methylamino)-4-oxobut-2-en-1-yl)carbamate (Scheme 4) for compound 335, Pd(dppf)Cl₂ for Pd₂(dba)₃.CHCl₃, using 3.0 equiv of KOH, and stirring at room temperature until completion. Before deprotection, the compound was stirring in methanol with 0.1 equiv of Pd/C and subjected to H₂ (g) until reduction of the double bond to deliver the title compound in 41.5 mg, 0.38% overall yield, as a yellow solid. ¹H NMR (300 MHz. Methanol-d₄) δ 8.58 (s, 1H), 7.92 (s, 1H), 7.80-7.70 (m, 3H), 7.52-7.49 (d, J=8.6 Hz, 1H), 7.33-7.21 (m, 5H), 7.10-7.07 (d, J=8.9 Hz, 1H), 4.62-4.58 (m, 2H), 3.55-3.46 (t, J=4.8 Hz, 2H), 3.17-3.09 (t, J=7.0 Hz, 2H), 2.70 (s, 3H), 2.59-2.51 (m, 2H), 2.44-2.39 (t, J=6.7 Hz, 2H), 2.01-1.92 (m, 2H), 1.02-0.97 (t, J=7.4 Hz, 3H). LCMS: 484.31 [M+H]⁺.

### Example 63: Synthesis of (E)-4-((2-(4-((E)-2-cyclopropyl-1-(3-fluoro-1H-indazol-5-yl)-2-phenylvinyl)phenoxy)ethyl)amino)-N-methylbut-2-enamide (Compound 63)

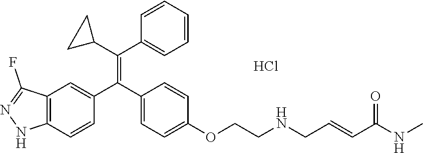 [SMILES: CNC(=O)/C=C/CNCCOc1ccc(/C(=C(\c2ccccc2)C2CC2)c2ccc3[nH]nc(F)c3c2)cc1 ; Cl]

Compound 63 was synthesized following the approach outlined in Scheme 3, omitting Step-4 and Step-5, by modifying: a) Step-3 by substituting 2.0 equiv of ethynylcyclopropane for ethynyltrimethylsilane and using 0.3 equiv of CuI, b) Step-6 by using 1.0 equiv of 4,4,5,5-tetramethyl-2-(tetramethyl-1,3,2-dioxaborolan-2-yl)-1,3,2-dioxaborolane, c) Step-7 by substituting tert-butyl (E)-(2-(4-iodophenoxy)ethyl)(4-(methylamino)-4-oxobut-2-en-1-yl)carbamate (Scheme 5) for compound 324, using 0.1 equiv of Pd(PPh₃)₂Cl₂, 2.5 equiv of Cs₂CO₃, a 5:1 ratio of 2-Methyl THF:H₂O, and stirring at 50° C. until completion, d) Step-8 by using 0.1 equiv of Pd(PPh₃)₂Cl₂, 7.0 equiv of KOH, and a 4:1 ratio of dioxane:H₂O, and e) Step-9 by using a 5:2 ratio of TFA:DCM to deliver the title compound in 13.7 mg, 0.15% overall yield, as a white solid. ¹H NMR (300 MHz, Methanol-d4) δ 7.64 (s, 1H), 7.42 (t, J=1.9 Hz, 2H), 7.17-7.03 (m, 6H), 6.87-6.84 (d, J=8.7 Hz, 2H), 6.70-6.63 (m, 2H), 6.28-6.23 (d, J=15.5 Hz, 1H), 4.13-4.10 (m, 2H), 3.84-3.82 (d, J=6.6 Hz, 2H), 3.38-3.35 (dd, J=11.2, 6.2 Hz, 2H), 2.79 (s, 3H), 1.76-1.74 (s, 1H), 0.65-0.60 (d, J=8.4 Hz, 2H). 0.35-0.32 (d, J=5.5 Hz, 2H). LCMS: 511 [M+H]⁺.

### Example 64: Synthesis of (E)-4-((2-(4-((E)-1-(3-fluoro-1H-indazol-5-yl)-4-hydroxy-2-phenylbut-1-en-1-yl)phenoxy)ethyl)amino)-N-methylbut-2-enamide (Compound 64)

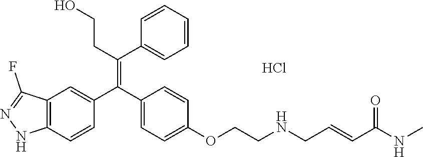 [SMILES: CNC(=O)/C=C/CNCCOc1ccc(/C(=C(/CCO)c2ccccc2)c2ccc3[nH]nc(F)c3c2)cc1 ; Cl]

Compound 64 was synthesized following the approach outlined in Scheme 3, omitting Step-4 and Step-5, by modifying: a) Step-3 by substituting 3.0 equiv of 2-(but-3-yn-1-yloxy)tetrahydro-2H-pyran for ethynyltrimethylsilane, using 0.6 equiv of CuI, 0.4 equiv of Xantphos, and 0.2 equiv of PdCl₂, b) Step-6 by substituting 3-fluoro-1-(tetrahydro-2H-pyran-2-yl)-5-(4-((tetrahydro-2H-pyran-2-yl)oxy)but-1-yn-1-yl)-1H-indazole for compound 322 and using 0.06 equiv of Pt(PPh₃)₄, c) Step-7 by substituting 0.7 equiv of tert-butyl (E)-(2-(4-iodophenoxy)ethyl)(4-(methylamino)-4-oxobut-2-en-1-yl)carbamate (Scheme 5) for compound 324, using 0.1 equiv of Pd(PPh₃)₂Cl₂, and a 5:1 ratio of 2-Methyl THF, d) Step-8 by using 1.3 equiv of bromobenzene, 7.0 equiv of KOH. 0.1 equiv of Pd(PPh₃)₂Cl₂, and e) Step-9 by first making a 0.4M solution with TFA and stirring at room temperature to remove the Boc group, then diluting with a small amount of THF and adding in saturated LiOH (to make a 0.08M solution) and stirring at 0° C. until completion to deliver the title compound in 49.0 mg, 0.66% overall yield, as an off-white solid. ¹H NMR (400 MHz, Methanol-d4) δ 7.63 (t, J=1.1 Hz, 1H), 7.45-7.40 (m, 1H), 7.35-7.32 (dd, J=8.7, 1.5 Hz, 1H), 7.19-7.12 (m, 5H), 6.90-6.88 (m, 2H), 6.73-6.67 (m, 3H), 6.31-6.27 (dt, J=15.3, 1.3 Hz, 1H), 4.17-4.15 (dd, J=5.6, 4.2 Hz, 2H), 3.88-3.86 (dd, J=6.9, 1.4 Hz, 2H), 3.54-3.50 (dd, J=7.9, 6.8 Hz, 2H), 3.42-3.40 (m, 2H), 2.82 (s, 3H), 2.76-2.73 (t, J=7.4 Hz, 2H). LCMS: 515 [M+H]⁺.

### Example 65: Synthesis of (E)-4-((2-(4-((E)-1-(3-fluoro-1H-indazol-5-yl)-4-methoxy-2-phenylbut-1-en-1-yl)phenoxy)ethyl)amino)-N-methylbut-2-enamide (Compound 65)

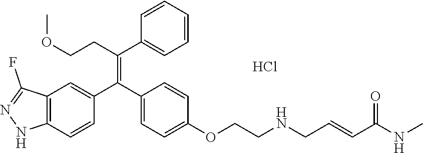 [SMILES: CNC(=O)/C=C/CNCCOc1ccc(/C(=C(/CCOC)c2ccccc2)c2ccc3[nH]nc(F)c3c2)cc1 ; Cl]

Compound 65 was synthesized following the approach outlined in Scheme 3, omitting Step-4 and Step-5, by modifying: a) Step-3 by substituting but-3-yn-1-ol for ethynyltrimethylsilane, using 0.2 equiv of PdCl₂, 0.4 equiv of Xantphos, 5.0 equiv of triethylamine, and 0.6 equiv of CuI, b) adding an additional step to form 3-fluoro-5-(4-methoxybut-1-yn-1-yl)-1-(tetrahydro-2H-pyran-2-yl)-1H-indazole (preparation shown below in Step-a), c) Step-6 by using 1.5 equiv of 4,4,5,5-tetramethyl-2-(tetramethyl-1,3,2-dioxaborolan-2-yl)-1,3,2-dioxaborolane and 0.1 equiv of Pt(PPh₃)₄, d) Step-7 by substituting tert-butyl (E)-(2-(4-iodophenoxy)ethyl)(4-(methylamino)-4-oxobut-2-en-1-yl)carbamate (Scheme 5) for compound 324, using 0.1 equiv of Pd(PPh₃)₂Cl₂, 3.0 equiv of Cs₂CO₃, a 5:1 ratio of 2-Methyl THF:H₂O, and stirring at 50° C. until completion, e) Step-8 by using 1.5 equiv of bromobenzene, 0.1 equiv of Pd(PPh₃)₂Cl₂, and 3.0 equiv of KOH, and f) Step-9 by using a 5:3 ratio of TFA:DCM to deliver the title compound in 181.0 mg, 1.52% overall yield, as an off-white solid. ¹H NMR (400 MHz, Methanol-d4) δ 7.67 (s, 1H), 7.43-7.41 (m, 1H), 7.34-7.32 (m, 1H), 7.21-7.10 (m, 5H), 6.89-6.87 (m, 2H), 6.75-6.68 (m, 3H), 6.34-6.30 (m, 1H), 4.18-4.16 (m, 2H), 3.89-3.87 (m, 2H), 3.43-3.40 (m, 2H), 3.35 (m, 2H), 3.22 (s, 3H), 2.83-2.75 (s, 5H). LCMS: 529.2 [M+H]⁺.

### Step-a: Synthesis of 3-fluoro-5-(4-methoxybut-1-yn-1-yl)-1-(tetrahydro-2H-pyran-2-yl)-1H-indazole

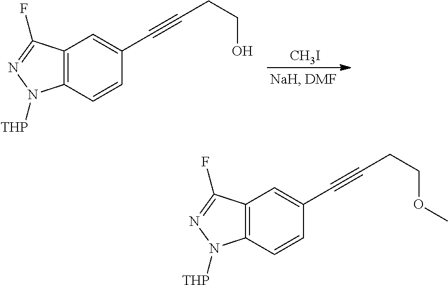

Into a 50-mL round-bottom flask purged and maintained with an inert atmosphere of nitrogen was placed 4-(3-fluoro-1-(tetrahydro-2H-pyran-2-yl)-1H-indazol-5-yl)but-3-yn-1-ol (1.5 g, 5.2 mmol, 1.00 equiv), and DMF (15 mL), then NaH (250 mg, 10.4 mmol, 2 equiv) was added slowly. The mixture was stirred at 0° C. for 30 min, then iodomethane (1.11 g, 7.8 mmol, 1.5 equiv) was added slowly. The resulting solution was stirred at 0° C. until completion. The reaction mixture was quenched by ice water (100 mL), extracted with 3×100 mL of ethyl acetate, and the organic layers were combined. The resulting organic was washed with 1×50 mL of brine, dried over anhydrous sodium sulfate, and concentrated under vacuum. The residue was applied onto a silica gel column eluting with petroleum ether/ethyl acetate (1:2) to deliver the title compound in 1.2 g (76%) as a yellow liquid. LCMS: 303.34 [M+H]⁺.

### Example 66: Synthesis of (E)-4-((2-(4-((E)-4-chloro-1-(3-fluoro-1H-indazol-5-yl)-2-phenylbut-1-en-1-yl)phenoxy)ethyl)amino)-N-methylbut-2-enamide (Compound 66)

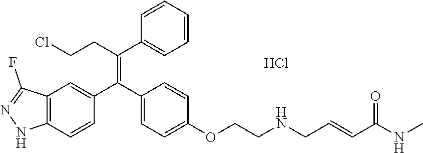 [SMILES: CNC(=O)/C=C/CNCCOc1ccc(/C(=C(/CCCl)c2ccccc2)c2ccc3[nH]nc(F)c3c2)cc1 ; Cl]

Compound 66 was synthesized following the approach outlined in Scheme 3, omitting Step-4 and Step-5, by modifying: a) Step-3 by substituting 4-chlorobut-1-yne for ethynyltrimethylsilane, using 0.2 equiv of PdCl₂, 5.0 equiv of triethylamine, and 0.3 equiv of CuI, b) Step-6 by using 0.1 equiv of Pt(PPh₃)₄, c) Step-7 by substituting tert-butyl (E)-(2-(4-iodophenoxy)ethyl)(4-(methylamino)-4-oxobut-2-en-1-yl)carbamate (Scheme 5) for compound 324, using 0.1 equiv of Pd(PPh₃)₂Cl₂, and stirring at 60° C. until completion, d) Step-8 by using 1.5 equiv of bromobenzene, 0.2 equiv of Pd(PPh₃)₂Cl₂, 7.0 equiv of KOH, and a 5:1 ratio of dioxane:H₂O, and e) Step-9 by using a 5:1 ratio of TFA:DCM to deliver the title compound in 31.8 mg, 0.54% overall yield, as an off-white solid. ¹H NMR (300 MHz, Methanol-d₄) δ 7.69 (s, 1H), 7.46-7.42 (m, 1H), 7.34-7.31 (dd, J=8.7, 1.5 Hz, 1H), 7.20-7.17 (d, J=8.7 Hz, 2H), 7.06-7.01 (m, 3H), 7.00-6.94 (m, 2H), 6.93-6.87 (m, 2H), 6.79-6.69 (m, 1H), 6.35-6.30 (d, J=15.3 Hz, 1H), 4.27-4.23 (m, 2H), 3.93-3.91 (d, J=6.9 Hz, 2H), 3.49-3.45 (m, 4H), 3.00-2.95 (t, J=7.1 Hz, 2H), 2.83 (s, 3H). LCMS: 533 [M+H]⁺.

### Example 67: Synthesis of (E)-4-((2-(4-((E)-1-(3-fluoro-1H-indazol-5-yl)-2-phenylpent-1-en-1-yl)phenoxy)ethyl)amino)-N-methylbut-2-enamide (Compound 67)

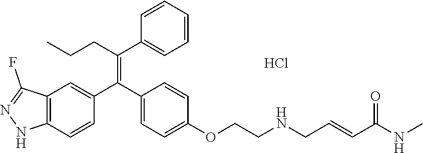 [SMILES: CCC/C(=C(/c1ccc(OCCNC/C=C/C(=O)NC)cc1)c1ccc2[nH]nc(F)c2c1)c1ccccc1 ; Cl]

Compound 67 was synthesized following the approach outlined in Scheme 3, omitting Step-4 and Step-5, by modifying: a) Step-3 by substituting 2.0 equiv of pent-1-yne for ethynyltrimethylsilane, using 5.0 equiv of triethylamine and 0.3 equiv of CuI, b) Step-6 by using 1.1 equiv of 4,4,5,5-tetramethyl-2-(tetramethyl-1,3,2-dioxaborolan-2-yl)-1,3,2-dioxaborolane, c) Step-7 by substituting 0.5 equiv of tert-butyl (E)-(2-(4-iodophenoxy)ethyl)(4-(methylamino)-4-oxobut-2-en-1-yl)carbamate for compound 324, using 0.1 equiv of Pd(PPh₃)₂Cl₂, 2.5 equiv of Cs₂CO₃, a 5:2 ratio of 2-Methyl THF:H₂O, and stirring at 50° C. until completion, d) Step-8 by using 2.0 equiv of bromobenzene, 7.0 equiv of KOH, and 0.1 equiv of Pd(PPh₃)₂Cl₂, and e) using a 1:1 ratio of TFA:DCM to deliver the title compound in 6.7 mg, 0.22% overall yield, as a white solid ¹H NMR (400 MHz, Methanol-d₄) δ 7.22 (d, J=8.6 Hz, 2H), 7.15-7.10 (m, 4H), 7.09-7.01 (m, 4H), 6.98-6.96 (m, 2H), 6.78-6.70 (m, 1H), 6.33-6.29 (d, J=15.2 Hz, 1H), 4.32-4.30 (t, J=4.9 Hz, 2H), 3.93-3.91 (d, J=6.9 Hz, 2H), 3.50-3.48 (d, J=4.8 Hz, 2H), 2.81 (s, 3H), 2.47-2.43 (m, 2H), 1.39-1.33 (m, 2H), 0.83-0.80 (t, =7.4 Hz, 3H). LCMS: 535.1 [M+Na]⁺.

### Example 68: Synthesis of (E)-4-((2-(4-((E)-1-(3-fluoro-1H-indazol-5-yl))-3-methyl-2-phenylbut-1-en-1-yl)phenoxy)ethyl)amino)-N-methylbut-2-enamide (Compound 68)

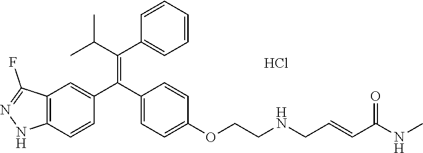 [SMILES: CNC(=O)/C=C/CNCCOc1ccc(/C(=C(\c2ccccc2)C(C)C)c2ccc3[nH]nc(F)c3c2)cc1 ; Cl]

Compound 68 was synthesized following the approach outlined in Scheme 3, omitting Step-4 and Step-5, by modifying: a) Step-3 by substituting 3-fluoro-5-iodo-1-(tetrahydro-2H-pyran-2-yl)-1H-indazole (preparation shown below in Step-a) for compound 319, 2.0 equiv of 3-methylbut-1-yne for ethynyltrimethylsilane, using 5.0 equiv of triethylamine, and 0.3 equiv of CuI, b) Step-6 by using 1.5 equiv of 4,4,5,5-tetramethyl-2-(tetramethyl-1,3,2-dioxaborolan-2-yl)-1,3,2-dioxaborolane and 0.1 equiv of Pt(PPh₃)₄, c) Step-7 by substituting tert-butyl (E)-(2-(4-iodophenoxy)ethyl)(4-(methylamino)-4-oxobut-2-en-1-yl)carbamate (Scheme 5) for compound 324, using 0.1 equiv of Pd(PPh₃)₂Cl₂, 2.5 equiv of Cs₂CO₃, a 5:1 ratio of 2-Methyl THF:H₂O, and stirring at 50° C. until completion, d) Step-8 by using 1.5 equiv of bromobenzene, 0.1 equiv of Pd(PPh₃)₂Cl₂, and 7.0 equiv of KOH, and e) Step-9 by using a 5:1 ratio of TFA:DCM to deliver the title compound in 20.3 mg, 0.03% overall yield, as an off-white solid. ¹H NMR (300 MHz, Methanol-d4) δ 7.32-7.29 (m, 2H), 7.20-7.02 (m, 10H), 6.81-6.71 (dt, J=15.4, 6.9 Hz, 1H), 6.36-6.31 (dt, J=15.3, 1.4 Hz, 1H), 4.35-4.31 (dd, J=5.7, 4.2 Hz, 2H), 3.95-3.91 (dd, J=7.0, 1.3 Hz, 2H), 3.53-3.49 (t, J=4.9 Hz, 2H), 3.11-3.06 (m, 1H), 2.83 (s, 3H), 1.00-0.98 (d, J=6.9 Hz, 6H). LCMS: 513 [M+H]⁺.

### Step-a: Synthesis of 3-fluoro-5-iodo-1-(tetrahydro-2H-pyran-2-yl)-1H-indazole

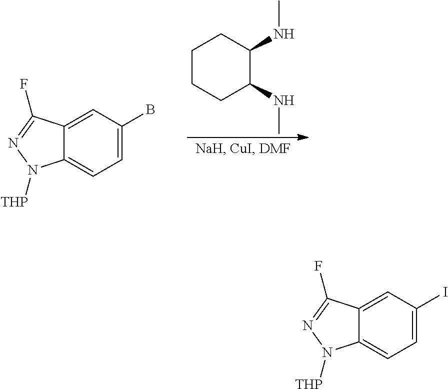

Into a 500-mL round-bottom flask purged with nitrogen was placed 5-bromo-3-fluoro-1-(tetrahydro-2H-pyran-2-yl)-1H-indazole (10 g, 33.21 mmol, 1.00 equiv), N,N-dimethylformamide (300 mL), NaI (30 g, 6.00 equiv), CuI (950 mg, 4.99 mmol, 0.15 equiv), and (1R,2S)—N¹,N²-dimethylcyclohexane-1,2-diamine (1.43 g, 10.05 mmol, 0.30 equiv). The resulting solution was stirred at 120° C. until completion. The reaction was then quenched by the addition of water (200 mL). The resulting solution was extracted with 3×200 mL of ethyl acetate, then the organic layer was washed with brine (100 mL), dried over anhydrous sodium sulfate, and concentrated under vacuum. The residue was applied onto a silica gel column with ethyl acetate/petroleum ether (1:20) to deliver the title compound in 9.8 g (78%) as light yellow oil. LCMS: 347 [M+H]⁺.

### Example 69: Synthesis of (E)-N-methyl-4-((2-((6-((E)-4,4,4-trifluoro-1-(3-fluoro-1H-indazol-5-yl)but-1-en-1-yl)pyridazin-3-yl)oxy)ethyl)amino)but-2-enamide (Compound 69)

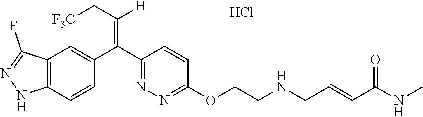 [SMILES: CNC(=O)/C=C/CNCCOc1ccc(/C(=C/CC(F)(F)F)c2ccc3[nH]nc(F)c3c2)nn1 ; Cl]

Compound 69 was synthesized following the approach outlined in Scheme 3, omitting Step-8, by modifying: a) Step-7 by substituting tert-butyl (E)-(2-((6-iodopyridazin-3-yl)oxy)ethyl)(4-(methylamino)-4-oxobut-2-en-1-yl)carbamate (preparation shown below in Steps-a-b) for compound 324, 0.1 equiv of Pd₂(dba)₃.CHCl₃ for Pd(PPh₃)₂Cl₂, using 3.0 equiv of Cs₂CO₃, adding in 0.2 equiv od Davephos, and using a 5:1 ratio of dioxane:H₂O instead of 2-Methyl THF:H₂O, and b) Step-9 by using a 1:1 ratio of TFA:DCM to deliver the title compound in 13.4 mg, 0.54% overall yield, as a brown solid. ¹H NMR (400 MHz, Methanol-d4) δ 8.47-8.40 (bs, 1H), 7.87-7.77 (m, 3H), 7.60-7.56 (s, 2H), 6.81-6.74 (m, 1H), 6.41-6.37 (d, J=15.3 Hz, 1H), 4.90 (s, 2H), 4.04-3.98 (m, 4H), 3.64-3.61 (s, 2H), 2.83 (s, 3H). LCMS: 479.10 [M+H]⁺, 501.10 [M+Na]⁺.

### Step-a: Synthesis of 2-((6-iodopyridazin-3-yl)oxy)ethan-1-amine hydrochloride

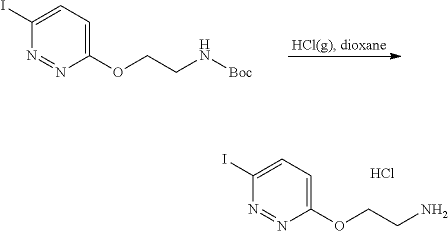 [SMILES: CC(C)(C)OC(=O)NCCOc1ccc(I)nn1 ; "HCl(g), dioxane" ; NCCOc1ccc(I)nn1 ; Cl]

Into a 500-mL round-bottom flask, was placed tert-butyl (2-((6-iodopyridazin-3-yl)oxy)ethyl)carbamate (10 g, 27.38 mmol, 1.00 equiv) (preparation shown in Example 54, Step-a) and hydrogen chloride (4M in dioxane) (100 mL). The resulting solution was stirred at room temperature until completion. The mixture was then concentrated under vacuum to deliver the title compound in 7.3 g (88%) as a yellow solid. The material was taken forward to the next step without further purification.

### Step-b: Synthesis of tert-butyl (E)-(2-((6-iodopyridazin-3-yl)oxy)ethyl)(4-(methylamino)-4-oxobut-2-en-1-yl)carbamate

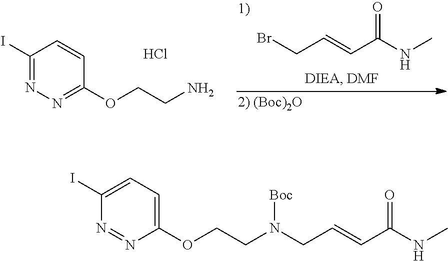

Into a 250-mL round-bottom flask was placed 2-((6-iodopyridazin-3-yl)oxy)ethan-1-amine hydrochloride (7.5 g, 24.87 mmol, 1.00 equiv) and N,N-dimethylformamide (100 mL). This was followed by the addition of DIEA (16 g, 123.80 mmol, 5.00 equiv) and then (E)-4-bromo-N-methylbut-2-enamide (4.4 g, 24.72 mmol, 1.00 equiv) with stirring at 0° C. in portions. The resulting solution was then stirred at room temperature until completion. Then (Boc)₂O (11 g, 50.40 mmol, 2.00 equiv) was added to the mixture. The resulting solution was then stirred at room temperature until completion. The reaction was then quenched by the addition of water (10) mL) and extracted with 3×100 mL of ethyl acetate, then the organic layers were combined, washed with brine (100 mL), and dried over anhydrous sodium sulfate. The residue was applied onto a silica gel column with ethyl acetate/petroleum ether (1:1) to deliver the title compound in 2.0 g (18%) as a brown solid. ¹H NMR (400 MHz, DMSO-d6) δ 8.00-7.91 (m, 2H), 7.02-6.96 (dd, J=17.3, 9.0 Hz, 1H), 6.53-6.49 (m, 1H), 5.90-5.86 (d, J=15.5 Hz, 1H), 4.52-4.48 (q, J=6.5, 5.4 Hz, 2H), 4.04-3.97 (m, 2H), 3.60-3.56 (t, J=5.4 Hz, 2H), 2.66-2.62 (d, J=4.5 Hz, 3H), 1.36-1.33 (m, 9H). LCMS: 463 [M+H]⁺.

### Example 70: Synthesis of (E)-1-(2-(4-(4,4,4-trifluoro-1-(3-fluoro-1H-indazol-5-yl)-2-phenylbut-1-en-1-yl)phenoxy)ethyl)pyrrolidin-2-one (Compound 70)

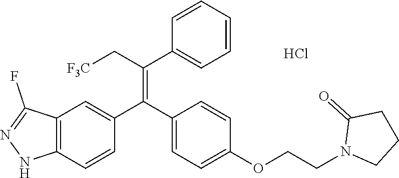 [SMILES: O=C1CCCN1CCOc1ccc(/C(=C(/CC(F)(F)F)c2ccccc2)c2ccc3[nH]nc(F)c3c2)cc1 ; Cl]

Compound 70 was synthesized following the approach outlined in Scheme 3 by modifying: a) Step-7 by substituting 0.8 equiv of 1-(2-(4-iodophenoxy)ethyl)pyrrolidin-2-one (preparation shown below in Steps-a-b) for compound 324, using 0.1 equiv of Pd(PPh₃)₂Cl₂ and a 5:1 ratio of 2-Methyl THF:H₂O, and stirring at 60° C. until completion, b) Step-8 by using 1.5 equiv of bromobenzene, 0.1 equiv of Pd(PPh₃)₂Cl₂, 7.0 equiv of KOH, and a 5:1 ratio of dioxane:H₂O, and c) Step-9 by using a 5:2 ratio of TFA:DCM to deliver the title compound in 246.0 mg, 1.56% overall yield, as a white solid. ¹H NMR (400 MHz. Methanol-d4) δ 7.60 (d, J=1.2 Hz, 1H), 7.50-7.42 (m, 1H), 7.25-6.97 (m, 8H), 6.83-6.81 (m, 1H), 6.61-6.59 (m, 1H), 4.17 (t, J=5.3 Hz, 1H), 4.00-3.98 (t, J=5.2 Hz, 1H), 3.67-3.49 (m, 4H), 3.44-3.36 (d, J=10.5 Hz, 2H), 2.37-2.29 (dt, J=21.4, 8.1 Hz, 2H), 2.02-1.95 (m, 2H). LCMS: 524.4 [M+H]⁺.

### Step-a: Synthesis of 2-(2-oxopyrrolidin-1-yl)ethyl methanesulfonate

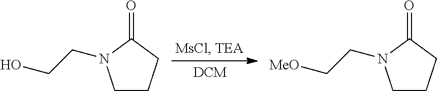 [SMILES: O=C1CCCN1CCO ; COCCN1CCCC1=O]

Into a 8-mL round-bottom flask was placed 1-(2-hydroxyethyl)pyrrolidin-2-one (100 mg, 0.77 mmol, 1.00 equiv), TEA (156.589 mg, 1.55 mmol, 2.00 equiv), and DCM (3 mL), followed by the addition of MsCl (97 mg, 1.10 equiv) at 0° C. The resulting solution was stirred at 25° C. and used directly for the next step without any further purification. LCMS: 208.1 [M+H]⁺.

### Step-b: Synthesis of 1-(2-(4-iodophenoxy)ethyl)pyrrolidin-2-one

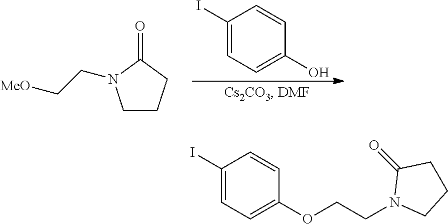 [SMILES: COCCN1CCCC1=O ; Oc1ccc(I)cc1 ; O=C1CCCN1CCOc1ccc(I)cc1]

Into a 40-mL round-bottom flask was placed 2-(2-oxopyrrolidin-1-yl)ethyl methanesulfonate (1 g, 4.83 mmol, 1.00 equiv), Cs₂CO₃ (3.14 g, 9.64 mmol, 2.00 equiv), N,N-dimethylformamide (10 mL), and 4-iodophenol (1.59 g, 7.23 mmol, 1.50 equiv). The resulting solution was stirred at 25° C. until completion. The solution was then diluted with 50 mL of water, then extracted with 3×50 mL of ethyl acetate. The organic layers were combined, washed with 50 mL of brine, dried over anhydrous sodium sulfate, and concentrated under vacuum. The residue was applied onto a silica gel column eluting with DCM/methanol (14:1) to deliver the title compound in 1.1 g (56%) as a white solid. ¹H NMR (400 MHz, Methanol-d4) δ 7.61-7.52 (m, 2H), 6.80-6.71 (m, 2H), 4.10 (t, J=5.3 Hz, 2H), 3.69-3.54 (m, 4H). 2.37 (t, J=8.1 Hz, 2H), 2.03 (qd, J=8.1, 6.8 Hz, 2H). LCMS: 322.0 [M+H]⁺.

### Example 71: Synthesis of (Z)—N-methyl-4-((2-((5-(4,4,4-trifluoro-1-(3-fluoro-1H-indazol-5-yl)-2-phenylbut-1-en-1-yl)pyridin-2-yl)oxy)ethyl)amino)butanamide (Compound 71)

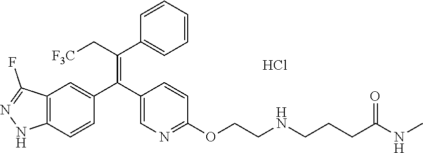 [SMILES: CNC(=O)CCCNCCOc1ccc(/C(=C(/CC(F)(F)F)c2ccccc2)c2ccc3[nH]nc(F)c3c2)cn1 ; Cl]

Compound 71 was synthesized following the approach outlined in Scheme 6, modifying Step-1 by substituting (E)-N-methyl-4-(2-(5-((Z)-4,4,4-trifluoro-1-(3-fluoro-1H-indazol-5-yl)-2-phenylbut-1-enyl)pyridin-2-yloxy)ethylamino)but-2-enamide (preparation shown in Example 3) for compound 336 and THF for MeOH to deliver the title compound in 62.0 mg, 29% overall yield, as a yellow solid. ¹H NMR (400 MHz, Methanol-d4) δ 7.73 (s, 1H), 7.66 (d, J=1.3 Hz, 1H), 7.53-7.50 (m, 1H), 7.35-7.31 (m, 2H), 7.27-7.24 (m, 4H), 7.23-7.20 (m, 1H), 6.68-6.66 (m, 1H), 4.48-4.46 (m, 2H), 3.50-3.35 (m, 4H), 3.15-3.07 (t, J=7.3 Hz, 2H), 2.70 (s, 3H), 2.39-2.35 (t, J=6.7 Hz, 2H), 1.96-1.91 (m, 2H). LCMS: 556 [M+H]⁺.

### Example 72: Synthesis of (E)-4-((2-((5-((Z)-4,4,4-trifluoro-1-(3-fluoro-1H-indazol-1-yl)-2-phenylbut-1-en-1-yl)pyridin-2-yl)oxy)ethyl)amino)but-2-enoic acid (Compound 72)

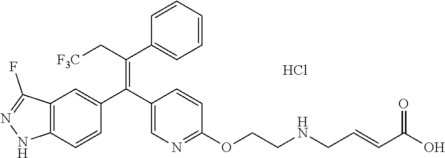 [SMILES: O=C(O)/C=C/CNCCOc1ccc(/C(=C(/CC(F)(F)F)c2ccccc2)c2ccc3[nH]nc(F)c3c2)cn1 ; Cl]

Compound 72 was synthesized following the approach outlined in Scheme 3 by modifying: a) Step-7 by substituting methyl (E)-4-((tert-butoxycarbonyl)(2-((5-iodopyridin-2-yl)oxy)ethyl)amino)but-2-enoate (preparation shown below in Steps-a-b) for compound 324, 0.1 equiv of Pd(ddpf)Cl₂ for Pd(PPh₃)₂Cl₂, using 2.5 equiv of Cs₂CO₃, a 5:1 ratio of dioxane:H₂O, and stirring at 50° C. until completion, b) Step-8 by using 0.1 equiv of Pd(ddpf)Cl₂ for Pd(PPh₃)₂Cl₂, 7.0 equiv of KOH, and a 5:1 ratio of dioxane:H₂O, and c) Step-9 by stirring with just TFA to deliver the title compound in 110.2 mg, 0.65% overall yield, as an off-white solid. ¹H NMR (400 MHz, DMSO-d6) δ 12.78 (s, 1H), 9.43 (s, 2H), 7.68-7.64 (m, 2H), 7.57-7.54 (dd, J=8.8, 2.4 Hz, 1H), 7.30-7.17 (m, 7H), 6.81-6.73 (d, J=15.6, 1H), 6.62-6.60 (d, J=8.6 Hz, 1H), 6.13-6.09 (m, 1H), 4.38-4.36 (t, J=5.2 Hz, 2H), 3.79 (d, J=6.0 Hz, 2H), 3.51-3.43 (m, 2H), 3.23 (s, 2H). LCMS: 541 [M+H]⁺.

### Step-a: Synthesis of methyl (E)-4-((tert-butoxycarbonyl)(2-((5-iodopyridin-2-yl)oxy)ethyl)ammo)but-2-enoate

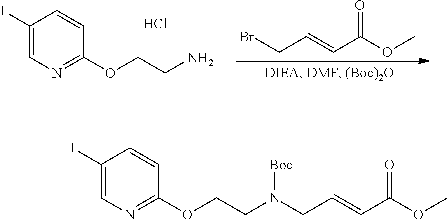

Into a 500-mL round-bottom flask, was placed 2-((5-iodopyridin-2-yl)oxy)ethan-1-amine hydrochloride (10 g, 29.67 mmol, 1.00 equiv) (Scheme 4, Steps-1-2) and N,N-dimethylformamide (200 mL). This was followed by the addition of DIEA (15 g, 116.06 mmol, 4.00 equiv) dropwise with stirring at 0° C. To this, methyl (E)-4-bromobut-2-enoate (3.7 g, 20.67 mmol, 0.70 equiv) was added dropwise. The resulting solution was stirred at room temperature until completion. To the mixture was added (Boc)₂O (13 g, 59.56 mmol, 2.00 equiv). The resulting solution was stirred at room temperature until completion. The reaction was then quenched by the addition of water (100 mL), extracted with 3×200 mL of ethyl acetate, and then the organic layers were combined and concentrated under vacuum. The crude product was purified by Flash-Prep-HPLC with Column C18 using (20%-95%) CH₃CN in water (NH₄HCO₃ 10 mmol/L) to deliver the title compound in 4.0 g (29%) as brown oil. LCMS: 463 [M+H]⁺.

### Example 73: Synthesis of (E)-4-((2-(4-((E)-1-(1H-indazol-5-yl-2-phenylbut-1-en-1-yl)phenoxy)ethyl)amino)but-2-enoic acid (Compound 73)

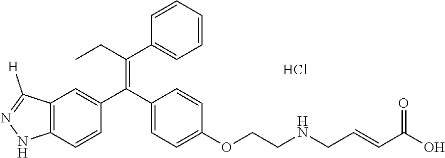 [SMILES: CC/C(=C(/c1ccc(OCCNC/C=C/C(=O)O)cc1)c1ccc2[nH]ncc2c1)c1ccccc1 ; Cl]

Compound 73 was synthesized following the approach outlined in Scheme 10, omitting Steps-1-3, by modifying: a) Step-4 by substituting (E)-4-((2-(4-((E)-1-(1H-indazol-5-yl)-2-phenylbut-1-en-1-yl)phenoxy)ethyl)amino)-N,N-dimethylbut-2-enamide (synthesized following the approach outlined in patent US 2016347717 A1) for compound 357, b) Step-5 by substituting 1.0 equiv of methyl (E)-4-bromobut-2-enoate for compound 359, using 3.0 equiv of DIEA and 2.0 equiv of (Boc)₂O, c) adding an additional step to form (E)-4-((2-(4-((E)-1-(1H-indazol-5-yl)-2-phenylbut-1-en-1-yl)phenoxy)ethyl)(tert-butoxycarbonyl)amino)but-2-enoic acid (preparation shown below in Step-a) and d) Step-6 by using a 10:3 ratio of TFA:DCM to deliver the title compound in 129.0 mg, 15% overall yield, as a red oil. ¹H NMR (400 MHz, Methanol-d4) δ 8.26 (s, 1H), 7.75 (s, 1H), 7.60-7.58 (m, 1H), 7.33-7.30 (m, 1H), 7.19-7.12 (m, 5H), 6.90-6.86 (m, 3H), 6.71-6.69 (m, 2H), 6.24-6.19 (m, 1H), 4.18-4.16 (m, 2H), 3.92-3.90 (m, 2H), 3.44-3.42 (m, 2H), 2.53-2.48 (m, 2H), 0.98-0.94 (t, J=7.4 Hz, 3H). LCMS: 468.0 [M+H]⁺.

### Step-a Synthesis of (E)-4-((2-(4-((E)-1-(1H-Indazol-5-yl)-2-phenylbut-1-en-1-yl)phenoxy)ethyl)(tert-butoxycarbonyl)amino)but-2-enoic acid

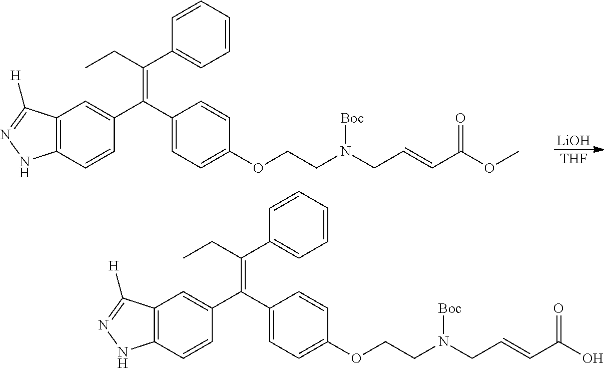

Into a 40-mL round-bottom flask was placed methyl (E)-4-((2-(4-((E)-1-(1H-indazol-5-yl)-2-phenylbut-1-en-1-yl)phenoxy)ethyl)(tert-butoxycarbonyl)amino)but-2-enoate (944 mg, 1.62 mmol, 1.00 equiv), THF (10 mL). LiOH (112.110 mg, 4.68 mmol, 3.00 equiv), and water (2 mL). The resulting solution was stirred for 3 h at 25° C. After completion, the PH of the solution was adjusted to 6 with 6M HCl, and then the solution was filtered to deliver the title compound in 600 mg (26%) as yellow oil. LCMS: 568.1 [M+H]⁺.

### Example 74: Synthesis of (E)-N-methyl-4-((2-((5-((Z)-4,4,4-trifluoro-1-(3-fluoro-1H-indazol-5-yl)-2-phenylbut-1-en-1-yl)pyrazin-2-yl)oxy)ethyl)amino)but-2-enamide (Compound 74)

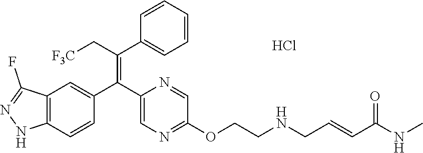 [SMILES: CNC(=O)/C=C/CNCCOc1cnc(/C(=C(/CC(F)(F)F)c2ccccc2)c2ccc3[nH]nc(F)c3c2)cn1 ; Cl]

Compound 74 was synthesized following the approach outlined in Scheme 3 by modifying: a) Step-7 by substituting tert-butyl (E)-(2-((5-iodopyrazin-2-yl)oxy)ethyl)(4-(methylamino)-4-oxobut-2-en-1-yl)carbamate (preparation shown below in Steps-a-e) for compound 324, using 0.1 equiv of Pd(PPh₃)Cl₂, substituting a 5:1 ratio of dioxane:H₂O for 2-Methyl THF:H₂O, and stirring at 50° C. until completion, b) Step-8 by using 1.5 equiv of bromobenzene, 0.1 equiv of KOH, and 0.1 equiv of Pd(PPh₃)Cl₂, and c) Step-9 by using a 1:1 ratio of TFA:DCM to deliver the title compound in 6.1 mg, 0.23% overall yield, as a yellow solid. ¹H NMR (400 MHz, Methanol-d₄) δ 8.14 (s, 1H), 7.76 (s, 1H), 7.67 (s, 1H), 7.52-7.49 (m, 1H), 7.41-7.38 (m, 1H), 7.27-7.20 (m, 5H), 6.73-6.66 (m, 1H), 6.32-6.28 (m, 1H), 4.52-4.50 (m, 2H), 3.88-3.86 (dd, J=6.9, 1.4 Hz, 2H), 3.54-3.42 (m, 4H), 2.81 (s, 3H). LCMS: 555 [M+H]⁺.

### Step-a: Synthesis of 2-((5-iodopyrazin-2-yl)oxy)ethan-1-ol

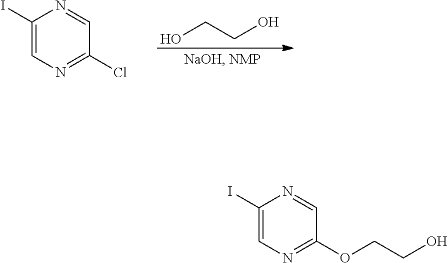

Into a 40-mL vial was placed 2-chloro-5-iodopyrazine (5.0 g, 20.80 mmol, 1.00 equiv), ethane-1,2-diol (3.36 g, 54.13 mmol, 2.60 equiv), sodium hydroxide (1.65 g, 41.25 mmol, 1.98 equiv), and NMP (5 mL). The resulting solution was stirred at 100° C. in an oil bath until completion. The reaction was then quenched by the addition of 200 mL water/ice. The solid was collected by filtration to deliver the title compound in 5.1 g (92%) as a light yellow solid. LCMS: 267 [M+H]⁺.

### Step-b: Synthesis of 2-((5-iodopyrazin-2-yl)oxy)ethyl methanesulfonate

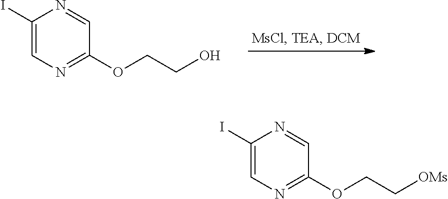 [SMILES: OCCOc1cnc(I)cn1 ; CS(=O)(=O)OCCOc1cnc(I)cn1]

Into a 500-mL 3-necked round-bottom flask was placed 2-((5-iodopyrazin-2-yl)oxy)ethan-1-ol (16 g, 60.14 mmol, 1.00 equiv), TEA (12 g, 118.59 mmol, 2.00 equiv), and DCM (300 mL). This was followed by the addition of MsCl (8.3 g, 1.20 equiv) dropwise with stirring at 0° C. The resulting solution was stirred at room temperature until completion. The reaction was then quenched by the addition of water/ice (100 mL), then extracted with 3×200 mL of ethyl acetate and washed with 100 mL brine. The organic layers were then combined, dried over anhydrous sodium sulfate, and concentrated under vacuum. The residue was applied onto a silica gel column with ethyl acetate/petroleum ether (2:1) to deliver the title compound in 16 g (77%) as a light brown solid. LCMS: 345 [M+H]⁺.

### Step-c: Synthesis of 2-(2-azidoethoxy)-5-iodopyrazine

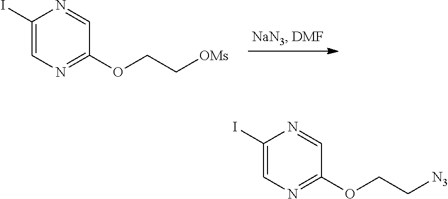 [SMILES: CS(=O)(=O)OCCOc1cnc(I)cn1 ; [N-]=[N+]=NCCOc1cnc(I)cn1]

Into a 500-mL round-bottom flask was placed 2-((5-iodopyrazin-2-yl)oxy)ethyl methanesulfonate (20 g, 58.12 mmol, 1.00 equiv), DMF (300 mL), and NaN₃ (7.5 g, 115.37 mmol, 2.00 equiv). The resulting solution was stirred at 80° C. in an oil bath until completion. The reaction was then quenched by the addition of 200 mL water, extracted with 3×100 mL of ethyl acetate, and washed with 100 mL brine. The organic layers were then combined, dried over anhydrous sodium sulfate, and concentrated under vacuum to deliver the title compound in 8 g (47%) as a brown solid. LCMS: 291 [M+H]⁺.

### Step-d. Synthesis of 2-((5-iodopyrazin-2-yl)oxy)ethan-1-amine

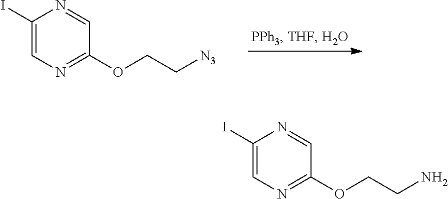 [SMILES: [N-]=[N+]=NCCOc1cnc(I)cn1 ; NCCOc1cnc(I)cn1]

Into a 250-mL round-bottom flask was placed 2-(2-azidoethoxy)-5-iodopyrazine (8 g, 27.49 mmol, 1.00 equiv), PPh₃ (14.4 g, 54.90 mmol, 2.00 equiv), THF (80 mL), and water (20 mL).

The resulting solution was stirred at room temperature until completion, then quenched by the addition of water (100 mL). The pH of the solution was then adjusted to 4-5 with hydrogen chloride, and then was extracted with 3×100 mL of Et₂O. The aqueous layer was isolated and sodium bicarbonate was used to adjust the pH of the solution to 7-8. The resulting solution was extracted with 3×100 mL of ethyl acetate and washed with 100 mL brine. Then the organic layers were combined, dried over anhydrous sodium sulfate, and concentrated under vacuum to deliver the title compound in 5.0 g (69%) as an off-white solid. LCMS: 266 [M+H]⁺.

### Step-e: Synthesis of tert-butyl (E)-(2-((5-iodopyrazin-2-yl)oxy)ethyl)(4-(methylamino)-4-oxobut-2-en-1-yl)carbamate

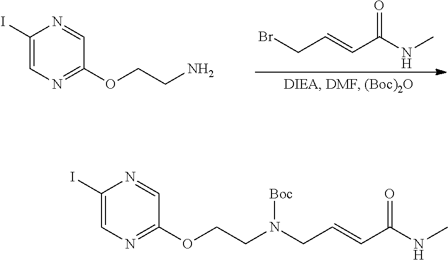

Into a 250-mL round-bottom flask was placed 2-((5-iodopyrazin-2-yl)oxy)ethan-1-amine (7.0 g, 26.41 mmol, 1.00 equiv) and DMF (100 mL). This was followed by the addition of DIEA (14 g, 108.33 mmol, 4.00) equiv), dropwise with stirring, at 0° C. To this solution was added (E)-4-bromo-N-methylbut-2-enamide (4.7 g, 26.40 mmol, 1.00 equiv), in portions, at 0° C. The resulting solution was stirred at room temperature until completion. Then (Boc)₂O (11.5 g, 52.82 mmol, 2.0 equiv) was added. The resulting solution was allowed to react, with stirring, at room temperature until completion. The reaction was then quenched by the addition of 100 mL water, extracted with 3×100 mL of ethyl acetate, and washed with 100 mL brine. Then the organic layers were combined, dried over anhydrous sodium sulfate, and concentrated under vacuum. The residue was applied onto a silica gel column with ethyl acetate/petroleum ether (10:1) to deliver the title compound in 4.0 g (33%) as a yellow solid. ¹H NMR (400 MHz, Chloroform-d) δ 8.31 (d, J=1.4 Hz, 1H), 8.08-8.02 (d, J=9.0 Hz, 1H), 6.78-6.75 (t, J=12.9 Hz, 1H), 5.87-5.75 (dd, J=30.3, 15.3 Hz, 1H), 5.69 (d, J=7.1 Hz. 1H), 4.43 (d, J=5.4 Hz, 2H), 4.05 (d, J=5.8 Hz, 2H), 3.60-3.58 (d, J=9.1 Hz, 2H), 2.90-2.87 (d, J=2.2 Hz, 3H), 1.43 (s, 9H). LCMS: 463 [M+H]⁺.

### Example 75: Preparation of Substituted Analogs

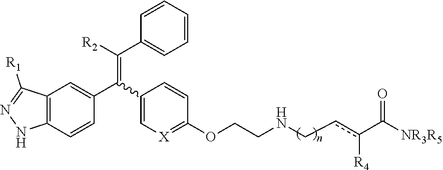

Compounds with the above general formula may be prepared by following a similar reaction sequence to that described in Scheme 1, wherein the R₂ substituent is introduced by using the appropriately substituted iodide in place of 1,1,1-trifluoro-2-iodoethane in Step 5, or by selection of the appropriately substituted TMS-acetylene in place of but-1-yn-1-yltrimethylsilane in Step 3.

The R₃, R₄, and R₅ substituents, the value of “n,” and the presence of a double or single bond at [symbol] are introduced or modified by using the appropriately substituted phenyl (X═C) or pyridyl (X═N) iodide in Step 6, for example:

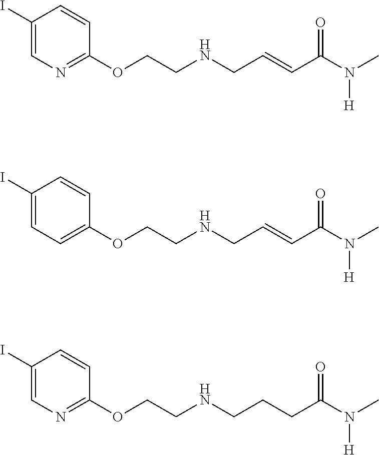 [SMILES: CNC(=O)/C=C/CNCCOc1ccc(I)cn1 ; CNC(=O)/C=C/CNCCOc1ccc(I)cc1 ; CNC(=O)CCCNCCOc1ccc(I)cn1]

The R₁ substituent can be varied by selection of the appropriately substituted starting material in Step 2, for example:

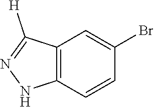

### Example 100—Compounds that Inhibit ERα^{WT/MUT} Activity In Vitro

### Cell Culture

MCF7 BUS cells (Coser, et al., (2003) PNAS 100(24): 13994-13999) were maintained in Dulbecco's Modified Eagle Medium supplemented with 10% FBS, 4 mM L-glutamine and 1× non-essential amino acids. Lenti-X 293T cells (Clontech, Cat #632180) were routinely cultured in Dulbecco's Modified Eagle Medium supplemented with 10% FBS.

Site-Direct Mutagenesis and Cell Line Engineering

The QuikChange II XL Site-Directed Mutagenesis Kit (Agilent Technologies, Cat #200523) was used to generate Y537S, Y537C, Y537N and D538G mutations within the ERα exon 8. Wild-type ESR1 cDNA (GeneCopoeia Inc., Cat # GC-A0322, accession no. NM 000125) was used as a template with the following mutagenesis primers (where the underlined nucleotides represent site mutations); Y537S: F-AAG AAC GTG GTG CCC CTC TCT GAC CTG CTG CTG GAG ATG (SEQ ID NO: 1), R-CAT CTC CAG CAG CAG GTC AGA GAG GGG CAC CAC GTT CTT (SEQ ID NO: 2); Y537N: F-AAG AAC GTG GTG CCC CTC AAT GAC CTG CTG CTG GAG ATG (SEQ ID NO: 3). R-CAT CTC CAG CAG CAG GTC ATT GAG GGG CAC CAC GTT CTT (SEQ ID NO: 4); Y537C: F-AAG AAC GTG GTG CCC CTC TGT GAC CTG CTG CTG GAG ATG (SEQ ID NO: 5), R-CAT CTC CAG CAG CAG GTC ACA GAG GGG CAC CAC GTT CTT (SEQ ID NO: 6); D538G: F-AAC GTG GTG CCC CTC TAT GGC CTG CTG CTG GAG ATG CTG (SEQ ID NO: 7), R-CAG CAT CTC CAG CAG CAG CCC ATA GAG GGG CAC CAC GTT (SEQ ID NO: 8). WT and mutant ESR1 cDNAs were cloned into the designation lentiviral vector pLenti6.3/V5-Dest (Invitrogen, Cat # V533-06). To make lentivirus, DNAs (WT and mutant ESR1) were co-transfected with packaging plasmids into Lenti-X 293T cells using TransIT (Mirus, Cat # MIR 2700). 48 h post-transfection, virus containing media was filtered and added to MCF7 cells in the presence of 8 μg/ml polybrene overnight. Two days following infection, cells were placed under selection with 10 μg/ml blasticidin for 2 weeks for stable expression.

In Vitro Proliferation Assays

MCF7-WT and -Y537S cells were seeded at 1500 cells/well in black-walled 96-well plates (assay plates, Costar, Cat #3904). In parallel, cells were also seeded in a separate 96-well plate (8 wells/cell line, control plate) for which a CTG (CellTiter-Glo@ Luminescent Viability Assay. Promega, Cat # G7572) was measured the following day (day 0 reading). The day 0 reading was used for the GI₅₀ calculation at the termination of the experiment. The day following seeding, compounds were added to assay plates. Briefly, a 1:4 serial dilution was prepared in DMSO at 200× final concentration for a total of 10 concentrations (9 dilutions containing compound and one is DMSO only). Serially diluted compounds were pipetted into medium to prepare a compound-medium mix at 10× final concentration. 10 μl of compound-medium mix was added to MCF7-WT and -Y537S cells at 3 wells/concentration (triplicate for each concentration). On day 3, media-compound was removed and replaced with fresh media/compound as described above. On day 6, CTG was measured and compared to day 0 readings from control plate to assess GI₅₀.

Results

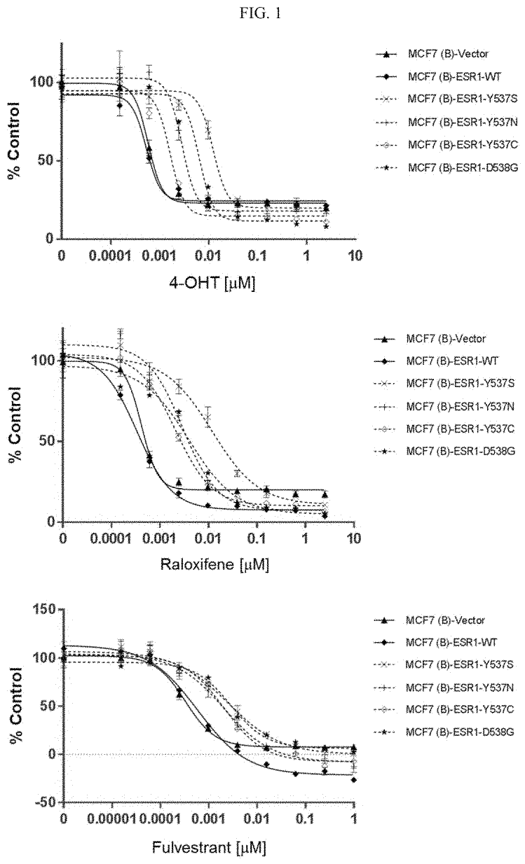

FIG. 1 shows that ectopic expression of ERα^{Y537S/N/C, D538G} in MCF7 cells conferred phenotypic resistance to currently marketed therapies tamoxifen (SERM), raloxifene (SERM) and fulvestrant (SERD). Similar observations were also recently published by several independent labs (Jeselsohn et al., (2014) Clin. Cancer Res. April 1; 20(7):1757-67: Toy et al., (2013) Nat Genet. 2013 December; 45(12): 1439-45; Robinson et al., (2013) Nat Genet December; 45(12):1446-51; Merenbakh-Lamin et al., (2013) Cancer Res. December 1; 73(23):6856-64; Yu et al., (2014) Science July 11; 345(6193):216-20). Having confirmed that ERα^{MUT} drive resistance to current endocrine therapies, identification of novel compounds that would reduce proliferation of the ERα^{MUT}-bearing MCF7 cells more efficaciously than the corresponding clinical compound 4-hydroxytamoxifen was sought. Using the WT and mutant viability assay as a screening tool, compounds were identified that were more potent towards the Y537S-bearing MCF7 line relative to 4-hydroxytamoxifen. The results of the viability assay screen are shown in Table 2. These assays were conducted with free base and/or salt forms of the compounds identified in the table.

TABLE 2
Viability Screen Results

| Compound # | MCF7 WT GI50 (nM) | MCF7 Y537S GI50 (nM) |
|---|---|---|
| 1 | 0.36 ± 0.16 (4) | 3.79 ± 1.92 (4) |
| 2 | 0.28 ± 0.15 (12) | 3.52 ± 2.18 (12) |
| 3 | 0.76 ± 0.44 (25) | 8.38 ± 6.81 (25) |
| 4 | 4.09 ± 2.74 (11) | 46.36 ± 23.23 (11) |
| 5 | 88.54 ± 42.72 (2) | 180.09 ± 227.37 (2) |
| 6 | 0.38 ± 0.00 (2) | 5.50 ± 0.47 (2) |
| 7 | 0.90 ± 0.78 (3) | 10.47 ± 6.60 (3) |
| 8 | 0.70 ± 0.27 (2) | 14.68 ± 0.55 (2) |
| 9 | 0.88 ± 1.18 (18) | 9.23 ± 8.19 (18) |
| 10 | 2.36 ± 2.81 (16) | 25.18 ± 27.68 (16) |
| 11 | 2.08 ± 1.55 (3) | 32.70 ± 22.16 (3) |
| 12 | 1.45 | 23.98 |
| 13 | 0.69 ± 0.57 (8) | 7.99 ± 5.47 (8) |
| 14 | 1.73 ± 0.14 (2) | 7.65 ± 3.38 (2) |
| 15 | 0.35 ± 0.07 (16) | 5.45 ± 3.93 (16) |
| 16 | 0.59 ± 0.51 (8) | 6.50 ± 6.96 (8) |
| 17 | 0.61 ± 0.11 (2) | 2.63 ± 0.59 (2) |
| 18 | 0.72 ± 0.24 (2) | 4.91 ± 0.65 (2) |
| 19 | 0.66 ± 0.05 (2) | 3.15 ± 0.01 (2) |
| 20 | 0.66 ± 0.13 (2) | 6.08 ± 2.69 (2) |
| 21 | 0.75 ± 0.38 (16) | 10.16 ± 11.24 (16) |
| 22 | 1.62 ± 0.87 (2) | 6.19 ± 4.84 (2) |
| 23 | 0.48 ± 0.21 (3) | 2.29 ± 1.80 (3) |
| 24 | 0.45 ± 0.11 (4) | 3.34 ± 1.14 (4) |
| 25 | 0.38 ± 0.16 (2) | 1.58 ± 0.23 (2) |
| 26 | 14.23 | 453.17 |
| 27 | 2.00 ± 1.46 (3) | 17.13 ± 10.09 (3) |
| 28 | 4.12 ± 1.60 (2) | 9.08 ± 5.26 (2) |
| 29 | 1.37 ± 0.60 (3) | 13.69 ± 6.83 (3) |
| 30 | 1.77 ± 0.39 (3) | 14.50 ± 4.80 (3) |
| 31 | 1.26 ± 1.51 (3) | 11.11 ± 16.06 (3) |
| 32 | 0.83 ± 0.56 (3) | 9.07 ± 4.74 (3) |
| 33 | 1.22 ± 0.46 (4) | 20.87 ± 13.84 (4) |
| 34 | 1.86 ± 1.24 (4) | 11.65 ± 5.29 (4) |
| 35 | 3.06 ± 0.25 (2) | 27.26 ± 19.93 (2) |
| 36 | 1.16 ± 0.48 (3) | 13.80 ± 1.57 (3) |
| 37 | 1.31 ± 0.32 (5) | 13.81 ± 9.61 (5) |
| 38 | 0.61 ± 0.16 (2) | 11.91 ± 4.13 (2) |
| 39 | 0.64 ± 0.23 (4) | 6.86 ± 1.59 (4) |
| 40 | 0.41 ± 0.07 (2) | 8.20 ± 2.67 (2) |
| 41 | 0.24 ± 0.04 (2) | 9.38 ± 7.61 (2) |
| 42 | 1.39 ± 0.56 (2) | 28.38 ± 32.65 (2) |
| 43 | 0.15 | 3.21 |
| 44 | 0.51 ± 0.29 (3) | 4.45 ± 6.12 (3) |
| 45 | 23.21 ± 9.42 (2) | 71.04 ± 8.63 (2) |
| 46 | 1.38 ± 0.44 (2) | 13.85 ± 5.10 (2) |
| 47 | 0.64 ± 0.40 (3) | 7.25 ± 8.77 (3) |
| 48 | 0.80 ± 0.14 (2) | 8.25 ± 2.60 (2) |
| 49 | 172.66 ± 51.68 (2) | 2,422.54 ± 709.43 (2) |
| 50 | 4.00 ± 1.16 (8) | 98.46 ± 81.50 (8) |
| 51 | 9.82 ± 1.92 (2) | 190.27 ± 50.08 (2) |
| 52 | 1.2 | 9.3 |
| 53 | 0.3 | 1.8 |
| 54 | 892.06 ± 527.65 (4) | 7,685.65 ± 3,298.37 (4) |
| 55 | 0.54 ± 0.10 (3) | 7.13 ± 1.13 (3) |
| 56 | 0.49 ± 0.24 (3) | 2.59 ± 2.37 (3) |
| 57 | 0.64 ± 0.38 (3) | 5.06 ± 5.72 (3) |
| 58 | 0.33 ± 0.12 (4) | 3.97 ± 2.25 (4) |
| 59 | 0.79 ± 0.71 (3) | 14.21 ± 12.08 (3) |
| 60 | 0.71 ± 0.53 (5) | 6.72 ± 4.49 (5) |
| 61 | 0.33 ± 0.02 (2) | 1.27 ± 0.17 (2) |
| 62 | 3.31 ± 0.43 (2) | 14.53 ± 4.32 (2) |
| 63 | 17.90 ± 8.66 (3) | 117.34 ± 11.59 (3) |
| 64 | 153.05 ± 27.39 (4) | 673.30 ± 167.03 (4) |
| 65 | 58.30 ± 48.14 (5) | 310.30 ± 139.48 (5) |
| 66 | 436.64 ± 55.20 (2) | >10,000.00 ± 0.00 (2) |
| 67 | 5.38 ± 0.66 (2) | 87.72 ± 55.02 (2) |
| 68 | 21.99 ± 2.66 (2) | 101.06 ± 53.04 (2) |
| 69 | 8,077.25 ± 1,000.73 (2) | 9,397.66 ± 851.84 (2) |
| 70 | 0.95 ± 0.25 (2) | 6.18 ± 2.51 (2) |
| 71 | 0.62 | 5.31 |
| 72 | 1.86 ± 0.87 (3) | 22.22 ± 14.27 (3) |
| 73 | 7.34 ± 0.58 (2) | 154.54 ± 60.12 (2) |
| 74 | 2.47 ± 0.18 (2) | 29.44 ± 11.51 (2) |

Results as presented in Table 2 are presented as the average of one or more trials with standard deviations where available. The number of trials for each compound is presented in parentheses following the value. Those skilled in the art would appreciate that the examples and embodiments reported herein are for illustrative purposes only. Various modifications or changes in light thereof will be suggested to persons skilled in the art, and those modifications or changes are included within the spirit and purview of this application and scope of the appended claims. For example, those skilled in the art will appreciate that GI50 values may vary depending on the lot of fetal bovine serum (FBS), among other factors, used to supplement the culture media, due to varying concentrations of estrogen between batches.

Compounds were tested as prepared in the Examples below with regard to use of free base or salt; results for Compound 3 and Compound 21 are presented as averages of both free base and HCl salt trials. Tests were not conducted for Compounds 75-89.

In Vivo Xenograft Methods

Methods and Materials

Although not wishing to be bound by theory, applicant appreciates that certain in vivo xenograft studies may be useful in identifying effective compounds. Such studies may be conducted, for example, using compounds reported herein and/or their salts. In studies reported herein, the hydrochloride salt form as described herein was used. The WHIM20 xenograft study reported below has not yet been conducted with compounds reported herein, but the Y537S positive ST941 PDx xenograft study and studies in the ERα wild-type MCF7 and ST1799 PDx models have been conducted with certain of the compounds as set forth below.

### Example 101—Y537S Positive ST941 PDX Xenograft Study

A Patient-Derived Xenograft (PDX) tumor model representing an ESR1-Y537S mutated human ER+ breast cancer, designated as ST941 PDX-Y537S, was propagated subcutaneously in immunocompromised mice. (Cf. Wick M J, et al., Establishment and characterization of ST941/C; an ESR1-mutant ER+ breast cancer cell line and xenograft from a patient with acquired resistance to endocrine therapy, Proceedings of the 2016 San Antonio Breast Cancer Symposium; 2016 Dec. 6-10; San Antonio, Tex. Philadelphia (Pa.): AACR; Cancer Res 2017; 77(4 Suppl):Abstract nr P3-04-26). The tumors were excised within 60 days of implantation and processed to mixed tumor fragments. Solid tumor tissues were depleted of necrotic components, cut into 70 mg fragments, mixed with matrigel and subcutaneously implanted into the right flank of 6-12 week old female athymic Nude (Crl:NU(NCr)-Foxn1nu) mice. The precise number of fragments and volume of matrigel were determined on a case by case basis. When the average tumor volume reaches approximately 125-250 mm³, animals were randomized prior to treatment. All of the primary human tumors utilized in this study had undergone approximately 5-7 passages in vivo.

Estrogen was not supplemented in the studies. All tested compounds were dosed orally every day at doses ranging from 3 to 30 mg/kg. The administration volume was calculated from the individual mouse body weights prior to dose administration. The body weights (BW) and tumor volumes (TV) were measured twice a week.

Tumor volumes (TV) were calculated based on the following formula:

TV=length×width²×0.5

length: largest diameter of tumor (mm)

width: diameter perpendicular to length (mm)

The Tumor Growth Inhibition % (TGI) was calculated according to the following formula:

$$Tumor Growth Inhibition \% ( TGI ) = \frac{Average Control TV Day X - Treatment TV Day X}{Average Control TV Day X} \times 100$$

Where Day X is the endpoint measurement.

### Example 101.1 Results for Y537S Positive ST941 PDx Xenograft Studies

Compound 3

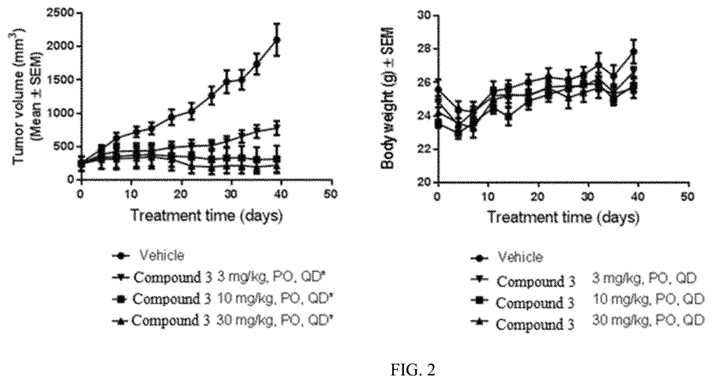

FIG. 2 shows the anti-tumor and body weight effects of Compound 3, prepared as a hydrochloride salt, in the ST941 PDX-Y537S model bearing a heterozygous ERα^{Y537S/WT} xenograft grown in immunocompromised mice. Compound 3 inhibited xenograft growth in a dose dependent manner with 3 mg/kg QD. 10 mg/kg QD and 30 mg/kg QD significantly inhibiting growth on day 39 compared to vehicle control (TGI of 63%, 85%, and 89%, and p<0.0001 for all doses, respectively). All doses and regimens were well tolerated with no significant body weight loss.

Compound 3 was given orally once daily for the duration of the study. Data represent the mean±SEM (Tumor Volume), or the mean±SEM (Body Weight) (N=8 for all groups). * p<0.0001 versus vehicle control on Day 39 (Two-Way ANOVA followed by the Dunnett multiple comparison post hoc test).

Compound 21

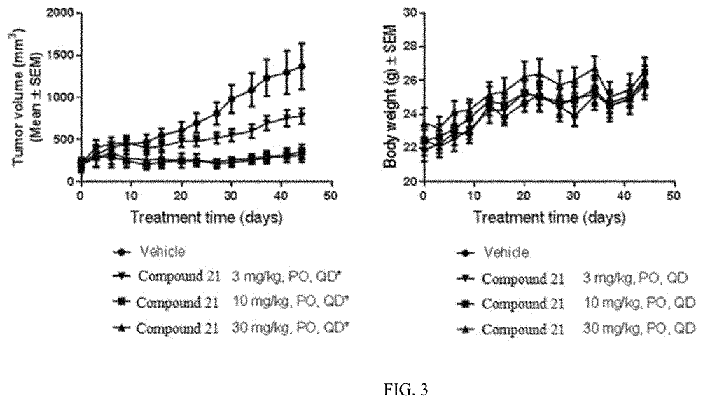

FIG. 3 shows the anti-tumor and body weight effects of compound Compound 21, prepared as an HCl salt, in the ST941 PDX-Y537S model bearing a heterozygous ERα^{Y537/WT} xenograft. Compound 21 dosed daily inhibited xenograft growth in a dose dependent manner with 3 mg/kg QD, 10 mg/kg QD, and 30 mg/kg QD treatments significantly inhibiting growth on day 44 relative to vehicle control (TGI of 43%, 74%, and 77%, and p<0.05, respectively). All doses and regimens were well tolerated with no significant body weight loss.

Compound 21 was given orally once daily for the duration of the study. Data represent the mean±SEM (Tumor Volume) or mean±SEM (Body Weight) (N=6 for all groups). * p<0.05 versus vehicle control on Day 44 (repeated measures t-test, Holm-Sidak method with α=0.05 without assuming a consistent SD).

### Example 102—MCF7Xenograft Studies

The ESR1 wild-type human ER+ breast cancer cell line MCF7 (ATCC) was cultured in DMEM media supplemented with 10% FBS at 37° C. in a 5% CO₂ atmosphere and kept in the exponential growth phase. The cells were collected in trypsin and re-suspended in a 1:1 mixture of matrigel and HBSS at a final concentration of 5×10⁷ cells/mL. A 0.2 mL aliquot of cells was injected subcutaneously into the 3^{rd} mammary fat pad of 6-8 week old female Balb/c nude mice, giving 1×10⁷ cells/mouse. When the average tumor volume reached approximately about 200 mm³, animals were randomized prior to treatment. Estrogen was supplemented for the duration of the study.

All of the compounds were dosed orally every day at doses ranging from 1 to 10 mg/kg. Each treatment was started on Day 0 and the administration schedule was continued for 28 days. The administration volume was calculated from the individual mouse body weights prior to dose administration. The body weights (BW) were measured daily while the tumor volumes were measured twice a week. Tumor volumes (TV) were calculated based on the above formula.

### Example 102.1 Results for MCF7Xenograft Studies

Compound 21

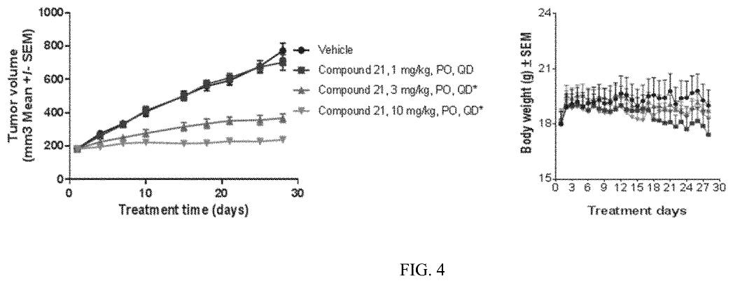

FIG. 4 shows the anti-tumor and body weight effects of Compound 21, prepared as an HCl salt, in the MCF7 tumor model bearing ERα^{WT/WT} xenograft. Compound 21 dosed daily inhibited xenograft growth in a dose dependent manner with 1 mg/kg QD, 3 mg/kg QD, and 10 mg/kg QD treatments inhibiting growth on day 28 relative to vehicle control (TGI of 9.2%, 52.4%, and 69.3%, and p<0.05 for 3 and 10 mg/kg groups, respectively). All doses and regimens were well tolerated with no significant body weight loss.

Compound 21 was given orally once daily for the duration of the study. Data represent the mean±SEM (Tumor Volume) or mean±SEM (Body Weight) (N=8 for all groups). * p<0.05 versus vehicle control on Day 28 (repeated measures t-test, Holm-Sidak method with α=0.05 without assuming a consistent SD).

### Example 103 WHIM20 Xenograft Studies

The Patient-Derived Xenograft (PDX) tumor model, WHIM20, representing an ESR1-Y537S mutated human ER+ breast cancer is propagated in mice. The tumors are excised and processed to mixed tumor fragments and the fragments are re-implanted subcutaneously into new recipient mice. Solid tumor tissues are depleted of necrotic components, cut into fragments, mixed with matrigel and subcutaneously implanted into the right flank of 6-8 week old female SCID-bg mice. The precise number of fragments and volume of matrigel are determined on a case by case basis. When the average tumor volume reaches approximately 200 mm³, animals are randomized prior to treatment. All of the primary human tumors utilized in this study undergo approximately 4 passages in vivo.

Estrogen is not supplemented in WHIM20 studies. Compounds are dosed orally every day at the indicated doses. Each treatment is started on Day 0 and the administration schedule is continued for the indicated days. The administration volume is calculated from the individual mouse body weights prior to dose administration. The body weights are measured daily while the tumor volumes are measured twice a week. Tumor volumes are calculated based on the previously described formula.

### Example 104 ERα^{WT} ST1799 PDX Xenograft Studies

A PDX tumor model representing an ESR1-WT human ER+ breast cancer, designated as ST1799 PDX-WT, was propagated subcutaneously in immunocompromised mice. (Cf. Wick M J, et al., Establishment and characterization of ESR1-mutant breast cancer PDX models, Proceedings of the Thirty-Eighth Annual CTRC-AACR San Antonio Breast Cancer Symposium: 2015 Dec. 8-12; San Antonio. Tex. Philadelphia (Pa.): AACR; Cancer Res 2016; 76(4 Suppl):Abstract nr P3-03-04.) The tumors were excised within 60 days of implantation and processed to mixed tumor fragments. Solid tumor tissues were depleted of necrotic components, cut into 70 mg fragments, mixed with matrigel and subcutaneously implanted into the right flank of 6-12 week old female athymic Nude (Crl:NU(NCr)-Foxn1nu) mice. The precise number of fragments and volume of matrigel were determined on a case by case basis. When the average tumor volume reached approximately 125-250 mm³, animals were randomized prior to treatment. Estrogen was supplemented for the duration of the study. Compound 21 was dosed orally every day at doses ranging from 1 to 30 mg/kg. The administration volume was calculated from the individual mouse body weights prior to dose administration. The body weights (BW) and tumor volumes (TV) were measured twice a week.

### Example 104.1 Results for ERα^{WT/WT} ST1799 PDX Xenograft Studies

Compound 21

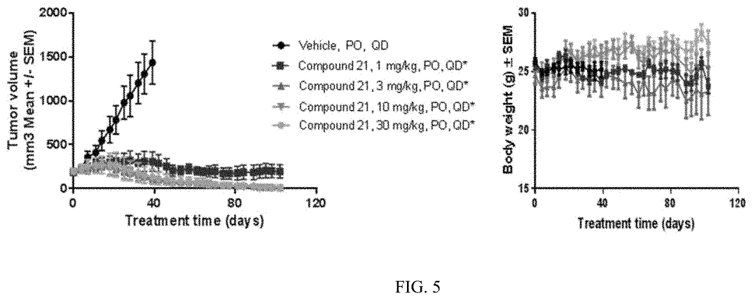

FIG. 5 shows the anti-tumor and body weight effects of Compound 21, prepared as an HCl salt, in a ST1799 PDX model bearing ERα^{WT/WT} xenograft. Compound 21 dosed daily inhibited xenograft growth in a dose dependent manner with 1 mg/kg QD, 3 mg/kg QD, 10 mg/kg QD, and 30 mg/kg QD treatments significantly inhibiting growth on day 39 relative to vehicle control (TGI of 78.5%, 92.3%, 93.1%, and 90.7%, and p<0.05, respectively). All doses and regimens were well tolerated with no significant body weight loss.

Compound 21 was given orally once daily for the duration of the study. Data represent the mean±SEM (Tumor Volume) or mean±SEM (Body Weight) (N=6 for all groups). * p<0.05 versus vehicle control on Day 39 (repeated measures t-test, Holm-Sidak method with α=0.05 without assuming a consistent SD).

### Example 105 Time-Dependent Inhibition Assays

To demonstrate that a compound is (or is not) a CYP inactivator, abbreviated experimental designs are commonly used for screening in drug development. One of these approaches uses multiple test compound concentrations at a single incubation time, e.g. the “IC₅₀ shift” approach. In an IC₅₀ shift experiment, the IC₅₀ is determined for a CYP marker activity before and after the test compound has been incubated with enzyme and the co-factor nicotinamide adenine dinucleotide phosphate (NADPH) for a set preincubation time (Grimm et al, 2009).

The IC₅₀ shift approach was used to determine whether compounds are time-dependent inhibitors of human CYP3A4 using liver microsomes (0.1 mg/mL). A 30-minute pre-incubation time point was selected, where compounds (9 concentrations, 0 to 30 μmol/L) are incubated at 37° C. in presence and absence of 1 mmol/L NADPH. Following the pre-incubation period, 5 μmol/L midazolam (the probe substrate) was added and formation of hydroxymidazolam was measured by high-performance liquid chromatography-mass spectrometry (LC-MS/MS) analysis following a 5 minute incubation period. Any decrease in the formation of hydroxymidazolam, in peak area ratios to vehicle control, was used to calculate three IC₅₀ values (0 min pre-incubation, 30 min pre-incubation with NADPH, and 30 min pre-incubation without NADPH). Whereas an IC₅₀ shift<3-fold is accepted as demonstration that a test compound does not possess a TDI risk, an IC₅₀ shift ≥3-fold after pre-incubation is indicative of a CYP3A4 TDI risk. Assays were performed in duplicate, and mifepristone was used as positive control. Results are shown in Table 3. In Table 3, “Yes” indicates a TDI shift greater than or equal to 3. “No” indicates a TDI shift greater than or equal to 1 and less than 3.

TABLE 3

| Compound | (IC₅₀ 0 min) | CYP3A4 |
|---|---|---|
| Number | (̅I̅C̅₅̅₀̅ 3̅0̅ m̅i̅n̅ +̅ N̅A̅D̅P̅H̅)̅ | TDI |
| 2 | 4.4 | Yes |
| 3 | 2.1 | No |
| 7 | 2.2 | No |
| 9 | 3.0 | Yes |
| 10 | 5.5 | Yes |
| 13 | 1.0 | No |
| 15 | 2.3 | No |
| 16 | 16.0 | Yes |
| 17 | 1.6 | No |
| 21 | 1.3 | No |
| 23 | 15.8 | Yes |
| 26 | 1.2 | No |
| 27 | 3.5 | Yes |
| 29 | 1.4 | No |
| 30 | 2.2 | No |
| 31 | 3.9 | Yes |
| 32 | 5.3 | Yes |
| 33 | 9.2 | Yes |
| 34 | 3.7 | Yes |
| 36 | 5.7 | Yes |
| 37 | 10.4 | Yes |
| 38 | 4.2 | Yes |
| 39 | 1.8 | No |
| 40 | 11.0 | Yes |
| 44 | 5.1 | Yes |
| 55 | 14.6 | Yes |
| 56 | 1.7 | No |
| 57 | 0.9 | No |
| 59 | 25.4 | Yes |
| 60 | 2.0 | No |
| 61 | 2.9 | No |

By way of comparison with the results above, a TDI assay was also conducted for the following compound, which is reported as Compound 69 of PCT International Application Publication No. WO/2016/196346:

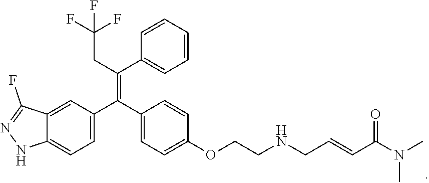 [SMILES: CN(C)C(=O)/C=C/CNCCOc1ccc(/C(=C(/CC(F)(F)F)c2ccccc2)c2ccc3[nH]nc(F)c3c2)cc1]

The IC₅₀ shift approach was also used to determine whether Compound 69 of PCT International Application Publication No. WO/2016/196346 is a time-dependent inhibitor of human CYP3A4 using liver microsomes. Similar to the experimental design described above, a 30-minute pre-incubation time point was selected. Minor variations to the method described above were that Compound 69 of PCT International Application Publication No. WO/2016/196346 was tested at 8 concentrations (0 to 10 μmol/L). Following the pre-incubation period, 3 μmol/L midazolam was added and formation of hydroxymidazolam was measured following a 2 minute incubation period. Since under these experimental conditions, the formation of hydroxymidazolam followed first order kinetics (considered as a linear process), these changes in assay design are not expected to impact the IC₅₀ determinations. In parallel, the IC₅₀ shift assay was also performed with 15.6 μmol/L testosterone (0.05 mg/mL liver microsomes, 10 minute incubation) as second probe substrate. Assays for both probe substrates were performed in triplicate, and mifepristone was used as positive control.

The CYP3A4 TDI shift result for Compound 69 of PCT International Application Publication No. WO/2016/196346 was greater than 3, indicating it to be a TDI risk.

It will now be apparent that new, improved, and nonobvious compositions have been described in this specification with sufficient particularity as to be understood by one of ordinary skill in the art. Moreover, it will be apparent to those skilled in the art that modifications, variations, substitutions, and equivalents exist for features of the compositions which do not materially depart from the spirit and scope of the embodiments disclosed herein. Accordingly, it is expressly intended that all such modifications, variations, substitutions, and equivalents which fall within the spirit and scope of the invention as defined by the appended claims shall be embraced by the appended claims.

1. A compound given by Formula I: 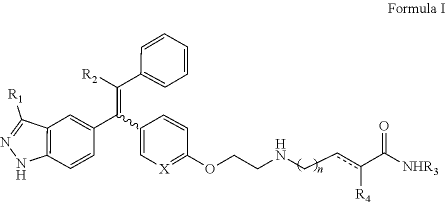
wherein:
R₁ is —H, —CH₃, or —F;
R₂ is —CH₂CH₃, —CH₂CF₃, or cyclobutyl;
R₃ is
i) selected from —H, —CH₃, and —CH₂CH₂OH, or
ii) forms a 5-7 membered heterocycloalkyl ring with R₄ and the N to which R₃ is attached;
wherein R₄ is —H when it does not form said 5-7-membered heterocycloalkyl ring with R₃;
X is N or C;
n is 1-2; and [symbol]
represents a single bond or a double bond;
or a pharmaceutically acceptable salt thereof.

2. (canceled)

3. The compound or pharmaceutically acceptable salt of claim 1, having the following stereochemistry:

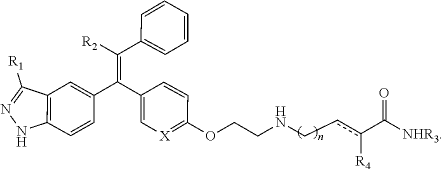

4. A compound given by Formula II: 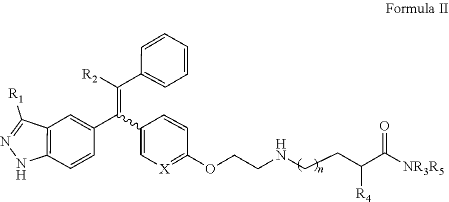
wherein:
R₁ is —H or —F;
R₂ is —CH₂CH₃, —CH₂CF₃, or cyclobutyl;
R₃
i) is selected from —H, —CH₃, and —CH₂CH₂OH, or
ii) forms a 4-6 membered heterocycloalkyl ring with R₅ and the N to which R₃ and R₅ are attached, optionally with an additional heteroatom in the 4-6 membered heterocycloalkyl ring; or
iii) forms a 5-7 membered heterocycloalkyl ring with R₄ and the N to which R₃ is attached;
wherein R₄ is —H when it does not form said 5-7-membered heterocycloalkyl ring with R₃;
wherein R₅ is —H, —CH₃, and —CH₂CH₂OH when it does not form said 4-6 membered heterocycloalkyl ring with R₃;
X is N or C; and
n is 1-2; or a pharmaceutically acceptable salt thereof.

5. The compound or pharmaceutically acceptable salt of claim 4, having the following stereochemistry:

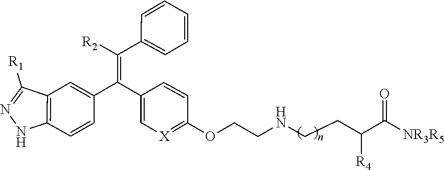

6. A compound of Formula III or pharmaceutically acceptable salt thereof: 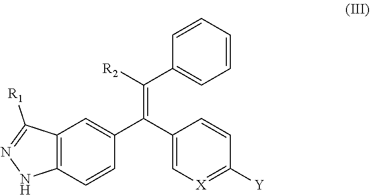
wherein R₁ is H or F;
R₂ is —CH₂CH₃, —CH₂CF₃, or cyclobutyl;
X is C or N;
and Y is selected from the group consisting of

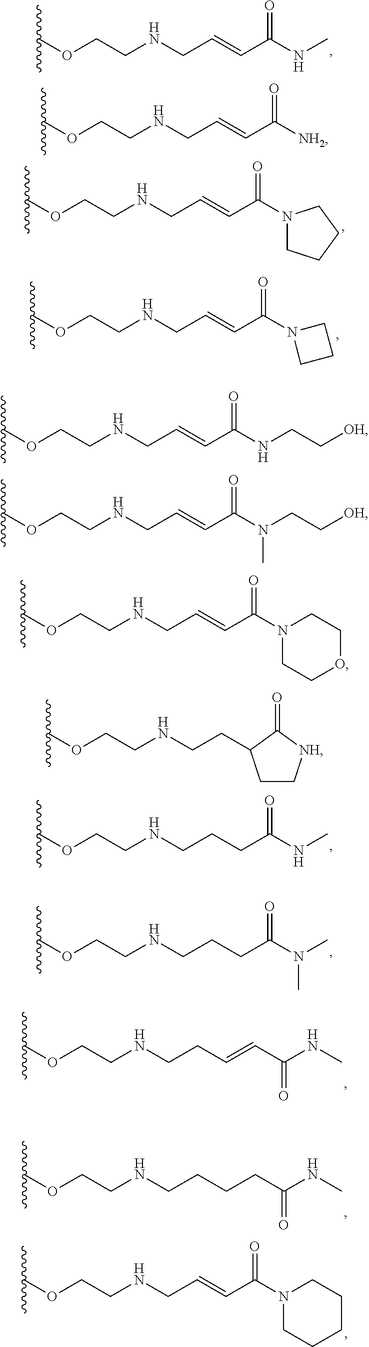

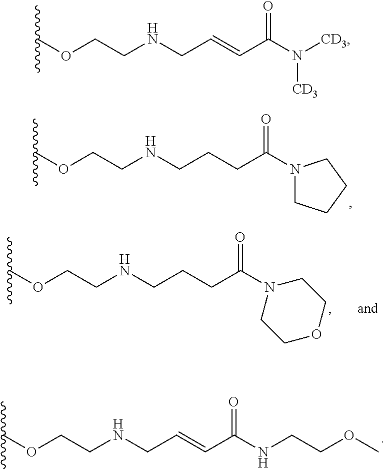

7. The compound or pharmaceutically acceptable salt of claim 6, wherein Y is selected from the group consisting of

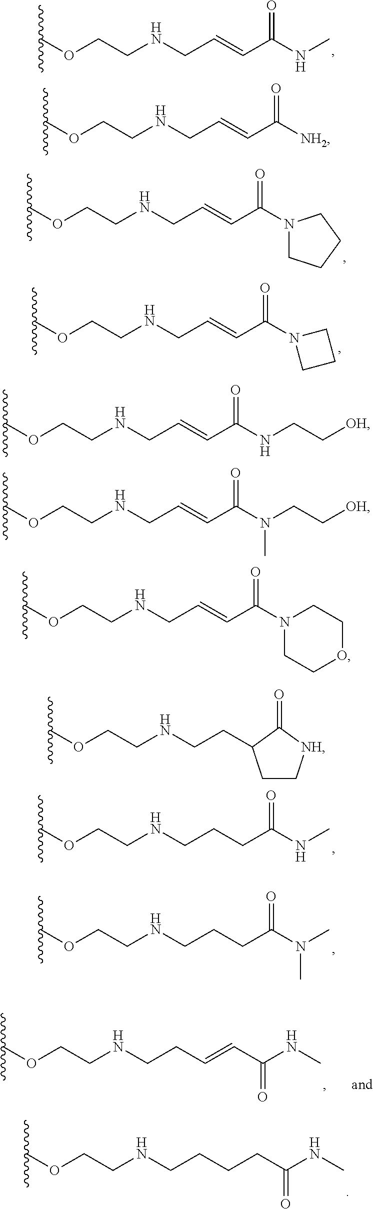

8. The compound or pharmaceutically acceptable salt of claim 3, wherein R₁ is —H or —F.

9-11. (canceled)

12. The compound or pharmaceutically acceptable salt of claim 3, wherein [symbol] represents a double bond.

13. The compound or pharmaceutically acceptable salt of claim 3, wherein n is 1.

14. The compound or pharmaceutically acceptable salt of claim 3, wherein R₃ is —CH₃.

15. A compound selected from the group consisting of: 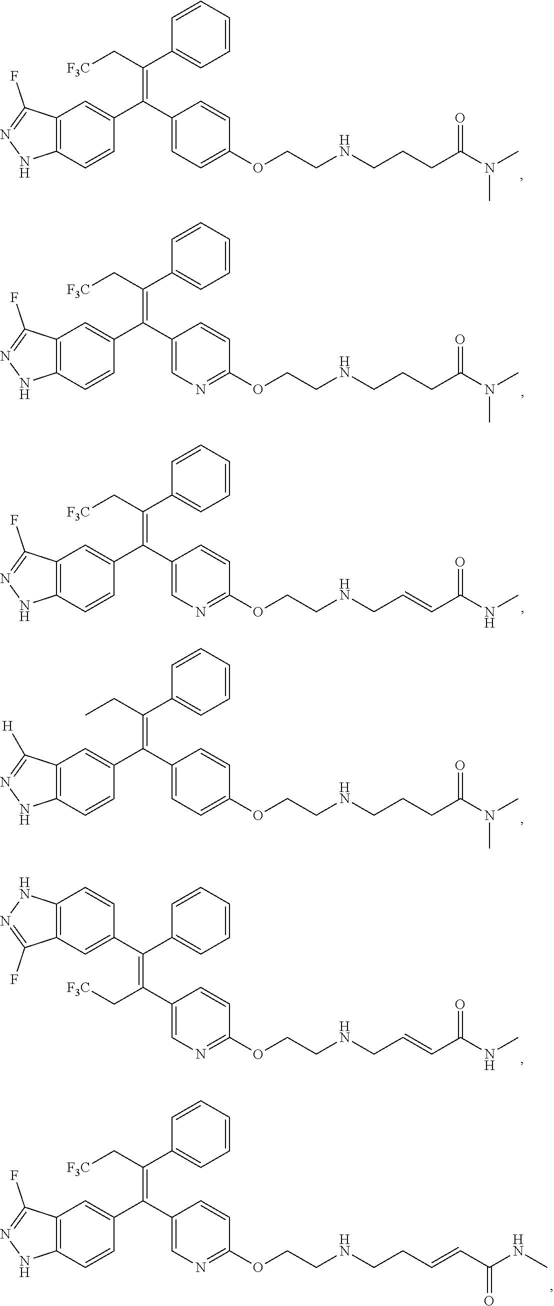 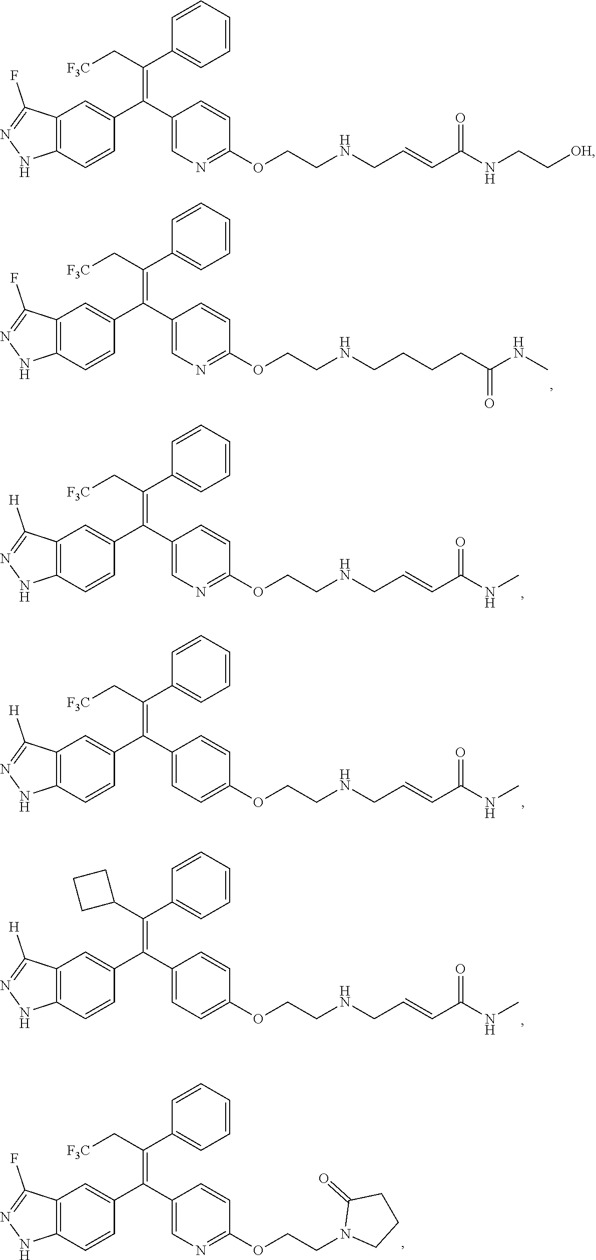 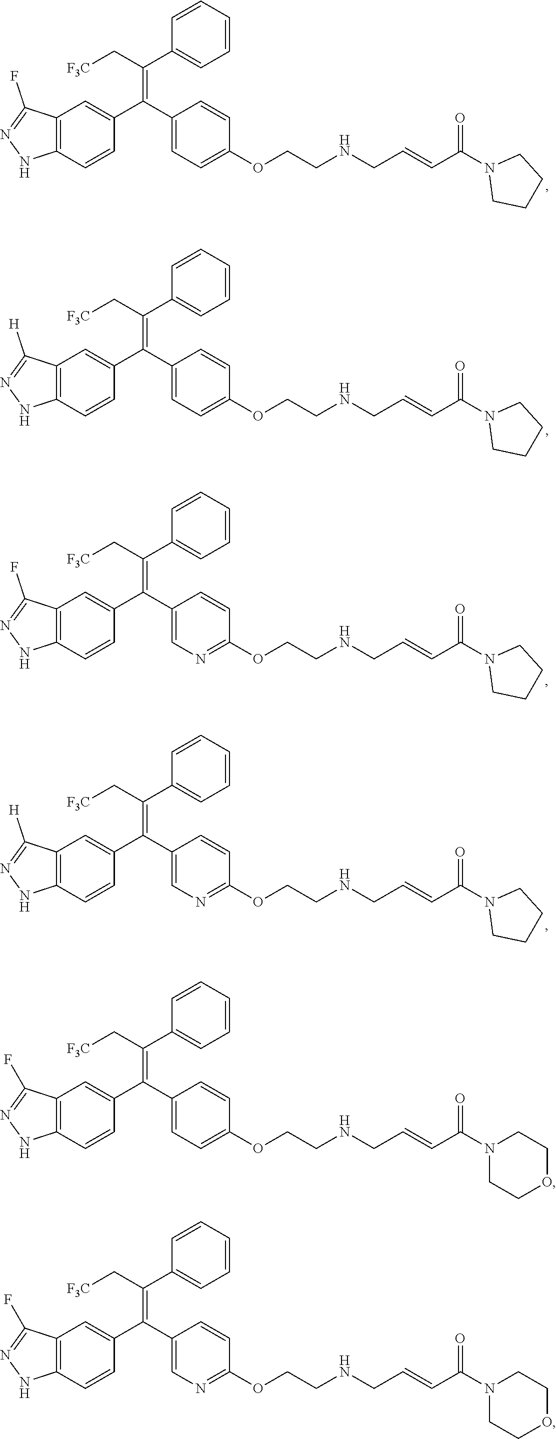 [SMILES: O=C(/C=C/CNCCOc1ccc(/C(=C(/CC(F)(F)F)c2ccccc2)c2ccc3[nH]nc(F)c3c2)cc1)N1CCCC1 ; O=C(/C=C/CNCCOc1ccc(/C(=C(/CC(F)(F)F)c2ccccc2)c2ccc3[nH]ncc3c2)cc1)N1CCCC1 ; O=C(/C=C/CNCCOc1ccc(/C(=C(/CC(F)(F)F)c2ccccc2)c2ccc3[nH]nc(F)c3c2)cn1)N1CCCC1 ; O=C(/C=C/CNCCOc1ccc(/C(=C(/CC(F)(F)F)c2ccccc2)c2ccc3[nH]ncc3c2)cn1)N1CCCC1 ; O=C(/C=C/CNCCOc1ccc(/C(=C(/CC(F)(F)F)c2ccccc2)c2ccc3[nH]nc(F)c3c2)cc1)N1CCOCC1 ; O=C(/C=C/CNCCOc1ccc(/C(=C(/CC(F)(F)F)c2ccccc2)c2ccc3[nH]nc(F)c3c2)cn1)N1CCOCC1] 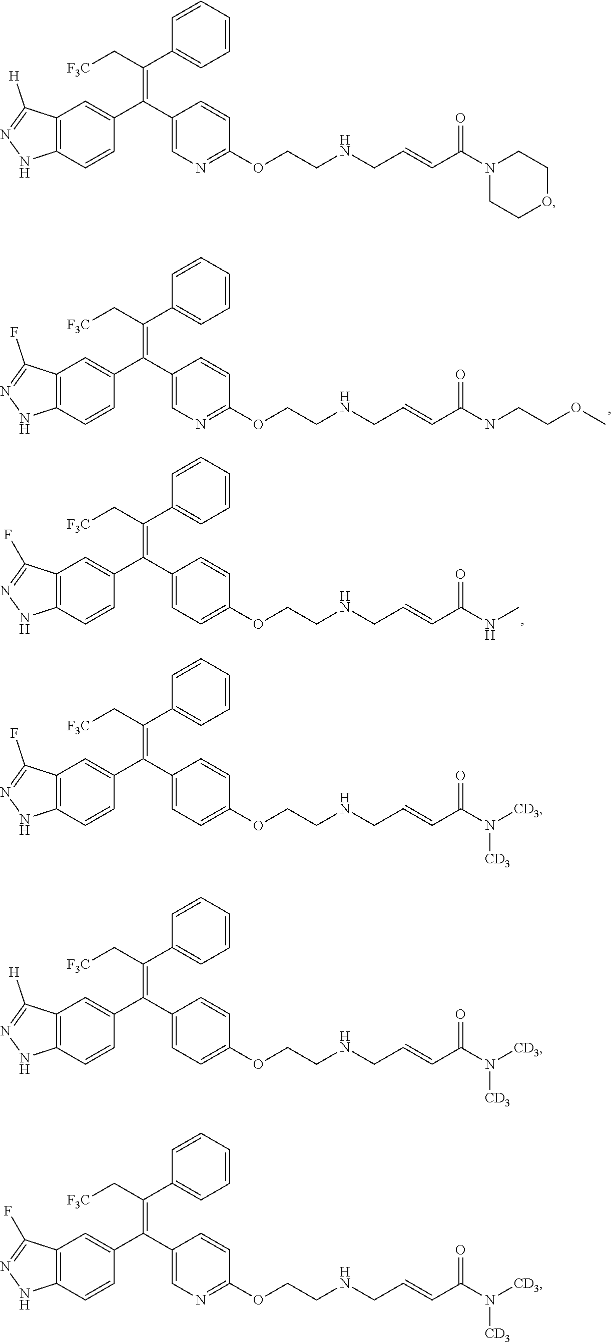 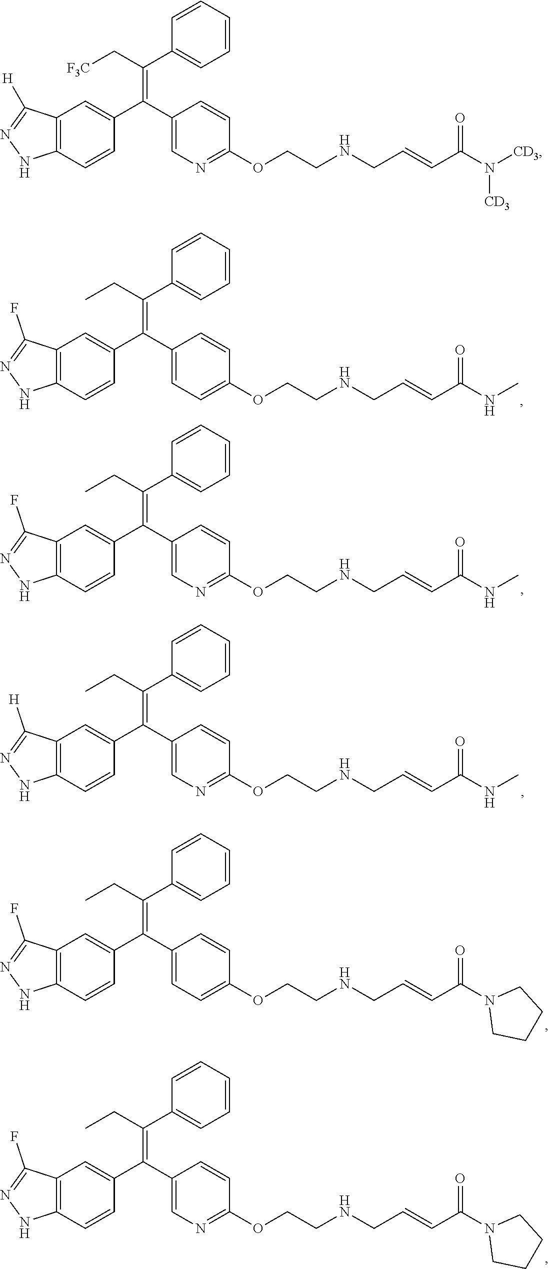 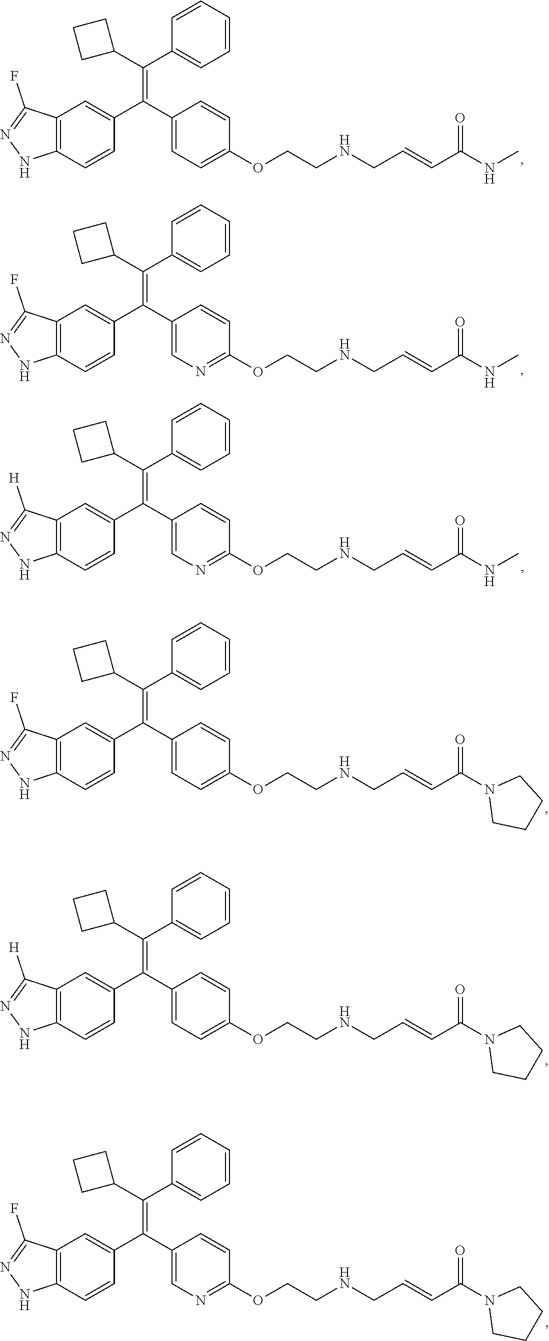 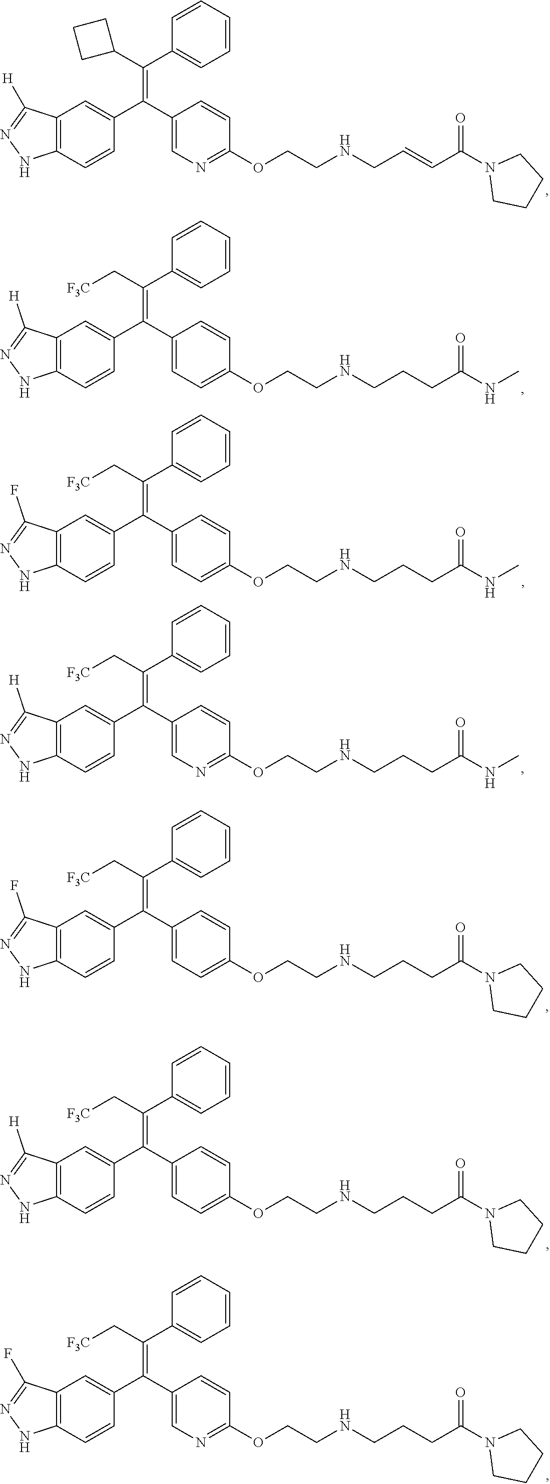 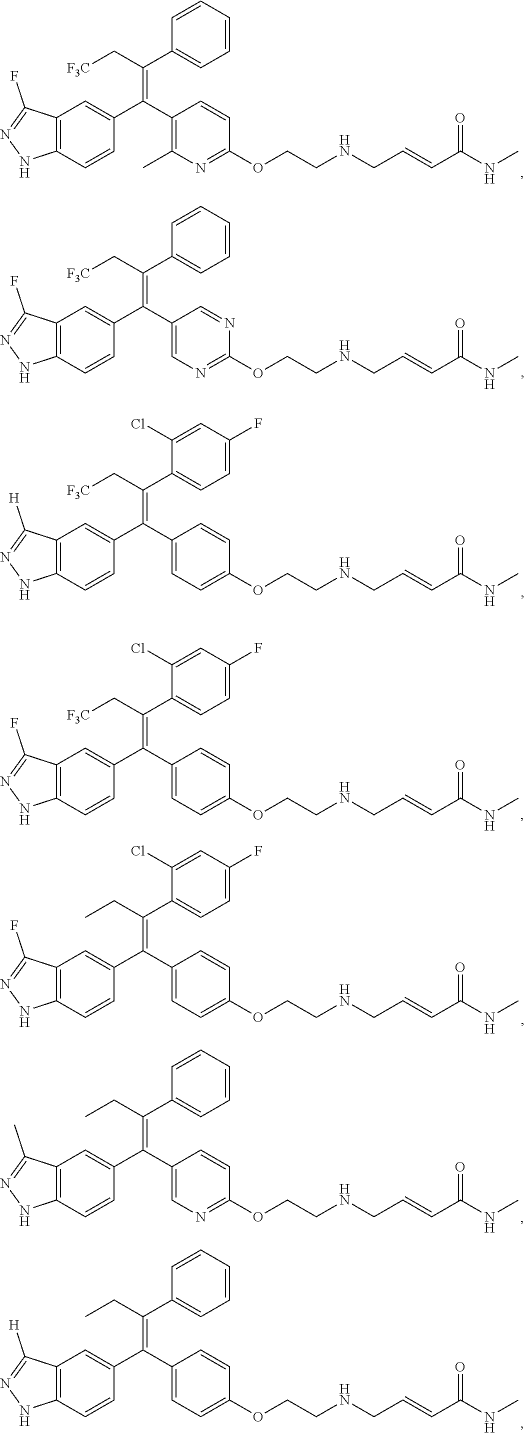 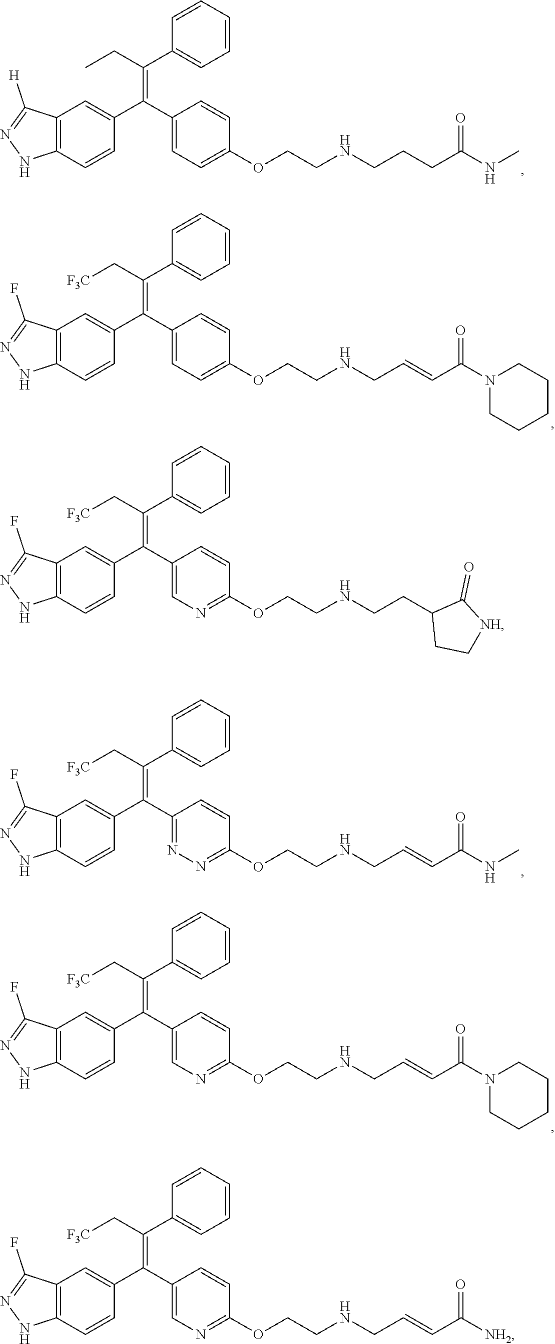 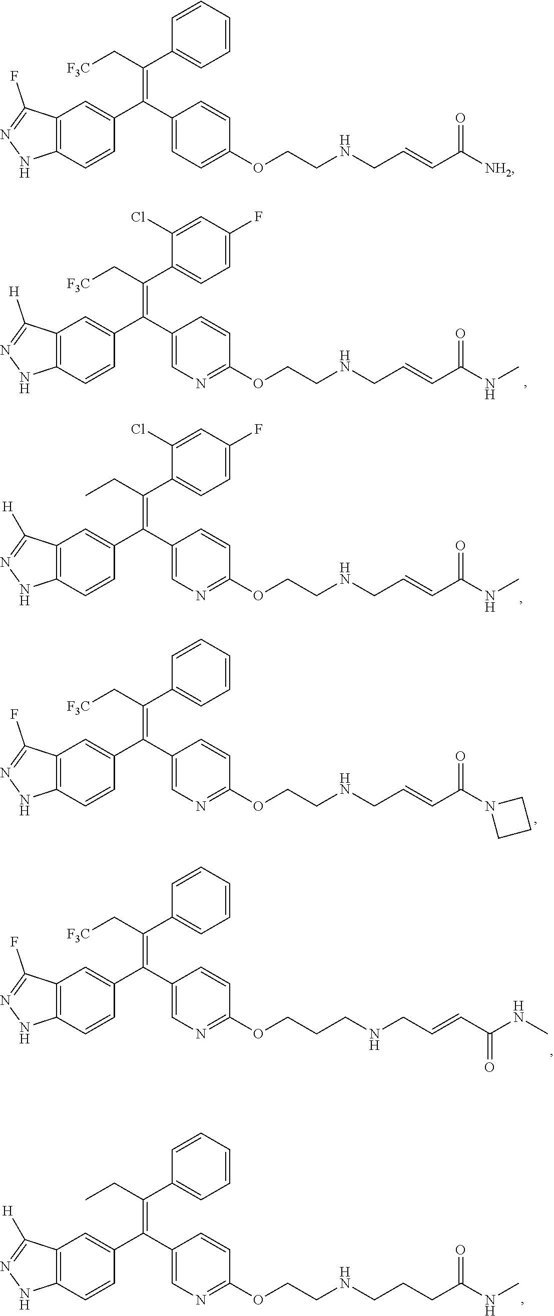 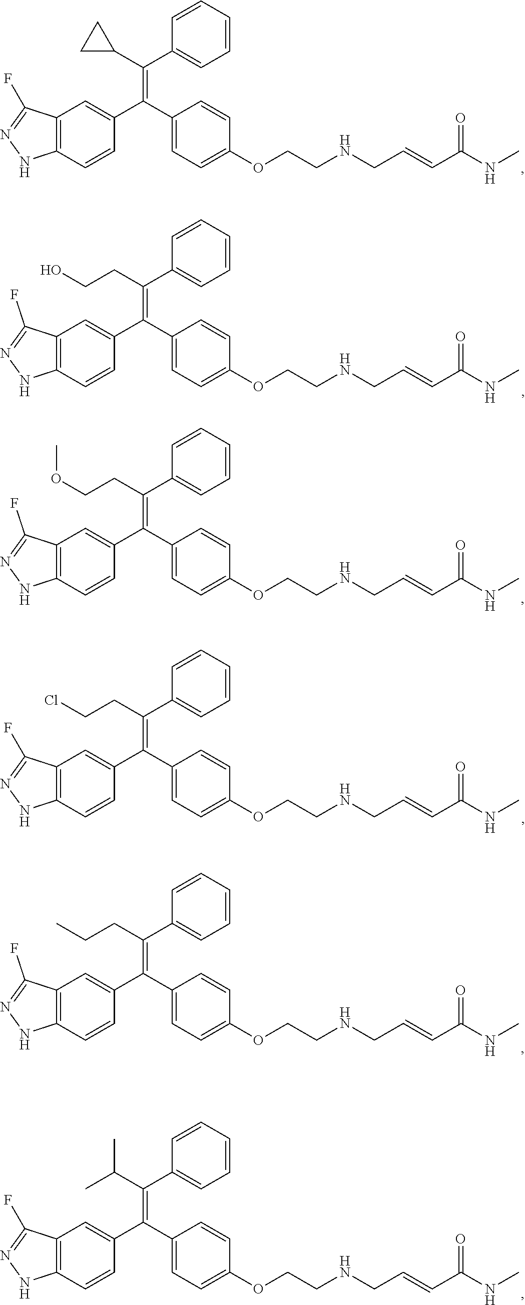 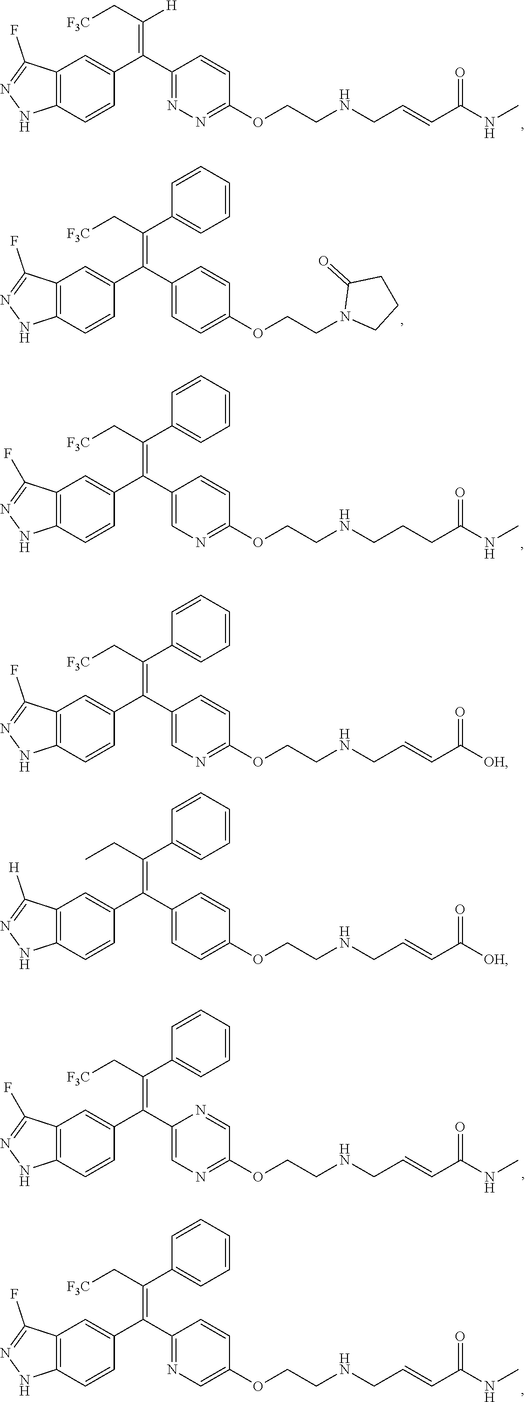 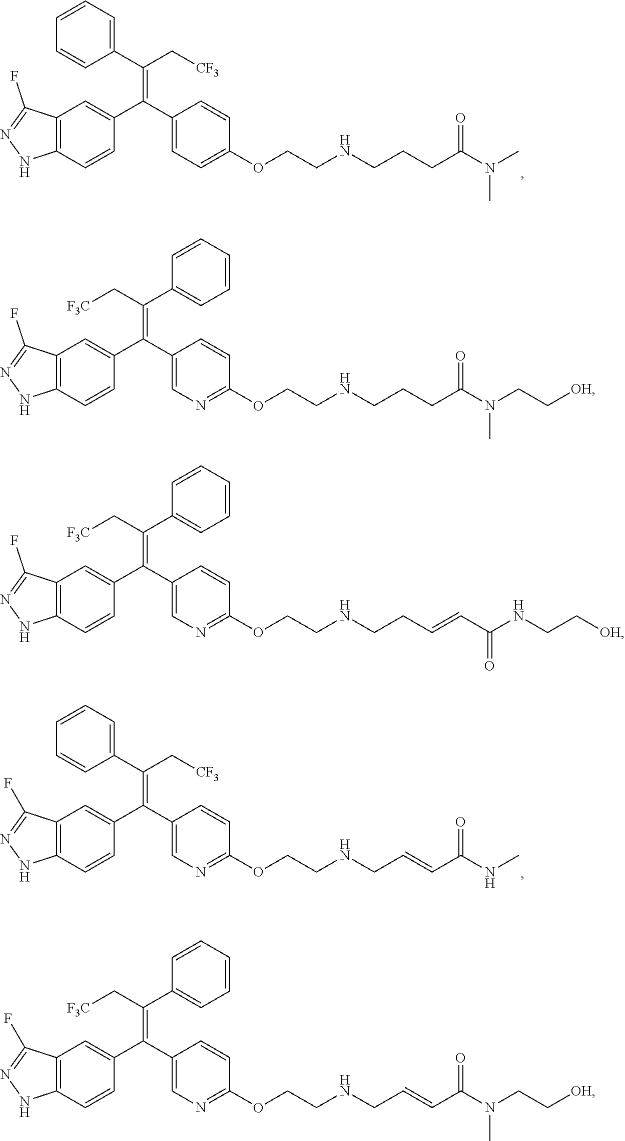 [SMILES: CN(C)C(=O)CCCNCCOc1ccc(/C(=C(\CC(F)(F)F)c2ccccc2)c2ccc3[nH]nc(F)c3c2)cc1 ; CN(CCO)C(=O)CCCNCCOc1ccc(/C(=C(/CC(F)(F)F)c2ccccc2)c2ccc3[nH]nc(F)c3c2)cn1 ; O=C(/C=C/CCNCCOc1ccc(/C(=C(/CC(F)(F)F)c2ccccc2)c2ccc3[nH]nc(F)c3c2)cn1)NCCO ; CNC(=O)/C=C/CNCCOc1ccc(/C(=C(\CC(F)(F)F)c2ccccc2)c2ccc3[nH]nc(F)c3c2)cn1 ; CN(CCO)C(=O)/C=C/CNCCOc1ccc(/C(=C(/CC(F)(F)F)c2ccccc2)c2ccc3[nH]nc(F)c3c2)cn1] 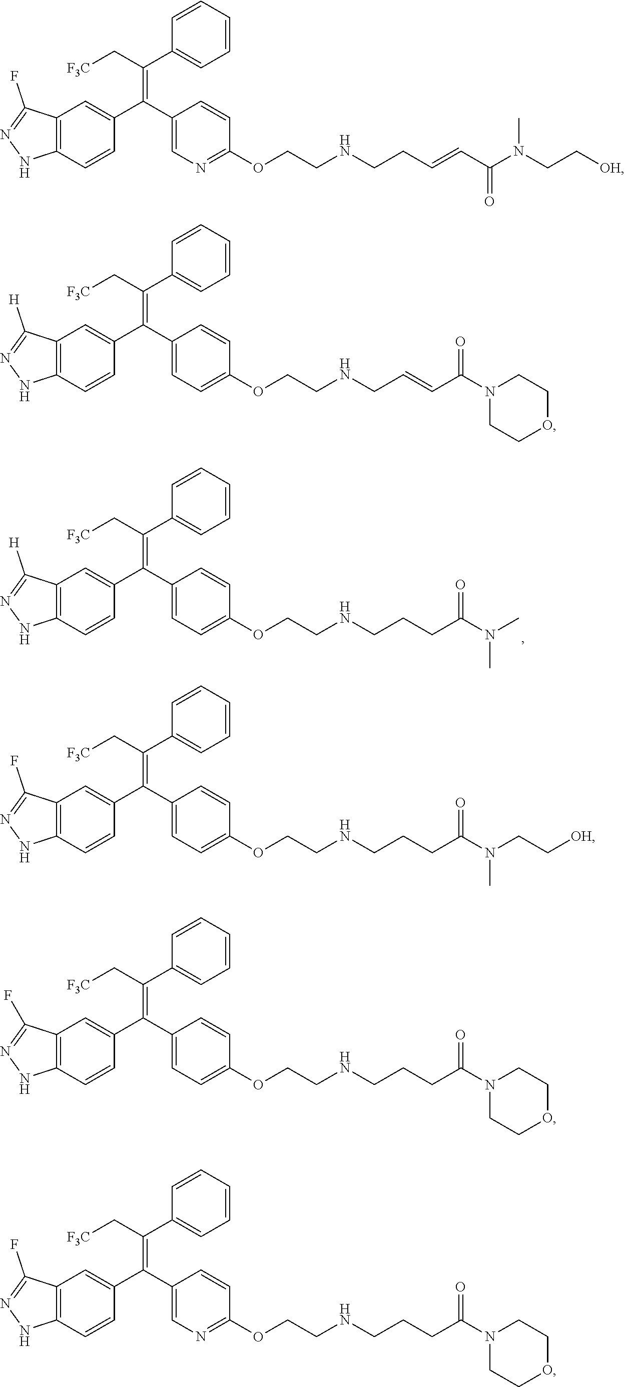 [SMILES: CN(CCO)C(=O)/C=C/CCNCCOc1ccc(/C(=C(/CC(F)(F)F)c2ccccc2)c2ccc3[nH]nc(F)c3c2)cn1 ; O=C(/C=C/CNCCOc1ccc(/C(=C(/CC(F)(F)F)c2ccccc2)c2ccc3[nH]ncc3c2)cc1)N1CCOCC1 ; CN(C)C(=O)CCCNCCOc1ccc(/C(=C(/CC(F)(F)F)c2ccccc2)c2ccc3[nH]ncc3c2)cc1 ; CN(CCO)C(=O)CCCNCCOc1ccc(/C(=C(/CC(F)(F)F)c2ccccc2)c2ccc3[nH]nc(F)c3c2)cc1 ; O=C(CCCNCCOc1ccc(/C(=C(/CC(F)(F)F)c2ccccc2)c2ccc3[nH]nc(F)c3c2)cc1)N1CCOCC1 ; O=C(CCCNCCOc1ccc(/C(=C(/CC(F)(F)F)c2ccccc2)c2ccc3[nH]nc(F)c3c2)cn1)N1CCOCC1] 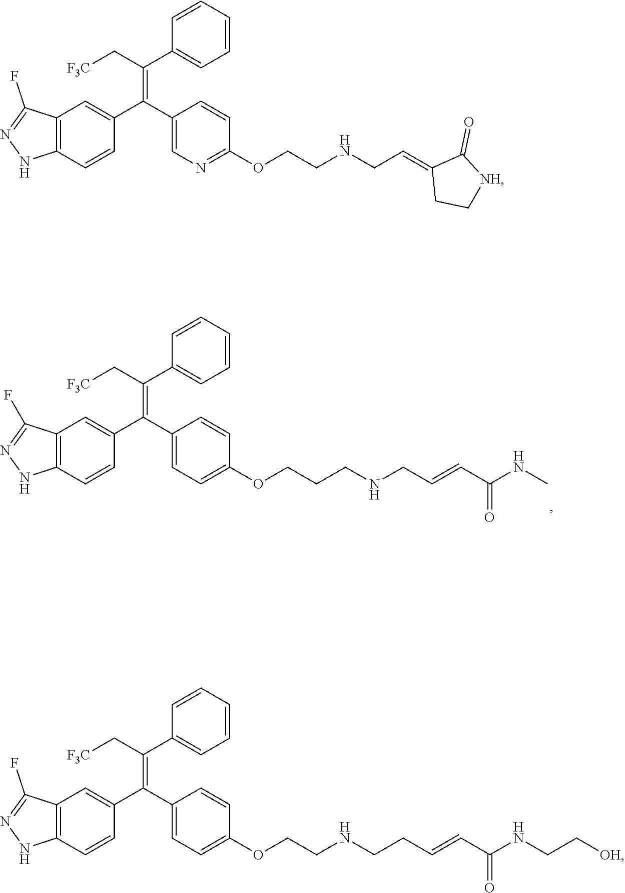 [SMILES: O=C1NCC/C1=C\CNCCOc1ccc(/C(=C(/CC(F)(F)F)c2ccccc2)c2ccc3[nH]nc(F)c3c2)cn1 ; CNC(=O)/C=C/CNCCCOc1ccc(/C(=C(/CC(F)(F)F)c2ccccc2)c2ccc3[nH]nc(F)c3c2)cc1 ; O=C(/C=C/CCNCCOc1ccc(/C(=C(/CC(F)(F)F)c2ccccc2)c2ccc3[nH]nc(F)c3c2)cc1)NCCO]
or a pharmaceutically acceptable salt thereof.

16-19. (canceled)

20. The compound of claim 3, having the following formula: 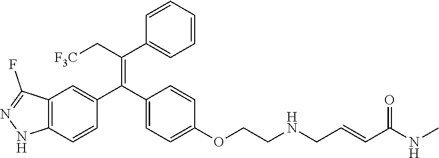 [SMILES: CNC(=O)/C=C/CNCCOc1ccc(/C(=C(/CC(F)(F)F)c2ccccc2)c2ccc3[nH]nc(F)c3c2)cc1]
or a pharmaceutically acceptable salt thereof.

21. The compound of claim 3, having the following formula:

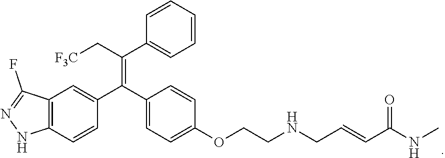 [SMILES: CNC(=O)/C=C/CNCCOc1ccc(/C(=C(/CC(F)(F)F)c2ccccc2)c2ccc3[nH]nc(F)c3c2)cc1]

22. A pharmaceutical composition comprising a compound or pharmaceutically acceptable salt of claim 20 and a pharmaceutically acceptable excipient.

23. A method of treating breast cancer comprising administering to a subject a pharmaceutical composition according to claim 22.

24. The method according to claim 23, wherein said breast cancer is an ER-positive breast cancer.

25. The method according to claim 24, where said subject expresses a mutant ER-α protein.

26-31. (canceled)

32. A pharmaceutical composition comprising a compound or pharmaceutically acceptable salt of claim 1 and a pharmaceutically acceptable excipient.

33. A method of treating breast cancer comprising administering to a subject a pharmaceutical composition according to claim 32.

34. The method according to claim 33, wherein said breast cancer is an ER-positive breast cancer.

35. The method according to claim 34, where said subject expresses a mutant ER-α protein.
